SEQUENCE LISTING (112 sequences)

SEQ ID NO: 1 - DNA, 35 bp, Artificial Sequence
  Description of Artificial Sequence Synthetic oligonucleotide
ttctccatacctgtttttctggatggagtaagacc

SEQ ID NO: 2 - DNA, 35 bp, Artificial Sequence
  Description of Artificial Sequence Synthetic oligonucleotide
actagtggggaagcttatcgatgataagctgtcaa

SEQ ID NO: 3 - DNA, 20 bp, Artificial Sequence
  Description of Artificial Sequence Synthetic oligonucleotide
gacgtgtgctcttccgatct

SEQ ID NO: 4 - DNA, 14 bp, Artificial Sequence
  Description of Artificial Sequence Synthetic oligonucleotide
  modified_base (2)..(7): a, c, t, g, unknown or other
  modified_base (9)..(14): a, c, t, g, unknown or other
cnnnnnngnnnnnn

SEQ ID NO: 5 - DNA, 21 bp, Artificial Sequence
  Description of Artificial Sequence Synthetic oligonucleotide
gatcgcctagacaactcctga

SEQ ID NO: 6 - DNA, 146 bp, Artificial Sequence
  Description of Artificial Sequence Synthetic polynucleotide
  modified_base (30)..(35): a, c, t, g, unknown or other
  modified_base (70)..(75): a, c, t, g, unknown or other
  modified_base (77)..(82): a, c, t, g, unknown or other
  modified_base (117)..(122): a, c, t, g, unknown or other
aatgatacggcgaccaccgagatctacacnnnnnnacactctttccctcaggagttgtctaggcgatccnnnnnngnnnnnnagatcggaagagcacacgtctgaactccagtcacnnnnnnatctcgtatgccgtcttctgcttg

SEQ ID NO: 7 - DNA, 33 bp, Artificial Sequence
  Description of Artificial Sequence Synthetic primer
acactctttccctcaggagttgtctaggcgatc

SEQ ID NO: 8 - DNA, 89 bp, Artificial Sequence
  Description of Artificial Sequence Synthetic oligonucleotide
agcaagcacgccttagtaacccggaattgcgtaagtctgccgccgatcgtgatgctgcctttgaaaaaattaatgaagcgcgcagtcca

SEQ ID NO: 9 - DNA, 89 bp, Artificial Sequence
  Description of Artificial Sequence Synthetic oligonucleotide
tggactgcgcgcttcattaattttttcaaaggcagcatcacgatcggcggcagacttacgcaattccgggttactaaggcgtgcttgct

SEQ ID NO: 10 - DNA, 20 bp, Artificial Sequence
  Description of Artificial Sequence Synthetic oligonucleotide
aaaataaggaggaaaaacat

SEQ ID NO: 11 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 12 - protein, 178 aa, Escherichia coli; identical to SEQ ID NO: 11

SEQ ID NO: 13 - protein, 178 aa, Escherichia coli; identical to SEQ ID NO: 11

SEQ ID NO: 14 - protein, 178 aa, Unknown; identical to SEQ ID NO: 11
  Description of Unknown Proteobacteria sequence

SEQ ID NO: 15 - protein, 178 aa, Escherichia sp.
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNVGGGQPQGGWGQPQQPQGGNQFSGGAQSRPQQSAPAAPSNEPPMDFDDDIPF

SEQ ID NO: 16 - protein, 178 aa, Shigella flexneri
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNKFSGGAQSRPQQSAPAAPSNEPPMDFDDDIPF

SEQ ID NO: 17 - protein, 178 aa, Escherichia coli
MASKGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 18 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYLEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 19 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSAQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 20 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAAGNIGGGQPQGGWGQPQQPQGGNQFSGGAQSRPQQSAPAAPSNEPPMDFDDDIPF

SEQ ID NO: 21 - protein, 178 aa, Escherichia coli
MASRGVNKVILVGNLGHDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 22 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQSGWGQPQQPQGGNQFSGGAQSRPQQSAPAAPSNEPPMDFDDDIPF

SEQ ID NO: 23 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGSWGQPQQPQGGNQFSGGAQSRPQQSAPAAPSNEPPMDFDDDIPF

SEQ ID NO: 24 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGSAQSRPQQSAPAAPSNEPPMDFDDDIPF

SEQ ID NO: 25 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGSNQFSGGAQSRPQQSAPAAPSNEPPMDFDDDIPF

SEQ ID NO: 26 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGSNIGGGQPQGGWGQPQQPQGGNQFSGGAQSRPQQSAPAAPSNEPPMDFDDDIPF

SEQ ID NO: 27 - protein, 178 aa, Escherichia coli
MASRGVNKVILVGNLGQDPEVRYMPNSGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 28 - protein, 178 aa, Escherichia sp.
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQSGGAPAGGNIGGGQPQGGWGQPQQPQGGNQFSGGAQSRPQQSAPAAPSNEPPMDFDDDIPF

SEQ ID NO: 29 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSTPAAPSNEPPMDFDDDIPF

SEQ ID NO: 30 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSCGAQSRPQQSAPAAPSNEPPMDFDDDIPF

SEQ ID NO: 31 - protein, 178 aa, Escherichia coli
  MOD_RES (111)..(111): Any amino acid
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMXMLGGRQGGGAPAGGNIGGGQPQGGWGQPQQPQGGNQFSGGAQSRPQQSAPAAPSNEPPMDFDDDIPF

SEQ ID NO: 32 - protein, 178 aa, Escherichia coli
MASRGVNKVILVGNLGQDPEVRYMPNGGAVANITLATSESWRDKATGEMKEQTEWHRVVLFGKLAEVVSEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 33 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGVQSRPQQSAPAAPSNEPPMDFDDDIPF

SEQ ID NO: 34 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGDAPAGGNIGGGQPQGGWGQPQQPQGGNQFSGGAQSRPQQSAPAAPSNEPPMDFDDDIPF

SEQ ID NO: 35 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQDGNQFSGGAQSRPQQSAPAAPSNEPPMDFDDDIPF

SEQ ID NO: 36 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYITEVVVNVGGTMQMLGGRQGGGAPAGGNIGGGQPQGGWGQPQQPQGGNQFSGGAQSRPQQSAPAAPSNEPPMDFDDDIPF

SEQ ID NO: 37 - protein, 178 aa, Shigella dysenteriae
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQLQGGWGQPQQPQGGNQFSGGAQSRPQQSAPAAPSNEPPMDFDDDIPF

SEQ ID NO: 38 - protein, 178 aa, Escherichia albertii
MASRGVNKVILVGNLGQDPEVRYMPNGGAVANITLATSESWRDKATGEMKEQTEWHRVVLFGKLAEVASEYLRKGSQVYIEGQLRTRKWTDQSGLDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 39 - protein, 178 aa, Escherichia coli
MASRGVNKVILVGNLGQDPEVRYMPNGGAVANITLATSESWRDKATGEMKEQTEWHRVVLFGKLAEVASEYLCKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 40 - protein, 178 aa, Escherichia coli
MASRGVNKVILVGNLGLDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 41 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQPAPAAPSNEPPMDFDDDIPF

SEQ ID NO: 42 - protein, 178 aa, Unknown; identical to SEQ ID NO: 37
  Description of Unknown Enterobacteriaceae sequence

SEQ ID NO: 43 - protein, 178 aa, Escherichia coli
MASRGVNKVILVGNLGQDPEVRYMPNGGAVANITLATSESWRDKATGEMKDQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSTPAAPSNEPPMDFDDDIPF

SEQ ID NO: 44 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRLQQSAPAAPSNEPPMDFDDDIPF

SEQ ID NO: 45 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQLQGGNQFSGGAQSRPQQSAPAAPSNEPPMDFDDDIPF

SEQ ID NO: 46 - protein, 178 aa, Escherichia sp.
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQSGGAPTGGNIGGGQPQGGWGQPQQPQGGNQFSGGAQSRPQQSAPAAPSNEPPMDFDDDIPF

SEQ ID NO: 47 - protein, 178 aa, Escherichia coli
MASRGVNKVILVGNLGQDPEVRYMPNGGAVANITLATSESWRDKATGEMKEQTEWHRVVLFGKLAEVASEYLRKGSQGYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 48 - protein, 178 aa, Escherichia coli
MASRGVNKVILVGNLGQDPEVRYMPNGGAVANITLATSESWRDKATGEMKEQTEWHRVVLFGKLAEGASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 49 - protein, 178 aa, Escherichia albertii
MASRGVNKVILVGNLGQDPEVRYMPNGGAVANITLATSESWRDKATGEMKEQTEWHRVVLFGKLAEVASEYLRKGSLVYIEGQLRTRKWTDQSGQDRYTTEVVVNVGGTMQMLGGRQSGGAPAGGNIGGGQPQGGWGQPQQPQGGNQFSGGAQSRPQQSAPAAPSNEPPMDFDDDIPF

SEQ ID NO: 50 - protein, 178 aa, Escherichia albertii
MASRGVNKVILVGNLGQDPEVRYMPNGGAVANITLATSEFWRDKATGEMKEQTEWHRVVLFGKLAEVASEYLRKGSQVYIEGQLRTRKWTDQSGQDRYTTEVVVNVGGTMQMLGGRQSGGAPAGGNIGGGQPQGGWGQPQQPQGGNQFSGGAQSRPQQSAPAAPSNEPPMDFDDDIPF

SEQ ID NO: 51 - protein, 177 aa, Citrobacter sp.
MASRGVNKVILVGNLGQDPEVRYMPNGGAVANITLATSESWRDKATGEMKEQTEWHRVVLFGKLAEVASEYLRKGSQVYIEGQLRTRKWTDQSGVEKYTTEVVVNVGGTMQMLGGRQGGGAPAGGNAGGGQQGGWGQPQQPQGGNQFSGGAQSRPQQSAPAAPSNEPPMDFDDDIPF

SEQ ID NO: 52 - protein, 178 aa, Citrobacter koseri
MASRGVNKVILVGNLGQDPEVRYMPNGGAVANITLATSESWRDKQTGEMKEQTEWHRVVLFGKLAEVASEYLRKGSQVYIEGQLRTRKWTDQSGQDKYTTEVVVNVGGTMQMLGGRQGGGAPAGGNMGGGQQQGGWGQPQQPQGGNQFSGGAQSRPQQQSAPAPSNEPPMDFDDDIPF

SEQ ID NO: 53 - protein, 171 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMD

SEQ ID NO: 54 - protein, 178 aa, Citrobacter koseri
MASRGVNKVILVGNLGQDPEVRYMPNGGAVANITLATSESWRDKQTGEMKEQTEWHRVVLFGKLAEVASEYLRKGSQVYIEGQLRTRKWTDQSGQDKYTTEVVVNVGGTMQMLGGRQGGGVPAGGNMGGGQQQGGWGQPQQPQGGNQFSGGAQSRPQQQSAPAPSNEPPMDFDDDIPF

SEQ ID NO: 55 - protein, 178 aa, Citrobacter koseri
MASRGVNKVILVGNLGQDPEVRYMPNGGAVANITLATSESWRDKQTGEMKEQTEWHRVVLFGKLAEVASEYLRKGSQVYIEGQLRTRKWTDQSGQDKYITEVVVNVGGTMQMLGGRQGGGAPAGGNMGGGQQQGGWGQPQQPQGGNQFSGGAQSRPQQQSAPAPSNEPPMDFDDDIPF

SEQ ID NO: 56 - protein, 171 aa, Shigella sp.
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQQPQGGNQFSGGAQSRPQQSAPAAPSNEPPMDFDDDIPF

SEQ ID NO: 57 - protein, 174 aa, Unknown
  Description of Unknown Enterobacteriaceae sequence
MASRGVNKVILVGNLGQDPEVRYMPNGGAVANITLATSESWRDKATGEMKEQTEWHRVVLFGKLAEVASEYLRKGSQVYIEGQLRTRKWTDQSGVEKYTTEVVVNVGGTMQMLGGRQGGGAPAGGGQQQGGWGQPQQPQGGNQFSGGAQSRPQQSAPAAPSNEPPMDFDDDIPF

SEQ ID NO: 58 - protein, 174 aa, Citrobacter freundii
MASRGVNKVILVGNLGQDPEVRYMPNGGAVANITLATSESWRDKATGEMKEQTEWHRVVLFGKLAEVASEYLRKGSQVYIEGQLRTRKWTDQSGVEKYTTEVVVNVGGTMQMLGGRQGGGAPAGGGQQQGGWGQPQQPQGGNQFSGGGQSRPQQSAPAAPSNEPPMDFDDDIPF

SEQ ID NO: 59 - protein, 174 aa, Citrobacter sp.
MASRGVNKVILVGNLGQDPEVRYMPNGGAVANITLATSESWRDKATGEMKEQTEWHRVVLFGKLAEVASEYLRKGSQVYIEGQLRTRKWTDQSGVEKYTTEVVVNVGGTMQMLGGRQGGGAPAGGGQQQGGWGQPQQPQGGNQFSGGEQSRPQQSAPAAPSNEPPMDFDDDIPF

SEQ ID NO: 60 - protein, 174 aa, Citrobacter youngae
MASRGVNKVILVGNLGQDPEVRYMPNGGAVANITLATSESWRDKATGEMKEQTEWHRVVLFGKLAEVAGEYLRKGSQVYIEGQLRTRKWTDQSGVEKYTTEVVVNVGGTMQMLGGRQGGGAPAGGGQQQGGWGQPQQPQGGNQFSGGAQSRPQQSAPAAPSNEPPMDFDDDIPF

SEQ ID NO: 61 - protein, 174 aa, Citrobacter werkmanii
MASRGVNKVILVGNLGQDPEVRYMPNGGAVANITLATSESWRDKATGEMKEQTEWHRVVLFGKLAEVASEYLRKGSQVYIEGQLRTRKWTDQSGVEKYTTEVVVNVGGTMQMLGGRQGGGAPAGGGQQQGGWGQPQQPQGGNQFSGGAQSRPQQSAPAAPSNEPSMDFDDDIPF

SEQ ID NO: 62 - protein, 174 aa, Citrobacter sp.
MASRGVNKVILVGNLGQDPEVRYMPNGGAVANITLATSESWRDKATGEMKEQTEWHRVVLFGKLAEVASEYLRKGSQVYIEGQLRTRKWTDQSGVEKYTTEVVVNVGGTMQMLGGRQGGGAPAGGGQQQGGWGQPQQPQGGNQFSGGAQSRLQQSAPAAPSNEPPMDFDDDIPF

SEQ ID NO: 63 - protein, 175 aa, Unknown
  Description of Unknown Enterobacteriaceae sequence
MASRGVNKVILVGNLGQDPEVRYMPNGGAVANITLATSESWRDKATGEMKEQTEWHRVVLFGKLAEVASEYLRKGSQVYIEGQLRTRKWTDQSGVEKYTTEVVVNVGGTMQMLGGRQGGGAPAGGGQQQGGWGQPQQPQGGNQFSGGAQSRPQQQSAPAAPSNEPPMDFDDDIPF

SEQ ID NO: 64 - protein, 174 aa, Citrobacter sp.
MASRGVNKVILVGNLGQDPEVRYMPNGGAVANITLATSESWRDKQTGEMKEQTEWHRVVLFGKLAEVASEYLRKGSQVYIEGQLRTRKWTDQSGQDKYTTEVVVNVGGTMQMLGGRQGGGAPAGGQQQQGGWGQPQQPQGGNQFSGGAQSRPQQQSAPAPSNEPPMDFDDDIPF

SEQ ID NO: 65 - protein, 174 aa, Unknown
  Description of Unknown Proteobacteria sequence
MASRGVNKVILVGNLGQDPEVRYMPSGGAVANITLATSESWRDKATGEMKEQTEWHRVVLFGKLAEVAGEYLRKGSQVYIEGQLRTRKWTDQSGQEKYTTEVVVNVGGTMQMLGGRQGGGAPAGGGQQQGGWGQPQQPQGGNQFSGGAQSRPQQQSAPAPSNEPPMDFDDDIPF

SEQ ID NO: 66 - protein, 155 aa, Escherichia coli
MPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 67 - protein, 174 aa, Enterobacter aerogenes
MASRGVNKVILVGNLGQDPEVRYMPSGGAVANFTLATSESWRDKQTGEMKEQTEWHRVVLFGKLAEVAGEYLRKGSQVYIEGQLRTRKWTDQSGQDKYTTEIVVNVGGTMQMLGGRQGGGAPASGGQQQGGWGQPQQPQGGNQFSGGAQSRPQQQAPAAPSNEPPMDFDDDIPF

SEQ ID NO: 68 - protein, 176 aa, Enterobacter cloacae
MASKGVNKVILVGNLGQDPEVRYLPSGGAVCSVTLATSESWRDKATGELKEQTEWHRVVLFGKLAEVAGEYLRKGSQVYIEGQLRTRKWTDQSGQEKYTTEVVVNVGGTMQMLGGRQGGGAPTGGSQNQQQGGWGRHQQPQGGNQFSGGAQSRPQQQSAPAPSNEPPMDLDDDIPF

SEQ ID NO: 69 - protein, 174 aa, Enterobacter cloacae
MASRGVNKVILVGNLGQDPEVRYMPSGGAVANITLATSESWRDKATGEMKEQTEWHRVVLFGKLAEVAGEYLRKGSQVYIEGQLRTRKWTDQSGAEKYTTEVVVNVGGTMQMLGGRQGGGAPAGGSQQQGGWGQPQQPQGGNQFSGGAQSRPQQQSAPAPSNEPPMDFDDDIPF

SEQ ID NO: 70 - protein, 178 aa, Klebsiella sp.
MASRGVNKVILVGNLGQDPEVRYMPSGGAVANITLATSESWRDKQTGEMKEQTEWHRVVLFGKLAEVAGEYLRKGSQVYIEGQLRTRKWTDQSGQEKYTTEVVVNVGGTMQMLGGRQGGGAPAGGNMGGGQQQGGWGQPQQPQGGNQFSGGAQSRPQQQSAPAPSNEPPMDFDDDIPF

SEQ ID NO: 71 - protein, 175 aa, Klebsiella oxytoca
MASRGVNKVILVGNLGQDPEVRYMPSGGAVANITLATSESWRDKATGEMKEQTEWHRVVLFGKLAEVAGEYLRKGSQVYIEGQLRTRKWTDQSGQEKYTTEVVVNVGGTMQMLGGRQQGASAPAGGGQQQGGWGQPQQPQGGNQFSGGAQSRPQQQAPAAPSNEPPMDFDDDIPF

SEQ ID NO: 72 - protein, 178 aa, Unknown
  Description of Unknown Enterobacteriaceae sequence
MASRGVNKVILVGNLGQDPEVRYMPSGGAVANITLATSESWRDKATGEMKEQTEWHRVVLFGKLAEVAGEYLRKGSQVYIEGQLRTRKWTDQSGAEKYTTEVVVNVGGTMQMLGGRQGGGAPAGGNMGGGQGQQGGWGQPQQPQGGNQFSGGAQSRPQQSAPAPSNEPPMDFDDDIPF

SEQ ID NO: 73 - protein, 178 aa, Enterobacter lignolyticus
MASRGVNKVILVGNLGQDPEVRYMPSGGAVANITLATSESWRDKQTGEMKEQTEWHRVVLFGKLAEVAGEYLRKGSQVYIEGQLRTRKWTDQSGQEKYTTEVVVNVGGTMQMLGGRQGGGASAGGNMGGGQQQGGWGQPQQPQGGNQFSGGAQSRPQQQSAPAPSNEPPMDFDDDIPF

SEQ ID NO: 74 - protein, 176 aa, Serratia marcescens
MASRGVNKVILVGNLGQDPEVRYMPNGGAVANITLATSESWRDKATGEQKEKTEWHRVVLFGKLAEVAGEYLRKGSQVYIEGSLQTRKWQDQSGQDRYTTEIVVNVGGTMQMLGGRQGGGAPAGQSAGGQSGWGQPQQPQGGNQFSGGQQQSRPAQNSAPATSNEPPMDFDDDIPF

SEQ ID NO: 75 - protein, 174 aa, Enterobacter cloacae
MASRGVNKVILVGNLGQDPEVRYMPSGGAVANITLATSESWRDKATGEMKEQTEWHRVVLFGKLAEVAGEYLRKGSQVYIEGQLRTRKWTDQSGQEKYTTEVVVNVGGTMQMLGGRQGSGAPAGGGQQQGGWGQPQQPQGGNQFSGGAQSRPQQQSAPAPSNEPPMDFDDDIPF

SEQ ID NO: 76 - protein, 176 aa, Enterobacter cloacae
MASKGVNKVILVGNLGQDPEVRYLPSGGAVCSVTLATSESWRDKATGELKEQTEWHRVVLFGKLAEVAGEYLRKGSQVYIEGQLRTRKWTDQSGQEKYTTEVVVNVGGTMQMLGGRQGGGAPAGGSQNQQQGGWGQPQQPQGGNQFSGGAQSRPQQQSAPAPSNEPPMDFDDDIPF

SEQ ID NO: 77 - protein, 175 aa, Unknown
  Description of Unknown Enterobacteriaceae sequence
MASRGVNKVILVGNLGQDPEVRYMPSGGAVANITLATSESWRDKATGEMKEQTEWHRVVLFGKLAEVAGEYLRKGSQVYIEGQLRTRKWTDQSGQEKYTTEVVVNVGGTMQMLGGRQQGAGAPAGGGQQQGGWGQPQQPQGGNQFSGGAQSRPQQQAPAAPSNEPPMDFDDDIPF

SEQ ID NO: 78 - protein, 175 aa, Unknown
  Description of Unknown Enterobacteriaceae sequence
MASRGVNKVILVGNLGQDPEVRYMPSGGAVANITLATSESWRDKATGEMKEQTEWHRVVLFGKLAEVAGEYLRKGSQVYIEGQLRTRKWTDQSGQEKYTTEIVVNVGGTMQMLGGRQQGAGAPAGGGQQQGGWGQPQQPQGGNQFSGGAQSRPQQQAPAAPSNEPPMDFDDDIPF

SEQ ID NO: 79 - protein, 176 aa, Enterobacter cloacae
MASKGVNKVILVGNLGQDPEVRYLPSGSAVCSVTLATSESWRDKATGELKEQTEWHRVVLFGKLAEVAGEYLRKGSQVYIEGQLRTRKWTDQSGQEKYTTEVVVNVGGTMQMLGGRQGGGAPAGGSQNQQQGGWGQPQQPQGGNQFSGGAQSRPQQQSAPAPSNEPPMDFDDDIPF

SEQ ID NO: 80 - protein, 175 aa, Klebsiella oxytoca
MASRGVNKVILVGNLGQDPEVRYMPSGGAVANITLATSESWRDKATGEMKEQTEWHRVVLFGKLAEVAGEYLRKGSQVYIEGQLRTRKWTDQSGQEKYTTEVVVNVGGTMQMLGGRQQGAGAPAGGGQQQGGWGQPQQPQGGNQYSGGAQSRPQQQAPAAPSNEPPMDFDDDIPF

SEQ ID NO: 81 - protein, 175 aa, Klebsiella oxytoca
MASRGVNKVILVGNLGQDPEVRYMPSGGAVANITLATSESWRDKATGEMKEQTEWHRVVLFGKLAEVAGEYLRKGSQVYIEGQLRTRKWTDQSGQEKYTTEVVVNVGGTMQMLGGRQQGAGAPAGGGQQQGGWGQPQQPQGGNQFSGGAQSRPQQQTPAAPSNEPPMDFDDDIPF

SEQ ID NO: 82 - protein, 184 aa, Pantoea sp.
MASRGVNKVILVGNLGQDPEVRYMPNGGAVANITLATSESWRDKQTGENKEITEWHRVVLFGKLAEVAGEYLRKGSQVYIEGQLRTRKWQDQGGQDRYTTEVVVNVGGTMQMLGGRQQGGASAGGAPMGGGQQSGGNNNGWGQPQQPQGGNQFSGGAQSRPQPQSAPASNNNEPPMDFDDDIPF

SEQ ID NO: 83 - protein, 175 aa, Klebsiella oxytoca
MASRGVNKVILVGNLGQDPEVRYMPSGGAVANITLATSESWRDKATGEMKEQTEWHRVVLFGKLAEVAGEYLRKGSQVYIEGQLRTRKWTDQSGQEKYTTEVVVNVGGTMQMLGGRQQGAGAPAGGGQQQGGWGQPQQPQGGNQFSGGAQSRPQQQAPAAPSNETPMDFDDDIPF

SEQ ID NO: 84 - protein, 176 aa, Unknown
  Description of Unknown Enterobacteriaceae sequence
MASRGVNKVILVGNLGQDPEVRYMPNGGAVANITLATSESWRDKATGEQKEKTEWHRVVLFGKLAEVAGEYLRKGSQVYIEGSLQTRKWQDQSGQDRYTTEIVVNVGGTMQMLGGRQGGGAPAGQSAGGQGGWGQPQQPQSGNQFSGGQQQSRPAQNSAPATSNEPPMDFDDDIPF

SEQ ID NO: 85 - protein, 174 aa, Klebsiella pneumoniae
MASRGVNKVILVGNLGQDPEVRYMPSGGAVANFTLATSESWRDKHTGEMKEQTEWHRVVLFGKLAEVAGEYLRKGSQVYIEGQLRTRKWTDQSGQDKYTTEVVVNVGGTMQMLGGRQGGGAPAGGGQQQGGWGQPQQPQGGNQFSGGAQSRPQQQAPAAPSNEPPMDFDDDIPF

SEQ ID NO: 86 - protein, 171 aa, Klebsiella pneumoniae
MASRGVNKVILVGNLGQDPEVRYMPSGGAVANFTLATSESWRDKQTGEMKEQTEWHRVVLFGKLAEVAGEYLRKGSQVYIEGQLRTRKWTDQSGQDKYTTEVVVNVGGTMQMLGGRQGGGAPAGGGQQQGGWGQPQGGNQFSGGAQSRPQQQAPAAPSNEPPMDFDDDIPF

SEQ ID NO: 87 - protein, 173 aa, Unknown
  Description of Unknown Enterobacteriaceae sequence
ASRGVNKVILVGNLGQDPEVRYMPSGGAVANFTLATSESWRDKQTGEMKEQTEWHRVVLFGKLAEVAGEYLRKGSQVYIEGQLRTRKWTDQSGQDKYTTEVVVNVGGTMQMLGGRQGGGAPAGGGQQQGGWGQPQQPQGGNQFSGGAQSRPQQQAPAAPSNEPPMDFDDDIPF

SEQ ID NO: 88 - protein, 174 aa, Klebsiella pneumoniae
MASRGVNKVILVGNLGQDPEVRYMPSGGAVANFTLATSESWRDKQTGEMKEQTEWHRVVLFGKLAEVAGEYLRKGSQVYIEGQLRTRKWTDQSGQDKYTTEVVVNVGGTMQMLGGRQGGGAPAGGGQQQGGWGQPQQPQGGNQFSGGAQSRPQQQAPAAPSNEPPMDFDDDIPF

SEQ ID NO: 89 - protein, 175 aa, Unknown
  Description of Unknown Gammaproteobacteria sequence
MASRGVNKVILVGNLGQDPEVRYMPNGGAVANITLATSESWRDKQTGEMKEQTEWHRVVLFGKLAEVASEYLRKGSQVYIEGQLRTRKWTDQSGVEKYTTEVVVNVGGTMQMLGGRQGGGAPAGGQQQQGGWGQPQQPQGGNQFSGGAQSRPQQQSAPAAPSNEPPMDFDDDIPF

SEQ ID NO: 90 - protein, 173 aa, Enterobacter aerogenes
MASRGVNKVILVGNLGQDPEVRYMPSGGAVANFTLATSESWRDKQTGEMKEQTEWHRVVLFGKLAEVAGEYLRKGSQVYIEGQLRTRKWTDQSGQDKYTTEIVVNVGGTMQMLGGRQGGGAPAGGQQQGGWGQPQQPQGGNQFSGGAQSRPQQQAPAAPSNEPPMDFDDDIPF

SEQ ID NO: 91 - protein, 174 aa, Enterobacter aerogenes
MASRGVNKVILVGNLGQDPEVRYMPSGGAVANFTLATSESWRDKQTGEMKEQTEWHRVVLFGKLAEVAGEYLRKGSQVYIEGQLRTRKWTDQSGQDKYTTEIVVNVGGTMQMLGGRQGGGAPAGGGQQQGGWGQPQQPQGGNQFSGGAQSRPQQQAPAAPSNEPPMDFDDDIPF

SEQ ID NO: 92 - protein, 177 aa, Serratia sp.
MASRGVNKVILVGNLGQDPEVRYMPNGGAVANITLATSESWRDKATGEQKEKTEWHRVVLFGKLAEVAGEYLRKGSQVYIEGSLQTRKWQDQSGQDRYTTEIVVNVGGTMQMLGGRQGGGAPAGQSAGGQGGWGQPQQPQGGNQFSGGQQQSRPAQNSAPAASSNEPPMDFDDDIPF

SEQ ID NO: 93 - protein, 174 aa, Yokenella regensburgei
MASRGVNKVILVGNLGQDPEVRYMPNGGAVANITLATSESWRDKATGEMKEQTEWHRVVLFGKLAEVAGEYLRKGSQVYIEGQLRTRKWTDQSGQEKYTTEIVVNVGGTMQMLGGRQQGGAPAGGGQQQGGWGQPQQPQGGNQFSGGAQSRPQQQSAPAPSNEPPMDFDDDIPF

SEQ ID NO: 94 - protein, 178 aa, Raoultella terrigena
MASRGVNKVILVGNLGQDPEVRYMPSGGAVANFTLATSESWRDKQTGEMKEQTEWHRVVLFGKLAEVAGEYLRKGSQVYIEGQLRTRKWTDQSGAEKYTTEIVVNVGGTMQMLGGRQGGGAPAGGGQQQGGWGQPQQPQQQPQGGNQFSGGAQSRPQQQAPAAPSNEPPMDFDDDIPF

SEQ ID NO: 95 - protein, 174 aa, Klebsiella pneumoniae
MASRGVNKVILVGNLGQDPEVRYMPSGGAVANFTLATSESWRDKQTGEMKEQTEWHRVVLFGKLAEVAGEYLRKGSQVYIEGQLRTRKWTDQSGQDKYTTEVVVNVGGTMQMLGGRQGGGAPAGGGQQQGGWGQPQQPQGGNQFSGGAQSRPQQQAPSAPSNEPPMDFDDDIPF

SEQ ID NO: 96 - protein, 181 aa, Yersinia sp.
MASRGVNKVILVGNLGQDPEVRYMPNGGAVANITLATSESWRDKATGEQKEKTEWHRVVLFGKLAEVAGEYLRKGSQVYIEGALQTRKWQDQSGQERYTTEVVVNVGGTMQMLGGRQGGGAPAGGSQQDGGAQGGWGQPQQPQGGNQFSGGQTSRPAQSAPAAQPQGGNEPPMDFDDDIPF

SEQ ID NO: 97 - protein, 174 aa, Klebsiella pneumoniae
MASRGVNKVILVGNLGQDPEVRYMPSGGAVANFTLATSESWRDKQTGEMKEQTEWHRVVLFGKLAEVAGEYLRKGSQVYIEGQLRTRKWTDQSGQDKYTTEVVVNVSGTMQMLGGRQGGGAPAGGGQQQGGWGQPQQPQGGNQFSGGAQSRPQQQAPAAPSNEPPMDFDDDIPF

SEQ ID NO: 98 - protein, 180 aa, Cronobacter condimenti
MASRGVNKVILVGNLGQDPEVRYMPNGGAVANLRLATSESWRDKQTGEMKEVTEWHSVVLYGKLAEVAGEYLRKGSQIYIEGQLRTRKWQDQSGQDRYSTEVVVNVGGTMQMLGGRQGGGAPAGGNMGGGQQQGGWGQPQQPQQQSGGAQFSGGAQSRPQQQAPAPSNEPPMDFDDDIPF

SEQ ID NO: 99 - protein, 174 aa, Klebsiella sp.
MASRGVNKVILVGNLGQDPEVRYMPSGGAVANFTLATSESWRDKQTGEMKEQTEWHRVVLFGKLAEVAGEYLRKGSQVYIEGQLRTRKWTDQSGQDKYTTEVVVNVGGTMQMLGGRQGGGAPAGGGQQQGGWGQPQQPQGGSQFSGGAQSRPQQQAPAAPSNEPPMDFDDDIPF

SEQ ID NO: 100 - protein, 174 aa, Klebsiella pneumoniae
MASRGVNKVILVGNLGQDPEVRYMPSGGAVANFTLATSESWRDKQTGEMKEQTEWHRVVLFGKLAEVAGEYLRKGSQVYIEGQLRTRKWTDQSGQDKYTTEVVVNVGGTMQMLGGRQGGGAPAGGGQQQGGWGQPQQPQGGNQFSGGAQSRPQQQAPAAPSNETPMDFDDDIPF

SEQ ID NO: 101 - protein, 176 aa, Trabulsiella guamensis
MASRGVNKVILVGNLGQDPEVRYMPNGGAVANITLATSESWRDKQTGEMKEQTEWHRVVLFGKLAEVASEYLRKGSQVYIEGQLRTRKWTDQSGVEKYTTEVVVNVGGTMQMLGGRQQGAGAPAGGGQQQQGGWGQPQQPQGGAQFSGGAQSRPQQQSAPAPSNEPPMDFDDDIPF

SEQ ID NO: 102 - protein, 176 aa, Enterobacter cloacae
MASKGVNKVILVGNLGQDPEVRYLPSGGAVCSVTLATSESWRDKATGELKEQTEWHRIVLFGKLAEVAGEYLRKGSQVYIEGQLRTRKWTDQSGQEKYTTEVVVNVGGTMQMLGGRQGGGAPAGGGQSQQHGGWGQYQHPQVGNQFSGGAQSRPQQQSAPAPSNEPPMDFDDDIPF

SEQ ID NO: 103 - protein, 177 aa, Trabulsiella odontotermitis
MASRGVNKVILVGNLGQDPEVRYMPNGGAVANITLATSESWRDKQTGEMKEQTEWHRVVLFGKLAEVASEYLRKGSQVYIEGQLRTRKWTDQSGVEKYTTEVVVNVGGTMQMLGGRQQGAGAPAGGGQPQQQGGWGQPQQPQGGAQFSGGAQSRPQQQSAPAPSNEPPMDFDDDIPF

SEQ ID NO: 104 - protein, 177 aa, Trabulsiella odontotermitis
MASRGVNKVILVGNLGQDPEVRYMPNGGAVANITLATSESWRDKQTGEMKEQTEWHRVVLFGKLAEVASEYLRKGSQVYIEGQLRTRKWTDQSGVEKYTTEVVVNVGGTMQMLGGRQQGAGAPAGGGQQQGGWGQPQQPQQQGGAQFSGGAQSRPQQQSAPAPSNEPPMDFDDDIPF

SEQ ID NO: 105 - protein, 174 aa, Kosakonia radicincitans
MASRGVNKVILVGNLGQDPEVRYMPSGGAVANITLATSESWRDKQTGEMKEQTEWHRVVLFGKLAEVAGEYLRKGSQVYIEGQLRTRKWTDQSGQEKYTTEVVVNVGGTMQMLGGRQGGGAPAGGGQQQGGWGQPQQPQGGNQFSGGAQSRPQQSSAPAPSNEPPMDFDDDIPF

SEQ ID NO: 106 - protein, 177 aa, Serratia marcescens
MASRGVNKVILVGNLGQDPEVRYMPNGGAVANITLATSESWRDKATGEQKEKTEWHRVVLFGKLAEVAGEYLRKGSQVYIEGSLQTRKWTDQAGVEKYTTEVVVNVGGTMQMLGGRQGGGAPAGQSAGGQGGWGQPQQPQGGNQFSGGQQQSRPAQNSAPAASSNEPPMDFDDDIPF

SEQ ID NO: 107 - protein, 175 aa, Kluyvera sp.
MASRGVNKVILVGNLGQDPEVRYMPNGGAVANITLATSESWRDKATGEMKEQTEWHRVVLFGKLAEVAGEYLRKGSQVYIEGQLRTRKWTDQSGAEKYTTEVVVNVGGTMQMLGGRQGGGAPAGGGQQQQGGWGQPQQPQGGNQFSGGAQSRPQQQSAPAPSNEPPMDFDDDIPF

SEQ ID NO: 108 - protein, 6 aa, Artificial Sequence
  Description of Artificial Sequence Synthetic 6xHis tag
HHHHHH

SEQ ID NO: 109 - protein, 8 aa, Artificial Sequence
  Description of Artificial Sequence Synthetic peptide
DFDDDIPF

SEQ ID NO: 110 - protein, 178 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQSQQPQGGNQFSGGAQSRPQQSAPAAPSNEPPMDFDDDIPF

SEQ ID NO: 111 - protein, 188 aa, Escherichia coli
MASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGWGQPQQPQGGNQFSGGAQSRPQQSAPAAPSNEPPMDFDDDIPF

SEQ ID NO: 112 - protein, 175 aa, Enterobacter asburiae
MASRGVNKVILVGNLGQDPEVRYMPSGGAVANITLATSESWRDKATGEMKEQTEWHRVVLFGKLAEVAGEYLRKGSQVYIEGQLRTRKWTDQSGAEKYTTEVVVNVGGTMQMLGGRQGGGTPAGGGQQQQGGWGQPQQPQGGNQFSGGAQSRPQQQSAPAPSNEPPMDFDDDIPF

(19) United States
(12) Patent Application Publication
(10) Pub. No.: US 2021/0332350 A1
(43) Pub. Date: Oct. 28, 2021

(54) Recombinase Genome Editing

(71) Applicant: President and Fellows of Harvard College

(72) Inventors: George M. Church, Brookline, MA (US); Christopher J. Gregg, Roslindale, MA (US); Marc J. Lajoie, Cambridge, MA (US); Xavier Rios Villanueva, Boston, MA (US)

(73) Assignees: President and Fellows of Harvard College, Cambridge, MA (US); President and Fellows of Harvard College, Cambridge, MA (US)

(21) Appl. No.: 16/075,281

(22) PCT Filed: Feb. 2, 2017
(86) PCT No.: PCT/US17/16184

§ 371 Date: Aug. 3, 2018

Related U.S. Application Data
(60) Provisional application No. 62/315,336, filed on Mar. 30, 2016.
(60) Provisional application No. 62/291,499, filed on Feb. 4, 2016.

(51) Int. Cl. C12N 15/10

(52) U.S. Cl. CPC C12N 15/1058

(57) ABSTRACT

A method of altering a target nucleic acid sequence within a cell is provided including providing the cell with a donor nucleic acid, providing the cell with a single strand annealing protein, and providing the cell with a single strand DNA binding protein, wherein one or more or both of the single strand annealing protein and the single strand DNA binding protein is foreign to the cell, and wherein the donor nucleic acid is recombined into the target nucleic acid.

### RELATED APPLICATION DATA

This application claims priority to U.S. Provisional Application No. 62/291,499 filed on Feb. 4, 2016 and to U.S. Provisional Application No. 62/315,336 filed on Mar. 30, 2016 which are hereby incorporated herein by reference in their entirety for all purposes.

### STATEMENT OF GOVERNMENT INTERESTS

This invention was made with government support under DE-FG02-02ER63445 awarded by the Department of Energy. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 2, 2017, is named 010498_00906-WO_SL.txt and is 161,995 bytes in size.

### FIELD

The present invention relates in general to genome editing methods that use foreign recombinases.

### BACKGROUND

Oligonucleotide-mediated recombination is used for genome engineering (see Carr and Church 2009) where mutation-encoding oligonucleotides modify the genome without the need for site-specific DNA-binding proteins. Such techniques may be used to generate large complex libraries of genetic variants. Such techniques may also be used to generate complex, user-defined genotypes at high efficiency in E. coli. (see Wang et al., 2009). Oligo-mediated recombination has enabled multiple synthetic biology applications such as genetically recoded organisms (Lajoie et al., 2013) and sensor-based metabolic pathway optimization (Raman et al., 2014).

Efforts at recombineering (i.e., recombination-mediated genetic engineering) (are present in (Van Pijkeren and Britton, 2012), (van Pijkeren et al., 2012), (van Kessel and Hatfull, 2007), (Binder et al., 2013), (Datta et al., 2008). In E. coli, oligo-mediated targeting is most commonly done via λ Red recombineering, where an oligo preferentially anneals to the lagging strand of the genome during DNA replication and incorporates into the daughter strand (Ellis et al., 2001a). This system is based on the phage λ Red operon normally expressed during the phage's lytic growth (Poteete, 2001) and promotes high-efficiency, targeted recombination between linear, single-stranded (Mosberg et al., 2010) DNA (ssDNA) and the host chromosome. The λ Red operon is composed of Red α, β and γ, also known as exo (a 5′→3′ exonuclease), beta (a single stranded annealing protein [SSAP]), and gam (a RecBCD nuclease complex inhibitor), respectively. λ β is necessary and sufficient to recombine ssDNA into the E. coli chromosome and itself improves recombination rates in by 1E4-fold (Ellis et al., 2001b). The β-mediated recombination is based on the input ssDNA both directing proper targeting and encoding mutations of interest.

### SUMMARY

The disclosure provides methods of optimizing genome editing in organisms, such as bacteria. The disclosure provides for the identification of recombinases that can be used for genome editing in organisms, such as bacteria. A recombinase may also be referred to herein as a single strand annealing protein. Genome editing includes the use of a recombinase to recombine genomic DNA to include a donor nucleic acid sequence such as a single stranded DNA (ssDNA). Such genome editing may be known in the art as “recombineering.” The disclosure provides for the identification and use of components sufficient to produce introduction of a foreign nucleic acid sequence into the genome of a cell. One or more or all of such components may be foreign to the cell. Such components include a recombinase (also referred to as a single strand annealing protein or SSAP) and a single-strand binding protein. The disclosure provides for the identification of one or more pairs of a recombinase and a single-stranded binding protein that can be used in genome editing to incorporate an ssDNA into a genome. A single stranded binding protein (SSB) or a single stranded annealing protein (SSAP) is one that participates in replication, repair or recombination. An exemplary recombinase used for recombineering is λ Red as described in (Carr et al., 2012; Lajoie et al., 2012; Miki et al., 2008; Mosberg et al., 2012; Wang et al., 2009, 2011). An exemplary single-stranded binding protein is single-strand DNA-binding protein (SSB), an example of which is found in E. coli. See Meyer R R, Laine P S (December 1990), Microbiol. Rev. 54 (4): 342-80. Other exemplary recombinases or single-strand DNA-binding proteins may be found in other bacteria and viruses. The disclosure provides that either one or both of a recombinase and a corresponding single-stranded binding protein is foreign to the organism which uses them for genome editing or into which they are provided. According to one aspect, the recombinase and a corresponding single-stranded binding protein are provided to a cell as native species or as a nucleic acid encoding the recombinase or the corresponding single-stranded binding protein for expression within the cell. The disclosure provides a method of genome editing by including one or more or both of a recombinase and a corresponding single-stranded DNA-binding protein into a cell where one or more or both of a recombinase and a corresponding single-stranded DNA binding protein is foreign to the cell and where a donor nucleic acid sequence is introduced into the genome of the cell. The disclosure provides that the combination of a recombinase and a corresponding single-stranded DNA binding protein provide the minimal functional units used by a cell to insert ssDNA into its genome. The recombinase and a corresponding single-stranded DNA binding protein may be evolved from the same or different organisms. However, at least one is foreign to the cell into which they are provided or are otherwise present.

The disclosure provides a library-based method of identifying candidate single-stranded annealing proteins for use in oligo-recombination. The disclosure provides a library-based method of identifying candidate single-stranded annealing proteins from various and diverse organisms for use in oligo-recombination.

The disclosure provides a method by which β anneals complementary ssDNA pre-coated with SSB which is dependent on the C-terminal 8 amino acid tail of SSB. The disclosure provides a method by which the C-terminus of λ β is involved in its interaction with SSB. The disclosure provides a method of co-expressing a low-activity SSAP and its corresponding SSB to achieve oligo recombination. The λ β-SSB is a minimal functional unit of recombination and constitutes a host interaction node regulating recombination frequencies.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 depicts in schematic a Serial Evolutionary Enrichment for Recombinases (SEER) workflow. A complex plasmid library bearing the synthetic recombinases to be tested was transformed into a reporter E. coli strain containing two chromosomally integrated broken antibiotic resistance markers, tolC and cat. These broken resistance markers can then be repaired by two serial oligo recombination and selection, effectively enriching for cells with functional recombinases. After this the selected plasmids were extracted and re-transformed into the original strain, completing two Rounds of Enrichment (RoE). After six RoE we directly PCR′d the barcode region from the recombinase insert with Illumina-compatible primers to prepare indexed libraries for deep sequencing analysis.

FIG. 2A-2C depict results of recombinase discovery using SEER in E. coli. Using the first library (72+10 recombinases), multiple library configurations including Library 1.1 (72 recombinases of unknown function), Library 1.2 (1.1+9 recombinases known to function to varying degrees in E. coli), and Library 1.3 (1.2+λ β) were prepared. SEER was conducted with these 3 library configurations. FIG. 2A: After 6 RoE, the frequencies of enriched SSAPs were quantified by Sanger sequencing of their corresponding barcodes from 48 clones. FIG. 2B: The Allele Recombination (AR) Frequency of the most abundant SSAPs were characterized from (A) by quantifying oligo recombination frequencies of fixing a tolC marker (using tolC-Lnull_revert) via serial dilution and colony counting. The data are presented as mean±Std. Error of the Mean with the scatter plotted and color coded to match (A). These data are the result of at least 4 biological replicates. FIG. 2C: Toxicity of SSAPs from (A) and (B) were tested using kinetic growth assays with (+L-Ara) and without L-arabinose (−L-Ara) induction. Two negative controls, pARC8.GFP and empty pARC8 were used. Growth kinetics were distilled into doubling time as calculated previously {24136967}. These data are the result of 3 biological replicates and are color coded to match (A) and (B).

FIG. 3A-3B depict Expanded Recombinase Search Space Using SEER in E. coli. In order to better sample the bacteriophage derived SSAP sequence space, a Hidden Markov Model-based search strategy using multiple known recombinases was used to generate the position matrix with which to search nucleotide databases. This new searched contributed 113 new SSAP, for a total recombinases library size of 195 members. FIG. 3A: Phylogenetic relationship between Single-strand annealing protein library members. The 6 clades of phage-derived SSAPs are color coded: redβ (red); sak (yellow); erf (light blue); gp2.5 (light green); sak4 (purple); and uvsX (orange). FIG. 3B: The population of recombinases was sequenced at each step of SEER, included before any enrichment (0 RoE). The population distribution of unique members was plotted as a stack plot with RoE on the x-axis. Over subsequent RoE, the population diversity of this SEER linage drops as the system converges on a solution.

FIG. 4A-4D depict characterizing the C-terminal of Beta. FIG. 4A: In order to test the λ β mediated interactions between SSAPs and SSB, the C-terminus of λ β protein was serially truncated into various fragments (177, 194, 211, 228, 245 amino acids, where 266 is wildtype). These variants were expressed on pARC8 and transformed into the SEER chassis to measure GFP reversion using GFP.r2_revert, followed by flow cytometric analysis of the GFP+ population (reported as % AR). FIG. 4B: Same as in (A), but using single alanine substitutions in λ β. FIGS. 3C and 3 D: Gel shift assay showing the ssDNA binding of a subset of beta mutations: wild type (red square); truncation mutant β1-194 (orange triangle); point mutants βK214A (blue circle); βK172A (green diamond). In addition the recombinase Q8 was included (purple square).

FIG. 5A-5C depict that beta interacts with SSB in a Mg²⁺-dependent reaction. FIG. 5A: Fluorescent oligo quenching assay. Briefly, two complementary oligos with compatible FITC fluorophore and quencher anneal and lead to a decay in the fluorescence intensity that can be tracked over time (1). Thus, fluorescence intensity will be proportional to the amount of starting substrate, while the remaining fraction will be the annealed product. If the oligos are coated with SSB prior mixing, they will be prevented from annealing unless additional factors are able to remove the inhibition (2). The following traces are representative examples of an experiment that was carried out at least 4 times. FIG. 5B: Two SSB-coated complementary oligos were incubated with λ (3 in the presence of 10 mM MgCl₂ (red curve) and 1 mM EDTA (blue curve) showing that λ β is capable of removing the annealing inhibition of SSB in a Mg′-dependent reaction. In addition, the assay was repeated with SSBdC8 coated oligos (green curve), which abolished the de-inhibitory activity λ β. This suggests that like in other SSB-interacting proteins, the C-terminal 8-amino acid tail of SSB is required for its interaction with λ β. FIG. 5C: Annealing of SSB-coated oligos in the presence of various SSAPs. Only full length λ β WT is capable of annealing SSB-coated oligos (red curve), while the 1-194 truncation is indistinguishable from the no SSAP control (orange vs. purple curve). The λ β K214A single-amino acid substitution also has a significantly impaired activity (green curve). In addition, we tested Q8KQW0 from Vibrio cholera, the most active recombinase from our initial library. Q8KQW0 showed moderate activity in the SSB-coated oligo annealing assay (blue curve).

FIG. 6A-6B depict that co-expression of species-matched SSAP-SSB pairs enable gain of recombinase function. FIG. 6A: To test the plausibility of bi-cistronic expression in an L-arabinose-based inducible pARC8 vector, a vector was synthesized that conferred both λ β and a spectinomycin resistance and the growth of that construct was tested under inducing conditions. Ecnr2 is the positive control in which the addition of arabinose produces no difference than with spectinomycin alone. The Beta.Spec bi-cistronic vector grows in the presence of arabinose (dark blue curve) shows increased expression than when it is simply induced with spec, alone. FIG. 6B: Given (A), bi-cistronic variants were constructed containing the indicated SSAP and the indicated SSB. These plasmid variants were transformed into the SEER chassis and performed-arabinose-based oligo recombinations to fix tolC, and quantified Allele Recombination (AR) Frequency as the number of colonies on selective media (SDS) divided by the number of colonies on non-selective media (carb). These data are presented as the mean AR Frequency and the error bars indicate the Std. Error of the Mean, and are the result of at least 4 biological replicates. Here, Lr=L. reuteri (light gray), Cg=C. glutamicum (dark gray), Ec=E. coli (white). The black column indicates the frequency of AR frequency of recombinase-deficient clones electroporated with the same oligos (background).

FIG. 7A-7D depict data regarding synthesis of metagenomic recombinase homologs.

FIG. 8A-7C depict data of results of recombinase discovery using the SEER method described herein in E. coli.

FIG. 9 depict data regarding synthesis of metagenomic recombinase homologs.

### DETAILED DESCRIPTION

The present disclosure provides methods of in vivo or ex vivo recombination-mediated genetic engineering including providing a cell, such as a prokaryotic cell or eukaryotic cell, with a recombinase and a single strand binding protein (i.e., single strand nucleic acid binding protein or a single strand DNA binding protein) and a donor nucleic acid (i.e., a single stranded nucleic acid, a single stranded DNA, a double stranded nucleic acid or a double stranded DNA), wherein either one or both of the recombinase and a single strand binding protein are foreign to the cell in which they are present. The pair of the recombinase and the single strand binding protein, in combination with the host cell's translational machinery, is sufficient to insert an ssDNA sequence into a target nucleic acid sequence within the cell.

The present disclosure provides methods of in vitro recombination-mediated genetic engineering including providing in a suitable in vitro environment a target nucleic acid sequence, a target cell's translational machinery (i.e., those proteins and other components responsible for translation within the cell), a recombinase and a single strand binding protein (i.e., single strand nucleic acid binding protein or a single strand DNA binding protein) and a donor nucleic acid (i.e., a single stranded nucleic acid, a single stranded DNA, a double stranded nucleic acid or a double stranded DNA), wherein either one or both of the recombinase and a single strand binding protein are foreign to the cell. The pair of the recombinase and the single strand binding protein, in combination with the host cell's translational machinery, is sufficient to insert an ssDNA sequence into a target nucleic acid sequence.

Cells according to the present disclosure include any cell into which foreign nucleic acids can be introduced and expressed as described herein. It is to be understood that the basic concepts of the present disclosure described herein are not limited by cell type. Cells according to the present disclosure include eukaryotic cells, prokaryotic cells, animal cells, plant cells, fungal cells, bacteria cells, archaeal cells, eubacterial cells and the like. Cells include eukaryotic cells such as yeast cells, plant cells, and animal cells. Particular cells include mammalian cells and human cells. Particular cells include stem cells, such as pluripotent stem cells, such as human induced pluripotent stem cells.

Target nucleic acids include any nucleic acid sequence into which a donor nucleic acid can be inserted or introduced or otherwise included. Target nucleic acids include genes. For purposes of the present disclosure, DNA, such as double stranded DNA, can include the target nucleic acid. Such target nucleic acids can include endogenous (or naturally occurring) nucleic acids and exogenous (or foreign) nucleic acids. The target nucleic acid sequence may be replicating DNA such as genomic DNA, mitochondrial DNA, viral DNA, exogenous DNA, a plasmid, a bacteriophage genome and other replicating DNA known to those of skill in the art.

The donor nucleic acid includes any nucleic acid to be inserted into a nucleic acid sequence as described herein. Foreign or exogenous nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

Standard recombinant DNA and molecular cloning techniques used herein are well known in the art and are described in Sambrook, J., Fritsch, E. F. and Maniatis, T., Molecular Cloning: A Laboratory Manual, 2^{nd} ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N.Y., (1984); and by Ausubel, F. M. et. al., Current Protocols in Molecular Biology, Greene Publishing and Wiley-Interscience (1987) each of which are hereby incorporated by reference in its entirety.

Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

Microorganisms may be genetically modified to delete genes or incorporate genes by methods known to those of skill in the art. Vectors and plasmids useful for transformation of a variety of host cells are common and commercially available from companies such as Invitrogen Corp. (Carlsbad, Calif.), Stratagene (La Jolla, Calif.), New England Biolabs, Inc. (Beverly, Mass.) and Addgene (Cambridge, Mass.).

Typically, the vector or plasmid contains sequences directing transcription and translation of a relevant gene or genes, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention including, but not limited to, lac, ara, tet, trp, IP_{L}, IP_{R}, T7, tac, and trc (useful for expression in Escherichia coli and Pseudomonas); the amy, apr, npr promoters and various phage promoters useful for expression in Bacillus subtilis, and Bacillus licheniformis; nisA (useful for expression in Gram-positive bacteria, Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)). Termination control regions may also be derived from various genes native to the preferred hosts.

Certain vectors are capable of replicating in a broad range of host bacteria and can be transferred by conjugation. The complete and annotated sequence of pRK404 and three related vectors-pRK437, pRK442, and pRK442(H) are available. These derivatives have proven to be valuable tools for genetic manipulation in Gram-negative bacteria (Scott et al., Plasmid 50(1):74-79 (2003)). Several plasmid derivatives of broad-host-range Inc P4 plasmid RSF1010 are also available with promoters that can function in a range of Gram-negative bacteria. Plasmid pAYC36 and pAYC37, have active promoters along with multiple cloning sites to allow for the heterologous gene expression in Gram-negative bacteria.

Chromosomal gene replacement tools are also widely available. For example, a thermosensitive variant of the broad-host-range replicon pWV101 has been modified to construct a plasmid pVE6002 which can be used to create gene replacement in a range of Gram-positive bacteria (Maguin et al., J. Bacteriol. 174(17):5633-5638 (1992)). Additionally, in vitro transposomes are available to create random mutations in a variety of genomes from commercial sources such as EPICENTRE® (Madison, Wis.).

Vectors useful for the transformation of E. coli are common and commercially available. For example, the desired genes may be isolated from various sources, cloned onto a modified pUC19 vector and transformed into E. coli host cells. Alternatively, the genes encoding a desired biosynthetic pathway may be divided into multiple operons, cloned onto expression vectors, and transformed into various E. coli strains.

The Lactobacillus genus belongs to the Lactobacillales family and many plasmids and vectors used in the transformation of Bacillus subtilis and Streptococcus may be used for Lactobacillus. Non-limiting examples of suitable vectors include pAM.beta.1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol. 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Several plasmids from Lactobacillus plantarum have also been reported (van Kranenburg R, Golic N, Bongers R, Leer R J, de Vos W M, Siezen R J, Kleerebezem M. Appl. Environ. Microbiol. 2005 March; 71(3): 1223-1230), which may be used for transformation.

Initiation control regions or promoters, which are useful to drive expression of the relevant pathway coding regions in the desired Lactobacillus host cell, may be obtained from Lactobacillus or other lactic acid bacteria, or other Gram-positive organisms. A non-limiting example is the nisA promoter from Lactococcus. Termination control regions may also be derived from various genes native to the preferred hosts or related bacteria.

The various genes for a desired pathway may be assembled into any suitable vector or vectors, such as those described above. A single vector need not include all of the genetic material encoding a complete pathway. One or more or a plurality of vectors may be used in any aspect of genetically modifying a cell as described herein. The codons can be optimized for expression based on the codon index deduced from the genome sequences of the host strain, such as for Lactobacillus plantarum or Lactobacillus arizonensis. The plasmids may be introduced into the host cell using methods known in the art, such as electroporation, as described in any one of the following references: Cruz-Rodz et al. (Molecular Genetics and Genomics 224:1252-154 (1990)), Bringel and Hubert (Appl. Microbiol. Biotechnol. 33: 664-670 (1990)), and Teresa Alegre, Rodriguez and Mesas (FEMS Microbiology Letters 241:73-77 (2004)). Plasmids can also be introduced to Lactobacillus plantatrum by conjugation (Shrago, Chassy and Dobrogosz Appl. Environ. Micro. 52: 574-576 (1986)). The desired pathway genes can also be integrated into the chromosome of Lactobacillus using integration vectors (Hols et al. Appl. Environ. Micro. 60:1401-1403 (1990); Jang et al. Micro. Lett. 24:191-195 (2003)).

Microorganisms which may serve as host cells and which may be genetically modified to produce recombinant microorganisms as described herein may include one or members of the genera Clostridium, Escherichia, Rhodococcus, Pseudomonas, Bacillus, Lactobacillus Saccharomyces, and Enterococcus. Particularly suitable microorganisms include Escherichia coli, Bacillus subtilis, and Saccharomyces cerevisiae.

Exemplary genus and species of bacteria cells for use in the methods described herein, for use in identifying corresponding phage, or for otherwise carrying out recombination-mediated genetic engineering include Acetobacter aurantius, Acinetobacter bitumen, Actinomyces israelii, Agrobacterium radiobacter, Agrobacterium tumefaciens, Anaplasma Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides, Bacteroides fragilis, Bacteroides gingivalis, Bacteroides melaninogenicus (also referred to as Prevotella melaninogenica), Bartonella, Bartonella henselae, Bartonella quintana, Bordetella, Bordetella bronchiseptica, Bordetella pertussis, Borrelia burgdorferi, Brucella abortus, Brucella melitensis, Brucella suis, Burkholderia, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila Chlamydophila pneumoniae (also known as Chlamydia pneumoniae) Chlamydophila psittaci (also known as Chlamydia psittaci), Clostridium, Clostridium botulinum, Clostridium difficile, Clostridium perfringens (also known as Clostridium welchii), Clostridium tetani, Corynebacterium, Corynebacterium diphtheriae, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans, Mycoplasma genitalium, Mycoplasma hominis, Mycoplasma penetrans, Mycoplasma pneumoniae, Neisseria, Neisseria gonorrhoeae, Neisseria meningitidis, Pasteurella, Pasteurella multocida, Pasteurella tularensis, Peptostreptococcus, Porphyromonas gingivalis, Prevotella melaninogenica (also known as Bacteroides melaninogenicus), Pseudomonas aeruginosa, Rhizobium radiobacter, Rickettsia, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Staphylococcus, Staphylococcus aureus, Staphylococcus epidermidis, Stenotrophomonas maltophilia, Streptococcus Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Treponema pallidum, Treponema denticola, Vibrio, Vibrio cholerae, Vibrio comma, Vibrio parahaemolyticus, Vibrio vulnificus, Wolbachia, Yersinia, Yersinia enterocolitica, Yersinia pestis, and Yersinia pseudotuberculosis, and other genus and species known to those of skill in the art.

Exemplary genus and species of yeast cells for use in the methods described herein, or for otherwise carrying out recombination-mediated genetic engineering include Saccharomyces, Saccharomyces cerevisiae, Torula, Saccharomyces boulardii, Schizosaccharomyces, Schizosaccharomyces pombe, Candida, Candida glabrata, Candida tropicalis, Yarrowia, Candida parapsilosis, Candida krusei, Saccharomyces pastorianus, Brettanomyces, Brettanomyces bruxellensis, Pichia, Pichia guilliermondii, Cryptococcus, Cryptococcus gattii, Torulaspora, Torulaspora delbrueckii, Zygosaccharomyces, Zygosaccharomyces bailii, Candida lusitaniae, Candida stellata, Geotrichum, Geotrichum candidum, Pichia pastoris, Kluyveromyces, Kluyveromyces marxianus, Candida dubliniensis, Kluyveromyces, Kluyveromyces lactis, Trichosporon, Trichosporon uvarum, Eremothecium, Eremothecium gossypii, Pichia stipitis, Candida milleri, Ogataea, Ogataea polymorpha, Candida oleophilia, Zygosaccharomyces rouxii, Candida albicans, Leucosporidium, Leucosporidium frigidum, Candida viswanathii, Candida blankii, Saccharaomyces telluris, Saccharomyces florentinus, Sporidiobolus, Sporidiobolus salmonicolor, Dekkera, Dekkera anomala, Lachancea, Lachancea kluyveri, Trichosporon, Trichosporon mycotoxinivorans, Rhodotorula, Rhodotorula rubra, Saccharomyces exiguus, Sporobolomyces koalae, and Trichosporon cutaneum, and other genus and species known to those of skill in the art.

Exemplary genus and species of fungal cells for use in the methods described herein, or for otherwise carrying out recombination-mediated genetic engineering include Sac fungi, Basidiomycota, Zygomycota, Chtridiomycota, Basidiomycetes, Hyphomycetes, Glomeromycota, Microsporidia, Blastocladiomycota, and Neocallimastigomycota, and other genus and species known to those of skill in the art.

Exemplary recombinases for use in the recombineering methods described herein are listed in Tables 1-6. Exemplary single strand binding proteins for use in the recombineering methods described herein are listed in Table 7. Table 8 is an exemplary list of single stranded binding homologs corresponding to the protein sequences referenced by Uniprot IDs. Exemplary pairs of single strand binding proteins and recombinases include SSB (WP_003669492.1) and DNA recombination protein 1 from Lactobacillus reuteri (WP_003668036.1); SSB (WP_011835834.1) from Lactococcus lactis and phage recombination protein bet from lactococcus phage phi31.1; SSB (WP_011015545.1) from Corynebacterium glutamicum and gp61 (NP_817738.1) from Mycobacteriophage Che9c; SSB (WP_003400534.1) from Mycobacterium tuberculosis and gp61 (NP_817738.1) from Mycobacteriophage Che9c; SSB (WP_011269089.1) and recT (YP_235897.1) from Pseudomonas syringae; and SSB (CQR83440.1) and λ β itself from Escherichia coli K-12 sp MG1655.

The disclosure provides the use of Multiplex Automated Genome Engineering (MAGE) to enable multiplexed genomic mutations in Escherichia coli. See Wang, H. H. et al. 2009. Programming cells by multiplex genome engineering and accelerated evolution. Nature. 460, 7257 (August 2009), 894-8 hereby incorporated by reference in its entirety; Wang, H. H. et al. 2012. Genome-scale promoter engineering by co-selection MAGE. Nature methods. 9, 6 (June 2012), 591-3; Lajoie, M. J. et al. 2013. Probing the limits of genetic recoding in essential genes. Science (New York, N.Y.). 342, 6156 (October 2013), 361-3; Lajoie, M. J. et al. 2013. Genomically recoded organisms expand biological functions. Science (New York, N. Y). 342, 6156 (October 2013), 357-60 each of which is hereby incorporated by reference in its entirety.

The disclosure provides the use of MAGE with the λ Red recombinase, λ β (Bet), a viral recombinase or homologs thereof or proteins having similar function to λ β (Bet), that when ectopically expressed improves the efficiency of recombination of single-stranded DNA oligonucleotides into the bacterial genome. See Lajoie, M. J. et al. 2012. Manipulating replisome dynamics to enhance lambda Red-mediated multiplex genome engineering. Nucleic acids research. 40, 22 (December 2012), e170 hereby incorporated by reference in its entirety. The disclosure provides a method referred to herein as Serial Evolutionary Enrichment for Recombinases (SEER) that enables the rapid discovery of Bet variants for use with MAGE in certain prokaryotic strains. A library of Bet homologs was built with homology searches across all known prokaryotic proteins, and curated to ensure large diversity (200 homologs). This library was then subjected to six successive rounds of selection in E. coli for improved recombineering activity, and characterized. Improved Bet homologs may be used for recomineering in Escherichia coli, Lactobacillus reuteri and Corynebacterium glutamicum.

The present disclosure provides that the molecular basis of Bet's recombinase function includes interaction with E. coli's single-stranded binding protein (SSB). Bet acts to specifically unload SSB from SSB-coated single-stranded DNA (ssDNA). This then enables strand-strand annealing, which is the mechanism by which ssDNA is incorporated into the replication fork in Bet-mediated recombineering. The present disclosure provides that an SSB taken from the same host organism as the Bet recombinase homolog improves the functioning of the Bet homolog in E. coli. Accordingly, the recombinase and/or the single strand binding protein are foreign to the cell in which they are present while facilitating incorporation of a donor nucleic acid into a target nucleic acid.

The following examples are set forth as being representative of the present disclosure. These examples are not to be construed as limiting the scope of the present disclosure as these and other equivalent embodiments will be apparent in view of the present disclosure, figures and accompanying claims.

### Example I

### Materials and Methods

### Strains & Culture Methods

The strains used in this work were derived from EcNR2 (EcNR2.dnaG_Q576A.tolC_mut.mutS::cat_mut.dlambda::zeoR) (Wang et al., 2009). Strains were grown in liquid culture using the Lennox formulation of lysogeny broth (LB^{L}) (Lennox, 1955) with the appropriate selective agents: carbenicillin (50 μg/mL), chloramphenicol (20 μg/mL), SDS (0.005% w/v), zeocin (100 μg/mL).

### Oligonucleotides, Polymerase Chain Reaction, Quikchanges, and Isothermal Assembly

Oligonucleotides were identified. PCR products used in transformations and recombinations were amplified using Kapa Biosystems, High-Fidelity polymerase, according to the manufacturer's instructions. Kapa 2G Fast ready mix was used to PCR screen the correct insertion in strains. Sanger sequencing of PCR products was carried out through a 3^{rd} party service (Genewiz, Inc.). To assemble multiple DNA sequences into a single contiguous sequence, or to assemble a circularized vector from linear vector backbone and insertion variants, isothermal assembly at 50° C. for 60 minutes was used based on published protocols (Gibson et al., 2009). When SNV variants of a given plasmid system (e.g., to introduce point mutations) were desired, the QuikChange II Lightning Kit (Agilent Technologies) was used with primers encoding the mutations of interest to generate the mutant strand, followed by dpnI-digest of the parental plasmid, according to the manufacturer's instructions.

### Transformations, A Red Recombinations, & MAGE

Transformations were conducted with Zymo Research's Mix & Go DH5a Z-competent E. coli, according to the manufacturer's protocol, except for the recovery step where the culture was recovered in 1 mL of LB^{L} for 1 hour before plating onto appropriate medium.

For recombinase discovery using the SEER method described herein and characterization, λ Red recombineering was implemented on episomal expression vector using 0.2% D-glucose to repress and 0.2% L-arabinose to induce expression (Datsenko and Wanner, 2000). An overnight growth culture was passaged 1:100 into 3 mL LB^{L} with 0.2% D-glucose. The cultures were then incubated at 34 C with rotation until the OD₆₀₀ ˜0.1 (˜1 hour). 2 mL culture was then washed twice with 2 mL water and resuspended in 2.5 mL LB^{L} with 0.2% L-arabinose. The cultures were then induced for 45 minutes while rotating at 34° C. followed by icing the culture and washing 0.980 mL of culture twice in ice-cold sterile water. Thereafter, pellets were resuspended with 50 μl of 1 μM ssDNA oligo, or 100 ng dsDNA PCR product, or both depending on the goal of the recombination. Cultures were electroporated at 1.8 kV, 200 ohms, 25 uF and recovered in 2 mL of LB^{L} for 2 hours.

### Synthesis of Metagenomic Recombinase Libraries

To generate a library of candidate SSAPs, two approaches to cataloging metagenomic homologs of λ β were pursued. For the first, an iterative PSI-blast was performed using λ (NP_040617.1) as the query. Candidates exhibited a bi-modal distribution where the first was SSAP-like, with sequence lengths from 500-1,050 bp (except for 4 candidates >1,050 bp.), and were annotated as recombination protein or unknown. The second were larger genes (1,200-1,500+bp), and largely annotated as ABC-related ATP binding cassettes. The latter were removed. Any SSAP-like candidates from E. coli were removed to minimize redundancy with λ β. Identical entries were removed.

In the second approach, multiple sequence alignment of NP_040617.1 (λ β), NP_930169.1 from Photorhabdus luminescens, Q9AKZ0 from Legionella pneumophila, Q8KQW0 from Virbio cholerae, Q9MBV8 from Lactococcus phage u136.2, YP_003084246.1 from Prochlorococcus siphovirus P-SS2, NP_815795.1 (EF2132) from Enterococcus faecalis, and NP_463513.1 from Listeria phage A118 were used to generate a Hidden Markov Model that described the weighted positional variance of these proteins. Non-redundant nucleotide and environmental metagenomic databases were queried using web-based search interface (Finn et al., 2011). Candidates were filtered based on gene size and ABC ATP-binding cassette annotation. Candidates that exhibited intra-sequence similarity of greater than 98% were removed from the group.

A number of other recombinases were added for synthesis, including candidates from other model organisms that have been previously shown to function in E. coli (NP_930169.1 from Photorhabdus luminescens, Q9AKZ0 from Legionella pneumophila, Q8KQW0 from Virbio cholerae, Q9MBV8 from Lactococcus phage u136.2, YP_003084246.1 from Cyanophage P-SS2, NP_815795.1 (EF2132) from Enterococcus faecalis (Datta et al., 2008), recT from E. coli K12 (B1XAU6), CG19468 from Drosophila Melanogaster (Eisen and Camerini-Otero, 1988), C7F4E8 from Prochlorococcus siphovirus P-SS2 (Sullivan et al., 2009), and NP_040617.1 (λ β itself) (P03698). In total, a library was created that contained 72 members from the first approach, 113 members from the second, and 10 members that were rationally added for a total of 195 recombinase homologs.

To prepare the library for synthesis (Gen9, Inc.), the protein coding sequence was reverse translated using optimized codon usage tables for E. coli. Upper bounds (>70%) and lower bounds (<30%) for GC-content of 100-mer windows were set and codon usage was manually messaged to meet these requirements. ATG was used for all starts codons. TAA was used for all stop codons. Upstream of the coding sequence, 35 bp of homology was added to support assembly with the pARC8 (Choe et al., 2005) vector (5′-TTCTCCATACCTGTTTTTCTGGATGGAGTAAGACC-3′)(SEQ ID NO:1). After the stop codon, the primer sequence of a Illumina-like primer, barcode region of interest that was unique to each library member, and the hybridization site for a reverse Illumina primer to support a PCR-based library preparation for high-throughput, Illumina sequencing was added (see below). Downstream of the barcode region, 35 bp of homology was added to support assembly with the pARC8 vector (5′-ACTAGTGGGGAAGCTTATCGATGATAAGCTGTCAA-3′)(SEQ ID NO:2). As a final synthesis requirement, synthons were manually redesigned, as needed, to avoid the following sequences: GGGGG, AAAAAAAA, CCCCCCCC, TTTTTTTT, GGTCTC, GAGACC.

Sythons were pooled at equimolar ratios and assembled in a complex isothermal assembly (Gibson et al., 2009) using a linear pARC8 vector backbone, which enabled episomal expression of the recombinase candidates under 0.2% L-arabinose at a copy number ˜10. Crude assemblies were transformed into Z-competent DH5a (Zymo Research) and plated onto LB^{L} agar containing carbenicillin to generate sufficient colonies for at least 10× coverage. The colonies were counted and scraped into LB^{L} plus carbenicillin for plasmid preparation. To verify the diversity of any library preparation, a plasmid-limiting transformation of the complex plasmid preparation into naïve DH5a using 1 ng, 100 pg, 10 pg, and 1 pg of plasmid was performed, followed by plating onto selective agar. 96 clones were picked from the most plasmid-limited conditions and inserts were identified using the barcode region of interest.

### Serial Evolutionary Enrichment for Recombinases (SEER)

To perform SEER (e.g., enrich for functional recombinases), oligo recombinations were leveraged to restore the coding region of a broken selectable marker followed by the respective selection as the mechanistic foundation for enrichment. In E. coli, multiple markers were used and inactivated, as such used MAGE to inactivate tolC^{WT}, mutS::cat, and 1984000::gfp_mut3b using oligo recombinations, followed by asPCR screening or replica plating to isolate the inactivated clones. These three inactivations generated EcNR2.mutS::cat_mut.tolC^{WT}_mut.1984000::gfp_mut3b_mut, which still contained the λ prophage and is competent for recombination. The entire prophage was then deleted in a dead-end recombination using a Δλ::zeoR PCR cassette, followed by selection on LB^{L} agar plus zeocin to create a recombinase-deficient chassis for SEER in E. coli.

After transforming the pARC8-based libraries into the naive SEER chassis, the libraries were induced using 0.2% L-arabinose and oligo recombinations were performed to fix a broken selectable marker or a mock, water-only recombination was performed. The tolC^{WT}_mut was fixed using tolC-Lnull_revert, followed by selection in LB^{L}+SDS. The mutS::cat_mut was fixed using cat_CS_restore followed by selection in LB^{L}+Cm. These two oligo recombination/selection steps constitute 2 Rounds of Enrichment (RoE), but exhausted the opportunities for selectable recombinations in the chassis. To continue, total plasmid preps were prepared from post-selection cultures after 2 RoE, and the preparation was transformed into a naïve chassis to conduct additional RoE's and facilitate convergence onto a solution.

### High-Throughput Sequencing

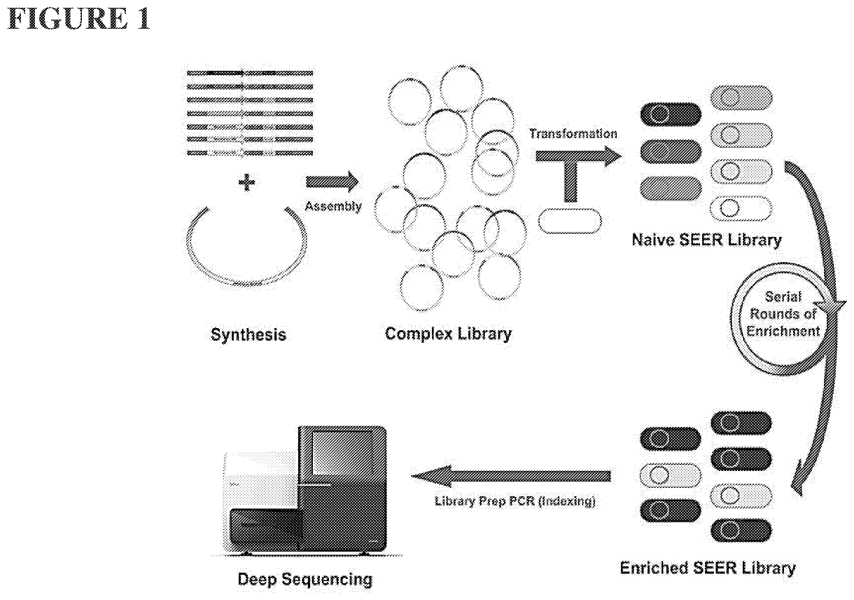

To support rapid and deep identification of recombinases, a custom Illumina sequencing platform was designed to leverage high-fidelity PCR to amplify the barcode region directly using large library size. After the stop (TAA), the seed sequence for barcoded Illumina p7 forward adapter (GACGTGTGCTCTTCCGATCT)(SEQ ID NO:3) was added, followed by two tandem 6-mer library IDs (cNNNNNNgNNNNNN)(SEQ ID NO:4), followed by the hybridization site for p5_alt (GATCGCCTAGACAACTCCTGA)(SEQ ID NO:5), a custom sequence chosen for minimal secondary structure (Kosuri et al., 2010; Xu et al., 2009). The p5_alt hyb site binds the barcoded, Illumina-compatible p5_alt reverse adapter, supporting robust amplification with few cycles. Libraries were amplified with Phusion (New England Biolabs) at 100 μL scale containing genomes from 10 μL of post-selection culture (10⁷-10⁸ unique clones) for 10-16 cycles. The expected amplicon size is 146 bp and follows the format 5′-AATGATACGGCGACCACCGAGATCTACACnnnnnnACACTCTTTCCCTCAGGAGTT GTCTAGGCGATCcNNNNNNgNNNNNNAGATCGGAAGAGCACACGTCTGAACTCC AGTCACnnnnnnATCTCGTATGCCGTCTTCTGCTTG-3′ (SEQ ID NO:6), where nnnnnn are 6-mer indices added in the PCR reaction (see Table S4) (Gregg et al., 2014). Magnetic bead-associated PEG was used to cleanup reactions (Rohland and Reich, 2012). The libraries were visualized for specificity and pooled to equimolar amounts depending on the number of indices (unique experimental conditions) being sequenced. MiSeq SE50 runs were carried out using the custom read primer (ACACTCTTTCCCTCAGGAGTTGTCTAGGCGATC)(SEQ ID NO:7) and standard indexing primer, and included a 30% PhiX spike-in to mitigate sequencing of largely constant regions. A diagram depicting the entire SEER workflow is shown at FIG. 1.

### Protein Purification

pARC8 was also leveraged for in vitro characterization and recombinant recombinase production. After cloning via isothermal assembly (Gibson et al., 2009) and adding an N-terminal 6×His tag (SEQ ID NO: 108) onto candidate proteins, the vector was transformed into NiCo21(DE3) competent E. coli (New England Biolabs). For protein production, 50 mL LB^{L} plus 25 μg/mL chloramphenicol was inoculated with 1:100 from overnight confluent cultures, themselves grown LB^{L} plus chloramphenicol plus 0.2% D-glucose. The 50 mL cultures were grown for 6 hours at 37° C. in LB^{L}+chloramphenicol, then induced using 0.1% L-arabinse. Cultures were spun down at 5,000 g for 10 minutes at 4° C. and the pellets were snap frozen in a dry ice ethanol bath. The pellets were thawed, then lysed using P-BER with Enzymes (Thermo Scientific) for 10 minutes at room temperature, according to the manufacturer's instructions. Lysates were mixed 1:1 with binding buffer (40 mM Imidazole, 500 nM NaCl, 50 mM Tris pH 7.4), spun down 10 mins 5,000 g 4° C. and the soluble fraction was added to a 20 mL column pre-loaded with 2 mL His GraviTrap Ni-NTA resin (GE Healthcare) that was pre-equilibrated with binding buffer. After binding, the columns were washed twice with 20 mL of wash buffer (100 mM Imidazole, 500 nM NaCl, 50 mM Tris pH 7.4), then eluted with 4 mL of elution buffer (500 mM Imidazole, 500 nM NaCl, 50 mM Tris pH 7.4) in 1 mL fractions. Protein concentration was quantified using the Qubit system (Life Technologies), and stability and purity was checked by SDS-PAGE (Bio-rad). The purest, most concentrated fractions were pooled and buffer exchanged with Zeba desalting columns 7K MWCO (ThermoFisher Scientific) into storage buffer (200 nM NaCl, 50 mM Tris pH 7.4, 1 mM DTT). Protein preps were concentrated using Amicon Ultra-4 10K centrifugal filters (Millipore), as needed.

### Oligonucleotide Quenching Assay

Fluorophore/quencher complementary oligos were ordered from IDT (5′-AGCAAGCACGCCTTAGTAACCCGGAATTGCGTAAGTCTGCCGCCGATCGTGATG CTGCCTTTGAAAAAATTAATGAAGCGCGCAGTCCA/6-FAM/-3′ (SEQ ID NO:8) and 5′-/IABkFQ/TGGACTGCGCGCTTCATTAATTTTTTCAAAGGCAGCATCACGATCGGCG GCAGACTTACGCAATTCCGGGTTACTAAGGCGTGCTTGCT-3′ (SEQ ID NO:9). For the SSB annealing assays, the 10 nM oligo solutions with 20 nM NaCl, 1 mM DTT, 50 mM Tris pH 7.4 were separately incubated with 500 nM SSB or SSBAC8 for 20 minutes at 37 C. The reactions were tracked in a Synergy H4 Hybrid Microplate Reader (Biotek) in half-area, low-bind black 96 well plates. The oligos were serially added to the plate, then the SSAP in the same buffer.

### Fluorescence Quenching-Based Annealed Fraction Estimate:

I=Fluorescence intensity at a given time is

I=F_{f}I_{f}+F_{b}I_{b}

Where F_{f}=Free fraction, I_{f}=Free intensity, F_{b}=Bound fraction, I_{b}=Bound fraction. For a DNA annealing assay, F_{f} is the substrate, and F_{b} is the product

$$\begin{matrix}At {t}_{o} \to {F}_{f} = 1 , {F}_{b} = 0 \\ {F}_{f} + {F}_{b} = 1 ; {F}_{f} = 1 - {F}_{b} so \\ I = {I}_{f} ( 1 - {F}_{b} ) + {F}_{b} {I}_{b} \\ {F}_{b} = \frac{I - {I}_{f}}{{I}_{b} - {I}_{f}} = 1 - \frac{I - {I}_{b}}{{I}_{f} - {I}_{b}} .\end{matrix}$$

This calculation is independent of experimental background (B) from the reader:

$${F}_{b} = \frac{( I - B ) - ( {I}_{f} - B )}{( {I}_{b} - B ) - ( {I}_{f} - B )} = \frac{I - {I}_{f}}{{I}_{b} - {I}_{f}}$$

I_{b} was estimated from the minimal steady-state fluorescence of annealed oligos in the presence of protein, while I_{f} was measured in parallel for each reaction using an unlabeled oligo instead for the quencher. This helped control for the variable background fluorescence of different protein solutions and the fluorescence decay of the FITC fluorophore over the time course measured. The reactions were tracked for an hour, measuring every 7 seconds. The naked-oligo experiments were done in a similar way, except no SSB was added during the pre-incubation step. Annealing and steady-state graphs were generated using GraphPad Prism 5.

### Example II

### Analysis of SEER Library 1 in E. coli

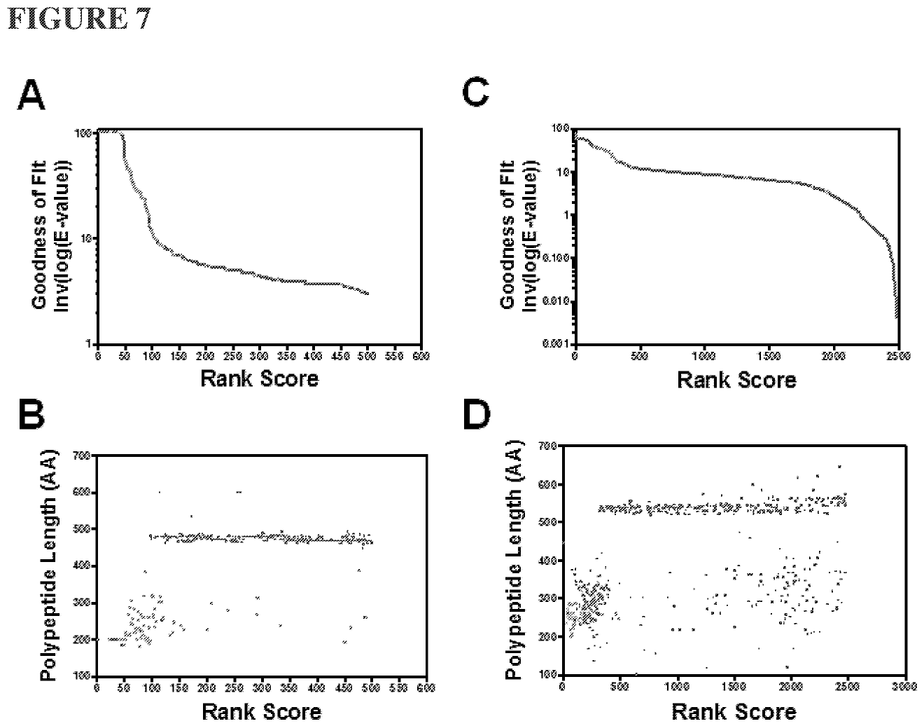

Phylogenetic analyses suggest that phage-derived SSAPs belong to six distinct families: redβ, erf, sak, sak4, uvsX, & gp2.5 (Iyer et al., 2002; Lopes et al., 2010). These recombinases are present in a variety of phages that exhibit both temperate and lytic lifestyles. The disclosure generates SSAP libraries that widely sample potential sequence space. An Iterated PSI-BLAST was used with the λ β amino acid sequence as the query, which produced a list of 500 candidates. From the initial hits, β homologs were removed from E. coli-derived phages, which were often identical to the query (e.g., E-value˜1⁻¹⁰⁴). By plotting the logarithm of the inverse of the E-value fit score for each candidate versus the rank score (FIG. 7A), it was visualize how the Goodness of Fit varied throughout the total list. In order to maximize the versatility of the library, both the highest ranked candidates and the tail-end of the distribution were sampled. A clear bi-modal distribution of predicted Polypeptide Length versus Rank Score (FIG. 7B) was identified, with the best hits (1st and 2nd deciles) exhibiting amino acid lengths around that of λ β (261 AA), whereas poorer hits (beyond the 2nd decile) were dominated by proteins of predicted length near 500 AA, which were largely annotated as ABC-related ATP binding cassettes. Sample hits <400 AA were then analyzed, regardless of Goodness of Fit, which are marked in light blue over the total candidate pool, marked in red (see FIG. 7A, 7B). This produced a pool of 77 candidates, of which 72 were successfully synthesized. Phylogenetic analysis suggested that the vast majority of these candidates fell into the redβ clade (0.943), except two homologs from the sak clade (P06778, NP005253777.1), two homologs from sak4 (YP_005321079.1, YP_002417106.1), and one from erf (YP_003467094.1). 10 homologs that have been previously shown to function in E. coli or other organisms (Datta et al., 2008; Eisen and Camerini-Otero, 1988; Sullivan et al., 2009) were synthesized. To understand the bias that known recombinases might impart on recombinase discovery, multiple library configurations which did not contain any known recombinases (Library 1.1), which contained the whole library minus λ β (Library 1.2), and which contained all 82 (including λ β, Library 1.3) were created. To perform SEER (see FIG. 1), 6 Rounds of Enrichment (RoE) were carried out with all three libraries and homologs were identified by Sanger sequencing.

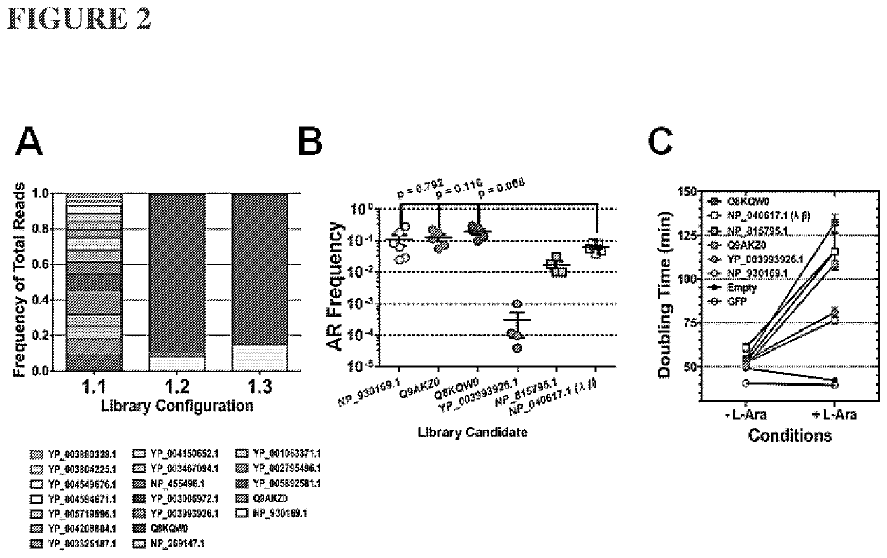

Sequencing showed that inclusion of known recombinases dramatically altered the solution at the 6th RoE (FIG. 2A), though the coarse distribution of recombinases was unchanged with or without λ β (FIG. 2A, compare 1.2 versus 1.3). The candidate top hits from Library 1.2/1.3 (NP_930169.1 from Photorhabdus luminescens, Q9AKZ0 from Legionella pneumophila, Q8KQW0 from Vibrio cholerae), were assembled along with NP_040617.1 (λ β), and two poorly-functional control recombinases from Library 1.1 (YP_003993926.1 from Halanaerobium hydrogeniformans, NP_815795.1 from Enterococcus faecalis) for direct quantification of allele recombination frequency (FIG. 2B). These experiments showed that Q8KQW0 from Vibrio cholerae performed significantly better at oligo recombination in E. coli (0.20±0.04, mean±SEM, **p=0.008 vs. NP_040617.1 [λ β]) than λ β (0.06±0.01). The other two candidates, NP_930169.1 (0.11±0.04, p=0.79) & Q9AKZ0 (0.13±0.03, p=0.116) were equivalent to λ β, suggesting multiple potential alternatives to λ β in E. coli. These findings quantitatively contrast previous work (Datta et al., 2008) where these particular recombinases performed significantly worse than λ β at oligo recombination in E. coli. Moreover, the same study showed that NP_815795.1 from Enterococcus faecalis performed slightly better than λ β in single test oligo recombinations at the galK locus, whereas in the present experiments, this recombinase is around 1 log worse (FIG. 2B, dark gray squares). Locus-to-locus variability (tolC on+strand at Chr. 3,176,137 here, versus galK on—strand at Chr. 789,202) and oligo length (90-mer's here, versus 70-mer's) may contribute to some of these differences.

The candidates were tested for toxicity using a kinetic growth assay of the candidates with and without L-arabinose induction. Doubling time was calculated and presented as the change without (‘−L-ara’) and with (‘+L-ara’) arabinose (FIG. 2C). Two negative controls were included that would not be expected to increase doubling time upon induction, pARC8.GFP (empty black circles) and an empty pARC8 vector (filled black circles). These pARC8 variants did not exhibit wildly different doubling times without arabinose (51.8±5.7 minutes, min: 40.5±0.5 minutes for GFP; max: 60.8±2.3 minutes for NP_040617.1 [λ β]), but do exhibit slower growth with arabinose (FIG. 2C). Candidates that support the highest recombination rate (λ β included, FIG. 2B) also exhibit the slowest doubling times upon induction (see squares), whereas less functional recombinases led to more modest increases in doubling time, and negative controls did not increase doubling time. These results demonstrate that some level of toxicity is accepted for robust recombination rates, and that the candidates identified offer desirable performance (FIG. 2B) with acceptable toxicity (FIG. 2C).

### Example III

### Deep Sequencing Analysis of SEER Library 2 in E. coli

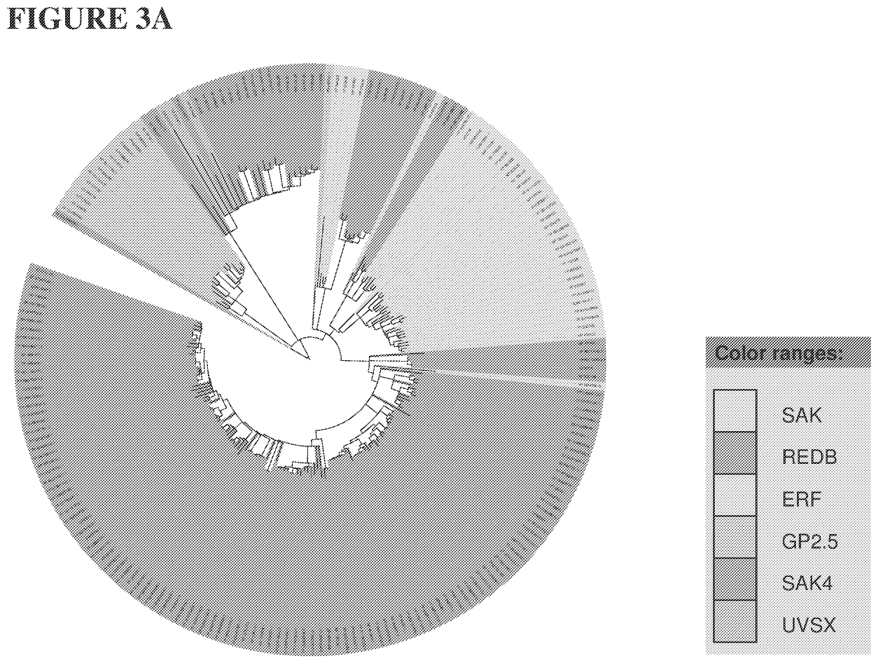

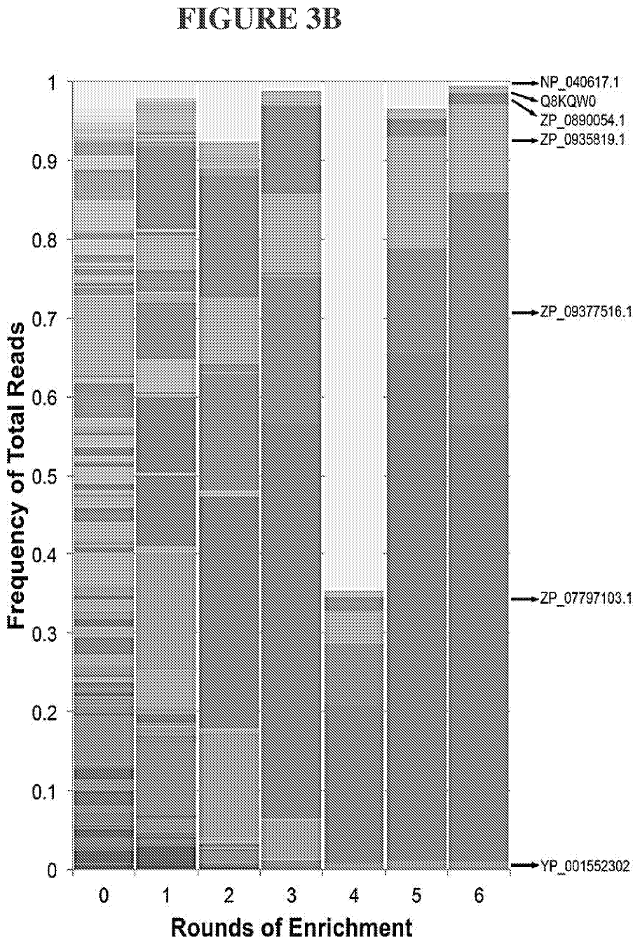

To develop a broader resource for recombinase discovery in non-standard model organisms and to sample the entirety of sequence space, a Hidden Markov Model-based search strategy was developed using multiple known recombinases to generate the position matrix with which to search nucleotide databases. This search returned ˜2,500 candidates that exhibited similar Goodness of Fit (FIG. 7C) and Polypeptide Length relationships (FIG. 7D) as the first library. Again, E. coli homologs were removed. Genes encoding less than 400 amino acids were the focus. The remaining distribution from the top 4 quintiles was sampled. Before synthesis, redundancy (defined as >98% amino acid identity) was removed which created a second library of 120 unique members, of which 113 were successfully synthesized using the same synthon design as Library 1. This new library (n=113) was combined with Library 1.3 (n=82) to generate Library 2 (n=195). A phylogenetic analysis is presented to show the diversity of SSAP clades covered by this new expansive library (FIG. 3A). Although Library 1.3 was heavily populated by members of redβ (0.943), the new library (n=113) has more balanced representation of all clades: redβ (0.400); erf (0.191); gp2.5 (0.127); sak (0.109); sak4 (0.146); and uvsX (0.027).

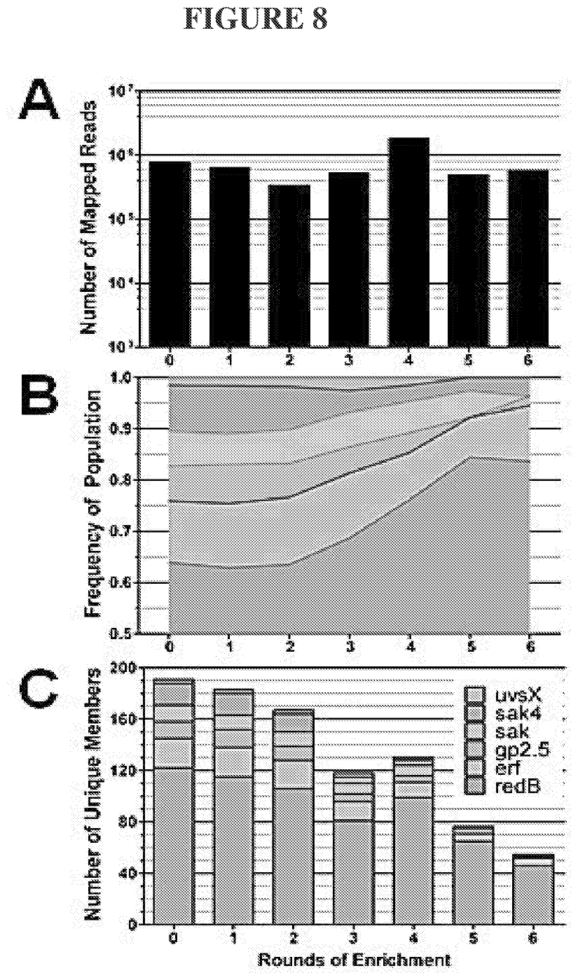

After complex assembly and verification of diversity, Library 2 was transformed and SEER in E. coli was performed. After the 6th RoE, the recombinase barcodes from 1E7 cells were PCR amplified before the first and after each RoE using indexed primers and sequenced using an Illumina MiSeq. After de-multiplexing and mapping, between 90.7 and 94.8% of raw reads corresponding to each RoE identically mapped to a recombinase barcode, resulting in a minimum read depth of 3.4E5 reads (see the 2nd RoE in FIG. 8A). Across all RoE, every recombinase was identified at least once throughout the experimental course.

193 of 195 total recombinases in the pre-SEER (0th RoE) population were identified, accurately reflecting the expected distribution of the clades in this library (FIG. 8B, 8C): redβ (0.63); erf (0.12); gp2.5 (0.07); sak (0.07); sak4 (0.09); and uvsX (0.2). There is notable skew in the population of library members, including the most abundant 31 recombinases (top 16%) accounting for 69% of the total reads, indicating significant assembly bias of some members over others. Over-abundant members included ZP_03935819.1_12 (0.100 of total reads), YP_950640_20 (0.062), EHN141107.1 (0.044), NP_040617.1 (λ β, 0.033), and two others greater than 0.03 frequency of total. Despite this skew, only 3/31 over-abundant recombinases emerged from the 6th RoE with a frequency of greater than 0.001: ZP_03935819.1_12 (redβ clade), 0.112; YP_001552302 (erf clade), 0.009; and NP_040617.1 (λ β), 0.005. These results suggest that some assembly bias can be tolerated by the power of serial enrichment.

Throughout the SEER workflow, the population diversity dropped from 193 at the 0th RoE to 55 unique members after the 6th RoE (FIG. 8C), though not strictly monotonically. Sampling bias in the 4th RoE (1.82E6 total reads) provided extra depth to identify 12 additional recombinases over those identified in reads from the 3rd RoE (0.52E6 total reads). Notably, 32 of the recombinases identified in the 4th RoE occurred below the frequency of detection of the 3rd RoE (1.91E-6), thus it is feasible to conclude that population diversity monotonically decreases with more RoE. Finally, relative clade abundance shifts in favor of redβ (FIG. 8B), whose members account for 0.639 of all reads at the 0th RoE and 0.840 of total at both the 5th and 6th RoE. At the 6th RoE, the erf clade accounts for 0.109 of total diversity, while sak4 (0.036) and gp2.5 (0.018) are also detected. Members of the uvsX clade are not detected after the 4th RoE, though at least 2 members were detected before that point, and were likely removed from the population by a sporadic spike in NP_040617.1 (λ β) abundance (0.645) at the 4th RoE. The abundance of NP_040617.1 (λ β) throughout the workflow (0.033, 0.017, 0.074, 0.010, 0.645, 0.033, 0.005, and Table 1) reflects this stark spike, however the population diversity doesn't reflect a bottleneck as diversity doesn't drop dramatically at the 4th RoE (FIG. 8C).

In terms of sheer abundance after the 6th RoE, desirable homologs included three redβ homologs, themselves accounting for 0.960 of all mapped reads: ZP_07797103.1 from Pseudomonas aeruginosa 39016, 0.556; ZP_09377516.1 from Hafnia alvei ATCC 51873, 0.292; and ZP_03935819.1 from Corynebacterium striatum ATCC 6940, 0.112 (FIG. 3A, Table 1). Using a phylogenetic analysis of the entire library (FIG. 3A), pair-wise comparisons of all members based on the branch length of the Newick string were computed. This showed that the 3 desirable homologs were in the 1st, 6th, and 6th closest deciles to NP_0406171.1 (λ β), highlighting the unpredictable nature of choosing candidate recombinases based on identity. The 6th, 7th, and 9th-most abundant recombinases at the 6th RoE (Table 1) were Q8KQW0 from Vibrio cholerae (0.009), NP_040617.1 (λ (3, 0.009), and Q9AKZ0 from Legionella pneumophila (0.005), corroborating results of SEER using Library 1.3 (FIG. 2A), and suggesting that desirable homologs from Library 2 may be equivalent or even better than λ β and/or Q8KQW0.

Enrichment factor (defined as freqₙ/freq₀ at nth RoE) is another way to consider relative performance that is less subject to skew at the 0th RoE (Table 2). At the 6th RoE, only 6 recombinases exhibited enrichment factors greater than 1.0 (Table 2), led by ZP_09377516.1 (170.1-fold) and ZP_07797103.1 (91.6-fold) that were the #2 and #1 most abundant recombinases at the 6th RoE. Also exhibiting enrichment factors >1 at the 6th RoE were Q8KQW0 (34.6-fold), Q9AKZ0 (2.80-fold), and ZP_08900554.1 from Gluconacetobacter oboediens 174Bp2 (3.53-fold). Enrichment factor at the 6th RoE is subject to complex population dynamics and propagation of sampling bias during the SEER workflow. For example, ZP_03935819.1, the most over-abundant recombinase in the starting pool (0.100) and the 3rd-most abundant recombinase at the end RoE (0.112), maintained its abundance through 6, suggesting that this candidate exhibited average performance within the context of the library. Enrichment factor is also presented after the 1st RoE (Table 3), which should be less subject to propagation biases, but more so to stochastic uncertainty. Here, only 16.6% of the pool expanded after one round, but eight candidates increased by at least 10-fold. However few persisted over all RoE. ZP_09377516.1 from Hafnia alvei, Q8KQW0 from Vibrio cholerae, & ZP_07797103.1 from Pseudomonas aeruginosa were advantageous. Two erf members, YP_001552302 from a Thalassaomonas phage, and YP_08900554.1 from an Enterobacter phage, start strong before declining in the face of many redβ competitors. These results offer a wealth of potential recombinases that could be leveraged in E. coli, but more importantly offer a representative workflow for recombinase discovery in non-standard model organisms.

### Example IV

### λ β Facilitates Annealing of Complementary SSB-Coated Oligonucleotides In Vitro

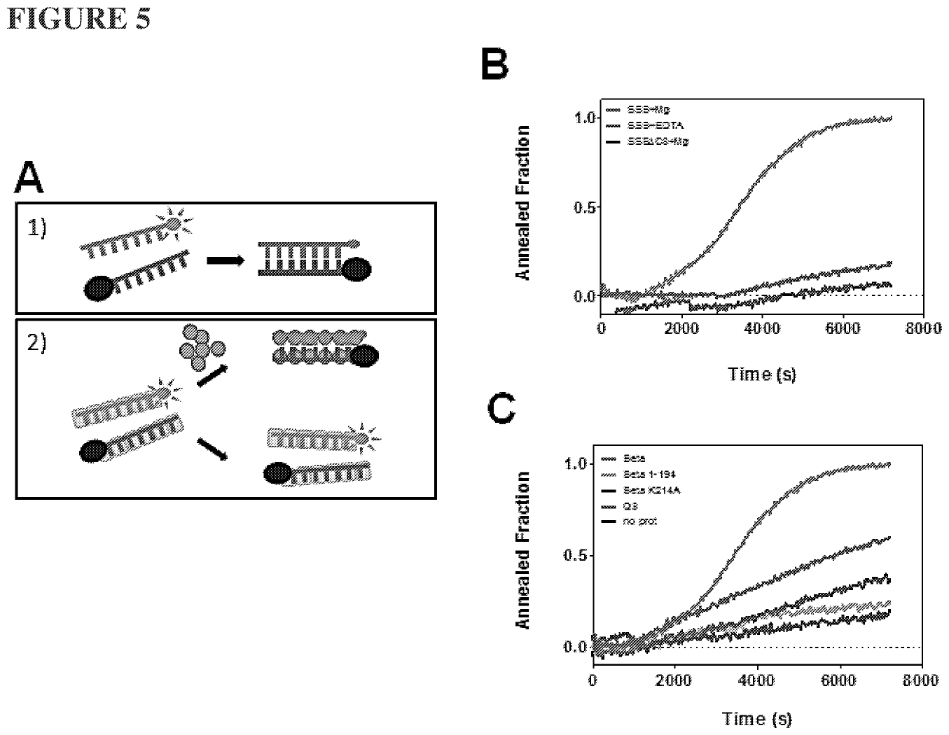

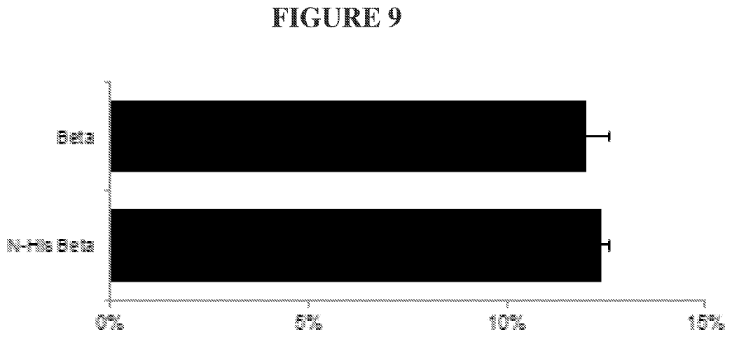

To understand more about the mechanisms of recombination, an in vitro oligo annealing assay was developed using two complementary 90mer oligos, one with a 3′-Fluorescein and the other with a 5′-Iowa Black FQ dark quencher (FIG. 5A). Upon mixing and incubating at 37° C., annealing reduces fluorescence over time. Annealing kinetics of the oligos is thus a platform with which the contributions of λ β or other SSAPs can be tested. To facilitate affinity purification, an N-terminal 6×His tag (SEQ ID NO: 108) was added and it was verified that the tag had no effect in its oligo recombination activity in E. coli (FIG. 9). Single stranded DNA binding proteins such as SSB protect ssDNA that is denatured during genome replication. Once bound, SSB inhibits complementary annealing {17272294}, until it is removed by the replisome or other interaction partners. Based on the functional analogy between λ β and RecO/Rad52 (which is a SSAP mediating annealing of complementary DNA strands and which is able to interact with the eukaryotic single-strand binding protein RPA) and the presence of SSB in some bacterial SSAP operons, the disclosure provides that an SSAP-SSB interaction represents a host-specific interaction node through which the SSAP interacts with the host system to facilitate recombination. An in vitro oligo quenching assay was set up using oligos pre-coated with SSB. In the absence of λ β, SSB-coated oligos support negligible annealing (FIG. 5C, purple curve). However, addition of λ β can overcome SSB pre-coating to facilitate annealing (FIG. 5B, 5C, red curve). Chelation of Mg²⁺ using EDTA significantly inhibits the annealing of SSB-coated oligos (FIG. 5B, blue curve), suggesting a Mg²⁺ requirement. To further characterize the λ β-SSB interaction, the effects of deleting eight C-terminal amino acids (DFDDDIPF (SEQ ID NO: 109)) of SSB (SSBAC8), previously shown to be dispensable for ssDNA binding but essential for its interaction with other proteins {8759000, 18937104}, and essential in vivo {8759000} was tested. λ β is not able to facilitate annealing of oligos that were pre-coated with SSBAC8 (FIG. 5B, green curve), suggesting that similar to other SSB-interacting proteins, λ β's interaction with SSB requires the SSB C-terminus. Compared to WT, the 1-194 truncation is unable to break the SSB inhibition, behaving very similarly to the SSB-only control (FIG. 5C, compare orange to purple curves). Similarly, K214A showed significantly decreased annealing of SSB-coated oligos (FIG. 5C, green curve).

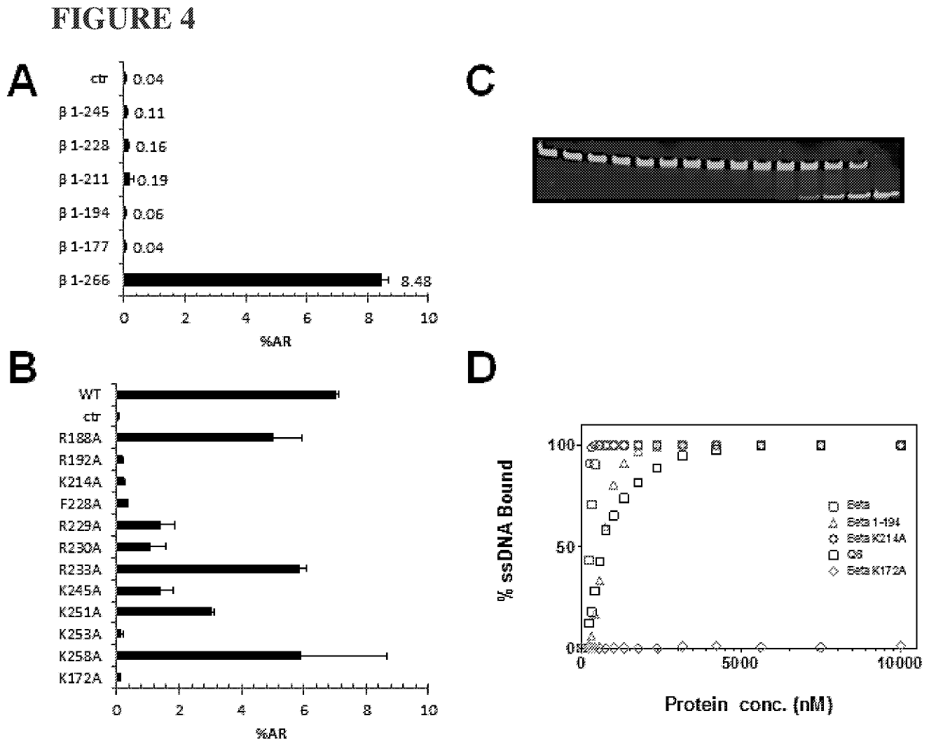

Q8KQW0 is an advantageous SSAP. This Vibrio cholerae SSAP showed slightly reduced ssDNA binding affinity compared to λ β (FIG. 4D, purple squares), and was able to anneal oligos coated with E. coli SSB (FIG. 5C, blue curve). Taken together, these results provide that λ β interacts with SSB-coated ssDNA to facilitate annealing, and that the C-terminus of λ β plays a significant role in this proposed interaction and this SSB-SSAP interaction is important for oligo recombination in vivo.

### Example V

### The C-Terminal of λ β is Required Oligo Recombination

To further understand the host tropism SSAPs exhibit, the C-terminus of λ β was studied. First, the 266 amino acid protein was serially truncated, generating fragments 1-245, 1-228, 1-211, 1-194 and 1-177, which is the smallest fragment previously found to be sufficient for DNA binding (Wu et al., 2006). Whereas full-length λ β is capable of achieving oligo recombinations at high frequencies (0.0848 in FIG. 4A), even the smallest C-terminal truncation tested 031-245) decreased function at least ˜77-fold, and larger serial truncations exhibit similar loss-of-function phenotypes. To further interrogate the C-terminus of λ β, K, R, & F amino acids were mutated to alanine and these variants were tested in oligo recombination. These mutations were chosen because similar substitution of basic and aromatic amino acids ablate the RecO-SSB interaction (Ryzhikov et al., 2011). Some of the mutations severely reduced recombination frequencies, especially R192A, K214A, & F228A (FIG. 4B). A negative control mutant K172A, previously shown to abolish DNA binding (Wu et al., 2006), also had negligible recombination rates. Since the mutation screen revealed that single amino acid substitutions in the C-terminus of λ β had dramatic functional consequences for oligo recombination frequencies in vivo, a subset of these mutants was characterized in vitro via a gel shift assay (FIG. 4C, 4D). In this assay K214A was undistinguishable from WT λ β at binding a ssDNA 90-mer, while the 1-194 truncation had somewhat reduced affinity. These results suggest that the λ β C-terminal truncation and point mutation have preserved DNA binding but impaired in vivo recombination, suggesting the C-terminus has a function distinct from DNA binding that is required for oligo recombination.

### Example V

### Porting a Species-Matched Heterologous SSB Enables Gain of Recombinase Function in E. coli

The in vitro data of FIG. 5B-C provides that the species tropism seen in SSAPs is based on its ability to interact with SSB from ssDNA in a given model organism. To further evaluate this, foreign SSAPs were tested for a gain of function when they were co-expressed with a phylogenetically-matched SSB homolog. To test this in E. coli, an inducible, bi-cistronic vector was generated to express a candidate SSAP and either a matched or mismatched SSB. As candidates, SSAPs and SSBs were selected from E. coli, Lactobacillus reuteri {22328729}, and Corynebacterium glutamicum {23630315}, which are known to enable recombineering in their respective host organisms. To facilitate bi-cistronic expression of both ORFs in our pARC8-based vector, an RBS-containing motif (aaaataAGGAGGAaaaacat)(SEQ ID NO:10) was added downstream of the SSAP stop codon and upstream of an aadA coding region, which confers spectinomycin resistance. This plasmid conferred spectinomycin resistance in the presence of 0.2% L-arabinose, albeit supporting growth rates that were significantly slower than chromosomal, monocistronic expression of an aadA cassette.

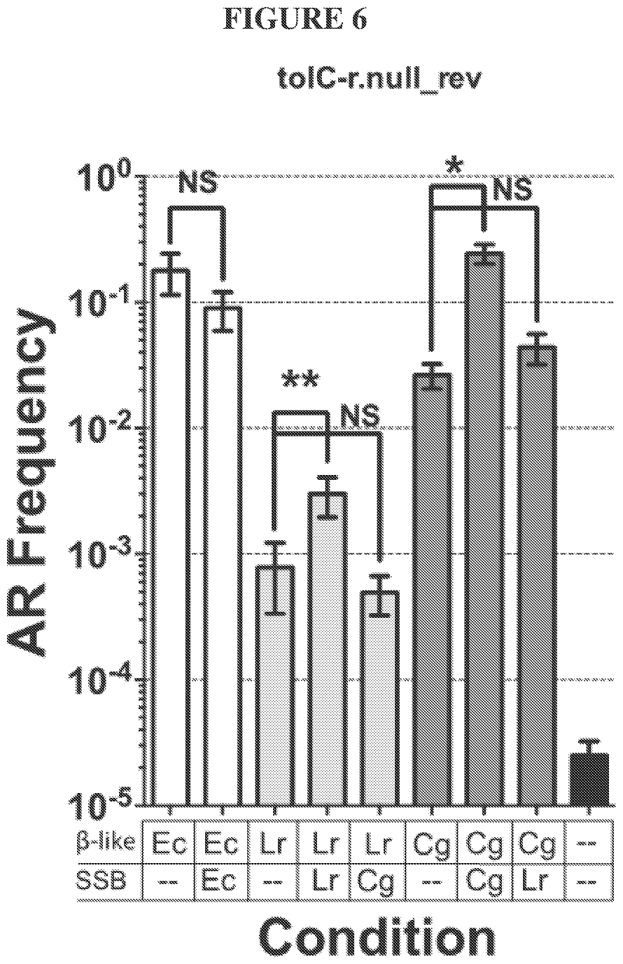

pARC8 variants were constructed containing SSAPs only, properly matched SSAP-SSB pairs (e.g., λ β+EcSSB, Lr.recT1+LrSSB, or Cg.recT+CgSSB), or mismatched SSAP/SSB pairs (e.g., Lr.recT1+CgSSB, or Cg.recT+LrSSB). Induction was performed using 0.2% L-arabinose and oligo recombinations were performed to quantify AR Frequency (FIG. 6). Background (no plasmid) AR frequency was measured at 2.51E-5±1.48E-5 in E. coli, whereas λ β supported AR frequencies of 1.79E-1±1.29E-1 (mean±std. dev., **p<0.01, pARC8.λ β vs. background), consistent with a ˜1E4-fold increase, similar to that seen previously using λ Red in E. coli {11381128}. Bi-cistronic expression of E. coli λ β+EcSSB did not have a significant effect on fitness or on AR frequency (0.90E-1±0.68E-1, p=0.20, pARC8.λ β vs. pARC8.λ β_EcSSB). Although maximal over-expression of a heterologous SSB (LrSSB or CgSSB) may lead to toxicity, bi-cistronic overexpression using this system did not have any apparent effect on fitness in E. coli, suggesting that their expression is tolerated, at least at this level.

L. reuteri recT1 supported only a 15-fold increase in recombination rate over background in E. coli (3.13E-4±2.10E-4, p=ns pARC8.LrrecT1 vs background). However adding its cognate SSB further increased recombination frequency by ˜10-fold (3.00E-3±2.09E-3, *p<0.05 pARC8.LrrecT1_LrSSB vs pARC8.LrrecT1). An unrelated SSB (pARC8.LrrecT1_CgSSB) did not support this gain of function phenotype (4.96E-4±2.95E-4, p=ns, pARC8.LrrecT1_CgSSB vs pARc8.LrrecT1), nor did co-expressing E. coli SSB, suggesting that a functional relationship must be maintained between recombinase and SSB in the context of the host organism. This pattern was maintained with C. glutamicum recT. Although Cg.recT supported 1E3-fold increase in recombination rates over background (2.64E-2±1.18E-2), adding its cognate SSB increased AR frequencies another ˜10-fold to 2.45E-1±8.68E-2 (*p<0.05, pARC8.CgrecT_CgSSB vs pARC8.CgrecT), whereas an unrelated SSB (pARC8.CgrecT_LrSSB) does not support any gain-of-function (4.40E-2±2.40E-2, p=ns, pARC8.CgrecT_LrSSB vs pARC8.CgrecT).

The disclosure provides that oligo recombination via heterologous SSAPs is enhanced by expressing its corresponding SSB, further highlighting the importance of the SSAP-SSB interaction. The disclosure identifies useful SSAP candidates other than λ β. The disclosure provides that the C terminus of λ β facilitates recombination. The disclosure provides that proper function of the SSAP C-terminus is required for the λ β-SSB interaction. The disclosure provides that λ β-SSB interaction requires the extreme C-terminus of SSB implicating a protein-protein interaction.

The disclosure provides methods of recombineering or genome editing using an SSAP paired with its phylogenetically-matched SSB homolog in a foreign host cell. A cell is genetically modified to include a nucleic acid encoding the SSAP and a nucleic acid encoding the SSB. The nucleic acids are expressed by the cell. The SSAP and the SSB interact and a single stranded DNA is included in the genome of the cell. The disclosure provides that an SSAP-SSB pair is a minimally functional set required to port recombineering into non-standard model organisms.

### Tables

TABLE 1
Exemplary Recombinases for Use in the Recombineering Methods Described herein.
Frequency of Recombinases in Library 2 throughout SEER Workflow. These data are
sorted by the frequency at 6 RoE from largest to smallest. This is a subset of
Table S1, including only the top 25 most abundant recombinases at the 6th RoE.

| | | Frequency of Recombinase in Population at Indicated RoE | | | | | | |
|---|---|---|---|---|---|---|---|---|
| Accession ID | Clade | 0 | 1 | 2 | 3 | 4 | 5 | 6 |
| ZP_07797103.1 | redB | 0.006069 | 0.087155 | 0.252425 | 0.496366 | 0.201227 | 0.645734 | 0.556229 |
| ZP_09377516.1 | redB | 0.001715 | 0.065338 | 0.148232 | 0.187097 | 0.075322 | 0.129997 | 0.291793 |
| ZP_03935819.1 | redB | 0.099945 | 0.043402 | 0.085366 | 0.101573 | 0.04141 | 0.141074 | 0.112076 |
| ZP_08900554.1 | redB | 0.004043 | 0.103575 | 0.147504 | 0.109183 | 0.016881 | 0.022072 | 0.014275 |
| YP_001552302 | erf | 0.010264 | 0.147288 | 0.130424 | 0.047892 | 0.007072 | 0.011151 | 0.009077 |
| Q8KQW0 | redB | 0.000247 | 0.004216 | 0.01591 | 0.011619 | 0.006185 | 0.011982 | 0.008571 |
| NP_040617.1 | redB | 0.032941 | 0.016828 | 0.073765 | 0.010528 | 0.645134 | 0.033432 | 0.004992 |
| ZP_04808991.1 | redB | 0.002255 | 0.004112 | 0.003903 | 0.001754 | 0.000846 | 0.000977 | 0.001477 |
| Q9AKZ0 | redB | 0.000225 | 0.007636 | 0.01179 | 0.006744 | 0.001488 | 0.001833 | 0.000631 |
| ZP_01947910.1 | redB | 0.002619 | 0.004029 | 0.004605 | 0.001445 | 0.001273 | 0.000281 | 0.000342 |
| ZP_06691943.1 | redB | 0.013573 | 0.001012 | 0.000515 | 6.11E−05 | 3.19E−05 | 0.000203 | 0.000127 |
| YP_001429745 | redB | 0.006977 | 0.00115 | 0.004247 | 0.000487 | 0.000224 | 0.000221 | 0.000108 |
| ZP_07463816.1 | redB | 0.014396 | 0.048178 | 0.008293 | 0.003843 | 0.000267 | 0.000344 | 9.07E−05 |
| NP_958297 | redB | 0.005724 | 0.000757 | 7.4E−05 | 3.05E−05 | 9.88E−06 | 7.44E−05 | 6.11E−05 |
| ZP_08564335.1 | redB | 0.006915 | 0.095269 | 0.040859 | 0.005662 | 0.000433 | 5.43E−05 | 2.44E−05 |
| YP_001285543 | erf | 0.004234 | 0.094347 | 0.013147 | 0.008766 | 0.000237 | 0.000193 | 1.22E−05 |
| YP_001285915 | erf | 0.017154 | 0.028527 | 0.002657 | 0.00034 | 5E−05 | 1.21E−05 | 8.72E−06 |
| YP_003006972.1 | redB | 0.000301 | 6.68E−05 | 2.96E−06 | 1.91E−06 | 1.98E−05 | 2.01E−06 | 5.23E−06 |
| YP_004150652.1 | redB | 0.001105 | 0.000227 | 4.15E−05 | 0 | 6.54E−05 | 1E−05 | 5.23E−06 |
| YP_112530 | erf | 0.010894 | 0.00159 | 0.000231 | 1.14E−05 | 5.49E−07 | 0 | 5.23E−06 |
| YP_005892581.1 | redB | 0.000372 | 5.29E−05 | 0.000107 | 1.91E−06 | 8.68E−05 | 1.61E−05 | 5.23E−06 |
| NP_958570 | erf | 0.006934 | 0.000561 | 9.77E−05 | 0 | 5.49E−07 | 2.01E−06 | 5.23E−06 |
| Q7N2Y8 | redB | 0.000445 | 1.55E−06 | 8.88E−06 | 0 | 8.24E−06 | 0 | 5.23E−06 |

TABLE 2
Exemplary Recombinases for Use in the Recombineering Methods Described
herein. Enrichment Factor of Recombinases from Library 2. Here, enrichment factor
is calculated as freqₙ/freq₀, where n is the RoE. These data are sorted
by the enrichment factor from the 6th RoE, from largest to smallest,
showing only recombinases where enrichment factor >1.

| | | Enrichment Factor (count(n)/count(0) at Indicated n | | | | | | |
|---|---|---|---|---|---|---|---|---|
| Accession ID | Clade | 0 | 1 | 2 | 3 | 4 | 5 | 6 |
| ZP_09377516.1 | redB | 1 | 38.09 | 86.42 | 109.08 | 43.91 | 75.79 | 170.12 |
| ZP_07797103.1 | redB | 1 | 14.36 | 41.59 | 81.79 | 33.16 | 106.40 | 91.65 |
| Q8KQW0 | redB | 1 | 17.04 | 64.29 | 46.95 | 24.99 | 48.42 | 34.64 |
| ZP_08900554.1 | redB | 1 | 25.62 | 36.48 | 27.00 | 4.18 | 5.46 | 3.53 |
| Q9AKZ0 | redB | 1 | 33.91 | 52.35 | 29.94 | 6.61 | 8.14 | 2.80 |
| ZP_03935819.1 | redB | 1 | 0.43 | 0.85 | 1.02 | 0.41 | 1.41 | 1.12 |
| YP_001552302 | erf | 1 | 14.35 | 12.71 | 4.67 | 0.69 | 1.09 | 0.88 |
| YP_003010343.1 | redB | 1 | 0 | 1.13 | 0.73 | 0.63 | 0 | 0.67 |
| ZP_04808991.1 | redB | 1 | 1.82 | 1.73 | 0.78 | 0.38 | 0.43 | 0.66 |
| NP_040617.1 | redB | 1 | 0.51 | 2.24 | 0.32 | 19.58 | 1.01 | 0.15 |

TABLE 3
Exemplary Recombinases for Use in the Recombineering Methods Described
herein. Enrichment Factor of Recombinases from Library 2. Here, enrichment factor
is calculated as freqₙ/freq₀, where n is the RoE. These data are sorted
by the enrichment factor from the 1st RoE, from largest to smallest,
showing only recombinases where enrichment factor >1.

| | | Enrichment Factor (freq(n)/freq(0) at Indicated n | | | | | | |
|---|---|---|---|---|---|---|---|---|
| Accession ID | Clade | 0 | 1 | 2 | 3 | 4 | 5 | 6 |
| ZP_09377516.1 | redB | 1 | 38.10 | 86.40 | 109.00 | 43.90 | 75.80 | 170.00 |
| Q9AKZ0 | redB | 1 | 33.90 | 52.40 | 29.90 | 6.61 | 8.14 | 2.80 |
| ZP_08900554.1 | redB | 1 | 25.60 | 36.50 | 27.00 | 4.18 | 5.46 | 3.53 |
| YP_001285543 | erf | 1 | 22.30 | 3.10 | 2.07 | 0.06 | 0.05 | 0 |
| Q8KQW0 | redB | 1 | 17.00 | 64.30 | 47.00 | 25.00 | 48.40 | 34.60 |
| ZP_07797103.1 | redB | 1 | 14.40 | 41.60 | 81.80 | 33.20 | 106.00 | 91.70 |
| YP_001552302 | erf | 1 | 14.40 | 12.70 | 4.67 | 0.69 | 1.09 | 0.88 |
| ZP_08564335.1 | redB | 1 | 13.80 | 5.91 | 0.82 | 0.06 | 0.01 | 0 |
| NP_815795.1 | redB | 1 | 4.83 | 2.77 | 0.22 | 0.04 | 0 | 0.01 |
| YP_003150705.1 | redB | 1 | 3.71 | 1.89 | 0.18 | 0.01 | 0 | 0 |
| ZP_07463816.1 | redB | 1 | 3.35 | 0.58 | 0.27 | 0.02 | 0.02 | 0.01 |
| YP_004875641.1 | redB | 1 | 3.27 | 0.65 | 0.05 | 0 | 0 | 0 |
| NP_463513.1 | redB | 1 | 2.51 | 0.50 | 0.01 | 0.01 | 0 | 0 |
| ZP_03706978.1 | redB | 1 | 2.04 | 0.24 | 0.01 | 0 | 0 | 0 |
| ZP_04808991.1 | redB | 1 | 1.82 | 1.73 | 0.78 | 0.38 | 0.43 | 0.66 |
| YP_002233655.1 | redB | 1 | 1.72 | 0.34 | 0.02 | 0 | 0 | 0 |
| YP_001285915 | erf | 1 | 1.66 | 0.16 | 0.02 | 0 | 0 | 0 |
| NP_076707 | redB | 1 | 1.60 | 0.20 | 0.05 | 0 | 0 | 0 |
| ZP_01947910.1 | redB | 1 | 1.54 | 1.76 | 0.55 | 0.49 | 0.11 | 0.13 |
| YP_002995484.1 | redB | 1 | 1.48 | 0.11 | 0.01 | 0 | 0 | 0 |
| YP_003084246.1 | redB | 1 | 1.46 | 0.28 | 0 | 0.05 | 0 | 0 |
| YP_001293439 | sak4 | 1 | 1.22 | 0.25 | 0.01 | 0 | 0 | 0 |
| NP_455496.1 | redB | 1 | 1.21 | 0.27 | 0.02 | 0.13 | 0.01 | 0 |
| YP_001409109.1 | redB | 1 | 1.19 | 2.26 | 0 | 10.90 | 1.54 | 0 |
| YP_004479394.1 | redB | 1 | 1.03 | 0.75 | 0 | 0 | 0.04 | 0 |

TABLE 4
Recombinases.
RECOMBINASES

| Saccharomyces cerevisiae | Homo sapiens | Saprospira grandis |
|---|---|---|
| Shewanella putrefaciens | Caldicellulosiruptor | Cyanophage pSS2 |
| | kristjanssonii | |
| Anaeroce/lum thermophilum | M yxococcus fulvus | Caldicellulosiruptor |
| | | hydrothermalis |
| Mycobacterium marinum | Photorhabdus luminescens | Myxococcus xanthus |
| Corallococcus coralloides | Persephone/la marina | Legionella pneumophila |
| Burkholderia pseudomallei | Stigmatella aurantiaca | Caldicellulosiruptor |
| | | saccharolyticus |
| Rhizobium loti | Vibrio cholerae | Listeria phage A118 |
| Salmonella newport | Xylanimonas cellulosilytica | Caldice/lulosiruptor |
| | | kronotskyensis |
| Delftia sp. | Paracoccus denitrificans | Pelobacter propionicus |
| Psychrobacter sp. | Clostridium cellulovorans | Bifidobacterium longum subsp. |
| | | infantis |
| Sinorhizobium meliloti | Campylobacter curvus | Corynebacterium variabile |
| Xylella fastidiosa | Photorhabdus luminescens | Escherichia coli |
| | subsp. laumondii | |
| Lactotoccus phage u/16.2 | Enterobacter sp. | Clostridium difficile |
| Paenibacillus sp. | A/lochromatium vinosum | Acidithiobacillus caldus |
| Vibrio splendidus | Carboxydothermus | Campylobacter jejuni subsp. |
| | hydrogenoformans | doylei |
| Soda/is glossinidius | Spirochaeta smaragdinae | Haemophilus parasuis serovar 5 |
| Gluconobacter oxydans | Halanaerobium sp. | Enterobacter cloacae subsp. |
| | | cloacae |
| Neisseria meningitidis | Enterobacter aerogenes | Yersinia pseudotuberculosis |
| serogroup A | | |
| Bartone/la grahamii | Bartone/la tribocorum | Shewanelasp. |
| Proteus mirabilis | Streptococcus pneumoniae | Pseudomonas fluorescens |
| Vibrio cholerae | Alkaliphilus metalliredigens | Streptococcus pyogenes serotype |
| serotype 01 | | M12 |
| Streptococcus pyogenes | Laribacter hongkongensis | Salmonella paratyphi B |
| serotype Ml | | |
| Salmonella heidelberg | Escherichia fergusonii | Hydrogenobacter thermophilus |
| Shigella dysenteriae serotype 1 | Salmonella typhi | Aggregatibacter aphrophilus |
| Listeria innocua serovar 6a | Drosophila melanogaster | Streptococcus pyogenes serotype |
| | | MS |
| Xenorhabdus bovienii | Streptococcus parauberis | Thermovibrio ammonificans |
| Streptococcus salivarius | Frateuria aurantia | Melissococcus plutonius |
| Avibacterium paragallinarum | Salmonella enterica subsp. | Haemophilus influenzae R3021 |
| | enterica serovar Typhi str. 404ty | |
| Xylella fastidiosa Ann- 1 | Bartone/la schoenbuchensis Rl | Enterobacteria phage lambda |
| Gluconacetobacter oboediens | Helicobacter cinaedi CCUG | Bartone/la sp. AR 15-3 |
| 174Bp2 | 18818 | |
| Listeria monocytogenes FSL | Listeria ivanovii FSL F6-596 | Paenibacillus larvae subsp. larvae |
| R2-503 | | B-3650 |
| Escherichia fergusonii ECD227 | Enterobacteria phage Min27 | Salmonella enterica subsp. |
| | | enterica serovar Rubislaw str. |
| | | A4-653 |
| Myxococcus fulvus HW-1 | Salmonella enterica subsp. | Salmonella enterica subsp. |
| | enterica serovar Javiana str. | enterica serovar Senftenberg str. |
| | GA_MM04042433 | A4-543 |
| Shigella dysenteriae | Salmonella enterica subsp. | Myxococcus xanthus DK 1622 |
| | enterica serovar Typhi str. AG3 | |
| Shigella dysenteriae 1617 | Caldicellulosiruptor | Caldicellulosiruptor |
| | hydrothermalis 108 | kristjanssonii 177R1B |
| Corallococcus cora/loides DSM | Stigmatella aurantiaca DW4/3-1 | Caldice/lulosiruptor |
| 2259 | | saccharolyticus DSM 8903 |
| Providencia rettgeri | Lactococcus phage phismq86 | Caldicellulosiruptor |
| | | kronotskyensis 2002 |
| Neisseria meningitidis WUE | Neisseria lactamica ¥92-1009 | Burkholderia pseudomallei |
| 2594 | | NCTC 13177 |
| Clostridium botulinum C str. | Clostridium cellulovorans 7438 | Xenorhabdus nematophila ATCC |
| Eklund | | 19061 |
| aribacter hongkongensis | Burkholderia pseudomallei 668 | Alkaliphilus metalliredigens |
| HLHK9 | | QYMF |
| Klebsiella pneumoniae | Vibrio cholerae 0139 | Yersinia ruckeri |
| Haemophilus haemolyticus | Cyanophage PSS2 | Providencia rettgeri DSM 1131 |
| M21639 | | |
| Lactococcus phage phiLC3 | Frateuria aurantia DSM 6220 | Haemophilus influenzae NT127 |
| Streptococcus phage 2167 | Aggregati bacter aphrophilus | Streptococcus pneumoniae EU- |
| | NJ8700 | NP01 |
| Me/issococcus plutonius ATCC | Streptococcus infantis SK970 | Salmonella enterica subsp. |
| 35311 | | enterica serovar Montevideo str. |
| | | SARB30 |
| Bacillus subtilis subsp. | Enterobacter cloacae subsp. | Photobacterium damselae subsp. |
| spizizenii TU-B-10 | cloacae ATCC 13047 | damselae |
| Neisseria bacilliformis ATCC | Clostridium botulinum C sir. | Enterobacter aerogenes KCTC |
| BAA-1200 | Stockholm | 2190 |
| Pseudomonas aeruginosa 39016 | Bartonella rochalimae ATCC | Fusobacterium sp. 3_1_5R |
| | BAA-1498 | |
| Comamonas testosteroni S44 | Pediococcus acidilactici DSM | Salmonella enterica subsp. |
| | 20284 | houtenae str. ATCC BAA-1581 |
| Sodalis glossinidius str. | Streptococcus parauberis KCTC | Bartonella tribocorum CIP |
| ‘morsitans’ | 11537 | 105476 |
| Klebsiella sp. 1_1_55 | Lactobacillus ruminis SPM0211 | Haemophilus |
| | | paraphrohaemolyticus HK411 |
| Clostridium sporogenes PA | Mannheimia haemolytica | Klebsiella pneumoniae subsp. |
| 3679 | PHL213 | rhinoscleromatis ATCC 13884 |
| Streptococcus phage 8140 | Sinorhizobium meliloti SMll | Streptococcus phage V22 |
| Clostridium botulinum El str. | Streptococcus phage 9429.2 | Spirochaeta smaragdinae DSM |
| ‘Bo NT E Beluga’ | | 11293 |
| Sinorhizobium meliloti AK83 | Streptococcus pyogenes str. | Carboxydothermus |
| | Manfredo | hydrogenoformans Z-2901 |
| Streptococcus pneumoniae | Klebsiella pneumoniae KCTC | Streptococcus salivarius JIM8777 |
| GA11426 | 2242 | |
| Acinetobacter sp. SH024 | Burkholderia sp. Chl-1 | Streptococcus suis 89/1591 |
| Mesorhizobium loti | Streptococcus suis SS12 | listeria monocytogenes FSL Nl- |
| MAFF303099 | | 017 |
| lodobacteriophage phiPLPE | Bartonella grahamii as4aup | Fusobacterium sp. 12_1B |
| Cryptobacterium curtum DSM | Bacteroides caccae ATCC | Leptotrichia goodfellowii F0264 |
| 15641 | 43185 | |
| Listeria innocua Clip11262 | Vibrio cholerae non-01/non- | Acinetobacter baumanniiAB900 |
| | 0139 | |
| Halanaerobium | Mycobacterium marinum M | Peptoniphilus duerdenii ATCC |
| hydrogeniformans | | BAA-1640 |
| Serratia symbiotica str. Tucson | Bifidobacterium longum subsp. | Campylobacter jejuni subsp. |
| | infantis 157F | doylei 269.97 |
| Xylella fastidiosa subsp. | Xylella fastidiosa 9a5c | Acinetobacter sp. P8-3-8 |
| fastidiosa GB514 | | |
| Streptococcus phage M102 | Campylobacter coli 1957 | Corynebacterium variabile DSM |
| | | 44702 |
| Collinsella stercoris DSM | Campylobacter curvus 525.92 | Campylobacter jejuni subsp. |
| 13279 | | jejuni 2008-988 |
| Campylobacter coli 67-8 | Campylobacter coli LMG 9860 | Campylobacter coli 2685 |
| Campylobacter coli 132-6 | Sebaldella termitidis ATCC | Xylanimonas cellulosilytica |
| | 33386 | DSM 15894 |
| Campylobacter coli 80352 | Collinsella aerofaciens ATCC | Aggregatibacter segnis ATCC |
| | 25986 | 33393 |
| Haemophilus parasuis SH0165 | Pelobacter propionicus DSM | Haemophilus parasuis 29755 |
| | 2379 | |
| Campylobacter upsaliensis | Acidithiobacillus caldus SM-1 | Acidithiobacillus caldus ATCC |
| RM3195 | | 51756 |
| Streptococcus gallolyticus | Yersinia pseudotuberculosis IP | Burkholderia pseudomallei |
| subsp. gallolyticus TX20005 | 32953 | Pasteur 52237 |
| Sinorhizobium meliloti | Vibrio furnissii CIP 102972 | Acetobacteraceae bacterium AT- |
| CCNWSX0020 | | 5844 |
| Helicobacter pullorum MIT 98- | Pseudomonas fluorescens | Photobacterium damselae subsp. |
| 5489 | SBW25 | damselae CIP 102761 |
| Sinorhizobium medicae | Hydrogenobacter thermophil us | Simkania negevensis z |
| WSM419 | TK-6 | |
| Rhizobium leguminosarum bv. | Persephonella marina EX-Hl | Thiocapsa marina 5811 |
| trifolii WSM2304 | | |
| Thiorhodococcus drewsii AZl | Desuifitobacterium | Allochromatium vinosum DSM |
| | metallireducens DSM 15288 | 180 |
| Vibrio splendidus LGP32 | Thermus aquaticus Y51MC23 | Enterobacter sp. 638 |
| Thiocystis violascens DSM 198 | Vibrio cholerae 1587 | Brevundimonas diminuta ATCC |
| | | 11568 |
| Delftia sp. Csl-4 | Corynebacterium diphtheriae C7 | Cronobacter phage ENT47670 |
| | (beta) | |
| Thiorhodovibri o sp. 970 | Paenibacillus sp. JDR-2 | Photorhabdus luminescens subsp. |
| | | laumondii TTOl |
| Psychrobacter sp. PRwf-1 | Aeromonas salmonicida subsp. | Gluconobacter oxydans 621H |
| | salmonicida 01-6526 | |
| Sporosarcina newyorkensis | Acidovorax delafieldii 2AN | Paenibacillus elgii B69 |
| 2681 | | |
| Methylomicrobium album BG8 | Enterobacteriacea e bacterium | Paracoccus denitrificans PD1222 |
| | 9_2_54FAA | |
| Commensalibacter intestini | Mobiluncus curtisii ATCC | Listeria monocytogenes str. 1/2a |
| A911 | 51333 | F6854 |
| Pseudomonas syringae pv. pisi | Listeria monocytogenes FSL J2- | Pseudomonas syringae pv. |
| str. 17048 | 003 | lachrymans str. M301315 |
| Xanthobacter autotrophicus Py2 | Ureaplasma parvum serovar 6 | Rhodococcus phage REQ3 |
| | str. ATCC 27818 | |
| Ureaplasma urealyticum serovar | Ureaplasma urealyticum serovar | Halomonas elongata DSM 2581 |
| 8 str. ATCC 27618 | 12 str. ATCC 33696 | |
| Klebsiella oxytoca 10-5250 | Paenibacillus larvae subsp. | Hafnia alvei ATCC 51873 |
| | larvae BRL-230010 | |
| Burkholderia cenocepacia J2315 | Gardnerella vaginalis 409-05 | Proteus penneri ATCC 35198 |
| Acinetobacter baumannii ATCC | Staphylococcus aureus subsp. | Pseudoalteromonas sp. BSi20495 |
| 19606 | aureus D139 | |
| Parabacteroides johnsoni i DSM | Citrobacter koseri ATCC BM- | Borrelia hermsii |
| 18315 | 895 | |
| Salmonella enterica subsp. | Salmonella phage SPN1S | Salmonella enterica subsp. |
| enterica serovar Mississippi str. | | enterica serovar Kentucky str. |
| A4-633 | | CDC 191 |
| Shigella dysenteriae CDC 74- | Corynebacterium striatum | Klebsiella sp. MS 92-3 |
| 1112 | ATCC 6940 | |
| Pantoea sp. aB | Proteus mirabilis ATCC 29906 | Providencia alcalifaciens DSM |
| | | 30120 |
| Escherichia phage TL-2011b | Clostridium methylpentosum | Erwinia phage phiEt88 |
| | DSM 5476 | |
| Gemella sanguinis M325 | Serratia odorifera DSM 4582 | Pseudomonas brassicacearum |
| | | subsp. brassicacearum NFM421 |
| Comamonas testosteroni KF-1 | Edwardsiella tarda ATCC 23685 | Brenneria sp. EniD312 |
| Legionella pneumophila | Enterococcus faecalis TX0630 | Edwardsiella ictaluri 93-146 |
| Edwardsiella tarda EIB202 | Acinetobacter radioresistens | Chelativorans sp. BNC1 |
| | SK82 | |
| Moraxella catarrhalis 101P30B1 | Clostridium botulinum B str. | Xenorhabdus bovienii SS-2004 |
| | Eklund 178 | |
| Erwinia billingiae Eb661 | EBPR podovirus 1 | Methylobacterium nodulans ORS |
| | | 2060 |
| Pantoea vagans C9-1 | Oceanicola sp. 5124 | Salmonella enterica subsp. |
| | | enterica serovar Gaminara str. |
| | | A4-567 |
| Klebsiella pneumoniae subsp. | Thermovibrio ammonificans | Corynebacterium glutamicum R |
| pneumoniae MGH 78578 | HB-1 | |
| Phage Gifsy-1 | Escherichia phage TL-2011c | Salmonella enterica subsp. |
| | | enterica serovar Typhimurium |
| Vibrio angustum S14 | Corynebacterium ulcerans BR- | Listeria monocytogenes F5L F2- |
| | AD22 | 515 |
| Mycobacterium abscessus M93 | Psychrobacter arcticus 273-4 | Ahrensia sp. R2A130 |
| Thermoanaerobacter italicus | Rhodomicrobium vannielii | Acinetobacter radioresistens |
| Ab9 | ATCC 17100 | SH164 |
| Photobacterium profundum SS9 | Segniliparus rotundus DSM | Megamonas funiformis YIT |
| | 44985 | 11815 |
| Pseudomonas syringae pv. | Pseudomonas syringae pv. | Moraxella catarrhalis BCl |
| syringae B728a | maculicola str. ES4326 | |
| Eubacterium saburreum DSM | Clostridium hathewayi DSM | achnospiraceae bacterium |
| 3986 | 13479 | 9_1_43BFAA |
| Eubacteriaceae bacterium | Burkholderia sp. CCGE1002 | Bacteroides sp. 2_1_33B |
| ACC19a | | |
| Nitratifractor salsuginis DSM | Jonesia denitrificans DSM | Agrobacterium tumefaciens |
| 16511 | 20603 | |
| Aeromonas hydrophila | butyrate-producing bacterium | Enterobacter hormaechei ATCC |
| | 553/4 | 49162 |
| Pseudomonas aeruginosa | Pseudomonas phage phi297 | Klebsiella variicola At-22 |
| Burkholderia sp. TJl49 | Klebsiella oxytoca 10-5243 | Burkholderia phymatum 5TM815 |
| Pseudomonas putida GB-1 | Klebsiella pneumoniae 342 | Shewanella putrefaciens 200 |
| Saprospira grandis str. Lewin | Mycobacterium abscessus M94 | Bradyrhizobium sp. STM 3843 |
| Bacillus subtilis subsp. natto | Sodalis phage phiSG1 | Parachlamydia acanthamoebae |
| BEST195 | | str. Hall's coccus |
| Achromobacter xylosoxidans | Desulfovibrio piger ATCC | Burkholderia dolosa AU0158 |
| AXX-A | 29098 | |
| delta proteobacterium NaphS2 | Vibrio caribbenthicus ATCC | Dyadobacter fermentans DSM |
| | BAA-2122 | 18053 |
| Kordia algicida OT-1 | Flavobacterium indicum | Vibrio harveyi 1DA3 |
| | GPTSAl00-9 | |
| Runella slithyformis DSM | Flavobacterium johnsoniae | Owenweeksia hongkongensis |
| 19594 | UW101 | DSM 17368 |
| Riemerella anatipestifer RA-GD | Flavobacteriales bacterium | Leadbetterella byssophila DSM |
| | ALC-1 | 17132 |
| Flavobacterium branchiophilum | Paenibacillus mucilaginosus | Pseudoalteromonas sp. BSi20439 |
| Fl-15 | K02 | |
| Burkholderia glumae BGRl | Weeksella virosa DSM 16922 | Myroides odoratimimus CCUG |
| | | 10230 |
| Cyclobacterium marinum DSM | Lacinutrix sp. 5H-3-7-4 | Bacteroides stercoris ATCC |
| 745 | | 43183 |
| Pseudomonas fluorescens Fl13 | Haliscomenobacter hydrossis | Pseudomonas putida BIRD-1 |
| | DSM 1100 | |
| Niastella koreensis GR20-10 | Vibrio cholerae LMA3984-4 | Muricauda ruestringensis DSM |
| | | 13258 |
| actococcus garvieae g2 | Burkholderia sp. CCGE1003 | Staphylococcus |
| | | pseudintermedius ED99 |
| Flavobacterium columnare | Bacillus selenitireducens | 5hewanella woodyi ATCC 51908 |
| ATCC 49512 | MLSlO | |
| Lactobacillus rhamnosus GG | Pedobacter saltans DSM 12145 | Flavobacterium psychrophilum |
| | | JIP02/86 |
| Borrelia burgdorferi B31 | Bacteriovorax marinus SJ | Burkholderia sp. 383 |
| Fibrobacter succinogenes subsp. | Cupriavidus taiwanensis LMG | Pseudomonas fluorescens Pf0-1 |
| succinogenes S85 | 19424 | |
| Pseudomonas mendocina NK- | Lactobacillus easel BD-11 | Alcanivorax borkumensis SK2 |
| 01 | | |
| Herminiimonas arsenicoxydans | Cellulophaga algicola DSM | Odoribacter laneus YIT 12061 |
| | 14237 | |
| Lactococcus lactis subsp. | Cupriavidus metallidurans | Coprococcus eutactus ATCC |
| cremoris CNCM 1-1631 | CH34 | 27759 |
| Lactobacillus plantarum WCFSl | Bacteroides sp. 1_1_30 | Clostridium acetobutylicum EA |
| | | 2018 |
| Lachnospiraceae bacterium | Streptococcus thermophilus | Clostridium sporogenes ATCC |
| ACC2 | CNCM 1-1630 | 15579 |
| Lactobacillus sakei subsp. sakei | Zobellia galactanivorans | Vibrio furnissii NCTC 11218 |
| 23K | | |
| Oceanimonas sp. GKl | Staphylococcus haemolyticus | Lactobacillus fermentum CECT |
| | JCSC1435 | 5716 |
| Halobacillus halophilus DSM | Chromohalobacter salexigens | Clostridium botulinum |
| 2266 | DSM 3043 | BKT015925 |
| Bordetella bronchiseptica RB50 | Providencia stuartii MRSN 2154 | Polynucleobacter necessarius |
| | | subsp. asymbioticus QLW- |
| | | PlDMWA-1 |
| Tannerella sp. | Alistipes indistinctus YIT 12060 | Pseudoalteromonas atlantica T6c |
| 6_1_58FAA_CT1 | | |
| Streptococcus mutans UA159 | Aliivibrio salmonicida LF11238 | Halomonas boliviensis LCl |
| gamma proteobacterium HdNl | Bacteroides dorei DSM 17855 | Streptococcus intermedius F0395 |
| Weissella koreensis KACC | Lactobacillus sp. 7_1_47FAA | Burkholderia sp. Yi23 |
| 15510 | | |
| Shewanella amazonensis SB2B | Lachnospiraceae bacterium | Pseudomonas syringae pv. |
| | 2_1_46FM | phaseolicola 1448A |
| Pseudomonas aeruginosa M18 | Streptococcus gallolyticus | Streptococcus pyogenes |
| | subsp. gallolyticus ATCC 43143 | MGAS1882 |
| Fusobacterium nucleatum | Sphingobacterium sp. 21 | Leptotrichia buccalis C-1013-b |
| subsp. animalis OT 420 | | |
| Oceanobacillus iheyensis | Clostridium hathewayi WAL- | Clostridium sp. L2-SO |
| HTE831 | 18680 | |
| Lactobacillus johnsonii DPC | Streptococcus suis A7 | Bordetella avium 197N |
| 6026 | | |
| Selenomonas noxia F0398 | Psychromonas ingrahamii 37 | Anaerostipes caccae DSM 14662 |
| Clostridium sp. BNL1100 | Coprococcus comes ATCC | Bordetella petrii DSM 12804 |
| | 27758 | |
| Staphylococcus aureus subsp. | Lactobacillus salivarius CECT | Aeromonas hydrophila subsp. |
| aureus JKD6159 | 5713 | hydrophila ATCC 7966 |
| Tannerella forsythia ATCC | Desulfotomaculum ruminis | Dorea formicigenerans |
| 43037 | DSM 2154 | 4_6_53AFAA |
| Pseudomonas stutzeri DSM | Desulfosporosinus orientis DSM | Enterococcus faecalis 62 |
| 4166 | 765 | |
| Eubacterium eligens ATCC | Enterococcus saccharolyticus | Pseudogulbenkiania sp. NH8B |
| 27750 | 30_1 | |
| Cronobacter sakazakii ATCC | Shewanella oneidensis MR-1 | Selenomonas ruminantium subsp. |
| BAA-894 | | lactilytica TAM6421 |
| Clostridium sp. SS2/1 | Lachnospiraceae bacterium | Johnsonella ignava ATCC 51276 |
| | 1_4_56FAA | |
| Aeromonas salmonicida subsp. | Ralstonia solanacearum Po82 | Dysgonomonas mossii DSM |
| salmonicida A449 | | 22836 |
| Parabacteroides merdae ATCC | Dysgonomonas gadei ATCC | Bacteroides coprocola DSM |
| 43184 | BAA-286 | 17136 |
| Cellvibrio japonicus Ueda107 | Lachnospiraceae bacterium | Spirochaeta africana DSM 8902 |
| | 5_1_57FAA | |
| Facklamia languida CCUG | gamma proteobacterium NORS- | Catonella morbi ATCC 51271 |
| 37842 | 3 | |
| Eubacterium ventriosum ATCC | Eikenella corrodens ATCC | Methylomonas methanica MC09 |
| 27560 | 23834 | |
| Citrobacter freundii | Blautia hydrogenotrophica DSM | Alteromonas macleodii str. ‘Deep |
| 4_7_47CFAA | 10507 | ecotype’ |
| Clostridium asparagiforme | Azotobacter vinelandii DJ | Lachnospiraceae bacterium |
| DSM 15981 | | 7_1_58FAA |
| Paenibacillus sp. oral taxon 786 | Alteromonas sp. SN2 | Desulfitobacterium hafniense |
| str. D14 | | Y51 |
| Geobacter metallireducens GS- | Lachnospiraceae bacterium | Methylomicrobium alcaliphilum |
| 15 | 3_1_46FAA | |
| Saccharophagus degradans 2-40 | Melissococcus plutonius | Taylorella asinigenitalis MCE3 |
| | DAT561 | |
| Lactobacillus delbrueckii subsp. | Oribacterium sp. ACBl | Bacteroides pectinophilus ATCC |
| bulgaricus 2038 | | 43243 |
| Salmonella enterica subsp. | z23:-- str. RSK2980 | Ruminococcus lactaris ATCC |
| arizonae serovar 62:z4 | | 29176 |
| Roseburia inulinivorans DSM | Neisseria flavescens | Paenibacillus terrae HPL-003 |
| 16841 | NRL30031/H210 | |
| Shewanella piezotolerans WP3 | Pediococcus claussenii ATCC | Chitinophaga pinensis DSM |
| | BAA-344 | 2588 |
| Gemella haemolysans M341 | Shewanella sediminis HAW- | Shewanella loihica PV-4 |
| | EB3 | |
| Clostridium hylemonae DSM | Lachnospiraceae bacterium | Heliobacterium modesticaldumlcel |
| 15053 | 4_1_37FAA | |
| Geobacter sulfurreducens PCA | Bacteroides capillosus ATCC | Shewanella violacea DSS12 |
| | 29799 | |
| Bacillus thuringiensis serovar | Shewanella baltica OS678 | Erysipelotrichaceae bacterium |
| konkukian str. 97-27 | | 21_3 |
| Spirochaeta coccoides DSM | Bacillus pseudofirmus OF4 | Shewanella baltica BA175 |
| 17374 | | |
| Prevotella sp. oral taxon | Clostridium bolteae ATCC | Bacillus sp. B14905 |
| 302 str. F0323 | BAA-613 | |
| Lachnospiraceae bacterium | Bacillus sp. JS | Abiotrophia defectiva ATCC |
| 3_1_57FAA_CT1 | | 49176 |
| Ruminococcus sp. | Coprobacillus sp. 8_2_54BFAA | Shewanella putrefaciens CN-32 |
| 5_1_39B_FAA | | |
| Neisseria lactamica 020-06 | Catenibacterium mitsuokai | Neisseria gonorrhoeae TCDC- |
| | DSM 15897 | NG08107 |
| Lachnospiraceae bacterium | Bacillus licheniformis DSM 13 = | Clostridium sp. 7_3_54FAA |
| 6_1_63FAA | ATCC 14580 | |
| Glaciecola nitratireducens | Listeria seeligeri serovar 1/2b | Escherichia phage phiVlO |
| FR1064 | str. SLCC3954 | |
| Eubacterium sp. 3_1_31 | Parvimonas micra ATCC 33270 | Erysipelothrix rhusiopathiae str. |
| | | Fujisawa |
| Treponema pallidum subsp. | Clostridium nexile DSM 1787 | Clostridium spiroforme DSM |
| pallidum DAL-1 | | 1552 |
| Pectobacterium atrosepticum | Sphaerochaeta globus str. | Rahnella aquatilis CIP 78.65 = |
| SCRl1043 | Buddy | ATCC 33071 |
| Clostridium kluyveri NBRC | Clostridium perfringens WAL- | Shuttleworthia satelles DSM |
| 12016 | 14572 | 14600 |
| Sphaerochaeta pleomorpha str. | Lactobacillus helveticus H10 | Prevotella micans F0438 |
| Grapes | | |
| Pantoea ananatis PA13 | Pantoea ananatis LMG 20103 | Leuconostoc mesenteroides |
| | | subsp. mesenteroides J18 |
| Anaeromyxobacter sp. Fw109-5 | Serratia sp. AS13 | Prevotella maculosa OT 289 |
| Acetobacterium woodii DSM | Roseburia hominis A2-183 | Mycoplasma fermentans PG18 |
| 1030 | | |
| Thiobacillus denitrificans | Yersinia enterocolitica subsp. | Desulfobacterium autotrophicum |
| ATCC 25259 | palearctica Yl | HRM2 |
| Sorangium cellulosum ‘So ce 56’ | Subdoligranulum sp. | Prevotella histicola F0411 |
| | 4_3_54A2FAA | |
| Arcobacter butzleri ED-1 | Eubacterium limosum KIST612 | Arcobacter nitrofigilis DSM |
| | | 7299 |
| Leuconostoc sp. C2 | Mycoplasma hyopneumoniae | Eubacterium haIIii DSM 3353 |
| | 168 | |
| Cellulophaga lytica DSM 7489 | Mycoplasma hyorhinis HUB-1 | Mycoplasma hyorhinis MCLD |
| Helcococcus kunzii ATCC | Campylobacter showae | Granulicella mallensis |
| 51366 | RM3277 | MP5ACTX8 |
| Helicobacter pylori 83 | Arcobacter sp. L | Gluconacetobacter xylinus |
| | | NBRC 3288 |
| Anaerotruncus colihominis | Treponema brennaborense DSM | Myroides odoratimimus CIP |
| DSM 17241 | 12168 | 101113 |
| Granulibacter bethesdensis | Bacteroides sp. 2_2_4 | Campylobacter jejuni |
| CGDNIHl | | |
| Bacteroides sp. D20 | Geobacillus thermodenitrificans | Bacteroides sp. 9_1_42FAA |
| | NG80-2 | |
| Microcystis aeruginosa PCC | Bacteroides fragilis 3_1_12 | lmtechella halotolerans Kl |
| 7806 | | |
| Acaryochloris marina | Caldilinea aerophila DSM | Planctomyces brasiliensis DSM |
| MBIC11017 | 14535 = NBRC 104270 | 5305 |
| Bacteroides sp. 01 | Trichodesmium erythraeum | Homo sapiens |
| | IMS101 Saccharomyces cerevisiae | |
| Saprospiragrandis | Shewanella putrefaciens | Caldicellulosiruptor kristjanssonii |
| Cyanophage pSS2 | Anaeroce/lum thermophilum | Myxococcus fulvus |
| Caldicellulosiruptor | Mycobacterium marinum | Photorhabdus luminescens |
| hydrothermalis | | |
| Myxococcus xanthus | Corallococcus coralloides | Persephone/la marina |
| Legionella pneumophila | Burkholderia pseudomallei | Stigmatella aurantiaca |
| Caldicellulosiruptor | Rhizobium loti | Vibrio cholerae |
| saccharolyticus | | |
| Listeria phage A118 | Salmonella newport | Xylanimonas cellulosilytica |
| Caldice/lulosiruptor | Delftia sp. | Paracoccus denitrificans |
| kronotskyensis | | |
| Pelobacter propionicus | Psychrobacter sp. | Clostridium cellulovorans |
| Bifidobacterium longum subsp. | Sinorhizobium meliloti | Campylobacter curvus |
| infantis | | |
| Corynebacterium variabile | Xylella fastidiosa | Photorhabdus luminescens subsp. |
| | | laumondii |
| Escherichia coli | Lactotoccus phage u/l6.2 | Enterobacter sp. |
| Clostridium difficile | Paenibacillus sp. | A/lochromatium vinosum |
| Acidithiobacillus caldus | Vibrio splendidus | Carboxydothermus |
| | | hydrogenoformans |
| Campylobacter jejuni subsp. | Soda/is glossinidius | Spirochaeta smaragdinae |
| doylei | | |
| Haemophilus parasuis serovar 5 | Gluconobacter oxydans | Halanaerobium sp. |
| Enterobacter cloacae subsp. | Neisseria meningitidis serogroup | Enterobacter aerogenes |
| cloacae | A | |
| Yersinia pseudotuberculosis | Bartone/la grahamii | Bartone/la tribocorum |
| Shewane/la sp. | Proteus mirabilis | Streptococcus pneumoniae |
| Pseudomonas fluorescens | Vibrio cholerae serotype O1 | Alkaliphilus metalliredigens |
| Streptococcus pyogenes | Streptococcus pyogenes | Laribacter hongkongensis |
| serotype M12 | serotype Ml | |
| Salmonella paratyphi B | Salmonella heidelberg | Escherichia fergusonii |
| Hydrogenobacter thermophilus | Shigella dysenteriae serotype 1 | Salmonella typhi |
| Aggregatibacter aphrophilus | Listeria innocua serovar 6a | Drosophila melanogaster |
| Streptococcus pyogenes | Xenorhabdus bovienii | Streptococcus parauberis |
| serotype MS | | |
| Thermovibrio ammonificans | Streptococcus salivarius | Frateuria aurantia |
| Melissococcus plutonius | Avibacterium paragallinarum | Salmonella enterica subsp. |
| | | enterica serovar Typhi str. 404ty |
| Haemophilus influenzae R3021 | Xylella fastidiosa Ann- 1 | Bartone/la schoenbuchensis R1 |
| Enterobacteria phage lambda | Gluconacetobacter oboediens | Helicobacter cinaedi CCUG |
| | 174Bp2 | 18818 |
| Bartone/la sp. AR 15-3 | Listeria monocytogenes FSL | Listeria ivanovii FSLF6-596 |
| | R2-503 | |
| Paenibacillus larvae subsp. larvae | Escherichia fergusonii ECD227 | Enterobacteria phage Min27 |
| B-3650 | | |
| Salmonella enterica subsp. | Myxococcus fulvus HW-1 | Salmonella enterica subsp. |
| enterica serovar Rubislaw str. | | enterica serovar Javiana str. |
| A4-653 | | GA_MM04042433 |
| Salmonella enterica subsp. | Shigella dysenteriae | Salmonella enterica subsp. |
| enterica serovar Senftenberg str. | | enterica serovar Typhi str. AG3 |
| A4-543 | | |
| Myxococcus xanthus DK 1622 | Shigella dysenteriae 1617 | Caldicellulosiruptor |
| | | hydrothermalis 108 |
| Caldicellulosiruptor | Corallococcus cora/loides DSM | Stigmatella aurantiaca DW4/3-1 |
| kristjanssonii 177R1B | 2259 | |
| Caldice/lulosiruptor | Providencia rettgeri | Lactococcus phage phismq86 |
| saccharolyticus DSM 8903 | | |
| Caldicellulosiruptor | Neisseria meningitidis WUE | Neisseria lactamica ¥92-1009 |
| kronotskyensis 2002 | 2594 | |
| Burkholderia pseudomallei | Clostridium botulinum C str. | Clostridium cellulovorans 7438 |
| NCTC 13177 | Eklund | |
| Xenorhabdus nematophila | aribacter hongkongensis | Burkholderia pseudomallei 668 |
| ATCC l9061 | HLHK9 | |
| Alkaliphilus metalliredigens | Klebsiella pneumoniae | Vibrio cholerae 0139 |
| QYMF | | |
| Yersinia ruckeri | Haemophilus haemolyticus | Cyanophage PSS2 |
| | M21639 | |
| Providencia rettgeri DSM 1131 | Lactococcus phage phiLC3 | Frateuria aurantia DSM 6220 |
| Haemophilus influenzae NT127 | Streptococcus phage 2167 | Aggregatibacter aphrophilus |
| | | NJ8700 |
| Streptococcus pneumoniae EU- | Me/issococcus plutonius ATCC | Streptococcus infantis SK970 |
| NP01 | 35311 | |
| Salmonella enterica subsp. | Bacillus subtilis subsp. spizizenii | Enterobacter cloacae subsp. |
| enterica serovar Montevideo str. | TU-B-10 | cloacae ATCC 13047 |
| SARB30 | | |
| Photobacterium damselae subsp. | Neisseria bacilliformis ATCC | Clostridium botulinum C sir. |
| damselae | BAA-1200 | Stockholm |
| Enterobacter aerogenes KCTC | Pseudomonas aeruginosa 39016 | Bartonella rochalimae ATCC |
| 2190 | | BAA-1498 |
| Fusobacterium sp. 3_1_5R | Comamonas testosteroni S44 | Pediococcus acidilactici DSM |
| | | 20284 |
| Salmonella enterica subsp. | Sodalis glossinidius str. | Streptococcus parauberis KCTC |
| houtenae str. ATCC BAA-1581 | ‘morsitans’ | 11537 |
| Bartonella tribocorum CIP | Klebsiella sp. 1_1_55 | Lactobacillus ruminis SPM0211 |
| 105476 | | |
| Haemophilus | Clostridium sporogenes PA | Mannheimia haemolytica |
| paraphrohaemolyticus HK411 | 3679 | PHL213 |
| Klebsiella pneumoniae subsp. | Streptococcus phage 8140 | Sinorhizobium meliloti SMll |
| rhinoscleromatis ATCC 13884 | | |
| Streptococcus phage V22 | Clostridium botulinum El str. | Streptococcus phage 9429.2 |
| | ‘BoNT E Beluga’ | |
| Spirochaeta smaragdinae DSM | Sinorhizobium meliloti AK83 | Streptococcus pyogenes str. |
| 11293 | | Manfredo |
| Carboxydothermus | Streptococcus pneumoniae | Klebsiella pneumoniae KCTC |
| hydrogenoformans Z-2901 | GA11426 | 2242 |
| Streptococcus salivarius | Acinetobacter sp. SH024 | Burkholderia sp. Chl-1 |
| JIM8777 | | |
| Streptococcus suis 89/1591 | Mesorhizobium loti | Streptococcus suis SS12 |
| | MAFF303099 | |
| listeriamonocytogenes FSL Nl- | lodobacteriophage phiPLPE | Bartonella grahamii as4aup |
| 017 | | |
| Fusobacterium sp. 12_1B | Cryptobacterium curtum DSM | Bacteroides caccae ATCC 43185 |
| | l5641 | |
| Leptotrichia goodfellowii | Listeria innocua Clip11262 | Vibrio cholerae non-01/non- |
| F0264 | | 0139 |
| Acinetobacter | Halanaerobium | Mycobacterium marinum M |
| baumanniiAB900 | hydrogeniformans | |
| Peptoniphilus duerdenii ATCC | Serratia symbiotica str. Tucson | Bifidobacterium longum subsp. |
| BAA-1640 | | infantis 157F |
| Campylobacter jejuni subsp. | Xylella fastidiosa subsp. | Xylella fastidiosa 9a5c |
| doylei269.97 | fastidiosa GB514 | |
| Acinetobacter sp. P8-3-8 | Streptococcus phage M102 | Campylobacter coli 1957 |
| Corynebacterium variabile | Collinsella stercoris DSM | Campylobacter curvus 525.92 |
| DSM44702 | 13279 | |
| Campylobacter jejuni subsp. | Campylobacter coli67-8 | Campylobacter coli LMG 9860 |
| jejuni 2008-988 | | |
| Campylobacter coli 2685 | Campylobacter coli 132-6 | Sebaldella termitidis ATCC |
| | | 33386 |
| Xylanimonas cellulosilytica | Campylobacter coli80352 | Collinsella aerofaciens ATCC |
| DSM 15894 | | 25986 |
| Aggregatibacter segnis ATCC | Haemophilus parasuis SH0165 | Pelobacter propionicus DSM |
| 33393 | | 2379 |
| Haemophilus parasuis 29755 | Campylobacter upsaliensis | Acidithiobacillus caldus SM-1 |
| | RM3195 | |
| Acidithiobacillus caldus ATCC | Streptococcus gallolyticus | Yersinia pseudotuberculosis IP |
| 51756 | subsp. gallolyticus TX20005 | 32953 |
| Burkholderia pseudomallei | Sinorhizobium meliloti | Vibrio furnissii CIP 102972 |
| Pasteur 52237 | CCNWSX0020 | |
| Acetobacteraceae bacterium | Helicobacter pullorum MIT 98- | Pseudomonas fluorescens |
| AT-5844 | 5489 | SBW25 |
| Photobacterium damselae subsp. | Sinorhizobium medicae | Hydrogenobacter thermophilus |
| damselae CIP 102761 | WSM419 | TK-6 |
| Simkania negevensis z | Rhizobium leguminosarum bv. | Persephonella marina EX-Hl |
| | trifolii WSM2304 | |
| Thiocapsa marina 5811 | Thiorhodococcus drewsii AZl | Desuifitobacterium |
| | | metallireducens DSM 15288 |
| Allochromatium vinosum DSM | Vibrio splendidus LGP32 | Thermus aquaticus Y51MC23 |
| 180 | | |
| Enterobacter sp. 638 | Thiocystis violascens DSM 198 | Vibrio cholerae 1587 |
| Brevundimonas diminuta ATCC | Delftia sp. Csl-4 | Corynebacterium diphtheriae C7 |
| 11568 | | (beta) |
| Cronobacter phage ENT47670 | Thiorhodovibrio sp. 970 | Paenibacillus sp. JDR-2 |
| Photorhabdus luminescens | Psychrobacter sp. PRwf-1 | Aeromonas salmonicida subsp. |
| subsp. laumondii TTOl | | salmonicida 01-6526 |
| Gluconobacter oxydans 621H | Sporosarcina newyorkensis | Acidovorax delafieldii 2AN |
| | 2681 | |
| Paenibacillus elgii B69 | Methylomicrobium album BG8 | Enterobacteriaceae bacterium |
| | | 9_2_54FAA |
| Paracoccus denitrificans | Commensalibacter intestini | Mobiluncus curtisii ATCC 51333 |
| PD1222 | A911 | |
| Listeria monocytogenes str. 1/2a | Pseudomonas syringae pv. pisi | Listeria monocytogenes FSLJ2- |
| F6854 | str. l7048 | 003 |
| Pseudomonas syringae pv. | Xanthobacter autotrophicus Py2 | Ureaplasma parvum serovar 6 str. |
| lachrymans str. M301315 | | ATCC 27818 |
| Rhodococcus phage REQ3 | Ureaplasma urealyticum serovar | Ureaplasma urealyticum serovar |
| | 8 str. ATCC 27618 | l2 str. ATCC 33696 |
| Halomonas elongata DSM 2581 | Klebsiella oxytoca 10-5250 | Paenibacillus larvae subsp. larvae |
| | | BRL-230010 |
| Hafnia alvei ATCC 51873 | Burkholderia cenocepacia J2315 | Gardnerella vaginalis 409-05 |
| Proteus penneri ATCC 35198 | Acinetobacter baumanniiATCC | Staphylococcus aureus subsp. |
| | 19606 | aureus D139 |
| Pseudoalteromonas sp. | Parabacteroides johnsoni i DSM | Citrobacter koseri ATCC BM- |
| BSi20495 | 18315 | 895 |
| Borrelia hermsii | Salmonella enterica subsp. | Salmonella phage SPN1S |
| | enterica serovar Mississippi str. | |
| | A4-633 | |
| Salmonella enterica subsp. | Shigella dysenteriae CDC 74- | Corynebacterium striatum ATCC |
| enterica serovar Kentucky str. | 11l2 | 6940 |
| CDC 191 | | |
| Klebsiella sp. MS 92-3 | Pantoea sp. aB | Proteus mirabilis ATCC 29906 |
| Providencia alcalifaciens DSM | Escherichia phage TL-2011b | Clostridium methylpentosum |
| 30120 | | DSM 5476 |
| Erwinia phage phiEt88 | Gemella sanguinis M325 | Serratia odorifera DSM 4582 |
| Pseudomonas brassicacearum | Comamonas testosteroni KF-1 | Edwardsiella tarda ATCC 23685 |
| subsp. brassicacearum NFM421 | | |
| Brenneria sp. EniD312 | Legionella pneumophila | Enterococcus faecalis TX0630 |
| Edwardsiella ictaluri 93-146 | Edwardsiella tarda EIB202 | Acinetobacter radioresistens |
| | | SK82 |
| Chelativorans sp. BNC1 | Moraxella catarrhalis 101P30B1 | Clostridium botulinum B str. |
| | | Eklund 178 |
| Xenorhabdus bovienii SS-2004 | Erwinia billingiae Eb661 | EBPR podovirus 1 |
| Methylobacterium nodulans | Pantoea vagans C9-1 | Oceanicola sp. 5124 |
| ORS 2060 | | |
| Salmonella enterica subsp. | Klebsiella pneumoniae subsp. | Thermovibrio ammonificans |
| enterica serovar Gaminara str. | pneumoniae MGH 78578 | HB-1 |
| A4-567 | | |
| Corynebacterium glutamicum R | Phage Gifsy-1 | Escherichia phage TL-2011c |
| Salmonella enterica subsp. | Vibrio angustum S14 | Corynebacterium ulcerans BR- |
| enterica serovar Typhimurium | | AD22 |
| Listeria monocytogenes F5L F2- | Mycobacterium abscessus M93 | Psychrobacter arcticus 273-4 |
| 515 | | |
| Ahrensia sp. R2A 130 | Thermoanaerobacter italicus | Rhodomicrobium vannielii |
| | Ab9 | ATCC 17100 |
| Acinetobacter radioresistens | Photobacterium profundum SS9 | Segniliparus rotundus DSM |
| SH164 | | 44985 |
| Megamonas funiformis YIT | Pseudomonas syringae pv. | Pseudomonas syringae pv. |
| 11815 | syringae B728a | maculicola str. ES4326 |
| Moraxella catarrhalis BCl | Eubacterium saburreum DSM | Clostridium hathewayi DSM |
| | 3986 | 13479 |
| achnospiraceae bacterium | Eubacteriaceae bacterium | Burkholderia sp. CCGE1002 |
| 9_1_43BFAA | ACC19a | |
| Bacteroides sp. 2_1_33B | Nitratifractor salsuginis DSM | Jonesia denitrificans DSM 20603 |
| | 16511 | |
| Agrobacterium tumefaciens | Aeromonas hydrophila | butyrate-producing bacterium |
| | | 553/4 |
| Enterobacter hormaechei ATCC | Pseudomonas aeruginosa | Pseudomonas phage phi297 |
| 49162 | | |
| Klebsiella variicola At-22 | Burkholderia sp. TJ149 | Klebsiella oxytoca 10-5243 |
| Burkholderia phymatum | Pseudomonas putida GB-1 | Klebsiella pneumoniae 342 |
| 5TM815 | | |
| Shewanella putrefaciens 200 | Saprospira grandis str. Lewin | Mycobacterium abscessus M94 |
| Bradyrhizobium sp. STM 3843 | Bacillus subtilis subsp. natto | Sodalis phage phiSG1 |
| | BEST195 | |
| Parachlamydia acanthamoebae | Achromobacter xylosoxidans | Desulfovibrio piger ATCC 29098 |
| str. Hall's coccus | AXX-A | |
| Burkholderia dolosa AU0158 | delta proteobacterium NaphS2 | Vibrio caribbenthicus ATCC |
| | | BAA-2122 |
| Dyadobacter fermentans DSM | Kordia algicida OT-1 | Flavobacterium indicum |
| 18053 | | GPTSAl00-9 |
| Vibrio harveyi 1DA3 | Runella slithyformis DSM | Flavobacterium johnsoniae |
| | 19594 | UW101 |
| Owenweeksia hongkongensis | Riemerella anatipestifer RA-GD | Flavobacteriales bacterium ALC- |
| DSM 17368 | | 1 |
| Leadbetterella byssophila DSM | Flavobacterium branchiophilum | Paenibacillus mucilaginosus K02 |
| 17132 | Fl-15 | |
| Pseudoalteromonas sp. | Burkholderia glumae BGRl | Weeksella virosa DSM 16922 |
| BSi20439 | | |
| Myroides odoratimimus CCUG | Cyclobacterium marinum DSM | Lacinutrix sp. 5H-3-7-4 |
| 10230 | 745 | |
| Bacteroides stercoris ATCC | Pseudomonas fluorescens Fl13 | Haliscomenobacter hydrossis |
| 43183 | | DSM 1100 |
| Pseudomonas putida BIRD-1 | Niastella koreensis GR20-10 | Vibrio cholerae LMA3984-4 |
| Muricauda ruestringensis DSM | actococcus garvieae g2 | Burkholderia sp. CCGE1003 |
| 13258 | | |
| Staphylococcus | Flavobacterium columnare | Bacillus selenitireducens MLSlO |
| pseudintermedius ED99 | ATCC 49512 | |
| 5hewanella woodyi ATCC | Lactobacillus rhamnosus GG | Pedobacter saltans DSM 12145 |
| 51908 | | |
| Flavobacterium psychrophilum | Borrelia burgdorferi B31 | Bacteriovorax marinus SJ |
| JIP02/86 | | |
| Burkholderia sp. 383 | Fibrobacter succinogenes subsp. | Cupriavidus taiwanensis LMG |
| | succinogenes S85 | 19424 |
| Pseudomonas fluorescens Pf0-1 | Pseudomonas mendocina NK-01 | Lactobacillus easel BD-11 |
| Alcanivorax borkumensis SK2 | Herminiimonas arsenicoxydans | Cellulophaga algicola DSM |
| | | 14237 |
| Odoribacter laneus YIT 12061 | Lactococcus lactis subsp. | Cupriavidus metallidurans CH34 |
| | cremoris CNCM 1-1631 | |
| Coprococcus eutactus ATCC | Lactobacillus plantarum WCFSl | Bacteroides sp. 1_1_30 |
| 27759 | | |
| Clostridium acetobutylicum EA | Lachnospiraceae bacterium | Streptococcus thermophilus |
| 2018 | ACC2 | CNCM 1-1630 |
| Clostridium sporogenes ATCC | Lactobacillus sakei subsp. sakei | Zobellia galactanivorans |
| 15579 | 23K | |
| Vibrio furnissii NCTC 11218 | Oceanimonas sp. GK1 | Staphylococcus haemolyticus |
| | | JCSC1435 |
| Lactobacillus fermentum CECT | Halobacillus halophilus DSM | Chromohalobacter salexigens |
| 5716 | 2266 | DSM 3043 |
| Clostridium botulinum | Bordetella bronchiseptica RB50 | Providencia stuartii MRSN 2154 |
| BKT015925 | | |
| Polynucleobacter necessarius | Tannerella sp. 6_1_58FAA_CT1 | Alistipes indistinctus YIT 12060 |
| subsp. asymbioticus QLW- | | |
| PlDMWA-1 | | |
| Pseudoalteromonas atlantica | Streptococcus mutans UA159 | Aliivibrio salmonicida LF11238 |
| T6c | | |
| Halomonas boliviensis LCl | gamma proteobacterium HdNl | Bacteroides dorei DSM 17855 |
| Streptococcus intermedius | Weissella koreensis KACC | Lactobacillus sp. 7_1_47FAA |
| F0395 | 15510 | |
| Burkholderia sp. Yi23 | Shewanella amazonensis SB2B | Lachnospiraceae bacterium |
| | | 2_1_46FM |
| Pseudomonas syringae pv. | Pseudomonas aeruginosa M18 | Streptococcus gallolyticus subsp. |
| phaseolicola 1448A | | gallolyticus ATCC 43143 |
| Streptococcus pyogenes | Fusobacterium nucleatum | Sphingobacterium sp. 21 |
| MGAS1882 | subsp. animalis OT 420 | |
| Leptotrichia buccalis C-1013-b | Oceanobacillus iheyensis | Clostridium hathewayi WAL- |
| | HTE831 | 18680 |
| Clostridium sp. L2-SO | Lactobacillus johnsonii DPC | Streptococcus suis A7 |
| | 6026 | |
| Bordetella avium 197N | Selenomonas noxia F0398 | Psychromonas ingrahamii 37 |
| Anaerostipes caccae DSM 14662 | Clostridium sp. BNL1100 | Coprococcus comes ATCC 27758 |
| Bordetella petrii DSM 12804 | Staphylococcus aureus subsp. | Lactobacillus salivarius CECT |
| | aureus JKD6159 | 5713 |
| Aeromonas hydrophila subsp. | Tannerella forsythia ATCC | Desulfotomaculum ruminis DSM |
| hydrophila ATCC 7966 | 43037 | 2154 |
| Dorea formicigenerans | Pseudomonas stutzeri DSM 4166 | Desulfosporosinus orientis DSM |
| 4_6_53AFAA | | 765 |
| Enterococcus faecalis 62 | Eubacterium eligens ATCC | Enterococcus saccharolyticus |
| | 27750 | 30_1 |
| Pseudogulbenkiania sp. NH8B | Cronobacter sakazakii ATCC | Shewanella oneidensis MR-1 |
| | BAA-894 | |
| Selenomonas ruminantium | Clostridium sp. SS2/1 | Lachnospiraceae bacterium |
| subsp. lactilytica TAM6421 | | 1_4_56FAA |
| Johnsonella ignava ATCC 51276 | Aeromonas salmonicida subsp. | Ralstonia solanacearum Po82 |
| | salmonicida A449 | |
| Dysgonomonas mossii DSM | Parabacteroides merdae ATCC | Dysgonomonas gadei ATCC |
| 22836 | 43184 | BAA-286 |
| Bacteroides coprocola DSM | Cellvibrio japonicus Ueda107 | Lachnospiraceae bacterium |
| 17136 | | 5_1_57FAA |
| Spirochaeta africana DSM 8902 | Facklamia languida CCUG | gamma proteobacterium NORS-3 |
| | 37842 | |
| Catonella morbi ATCC 51271 | Eubacterium ventriosum ATCC | Eikenella corrodens ATCC 23834 |
| | 27560 | |
| Methylomonas methanica | Citrobacter freundii | Blautiahydrogenotrophica DSM |
| MC09 | 4_7_47CFAA | 10507 |
| Alteromonas macleodii str. | Clostridium asparagiforme | Azotobacter vinelandii DJ |
| ‘Deep ecotype’ | DSM 15981 | |
| Lachnospiraceae bacterium | Paenibacillus sp. oral taxon 786 | Alteromonas sp. SN2 |
| 7_1_58FAA | str. D14 | |
| Desulfitobacterium hafniense | Geobacter metallireducens GS- | Lachnospiraceae bacterium 3_1_46FAA |
| Y51 | 15 | |
| Methylomicrobium alcaliphilum | Saccharophagus degradans 2-40 | Melissococcus plutonius |
| | | DAT561 |
| Taylorella asinigenitalis MCE3 | Lactobacillus delbrueckii subsp. | Oribacterium sp. ACB1 |
| | bulgaricus 2038 | |
| Bacteroides pectinophilus ATCC | Salmonella enterica subsp. | z23:-- str. RSK2980 |
| 43243 | arizonae serovar 62:z4 | |
| Ruminococcus lactaris ATCC | Roseburia inulinivorans DSM | Neisseria flavescens |
| 29176 | 16841 | NRL30031/H210 |
| Paenibacillus terrae HPL-003 | Shewanella piezotolerans WP3 | Pediococcus claussenii ATCC |
| | | BAA-344 |
| Chitinophaga pinensis DSM | Gemella haemolysans M341 | Shewanella sediminis HAW-EB3 |
| 2588 | | |
| Shewanella loihica PV-4 | Clostridium hylemonae DSM | Lachnospiraceae bacterium |
| | 15053 | 4_1_37FAA |
| Heliobacterium modesticaldum | Geobacter sulfurreducens PCA | Bacteroides capillosus ATCC |
| 1cel | | 29799 |
| Shewanella violacea DSS12 | Bacillus thuringiensis serovar | Shewanella baltica OS678 |
| | konkukian str. 97-27 | |
| Erysipelotrichaceae bacterium | Spirochaeta coccoides DSM | Bacillus pseudofirmus OF4 |
| 21_3 | 17374 | |
| Shewanella baltica BA175 | Prevotella sp. oral taxon 302 str. | Clostridium bolteae ATCC BAA- |
| | F0323 | 613 |
| Bacillus sp. B14905 | Lachnospiraceae bacterium | Bacillus sp. JS |
| | 3_1_57FAA_CT1 | |
| Abiotrophia defectiva ATCC | Ruminococcus sp. 5_1_39B_FAA | Coprobacillus sp. 8_2_54BFAA |
| 49176 | | |
| Shewanella putrefaciens CN-32 | Neisseria lactamica 020-06 | Catenibacterium mitsuokai DSM |
| | | 15897 |
| Neisseria gonorrhoeae TCDC- | Lachnospiraceae bacterium | Bacillus licheniformis DSM 13 = |
| NG08107 | 6_1_63FAA | ATCC 14580 |
| Clostridium sp. 7_3_54FAA | Glaciecola nitratireducens | Listeria seeligeri serovar 1/2b str. |
| | FR1064 | SLCC3954 |
| Escherichia phage phiVlO | Eubacterium sp. 3_1_31 | Parvimonas micra ATCC 33270 |
| Erysipelothrix rhusiopathiae str. | Treponema pallidum subsp. | Clostridium nexile DSM 1787 |
| Fujisawa | pallidum DAL-1 | |
| Clostridium spiroforme DSM | Pectobacterium atrosepticum | Sphaerochaeta globus str. Buddy |
| 1552 | SCR11043 | |
| Rahnella aquatilis CIP 78.65 = | Clostridium kluyveri NBRC | Clostridium perfringens WAL- |
| ATCC 33071 | 12016 | 14572 |
| Shuttleworthia satelles DSM | Sphaerochaeta pleomorpha str. | Lactobacillus helveticus H10 |
| 14600 | Grapes | |
| Prevotella micans F0438 | Pantoea ananatis PA13 | Pantoea ananatis LMG 20103 |
| Leuconostoc mesenteroides | Anaeromyxobacter sp. Fw109-5 | Serratia sp. AS13 |
| subsp. mesenteroides J18 | | |
| Prevotella maculosa OT 289 | Acetobacterium woodii DSM | Roseburia hominis A2-183 |
| | 1030 | |
| Mycoplasma fermentans PG18 | Thiobacillus denitrificans | Yersinia enterocolitica subsp. |
| | ATCC 25259 | palearctica Yl |
| Desulfobacterium | Sorangium cellulosum ‘So ce 56’ | Subdoligranulum sp. |
| autotrophicum HRM2 | | 4_3_54A2FAA |
| Prevotella histicola F0411 | Arcobacter butzleri ED-1 | Eubacterium limosum KIST612 |
| Arcobacter nitrofigilis DSM | Leuconostoc sp. C2 | Mycoplasma hyopneumoniae 168 |
| 7299 | | |
| Eubacterium haIIii DSM 3353 | Cellulophaga lytica DSM 7489 | Mycoplasma hyorhinis HUB-1 |
| Mycoplasma hyorhinis MCLD | Helcococcus kunzii ATCC | Campylobacter showae RM3277 |
| | 51366 | |
| Granulicella mallensis | Helicobacter pylori 83 | Arcobacter sp. L |
| MP5ACTX8 | | |
| Gluconacetobacter xylinus | Anaerotruncus colihominis | Treponema brennaborense DSM |
| NBRC 3288 | DSM 17241 | 12168 |
| Myroides odoratimimus CIP | Granulibacter bethesdensis | Bacteroides sp. 2_2_4 |
| 101113 | CGDNIHl | |
| Campylobacter jejuni | Bacteroides sp. D20 | Geobacillus thermodenitrificans |
| | | NG80-2 |
| Bacteroides sp. 9_1_42FAA | Microcystis aeruginosa PCC | Bacteroides fragilis 3_1_12 |
| | 7806 | |
| lmtechella halotolerans K1 | Acaryochloris marina | Caldilinea aerophila DSM 14535 = |
| | MBIC11017 | NBRC 104270 |
| Planctomyces brasiliensis DSM | Bacteroides sp. 01 | Trichodesmium erythraeum |
| 5305 | | IMS101 |

TABLE 5
Recombinases.
RECOMBINASES

| | |
|---|---|
| YML032 | Saccharomyces cerevisiae |
| P433S1 | Homo sapiens |
| H6L1F4 | Saprospira grandis |
| E6XGY4 | Shewanella putrefaciens |
| E4SSA7 | Caldicellulosiruptor kristjanssonli |
| C7F4E8 | Cyanophage pSS2 |
| B9MNF3 | Anaerocellum thermophilum |
| F8CQD8 | Myxococcus fulvus |
| E4QCOO | Caldicellulosi ruptor hydrothermalis |
| B2HP13 | Mycobacterium marinum |
| Q7N2Y8 | Photorhabdus luminescens |
| Q1DAT2 | Myxococcus xanthus |
| H8MHN3 | Corallococcus coralloides |
| COQSA2 | Persephonella marina |
| Q9AKZO | Legionella pneumophila |
| A3NMOO | Burkholderia pseudomallei |
| Q08VK7 | Stigmatella aurantiaca |
| A4XJGS | Caldicellulosiruptor saccharolyticus |
| Q984J6 | Rhizobium loti |
| Q8KQWO | Vibrio cholerae |
| Q9T172 | Listeria phage A118 |
| A41VH9 | Salmonella newport |
| D1BWP7 | Xylanlmonas cellulosilytica |
| E4SEV3 | Caldicellulosiruptor kronotskyensis |
| F6AU64 | Delftia sp. |
| A1B8G1 | Paracoccus denltrificans |
| A1AQ73 | Pelobacter propionicus |
| ASWFS1 | Psychrobacter sp. |
| D9SWGO | Clostridlum cellulovorans |
| E8MTD2 | Bifidobacterium longum subsp. infantis |
| F6DWY3 | Sinorhizobium melilotl |
| A7HOW7 | Campylobacter curvus |
| GOHBS6 | Corynebacterium variabile |
| Q87CQ1 | Xylella fastidiosa |
| Q7N2Y8 | Photorhabdus luminescens subsp. laumondii |
| BlXAU6 | Escherichia coli |
| Q9MBV8 | Lactotoccus phage ull6.2 |
| A4W7K6 | Enterobacter sp. |
| Q18AA7 | Clostridium difficile |
| C6CX43 | Paenibacillus sp. |
| D3RWF4 | Allochromatium vinosum |
| F9ZNV9 | Acidithiobacillus caldus |
| B7VNT1 | Vibrio splendidus |
| Q3ABHS | Carboxydothermus hydrogenoformans |
| A7H394 | Campylobacter jejuni subsp. doylei |
| Q2NSA3 | Sodalis glossinidius |
| ElR1F4 | Spirochaeta smaragdinae |
| B8F617 | Haemophilus parasuis serovar S |
| QSFN39 | Gluconobacter oxydans |
| E4RNA4 | Halanaerobium sp. |
| DSCJK9 | Enterobacter cloacae subsp. cloacae |
| E7BFDS | Neisseria meningitidis serogroup A |
| GOEOR4 | Enterobacter aerogenes |
| Q66Bl7 | Yersinia pseudotuberculosis |
| C6ACZ8 | Bartonella grahamii |
| A91YA2 | Bartone/la tribocorum |
| A1RH51 | Shewanella sp. |
| B4EWR5 | Proteus mlrabilis |
| ClCGll | Streptococcus pneumoniae |
| C3KA14 | Pseudomonas fluorescens |
| C3NU24 | Vibrio cholerae serotype 01 |
| A6TRD8 | Alkaliphilus metalliredigens |
| QlCQT2 | Streptococcus pyogenes serotype M12 |
| Q9A029 | Streptococcus pyogenes serotype Ml |
| C1D7P7 | Laribacter hongkongensis |
| A9MSD6 | Salmonella paratyphi B |
| B4TDT1 | Salmonella heidelberg |
| 87LNE9 | Escherichia fergusonii |
| D3DGM9 | Hydrogenobacter thermophilus |
| Q32GM7 | Shigella dysenteriae serotype 1 |
| Q8Z7Y3 | Salmonella typhi |
| C6AM23 | Aggregatibacter aphrophilus |
| Q926A1 | listeria innocua serovar 6a |
| CG19468 | Drosophila melanogaster |
| A2RDQ6 | Streptococcus pyogenes serotype MS |
| D3V014 | Xenorhabdus bovienii |
| F5ZL14 | Streptococcus parauberis |
| E8T306 | Thermovibrio ammonificans |
| F8LPH2 | Streptococcus sa livarius |
| H8L634 | Frateuria aurantia |
| F3YBJO | Melissococcus plutonius |
| AFA45162.1 | Avibacterium paragallinarum |
| ZP_03338172.1 | Salmonella enterica subsp. |
| | enterica serovar Typhi str. 404ty |
| ZP_01797282.1 | Haemophilus influenzae R3021 |
| ZP_00683037.1 | Xylella fastidiosa Ann-1 |
| CBl82624.1 | Bartonella schoenbuchensfs R1 |
| CAA23976.1 | Enterobacteria phage lambda |
| ZP_08900462.1 | Gluconacetobacter oboediens 174Bp2 |
| ZP_07805046.1 | Helicobacter cinaedi CCUG 18818 |
| CBl78688.1 | Bartonella sp. AR 15-3 |
| EEW20557.1 | Listeria monocytogenes FSL R2-503 |
| ZP_07873574.1 | listeria ivanovii FSL F6-596 |
| ZP_09071954.1 | Paenibacillus larvae subsp. larvae B-3650 |
| EGC95709.1 | Escherichia fergusonii ECD227 |
| yp_001648906.1 | Enterobacteria phage Min27 |
| EHC90429.1 | Salmonella enterica subsp. enterica |
| | serovar Rubislaw str. A4-653 |
| YP_004666545.1 | Myxococcus fulvus HW-1 |
| ZP_03220723.1 | Salmonella enter/ca subsp. enterica |
| | serovar Javiana str. |
| | GA_MM04042433 |
| EHC91146.1 | Salmonella enterica subsp. enterica |
| | serovar Senftenberg str. A4543 |
| AAF28115.1, AF153317_10 | Shigella dysenteriae |
| ZP_06538095.1 | Salmonella enterica subsp. enterica |
| | serovar Typhi str. AG3 |
| YP_630241.1 | Myxococcus xanthus DK 1622 |
| ZP_07683610.1 | Shigella dysenteriae 1617 |
| YP_003991543.1 | Caldicellulosiruptor hydrothermalis 108 |
| YP_004027154.1 | Caldicellulosiruptor kristjanssonH 177R1B |
| YP_005368024.1 | Corallococcus coralloides DSM 2259 |
| YP_003952450.1 | Stigmatella aurantiaca DW4/3-1 |
| YP_001180241.1 | Caldicellulosiruptor saccharolyticus DSM 8903 |
| AAM08027.1 | Providencia rettgeri |
| ABD63859.1 | Lactococcus phage phismq86 |
| yp_004023409.1 | Caldicellulosiruptor kronotskyensis 2002 |
| YP_005892581 .1 | Neisseria meningitidis WUE 2594 |
| CBX21356.1 | Neisseria lactamica Y92-1009 |
| ZP_02494581 .1 | Burkholderia pseudomallei NCTC 13177 |
| EDS78268.1 | Clostridium botulinum C str. Eklund |
| yp_003845006.1 | Clostridium cellulovorans 7438 |
| yp_003662329.1 | Xenorhabdus nematophila ATCC 19061 |
| yp_002795496.1 | laribacter hongkongensis HLHK9 |
| yp_001063371.1 | Burkholderia pseudomallei 668 |
| YP_001320415.1 | Alkaliphilus metalliredigens QYMF |
| AEU09829.1 | Klebsiella pneumoniae |
| ABY28344.1 | Vibrio cholerae 0139 |
| YP_001101797.1 | Yersinia ruckeri |
| EGT79392.1 | Haemophilus haemolyticus M21639 |
| yp_003084246.1 | Cyanophage PSS2 |
| ZP_06124041.2 | Providencia rettgeri DSM 1131 |
| NP_996684.1 | lactococcus phage phiLC3 |
| YP_005377560.1 | Frateuria aurantia DSM 6220 |
| ZP_05850920.1 | Haemophilus influenzae NT127 |
| CBW39270.1 | Streptococcus phage 2167 |
| ABW02831.1 | Aggregatibacter aphrophilus NJ8700 |
| EHE64346.1 | Streptococcus pneumoniae EU-NPOl |
| yp_004456677.1 | Melissococcus plutonius ATCC 35311 |
| EGV02688.1 | Streptococcus infantis SK970 |
| EHL46421.1 | Salmonella enterica subsp. enterica |
| | serovar Montevideo str. SARB30 |
| yp_004875641.1 | Baciffus subtilis subsp. spizizenii TU-B-10 |
| yp_003611805.1 | Enterobacter cloacae subsp. cloacae ATCC 13047 |
| yp_005352506.1 | Photobacterium damselae subsp. damselae |
| ZP_08247669.1 | Neisseria bacilliformis ATCC BAA-1200 |
| EG086949.1 | Clostridium botulinum C str. Stockholm |
| yp_004594671.1 | Enterobacter aerogenes KCTC 2190 |
| ZP_07797103.1 | Pseudomonas aeruginosa 39016 |
| CBl78049.1 | Bartonella rochalimae ATCC BAA-1498 |
| ZP_07922626.1 | Fusobacterium sp. 3_1_5R |
| ZP_07042769.1 | Comamonas testosteroni 544 |
| 2P_07367149.1 | Pediococcus acidilactici DSM 20284 |
| EHY70960.1 | Salmonella enterica subsp. houtenae str. ATCC BAA-1581 |
| yp-455377.1 | Sodalis glossinidius str. ‘morsitans 1 |
| y p _004479394.1 | Streptococcus parauberis KCTC 11537 |
| yp_001610320.1 | Bartonella tribocorum CIP 105476 |
| ZP_06552348.1 | Klebsiella sp. 1_1_55 |
| ZP_08564335.1 | lactobacillus ruminis SPM0211 |
| ZP_10073638.1 | Haemophilus paraphrohaemolyticus HK411 |
| EHN14107.1 | Clostridium sporogenes PA 3679 |
| ZP_04979099.1 | Mannheimia haemolytica PHL213 |
| ZP_06016835.1 | Klebsiella pneumoniae subsp. rhinoscferomatis ATCC 13884 |
| CBW39216.1 | Streptococcus phage 8140 |
| yp_005719596.1 | Sinorhizobium me/iloti SM11 |
| CBW38953.1 | Streptococcus phage V22 |
| ZP_04822781.1 | Clostridium botulinum El str, 1BoNT E Beluga’ |
| yp_596543.1 | Streptococcus phage 9429.2 |
| YP_003804225.1 | Spirochaeta smaragdinae DSM 11293 |
| yp_004549676.1 | Sinorhizobium meliloti AK83 |
| VP_001128217.1 | Streptococcus pyogenes str. Manfredo |
| yp_360509.1 | Carboxydothermus hydrogenoformans Z-2901 |
| EHE76000.1 | Streptococcus pneumoniae GA11426 |
| YP_005953460.1 | Klebsiella pneumoniae KCTC 2242 |
| CCB95242.1 | Streptococcus salivarius JIM8777 |
| ZP_06691943.1 | Acinetobacter sp. SH024 |
| ZP_10036178.1 | Burkholderia sp. Chl-1 |
| ZP_03625981 .1 | Streptococcus suis 89/1591 |
| BAB53634.1 | Mesorhizobium loti MAFF303099 |
| YP_006078894.1 | Streptococcus suis 5512 |
| ZP_07075805.1 | listeria monocytogenes FSL N l-017 |
| yp_002128512.1 | lodobacteriophage phiPLPE |
| YP_002971742.1 | Bartonella grahamii as4aup |
| ZP_09588597.1 | Fusobacterium sp. 12_1B |
| YP_003150705.1 | Cryptobacterium curtum DSM 15641 |
| ZP_01960732.1 | Bacteroides caccae ATCC 43185 |
| ZP_06011746.1 | Leptotrichia goodfellowii F0264 |
| NP_471091.1 | listeria innocua Cli p11262 |
| AAX89425.1 | Vibrio cholerae non-01/non-0139 |
| ZP_04661743.1 | Acinetobacter baumannii AB900 |
| YP_003993926.1 | Halanaerobium hydrogeniformans |
| YP_001852190.1 | Mycobacterium marinum M |
| ZP_07400314.1 | Peptoniphilus duerdenii ATCC BAA-1640 |
| ZP_08039150.1 | Serratia symbiotica str. Tucson |
| YP_004208804.1 | Bifidobacterium longum subsp. infantis 157F |
| YP_001397974.1 | Campylobacter jejuni subsp. doylei 269.97 |
| YP_006001389.1 | Xylella fastidiosa subsp. fastidiosa GB514 |
| AAF84457.1, AE003991_9 | Xylella fastidiosa 9a5c |
| ZP_09143689.1 | Acinetobacter sp. P8-3-8 |
| ABD48929.1 | Streptococcus phage M102 |
| EIA80005.1 | Campylobacter coli 1957 |
| YP_004759254.1 | Corynebacterium variabile DSM 44702 |
| ZP_03297235.1 | Collinsella stercoris DSM 13279 |
| YP_001409109.1 | Campylobacter curvus 525.92 |
| EIB51049.1 | Campylobacter jejuni subsp. jejuni 2008-988 |
| EIA84440.1 | Campylobacter coli 67-8 |
| EIB08252.1 | Campylobacter coli LMG 9860 |
| EIA50333.1 | Campylobacter coli 2685 |
| EIA74674.1 | Campylobacter coli 132-6 |
| YP_003308034.1 | Sebaldella termitidis ATCC 33386 |
| YP_003325187.1 | Xylanimonas cellulosilytica DSM 15894 |
| EIA59740.1 | Campylobacter coli 80352 |
| ZP_01771318.1 | Collinsella aerofaciens ATCC 25986 |
| ZP_07889816.1 | Aggregatibacter segnls ATCC 33393 |
| YP_002475717.1 | Haemophilus parasuis SH0165 |
| YP_901551.1 | Pelobacter propionicus DSM 2379 |
| ZP_02479394.1 | Haemophilus parasuis 29755 |
| ZP_00372172.1 | Campylobacter upsaliensis RM3195 |
| YP_004748640.1 | Acidithiobacillus caldus SM-1 |
| ZP_05294088.1 | Acidithiobacillus caldus ATCC 51756 |
| ZP_07463816.1 | Streptococcus gallolyticus subsp. gallolyticus TX20005 |
| YP_070310.1 | Yersinia pseudotuberculosis IP 32953 |
| ZP_04898500.1 | Burkholderia pseudomallei Pasteur 52237 |
| EHK78038.1 | Sinorhizobium meliloti CCNWSX0020 |
| ZP_05880433.1 | Vibrio furnissii CIP 102972 |
| ZP_09397479.1 | Acetobacteraceae bacterium AT-5844 |
| ZP_04809499.1 | Helicobacter pullorum MIT 98-5489 |
| YP_002872416.1 | Pseudomonas fluorescens SBW25 |
| ZP_06157475.1 | Photobacterium damselae subsp. damselae CIP 102761 |
| YP_001327594.1 | Sinorhizobium medlcae WSM419 |
| YP_005511208.1 | Hydrogenobacter thermophilus TK-6 |
| YP_004662889.1 | Simkania negevensis Z |
| YP_002281965.1 | Rhizobium leguminosarum bv. trifolii WSM2304 |
| YP_002731548.1 | Persephonella marina EX-Hl |
| ZP_08768668.1 | Thiocapsa marina 5811 |
| ZP_08824405.1 | Thiorhodococcus drewsii AZl |
| ZP_08977565.1 | Desulfitobacterium metalllreducens DSM 15288 |
| YP_003456916.1 | Allochromatium vinosum DSM 180 |
| YP_002417106.1 | Vibrio splendidus LGP32 |
| ZP_03497413.1 | Thermus aquaticus Y51MC23 |
| YP_001175737.1 | Enterobacter sp. 638 |
| ZP_08926729.1 | Thiocystis violascens DSM 198 |
| ZP_01947910.1 | Vibrio cholerae 1587 |
| ZP_08267258.1 | Brevundimonas diminuta ATCC 11568 |
| YP_004488616.1 | Delftia sp. Csl-4 |
| YP_005162586.1 | Corynebacterium diphtheriae C7 (beta) |
| ADZ13631.1 | Cronobacter phage ENT47670 |
| ZP_09809048.1 | Thiorhodovibrio sp. 970 |
| YP_003010343.1 | Paenibacillus sp. JDR-2 |
| NP_930169.1 | Photorhabdus luminescens subsp. laumondii TTOl |
| yp_001280242.1 | Psychrobacter sp. PRwf-1 |
| EHl54313.1 | Aeromonas salmonicida subsp. salmonicida 01-8526 |
| YP_192864.1 | Gluconobacter oxydans 621H |
| ZP_08680759.1 | Sporosarcina newyorkensis 2681 |
| ZP_04762755.1 | Acldovorax delafieldil 2AN |
| ZP_09077613.1 | Paenibacil/us elgii 869 |
| ZP_09899678.1 | Methylomicrobium album BG8 |
| ZP_07949994.1 | Enterobacteriaceae bacterium 9_2_54FAA |
| YP_917501.1 | Paracoccus denitrificans PD1222 |
| ZP_09013441.1 | Commensalibacter intestini A911 |
| ZP_07908870.1 | Mobiluncus curtisil ATCC 51333 |
| ZP_00235039.1 | listeria monocytogenes str. 1/2a F6854 |
| EGH43274.1 | Pseudomonas syringae pv. pisi str. 1704B |
| ZP_05297269.1 | Listeria monocytogenes FSL J2-003 |
| EGH87883.1 | Pseudomonas syringae pv, lachrymans str. M301315 |
| YP_001418489.1 | Xanthobacter autotrophicus Py2 |
| ZP_02971366.1 | Ureaplasma parvurn serovar 6 str. ATCC 27818 |
| YP_005087203.1 | Rhodococcus phage REQ3 |
| ZP_03771988.1 | Ureaplasma urealyticum serovar 8 str. ATCC 27618 |
| EDX53145.1 | Ureaplasma urealyticum serovar 12 str. ATCC 33696 |
| YP_003897727.1 | Halomonas elongata DSM 2581 |
| EHT14033.1 | Klebsiella oxytoca 10-5250 |
| ZP_02327781 .1 | Paenibacillus larvae subsp. larvae BRL-230010 |
| ZP_09377516.1 | Hafnia alvei ATCC 51873 |
| YP_002233655.1 | Burkholderia cenocepacia J2315 |
| YP_003374177.1 | Gardnerella vaginalis 409-05 |
| ZP_03805638.1 | Proteus penneri ATCC 35198 |
| ZP_05830211 .1 | Acinetobacter baumannii ATCC 19606 |
| ZP_06324416.1 | Staphylococcus aureus subsp. aureus 0139 |
| ZP_09243850.1 | Pseudoalteromonas sp. BSi20495 |
| ZP_03477183.1 | Parabacteroides johnsonii DSM 18315 |
| YP_001453480.1 | Citrobacter koseri ATCC BAA-895 |
| AAG00321.1 | Borrelia hermsii |
| EHC73330.1 | Salmonella enterica subsp. enterica serovar |
| | Mississippi str. A4-633 |
| VP_005098013.1 | Salmonella phage SPNlS |
| EDZ19028.1 | Salmonella enterica subsp, enterica |
| | serovar Kentucky str. CDC 191 |
| EFW50178.1 | Shigella dvsenteriae CDC 74• 1112 |
| ZP_03935819.1 | Corynebacterium striatum ATCC 6940 |
| ZP_08306099.1 | Klebsiella sp. MS 92-3 |
| ZP_07380439.1 | Pantoea sp. aB |
| ZP_03840270.1 | Proteus mirabilis ATCC 29906 |
| ZP_03318967.1 | Providencia alcalifaciens DSM 30120 |
| AEW24552.1 | Escherichia phage TL-2011b |
| ZP_03706978.1 | Clostridium methylpentosum DSM 5476 |
| VP_004327343.1 | Erwinia phage phiEt88 |
| ZP_08261052.1 | Gemella sanguinis M325 |
| ZP_06640797 .1 | Serratia odorifera DSM 4582 |
| VP_004355228.1 | Pseudomonas brassicacearum subsp. |
| | brassicacearum NFM421 |
| ZP_03544152.1 | Comamonas testosteroni KF-1 |
| ZP_06714885.1 | Edwardsiella tarda ATCC 23685 |
| ZP_09015869.1 | Brenneria sp. EniD312 |
| CAC33455.1 | Legionella pneumophila |
| EFU90864.1 | Enterococcus faecalis TX0630 |
| yp_002933453.1 | Edwardsiella ictaluri 93-146 |
| VP_003295659.1 | Edwardsiella tarda EIB202 |
| ZP_05361195.1 | Acinetobacter radioresistens SK82 |
| yp_673748.1 | Chelativorans sp. BNCl |
| EGE24382.1 | Moraxella catarrhalis 101P30Bl |
| VP_001886967.1 | Clostridium botulinum B str. Eklund 17B |
| yp_003467094.1 | Xenorhabdus bovieniiSS-2004 |
| yp_003742593.1 | Erwinia billingiae Eb661 |
| AEl70845.1 | EBPR podovirus 1 |
| VP_002502020.1 | Methylobacterium nodulans ORS 2060 |
| VP_003931279.1 | Pantoea vagans C9-1 |
| ZP_09514368.1 | Oceanicola sp. 5124 |
| EHC38944.1 | Salmonella enterica subsp, enterica |
| | serovar Gaminara str. A4-567 |
| VP_001335044.1 | Klebsiella pneumoniae subsp. |
| | pneumoniae MGH 78578 |
| VP_004150652.1 | Thermovibrio ammonificans HB-1 |
| YP_001139941.1 | Corynebacterium glutamicum R |
| NP_461568.2 | Phage Gifsy-1 |
| AEW24678.1 | Escherichia phage TL-201lc |
| AAC26069.1 | Salmonella enterica subsp. |
| | enterica serovar Typhimurium |
| ZP_01237386.1 | Vibrio angustum 514 |
| VP_004630586.1 | Corynebacterium ulcerans BR-AD22 |
| ZP_05289346.1 | Listeria monocytogenes FSL F2-515 |
| EIC64366.1 | Mycobacterium abscessus M93 |
| VP_264268.1 | Psychrobacter arcticus 273-4 |
| ZP_07375909.1 | Ahrensia sp. R2A130 |
| VP_003477226.1 | Thermoanaerobacter italicus Ab9 |
| VP_004011824.1 | Rhodomicrobium vannielii ATCC 17100 |
| ZP_06074083.1 | Acinetobacter radioresistens SH164 |
| VP_132232.1 | Photobacterium profundum SS9 |
| VP_003657357.1 | Segniliparus rotundus DSM 44985 |
| ZP_09732369.1 | Megamonas funiformis Y/T 11815 |
| VP_235897.1 | Pseudomonas syringae pv. syrlngae B728a |
| EGH59138.1 | Pseudomonas syringae pv. maculicola str. ES4326 |
| EGE19718.1 | Moraxella catarrhalis BCl |
| ZP_07903104.1 | Eubacterium saburreum DSM 3986 |
| EFC94742.1 | Clostridium hathewayi DSM 13479 |
| ZP_08334226.1 | Lachnospiraceae bacterium 9_1_43BFAA |
| ZP_09322413.1 | Eubacteriaceae bacterium ACC19a |
| VP_003609870.1 | aurkholderia sp. CCGE1002 |
| ZP_060741S5.1 | Bacteroides sp. 2_1_33B |
| YP_004168204.1 | Nitratifractor salsuginis DSM 16511 |
| YP_003162220.1 | Jonesia denitrificans DSM 20603 |
| AAA22084.1 | Agrobacterium tumefaciens |
| AAS46728.1 | Aeromonas hydrophila |
| CBL42165.1 | butyrate-producing bacterium SS3/4 |
| ZP_08499194.1 | Enterobacter hormaechei ATCC 49162 |
| ACD38903.1 | Pseudomonas aeruginosa |
| yp_005098041.1 | Pseudomonas phage phi297 |
| yp_003438695.1 | Klebsiella variicola At-22 |
| EGD06615.1 | Burkholderia sp. TJl49 |
| EHS95796.1 | Klebsiella oxytoca 10-5243 |
| yp_001863170.1 | Burkholderia phymatum STM815 |
| yp_001667964.1 | Pseudomonas putida GB-1 |
| yp_002238960.1 | Klebsiella pneumoniae 342 |
| yp_006011723.1 | Shewanella putrefaciens 200 |
| yp_005321079.1 | Saprospfra grandis str. Lewin |
| EIC70403.1 | Mycobacterium abscessus M94 |
| ZP_09433274.1 | Bradyrhizobium sp. STM 3843 |
| 8A185059.1 | Bacillus subtilis subsp. natto BEST195 |
| yp_516203.1 | Sodalis phage phiSGl |
| ZP_06299504.1 | Parachlamydia acanthamoebae str. Hall's coccus |
| EGP42731.1 | Achromobacter xylosoxidans AXX-A |
| ZP_03311406.1 | Desulfovibrio piger ATCC 29098 |
| ZP_04945782.1 | Burkholderia dolosa AU0158 |
| ZP_07200498.1 | delta proteobacterium NaphS2 |
| ZP_07741429.1 | Vibrio caribbenthicus ATCC BAA-2122 |
| yp_003084663.1 | Dyadobacterfermentans DSM 18053 |
| ZP_02163271.1 | Kordia algicida OT-1 |
| yp_005357245.1 | Flavobacterium indicum G PTSAl00-9 |
| ZP_06177392.1 | Vibrio harveyi 1DA3 |
| YP_004655025.1 | Runella slithyformis DSM 19594 |
| yp_001196868.1 | Flavobacterium johnsoniae UW101 |
| yp_004988339.1 | Owenweeksia hongkongensis DSM 17368 |
| yp_006017652.1 | Riemerella anatipestifer RA-GD |
| ZP_02183546.1 | Flavobacteriales bacterium ALC-1 |
| yp_003998593.1 | Leadbetterella byssophila DSM 17132 |
| YP_004843584.1 | Flavobacterium branchiophilum FL-15 |
| YP_006190082.1 | Paenibacillus mucilaginosus K02 |
| ZP_09236879.1 | Pseudoafteromonas sp. BSi20439 |
| yp_002911448.1 | Burkholderia glumae BGRl |
| yp_004238525.1 | Weeksella virosa DSM 16922 |
| ZP_09524021.1 | Myroides odoratimimus CCUG 10230 |
| YP_004776003.1 | Cyclobacterium marinum DSM 745 |
| YP_004581188.1 | Lacinutrix sp. SH-3-7-4 |
| ZP_02435739.1 | Bacteroides stercoris ATCC 43183 |
| yp_005208095.1 | Pseudomonas fluorescens F113 |
| YP_004445529.1 | Haliscomenobacter hydrossis DSM 1100 |
| yp_005930618.1 | Pseudomonas putida BIRD-1 |
| YP_005006679.1 | Niastella koreensis GR20-10 |
| yp_005633953.1 | Vibrio cholerae LMA3984-4 |
| yp_004789848.1 | Muricauda ruestringensis DSM 13258 |
| yp_005869785.1 | Lactococcus garvieae Lg2 |
| YP_003907168.1 | Burkholderia sp. CCGE1003 |
| YP_006015496.1 | Staphylococcus pseudintermedius ED99 |
| YP_004941085.1 | Flavobacterium columnare ATCC 49512 |
| YP_003700049.1 | Bacillus selenitireducens MLS10 |
| YP_001760744.1 | Shewanella woodyi ATCC 51908 |
| yp_005865109.1 | lactobacillus rhamnosus GG |
| yp_004272912.1 | Pedobacter saltans DSM 12145 |
| yp_001295947.1 | Flavobacterium psychrophilum JIP02/86 |
| NP_212876.2 | Borrelia burgdorferi B31 |
| yp_005034813.1 | Bacteriovorax marinus SJ |
| yp_369306.1 | Burkholderia sp. 383 |
| YP_005820203.1 | Fibrobacter succinogenes subsp. succinogenes 585 |
| yp_002005623.1 | Cupriavidus taiwanensis LMG 19424 |
| yp_348340.1 | Pseudomonas fluorescens Pf0-1 |
| yp_004380540.1 | Pseudomonas mendocina NK-01 |
| yp_005858762.1 | Lactobacillus casei BD-11 |
| YP_692011.1 | Alcanivorax borkumensis SK2 |
| yp_001099394.1 | Herminiimonas arsenicoxydans |
| YP_004166442.1 | Cellulophaga algicola DSM 14237 |
| ZP_09641960.1 | Odoribacter laneus YIT 12061 |
| EHE92415.1 | Lactococcus lactis subsp. cremoris CNCM 1-1631 |
| YP_583727.1 | Cupriavidus metallidurans CH34 |
| ZP_02206967.1 | Coprococcus eutactus ATCC 27759 |
| y p _004890503.1 | Lactobacillus plantarum WCFSl |
| ZP_08586327.1 | Bacteroides sp. 1_1_30 |
| YP_005672087.1 | Clostridium acetobutylicum EA 2018 |
| ZP_09521413.1 | Lachnospiraceae bacterium ACC2 |
| EHE89971.1 | Streptococcus thermophilus CNCM 1-1630 |
| ZP_02996105.1 | Clostridium sporogenes ATCC 15579 |
| yp_396489.1 | Lactobacillus sakei subsp. sakei 23K |
| YP_004739144.1 | Zobelfia galactanivorans |
| y p_004993437.1 | Vibrio furnissii NCTC 11218 |
| y p_005092407.1 | Oceanimonas sp, GK1 |
| yp_253435.1 | Staphylococcus haemolyticus JCSC1435 |
| yp_005848993.1 | Lactobacillus fermentum CECT 5716 |
| yp_006180809.1 | Halobacillus halophilus DSM 2266 |
| yp_574526.1 | Chromohalobacter salexigens DSM 3043 |
| YP_004396695.1 | Clostridium botulinum BKT015925 |
| NP_889567.1 | Bordetella bronchiseptica RB50 |
| YP_006217717.1 | Providencia stuartii MRSN 2154 |
| yp_001155602.1 | Polynucleobacter necessarius subsp. |
| | asymbioticus QLW-PlDMWA-1 |
| ZP_09337816.1 | Tannerella sp. 6_1_58FAA_CT1 |
| ZP_09023107.1 | Alistipes indistinctus YIT 12060 |
| yp_661287.1 | Pseudoalteromonas atlantica T6c |
| NP_722442.1 | Streptococcus mutans UA159 |
| YP_002265207.1 | Aliivibrio salmonicida LF11238 |
| ZP_09188055.1 | Halomonas boliviensis LC1 |
| yp_003811665.1 | gamma proteobacterium HdNl |
| ZP_03302243.1 | Bacteroides dorei DSM 17855 |
| EHG11465.1 | Streptococcus intermedius F0395 |
| YP_004726213.1 | Weissella koreensis KACC 15510 |
| ZP_08890725.1 | Lactobacillus sp. 7_1_47FAA |
| yp_004977003.1 | Burkholderia sp. Yl23 |
| yp_927263.1 | Shewanella amazonensis SB2B |
| ZP_08339829.1 | Lachnospiraceae bacterium 2_1_46FAA |
| yp_274842.1 | Pseudomonas syringae pv. phaseolicola 1448A |
| YP_005975666.1 | Pseudomonas aeruginosa M18 |
| YP_006035100.1 | Streptococcus gallolyticus subsp. gallolyticus ATCC 43143 |
| YP_005412494.1 | Streptococcus pyogenes MGAS1882 |
| EH078754.1 | Fusobacterium nucleatum subsp. animalis OT 420 |
| YP_004317691.1 | Sphingobacterium sp. 21 |
| YP_003163767.1 | Leptotrichia buccalis C-1013-b |
| N P_693974.1 | Oceanobacillus iheyensis HTE831 |
| ZP_09148363.1 | Clostridium hathewayi WAL-18680 |
| ZP_02073778.1 | Clostridium sp. L2-50 |
| VP_005862099.1 | Lactobacillus johnsonii DPC 6026 |
| yp_006085650.1 | Streptococcus suis A7 |
| YP_786508.1 | Bordetella avium 197N |
| EHG 25612.1 | Selenomonas noxia F0398 |
| YP_942451.1 | Psychromonas ingrahamii 37 |
| ZP_02421015.1 | Anaerostipes caccae DSM 14662 |
| yp_005147109.1 | Clostridium sp. BN LllOO |
| ZP_03800678.1 | Coprococcus comes ATCC 27758 |
| YP_001631170.1 | Bordetella petrii DSM 12804 |
| yp_005739407.1 | Staphylococcus aureus subsp. aureus JKD6159 |
| YP_005863385.1 | Lactobacillus salivarius CECT 5713 |
| yp_856547.1 | Aeromonas hydrophila subsp. hydrophila ATCC 7966 |
| YP_005014978.1 | Tannerella forsythia ATCC 43037 |
| YP_004543651.1 | Desulfotomaculum ruminis DSM 2154 |
| ZP_08850119.1 | Dorea formicigenerans 4_6_53AFAA |
| yp_005938868.1 | Pseudomonas stutzeri DSM 4166 |
| yp_004971910.1 | Desu/fosporosinus orientis DSM 765 |
| YP_005704426.1 | Enterococcus faecalis 62 |
| yp_002930165.1 | Eu bacterium eligens ATCC 27750 |
| ZP_09112782.1 | Enterococcus saccharolyticus 30_1 |
| yp_004846059.1 | Pseudogu lbenkiania sp. NH8B |
| yp_001438598.1 | Cronobacter sakaza kii ATCC BAA-894 |
| N P_718112.1 | Shewanella oneidensis MR-1 |
| yp_005433748.1 | Selenomonas ruminantium subsp. lactilytica TAM6421 |
| ZP_02438733.1 | Clostridium sp. 552/1 |
| ZP_08616825.1 | lachnospiraceae bacterium 1_4_56FAA |
| ZP_09154646.1 | Johnsonella ignava ATCC 51276 |
| yp_001141717.1 | Aeromonas salmonicida subsp. salmonicida A449 |
| yp_006029734.1 | Ralstonia solanacearum Po82 |
| ZP_08470204.1 | Oysgonomonas mossii DSM 22836 |
| ZP_02033987.1 | Para bacteroides merdae ATCC 43184 |
| ZP_08474773.1 | Dysgonomonas gadei ATCC BAA-286 |
| ZP_03010636.1 | Bacteroides coprocola DSM 17136 |
| yp_001983252.1 | Cellvibrio japonicus Ueda107 |
| ZP_08603145.1 | lachnospiraceae bacterium 5_1_57FAA |
| YP_005474943.1 | Spirocha eta africa na DSM 8902 |
| ZP_09736507.1 | Facklam ia languida CCUG 37842 |
| ZP_05127187.1 | gamma proteobacterium NORS-3 |
| ZP_04449563.1 | Catonella morbi ATCC 51271 |
| ZP_02024907.1 | Eubacterium ventriosum ATCC 27560 |
| ZP_03712468.1 | Eikenel la corrodens ATCC 23834 |
| YP_004512397.1 | Methylomonas methanica MC09 |
| ZP_09334735.1 | Citrobacter freundii 4_7_47CFAA |
| ZP_03781365.1 | Blautia hvdrogenotrophica DSM 10507 |
| VP_004426052.1 | Alteromonas macleodii str. ‘Deep ecotype’ |
| ZP_03762445.1 | Clostridium asparagiforme DSM 15981 |
| VP_002800018.1 | Azotobacter vinelandii DJ |
| ZP_09532735.1 | Lachnospiraceae bacterium 7_1_58FAA |
| ZP_04854710.1 | Paenibacillus sp. oral taxon 786 str. 014 |
| VP_004468160.1 | Alteromonas sp. SN2 |
| VP_520049.1 | Desulfitobacterium hafniense YSl |
| VP_385574.1 | Geobacter metallireducens GS-15 |
| ZP_08336670.1 | lachnospiraceae bacterium 3_1_46FAA |
| VP_004915903.1 | Methvlom icrobium a lca liphilum |
| VP_527033.1 | Saccharophagus degradans 2-40 |
| YP_005318817.1 | Me/issococcus plutonius DAT561 |
| VP_004874525.1 | Tavlorella asi nigenita lis MCE3 |
| YP_005851623.1 | lactobacillus delbrueckii subsp. bulga ricus 2038 |
| ZP_09326776.1 | Oribacterium sp. ACB1 |
| ZP_03461890.1 | Bacteroides pectinophilus ATCC 43243 |
| VP_001571105.1 | Salmonella enterica subsp. arizonae |
| | serovar 62:z4, z23:-- str. RSK2980 |
| ZP_03166627.1 | Ruminococcus lactaris ATCC 29176 |
| ZP_03755701.1 | Roseburia inulinivorans DSM 16841 |
| ZP_03719260.1 | Neisseria flavescens N RL30031/H 210 |
| VP_005073546.1 | Paenibacillus terrae H PL-003 |
| VP_002312664.1 | Shewanella piezotolerans WP3 |
| VP_005005652.1 | Pediococcus cfaussenii ATCC BAA-344 |
| VP_003126133.1 | Chitinophaga pinensis DSM 2588 |
| ZP_08258595.1 | Gemella haemolvsans M341 |
| VP_001473852.1 | Shewanella sediminis HAW-EB3 |
| yp_001093802.1 | Shewanella loihica PV-4 |
| ZP_03777231.1 | Clostridium hvlemonae DSM 15053 |
| ZP_08149693.1 | Lachnospiraceae bacterium 4_1_37FAA |
| VP_001681197.1 | Heliobacterium modesticaldum leel |
| N P_951231.1 | Geobacter sulfurreducens PCA |
| ZP_02035690.1 | Bacteroides ca pillosus ATCC 29799 |
| VP_003556874.1 | Shewanel la violacea DSS12 |
| VP_037013.1 | Bacil l us thuringiensis serovar konkukian str. 9727 |
| VP_005272800.1 | Shewanella baltica 05678 |
| ZP_09537413.1 | Erysipelotrichaceae bacterium 21_3 |
| VP_00441 1234.1 | Spirochaeta coccoides DSM 17374 |
| VP_003428708.1 | Bacillus pseudofirmus OF4 |
| yp_006020740.1 | Shewanella ba ltica BA175 |
| ZP_09120679.1 | Prevotella sp. oral taxon 302 str. F0323 |
| ZP_02084161.1 | Clostridium bolteae ATCC BAA-613 |
| ZP_01725297.1 | Bacillus sp. 814905 |
| ZP_08604142.1 | Lachnospiraceae bacterium 3_1_57FAA_CT1 |
| yp_006231372.1 | Bacillus sp. JS |
| ZP_04451575.1 | Abiotrophia defectiva ATCC 49176 |
| ZP_04855253.1 | Ruminococcus sp. 5_1_39B_FAA |
| ZP_09697329.1 | Coprobacil l us sp. 8_2_54BFAA |
| VP_001182934.1 | Shewanella putrefaciens CN-32 |
| VP_004048639.1 | Neisseria lactamica 020-06 |
| ZP_03682223.1 | Catenibacterium mitsuokai DSM 15897 |
| y p _005889683.1 | Neisseria gonorrhoeae TCDC-NGD8107 |
| ZP_08331597.1 | Lachnospiraceae bacterium 6_1_63FAA |
| y p_091248.1 | Bacillus licheniformis DSM 13 • ATCC 14580 |
| ZP_09049102.1 | Clostridium sp. 7_3_54FAA |
| VP_004871614.1 | Glaciecola nitratireducens FR1064 |
| VP_003464587.1 | Listeria seeligeri serovar 1/2b str. SLCC3954 |
| VP_512292.1 | Escherichia phage phiVlO |
| ZP_09545227.1 | Eubacterium sp. 3_1_31 |
| ZP_02094048.1 | Parvimonas micra ATCC 33270 |
| VP_004560942.1 | Erysipelothrix rhusiopathiae str. Fujisawa |
| VP_005224087.1 | Treponema pallidum subsp. pallidum DAL-1 |
| ZP_03290838.1 | Clostridium nexile DSM 1787 |
| ZP_02867996.1 | Clostridium spiroforme DSM 1552 |
| VP_051100.1 | Pectobacterium atrosepticum SCRl1043 |
| VP_004247313.1 | Sphaerochaeta globus str. Buddy |
| VP_005199476.1 | Rahnella aquatilis CIP 78.65 = ATCC 33071 |
| VP_002472037.1 | Clostridium kluyveri NBRC 12016 |
| EHP49112.1 | Clostridium perfringens WAL-14572 |
| ZP_04454959.1 | Shuttleworthia satelles DSM 14600 |
| VP_005061390.1 | Sphaerochaeta pleomorpha str. Grapes |
| VP_005850725.1 | Lactobacillus helveticus H lO |
| ZP_09590809.1 | Prevotella micans F0438 |
| VP_005993583.1 | Pantoea ananatis PA13 |
| VP_003519587.1 | Pantoea ananatis LMG 20103 |
| VP_005174459.1 | Leuconostoc mesenteroides subsp. mesentero1des 118 |
| VP_001377632.1 | Anaeromyxobacter sp. Fw109-5 |
| yp_006024370.1 | Serratia sp. AS13 |
| ZP_09554100.1 | Prevotella maculosa OT 289 |
| VP_005269748.1 | Acetobacterium woodii DSM 1030 |
| VP_004838017.1 | Roseburia hominis A2-183 |
| BAH69619.1 | Mycoplasma fermentans PG18 |
| VP_313909.1 | Thiobacillus denitrificans ATCC 25259 |
| VP_006005235.1 | Yersinia enterocoJitica subsp. palearctica Yll |
| VP_002603986.1 | Desulfobacterium autotrophicum HRM2 |
| yp_001616277.1 | Sorangium cellulosum 1 $0 ce 561 |
| ZP_09342658.1 | Subdoligranulum sp. 4_3_54A2FAA |
| ZP_09103791.1 | Prevotella histicola F0411 |
| VP_005538542.1 | Arcobacter butzleri ED-1 |
| YP_003960581.1 | Eubacterium limosum KlST612 |
| VP_003655761 .1 | Arcobacter nitrofigilis DSM 7299 |
| VP_004704993.1 | Leuconostoc sp. C2 |
| VP_005888312.1 | Mycoplasma hyopneumoniae 168 |
| ZP_03717663.1 | Eubacterium hallii DSM 3353 |
| VP_004263391.1 | Cellulophaga lytica DSM 7489 |
| VP_003856575.1 | Mycoplasma hyorhinis HUB-1 |
| VP_005905159.1 | Mycoplasma hyorhinis MCLD |
| ZP_09738126.1 | Helcococcus kunzii ATCC 51366 |
| ZP_05364429.1 | Campylobacter showae RM3277 |
| VP_005059662.1 | Granulicella mallensis MP5ACTX8 |
| VP_005785121.1 | Helicobacter pylori 83 |
| VP_005553834.1 | Arcobacter sp, L |
| VP_004868821.1 | Gluconacetobacter xylinus NBRC 3288 |
| ZP_02442082.1 | Anaerotruncus colihominis DSM 17241 |
| VP_004438805.1 | Treponema brennaborense DSM 12168 |
| EH012592.1 | Myroides odoratimimus CIP 101113 |
| VP_744021.1 | Granulibacter bethesdensis CGDN/Hl |
| ZP_04550622.1 | Bacteroides sp. 2_2_4 |
| AAF82113.1 | Campylobacter je juni |
| ZP_06202195.1 | Bacteroides sp. 020 |
| yp_001125680.1 | Geobacillus thermodenitrificans NGS0-2 |
| ZP_04540182.1 | Bacteroides sp. 9_1_42FAA |
| CA090389.1 | Microcystis aeruginosa PCC 7806 |
| ZP_07811148.1 | Bacteroides fragilis 3_1_12 |
| ZP_09999130.1 | l mtechella ha lotolera ns Kl |
| yp_001516275.1 | Acaryochloris marina MBlC11017 |
| yp_005442188.1 | Caldilinea aerophila DSM 14535 = N BRC 104270 |
| yp_004270153.1 | Planctomyces brasiliensis DSM 5305 |
| ZP_08791189.1 | Bacteroides sp. Dl |
| YP_722968.1 | Trichodesmium erythraeum 1MS101 |

TABLE 6
Recombinases.
RECOMBINASES

| | |
|---|---|
| ZP_04451575.1 | Abiotrophia defectiva ATCC 49176 |
| VP_001516275.1 | Acaryochloris marina MBIC11017 |
| ZP_09397479.1 | Acetobacteraceae bacterium AT-5844 |
| VP_005269748.1 | Acetobacterium woodii DSM 1030 |
| EGP42731.1 | Achromobacter xylosoxidans AXX-A |
| ZP_05294088.1 | Acidithiobacillus ca Idus ATCC 51756 |
| VP_004748640.1 | Acidithiobacillus caldus SM-1 |
| ZP_04762755.1 | Acidovorax delafieldii 2AN |
| ZP_04661743.1 | Acinetobacter baumannii AB900 |
| ZP_05830211.1 | Acinetobacter baumannii ATCC 19606 |
| ZP_06074083.1 | Acinetobacter radioresistens SH164 |
| ZP_06074084.1 | Acinetobacter radioresistens SH164 |
| ZP_05360445.1 | Acinetobacter radioresistens SK82 |
| ZP_05361614.1 | Acinetobacter radioresistens SK82 |
| ZP_05361195.1 | Acinetobacter radioresistens SK82 |
| ZP_09143689.1 | Acinetobacter sp. P8-3-8 |
| ZP_09142708.1 | Acinetobacter sp. P8-3-8 |
| ZP_06691943.1 | Acinetobacter sp. SH024 |
| VP_002995586.1 | Aeromonas hydrophila |
| AAS46728.1 | Aeromonas hydrophila |
| VP_856547.1 | Aeromonas hydrophila subsp. hydrophila ATCC 7966 |
| EHl54313.1 | Aeromonas salmonicida subsp. salmonicida 01-B526 |
| VP_001141717.1 | Aeromonas salmonicida subsp. salmonicida A449 |
| VP_003006972.1 | Aggregatibacter aphrophilus NJ8700 |
| ABW02831.1 | Aggregatibacter aphrophilus NJ8700 |
| ZP_07889816.1 | Aggregatibacter segnis ATCC 33393 |
| AAA22084.1 | Agrobacteri um tumefaciens |
| ZP_07375909.1 | Ahrensia sp. R2A130 |
| VP_692011.1 | Alcanivorax borkumensis SK2 |
| VP_002265207.1 | Aliivibrio salmonicida LFl1238 |
| VP_002264570.1 | Aliivibrio salmonicida LFl1238 |
| ZP_09023107.1 | Alistipes indistinctus VIT 12060 |
| VP_001320415.1 | Alkaliphilus metalliredigens QVMF |
| VP_001321158.1 | Alkaliphilus metalliredigens QVMF |
| VP_003456916.1 | Allochromati um vinosum DSM 180 |
| VP_004426052.1 | Alteromonas macleodiistr. ‘Deep ecotype’ |
| VP_004427628.1 | Alteromonas macleodiistr. ‘Deep ecotype’ |
| VP_004468160.1 | Alteromonas sp. SN2 |
| VP_001377632.1 | Anaeromyxobacter sp. Fw109-5 |
| ZP_02421015.1 | Anaerostipes caccae DSM 14662 |
| ZP_02442082.1 | Anaerotruncus colihominis DSM 17241 |
| VP_005538542.1 | Arcobacter butzleri ED-1 |
| VP_003655761.1 | Arcobacter nitrofigilis DSM 7299 |
| VP_005553834.1 | Arcobacter sp. L |
| AFA45162.1 | Avibacterium paragallinarum |
| VP_002800018.1 | Azotobacter vinelandii DJ |
| VP_091248.1 | Bacillus licheniformis DSM 13 = ATCC 14580 |
| VP_003428708.1 | Bacillus pseudofirmus OF4 |
| VP_003700049.1 | Bacillus selenitireducens MLS10 |
| ZP_01725297.1 | Bacillus sp. B14905 |
| VP_006231372.1 | Bacillus sp. JS |
| BAl85059.1 | Bacillus subtilis subsp. natto BEST195 |
| YP_004875641.1 | Bacillus subtilis subsp. spizizenii TU-B-10 |
| yp_037013.1 | Bacillus thuringiensis serovar konkukian str. 97-27 |
| YP_005034813.1 | Bacteriovorax marinus SJ |
| yp_005036300.1 | Bacteriovorax marinus SJ |
| ZP_01960732.1 | Bacteroides caccae ATCC 43185 |
| ZP_02035690.1 | Bacteroides capillosus ATCC 29799 |
| ZP_03010636.1 | Bacteroides coprocola DSM 17136 |
| ZP_03302243.1 | Bacteroides dorei DSM 17855 |
| ZP_07811148.1 | Bacteroides fragilis 3_1_12 |
| ZP_03461890.1 | Bacteroides pectinophilus ATCC 43243 |
| ZP_08S86327.1 | Bacteroides sp. 1_1_30 |
| ZP_06074155.1 | Bacteroides sp. 2_1_338 |
| ZP_04550622.1 | Bacteroides sp. 2_2_4 |
| ZP_04540182.l | 8acteroides sp. 9_1_42FAA |
| ZP_08791189.1 | 8acteroides sp. Dl |
| ZP_06202195.1 | 8acteroides sp. D20 |
| ZP_02435739.l | 8acteroides stercoris ATCC 43183 |
| ZP_02434797.1 | 8acteroides stercoris ATCC 43183 |
| YP_002971742.1 | 8artonella grahamii as4aup |
| CBl78049.1 | Bartonella rochalimae ATCC BAA-1498 |
| (8182624.1 | 8artonella schoenbuchensis Rl |
| C8182531.1 | Bartonella schoenbuchensis Rl |
| CBl81728.1 | 8artonella schoenbuchensis Rl |
| CBl82748.1 | 8artonella schoenbuchensis Rl |
| (8182323.1 | 8artonella schoenbuchensis Rl |
| C8178688.1 | Bartonella sp. AR 15-3 |
| yp_001610320.1 | Bartonella tribocorum CIP 105476 |
| yp_001609377.1 | 8artonella tribocorum CIP 105476 |
| yp_004208804.1 | 8ifidobacterium longum subsp. infantis 157F |
| ZP_03781365.1 | Blautia hydrogenotrophica DSM 10507 |
| yp_786508.1 | Bordetella avium 197N |
| NP_889567.1 | 8ordetella bronchiseptica RB50 |
| YP_001631170.1 | Bordetella petrii DSM 12804 |
| NP_212876.2 | Borrelia burgdorferi B31 |
| AAG00321.1 | Borrelia hermsii |
| ZP_09433274.1 | Bradyrhizobium sp. STM 3843 |
| ZP_09015869.1 | Brenneria sp. EniD312 |
| ZP_08267258.1 | Brevundimonas diminuta ATCC 11568 |
| YP_002233655.1 | 8urkholderia cenocepacia J2315 |
| ZP_04945782.1 | Burkholderia dolosa AU0158 |
| YP_002911448.1 | Burkholderia glumae 8GR1 |
| YP_001863170.1 | Burkholderia phymatum STM815 |
| VP_001063371.1 | Burkholderia pseudomallei 668 |
| ZP_02494581.1 | Burkholderia pseudomallei NCTC 13177 |
| ZP_04898500.1 | Burkholderia pseudomallei Pasteur 52237 |
| YP_369306.1 | Burkholderia sp. 383 |
| YP_003609870.1 | 8urkholderia sp. CCGE1002 |
| YP_003907168.1 | Burkholderia sp. CCGE1003 |
| ZP_10036178.1 | 8urkholderia sp. Chl-1 |
| EGD06615.1 | 8urkholderia sp. TJl49 |
| VP_004977003.1 | Burkholderia sp. Vl23 |
| CBL42165.1 | butyrate-producing bacterium SS3/4 |
| VP_003991543.1 | Caldicellulosiruptor hydrothermalis 108 |
| VP_004027154.1 | Caldicellulosiruptor kristjanssonii 177R1B |
| VP_004023409.1 | Caldicellulosiruptor kronotskyensis 2002 |
| VP_001180241.1 | Caldicellulosiruptor saccharolyticus DSM 8903 |
| VP_001181365.1 | Caldicellulosiruptor saccharolyticus DSM 8903 |
| VP_005442188.1 | Caldilinea aerophila DSM 14535 = NBRC 104270 |
| EIA74674.1 | Campylobacter coli 132-6 |
| EIA80005.1 | Campylobacter coli 1957 |
| EIA50333.1 | Campylobacter coli 2685 |
| EIA84440.1 | Campylobacter coli 67-8 |
| EIA59740.1 | Campylobacter coli 80352 |
| EIB08252.1 | Campylobacter coli LMG 9860 |
| VP_001409109.1 | Campylobacter curvus 525.92 |
| AAF82113.l | Campylobacter jejuni |
| VP_001397974.1 | Campylobacter jejuni subsp. doylei 269.97 |
| EIB51049.1 | Campylobacter jejuni subsp. jejuni 2008-988 |
| ZP_05364429.1 | Campylobacter showae RM3277 |
| ZP_00372172.1 | Campylobacter upsaliensis RM3195 |
| VP_360509.1 | Carboxydothermus hydrogenoformans Z-2901 |
| ZP_03682223.1 | Catenibacterium mitsuokai DSM 15897 |
| ZP_04449563.1 | Catonella morbi ATCC 51271 |
| VP_004166442.1 | Cellulophaga algicola DSM 14237 |
| VP_004166181.1 | Cellulophaga a/gico/a DSM 14237 |
| VP_004263391.1 | Cellulophaga lytica DSM 7489 |
| VP_0019832S2.1 | Cellvibrio japonicus Ueda107 |
| VP_673748.1 | Chelativorans sp. BNCl |
| VP_003126133.1 | Chitinophaga pinensis DSM 2588 |
| VP_574526.1 | Chromohalobacter sa/exigens DSM 3043 |
| ZP_09334735.1 | Citrobacter freundii 4_7_47CFAA |
| VP_001453480.i | Citrobacter koseri ATCC BAA-895 |
| VP_005672087.1 | Clostridium acetobutylicum EA 2018 |
| ZP_03762445.1 | Clostridium asparagiforme DSM 15981 |
| ZP_02084161.1 | Clostridium bolteae ATCC BAA-613 |
| VP_001886967.1 | Clostridium botulinum B str. Eklund 178 |
| VP_004396695.1 | Clostridium botulinum BKT015925 |
| EDS78268.1 | Clostridium botulinum C str. Eklund |
| EDS76145.l | Clostridium botulinum C str. Eklund |
| EG086949.l | Clostridium botulinum C str. Stockholm |
| ZP_04822781.1 | Clostridium botulinum El str. ‘BoNT E Beluga’ |
| VP_003845006.1 | Clostridium cellulovorans 7438 |
| EFC94742.1 | Clostridium hathewayi DSM 13479 |
| EFD01272.1 | Clostridium hathewayi DSM 13479 |
| ZP_09148363.1 | Clostridium hathewayi WAL-18680 |
| ZP_03777231.1 | Clostridium hylemonae DSM 15053 |
| VP_002472037.1 | Clostridium kluyveri NBRC 12016 |
| ZP_03706978.1 | Clostridium methylpentosum DSM 5476 |
| ZP_03706281.1 | Clostridium methylpentosum DSM 5476 |
| ZP_03290838.1 | Clostridium nexile DSM 1787 |
| EHP49112.1 | •clostridium perfringens WAL-14572 |
| ZP_09049102.1 | Clostridium sp. 7_3_54FAA |
| yp_005147109.1 | Clostridium sp. BNLllOO |
| ZP_02073778.1 | C/ostridium sp. L2-50 |
| ZP_02438733.1 | Clostridium sp. SS2/1 |
| ZP_02867996.1 | Clostridium spiroforme DSM 1552 |
| ZP_02996105.1 | C/ostridium sporogenes ATCC 15579 |
| EHN14107.1 | C/ostridium sporogenes PA 3679 |
| ZP_01771318.1 | Collinsel/a aerofaciens ATCC 25986 |
| ZP_03297235.1 | Collinsella stercoris DSM 13279 |
| ZP_03544152.1 | Comamonas testosteroni KF-1 |
| ZP_07042769.1 | Comamonas testosteroni S44 |
| ZP_09013441.1 | Commensa /ibacter intestlni A911 |
| ZP_ 09697329.1 | Coprobacillus sp. 8_2_54BFAA |
| ZP_03800678.1 | Coprococcus comes ATCC 27758 |
| ZP_02206967.1 | Coprococcus eutactus ATCC 27759 |
| yp_005368024.1 | Corallococcus coralloides DSM 2259 |
| yp_005162586.1 | Corynebacterium diphtheriae C7 (beta) |
| yp_001139941.1 | Corynebacterium glutamlcum R |
| ZP_03935819.1 | Corynebacterium striatum ATCC 6940 |
| yp_004630586.1 | Corynebacterium ulcerans BR-AD22 |
| yp_004759254.1 | Corynebacterium variabile DSM 44702 |
| ADZ13631.1 | Cronobacter phage ENT47670 |
| yp_001438598.1 | Cronobacter sakazakii ATCC BAA-894 |
| yp_003150705.1 | Cryptobacterium curtum DSM 15641 |
| yp_583727.1 | Cupriavidus metallidurans CH34 |
| yp_002005623.1 | Cupriavidus taiwanensis LMG 19424 |
| YP_003084246.1 | Cyanophage PSS2 |
| ACY75805.1 | Cyanophage PSS2 |
| yp_004776003.1 | Cyclobacterium marinum DSM 745 |
| yp_004488616.1 | Delftia sp. Csl-4 |
| ZP_07200498.1 | delta proteobacterium NaphS2 |
| yp_520049.1 | Desulfitobacterium hafniense Y51 |
| ZP_08977565.1 | Desulfitobacterium metallireducens DSM 15288 |
| yp_002603986.1 | Desulfobacterium autotrophicum HRM2 |
| yp_004971910.1 | Desulfosporosinus orientis DSM 765 |
| yp_004543651.1 | Desulfotomaculum ruminis DSM 2154 |
| ZP_03311406.1 | Desulfovibrio piger ATCC 29098 |
| ZP_08850119.1 | Dorea formicigenerans 4_6_53AFAA |
| yp_003084663.1 | Dyadobacter fermentans DSM 18053 |
| yp_003088487.1 | Dyadobacter fermentans DSM 18053 |
| ZP_08474773.1 | Dysgonomonas gadei ATCC BAA-286 |
| ZP_08473950.1 | Dysgonomonas gadei ATCC BAA-286 |
| ZP_08470204.1 | Dysgonomonas mossii DSM 22836 |
| AE /70845.1 | EBPR podovirus 1 |
| yp_002933453.1 | Edwardsie/la ictaluri 93-146 |
| yp_002934200.1 | Edwardsiel/a ictaluri 93-146 |
| ZP_06714885.1 | Edwardsiella tarda ATCC 23685 |
| yp_003295659.1 | Edwardsiella tarda EIB202 |
| ZP_03712468.1 | Eikenella corrodens ATCC 23834 |
| YP_004594671.1 | Enterobacter aerogenes KCTC 2190 |
| yp_004594431.1 | Enterobacter aerogenes KCTC 2190 |
| YP_003611805.1 | Enterobacter cloacae subsp. cloacae ATCC 13047 |
| ZP_08499194.1 | Enterobacter hormaechei ATCC 49162 |
| YP_001175737.1 | Enterobacter sp. 638 |
| CAA23976.1 | Enterobacteria phage lambda |
| yp_001648906.1 | Enterobacteria phage Min27 |
| ZP_07949994.1 | Enterobacteriaceae bacterium 9_2_54FAA |
| YP_005704426.1 | Enterococcus faecalis 62 |
| EFU90864.1 | Enterococcus faecalis TX0630 |
| ZP_09112782.1 | Enterococcus saccharolyticus 30_1 |
| yp_003742593.1 | Erwinia billingiae Eb661 |
| yp_003740823.1 | Erwinia billingiae Eb661 |
| yp_004327343.1 | Erwinia phage phiEt88 |
| YP_004560942.1 | Erysipelothrix rhusiopathiae str. Fujisawa |
| ZP_09537413.1 | Erysipelotrichaceae bacterium 21_3 |
| EGC95709.1 | Escherichia fergusonii ECD227 |
| YP_512292.1 | Escherichia phage phiVlO |
| AEW24552.1 | Escherichia phage TL-20l b |
| AEW24678.1 | Escherichia phage TL-20llc |
| ZP_09322413.1 | Eubacteriaceae bacterium ACC19a |
| yp_002930165.1 | Eubacterium eligens ATCC 27750 |
| ZP_03717663.1 | Eubacterium hallii DSM 3353 |
| YP_003960581.1 | Eubacterium limosum KIST612 |
| ZP_07903104.1 | Eubacterium saburreum DSM 3986 |
| ZP_09545227.1 | Eubacterium sp. 3_1_31 |
| ZP_02024907.1 | Eubacterium ventriosum ATCC 27560 |
| ZP_09736507.1 | Facklamia languida CCUG 37842 |
| yp_005820203.1 | Fibrobacter succinogenes subsp. succinogenes S85 |
| ZP_02183546.1 | Flavobacteriales bacterium ALC-1 |
| yp_004843584.1 | Flavobacterium branchiophilum FL-15 |
| yp_004843521.1 | Flavobacterium branchiophilum FL-15 |
| YP_004941085.1 | Flavobacterium columnare ATCC 49512 |
| yp_004941882.1 | Flavobacterium columnare ATCC 49512 |
| yp_005357245.1 | Flavobacterium indicum GPTSAl00-9 |
| YP_005357968.1 | Flavobacterium indicum GPTSAl00-9 |
| yp_001196868.1 | Flavobacterium johnsoniae UW101 |
| yp_001195256.1 | Flavobacterium johnsoniae UW101 |
| yp_001295947.1 | Flavobacterium psychrophilum J/POZ/86 |
| yp_001296638.1 | Flavobacterium psychrophilum JIP02/86 |
| yp_005377560.1 | Frateuria aurantia DSM 6220 |
| EH078754.1 | Fusobacterium nucleatum subsp. animalis OT 420 |
| ZP_09588597.1 | Fusobacterium sp. 12_1B |
| ZP_09586364.1 | Fusobacterium sp. 12_1B |
| ZP_07922626.1 | Fusobacterium sp. 3_1_5R |
| YP_003811665.1 | gamma proteobacterium HdNl |
| ZP_05127187.1 | gamma proteobacterium NOR5-3 |
| yp_003374177.1 | Gardnerella vaginalis 409-05 |
| ZP_08258595.1 | Gemella haemolysans M341 |
| ZP_08261052.1 | Gemella sanguinis M325 |
| yp_001125680.1 | Geobacillus thermodenitrificans NG80-2 |
| yp_385574.1 | Geobacter metallireducens GS-15 |
| NP_951231.1 | Geobacter sulfurreducens PCA |
| YP_004871614.l | Glaciecola nitratireducens FR1064 |
| ZP_08900462.l | Gluconacetobacter oboediens 174Bp2 |
| ZP_08900481.l | Gluconacetobacter oboediens 174Bp2 |
| ZP_08900554.l | Gluconacetobacter oboediens 174Bp2 |
| yp_004868821.l | Gluconacetobacter xylinus NBRC 3288 |
| yp_192864.1 | Gluconobacter oxydans 621H |
| VP_191186.1 | Gluconobacter oxydans 621H |
| VP_744021.1 | Granulibacter bethesdensis CGDNIHl |
| YP_005059662.l | Granulicella mallensis MP5ACTX8 |
| EGT79392.1 | Haemophilus haemolyticus M21639 |
| ZP_05850920.1 | Haemophilus influenzae NT127 |
| ZP_01797282.1 | Haemophilus influenzae R3021 |
| ZP_01797281.1 | Haemophilus influenzae R3021 |
| ZP_10073638.1 | Haemophilus paraphrohaemolyticus HK411 |
| ZP_02479394.1 | Haemophilus parasuis 29755 |
| YP_002475717.l | Haemophilus parasuis SH0165 |
| ZP_09377516.l | Hafnia alvei ATCC 51873 |
| YP_003993926.1 | Halanaerobium hydrogeniformans |
| yp_004445529.1 | Haliscomenobacter hydrossis DSM llOO |
| yp_006180809.1 | Halobacillus halophilus DSM 2266 |
| ZP_09188055.1 | Halomonas boliviensis LCl |
| yp_003897727.1 | Halomonas elongata DSM 2581 |
| yp_003897090.l | Halomonas elongata DSM 2581 |
| ZP_09738126.l | Helcococcus kunzii ATCC 51366 |
| ZP_07805046.1 | Helicobacter cinaedi CCUG 18818 |
| ZP_04809499.1 | Helicobacter pullorum MIT 98-5489 |
| ZP_04808991.l | Helicobacter pullorum MIT 98-5489 |
| YP_005785121.1 | Helicobacter pylori 83 |
| yp_001681197.1 | Heliobacterium modesticaldum lcel |
| yp_001099394.l | Herminiimonas arsenicoxydans |
| yp_005511208.l | Hydrogenobacter thermophilus TK-6 |
| ZP_09999130.1 | lmtechella halotoferans Kl |
| yp_002128512.l | lodobacteriophage phiPLPE |
| ZP_09154646.l | Johnsonella ignava ATCC 51276 |
| yp_003162220.l | Jonesia denitrificans DSM 20603 |
| EHS95796.1 | Klebsiella oxytoca 10-5243 |
| EHT14033.1 | Klebsiella oxytoca 10-5250 |
| AEU09829.1 | Klebsiella pneumoniae |
| yp_002238960.l | Klebsiella pneumoniae 342 |
| YP_005953460.1 | Klebsiella pneumoniae KCTC 2242 |
| YP_005953897.1 | Klebsiella pneumoniae KCTC 2242 |
| YP_001335044.1 | Klebsiella pneumoniae subsp. pneumoniae MGH 78578 |
| ZP_06016835.1 | Klebsiella pneumoniae subsp. rhinoscleromatis ATCC 13884 |
| ZP_06552348.1 | Klebsiella sp. 1_1_55 |
| ZP_08306099.1 | Klebsiella sp. MS 92-3 |
| yp_003438695.1 | Klebsiella variicola At-22 |
| ZP_02163271.1 | Kordia algicida OT-1 |
| ZP_08616825.l | Lachnospiraceae bacterium _4_56FAA |
| ZP_08339829.l | Lachnospiraceae bacterium 2_1_46FAA |
| ZP_08336670.1 | Lachnospiraceae bacterium 3_1_46FAA |
| ZP_08604142.1 | Lachnospiraceae bacterium 3_1_57FAA_CT1 |
| ZP_08149693.1 | Lachnospiraceae bacterium 4_1_37FAA |
| ZP_08603145.1 | Lachnospiraceae bacterium 5_1_57FAA |
| ZP_08331597.1 | Lachnospiraceae bacterium 6_1_63FAA |
| ZP_09532735.1 | Lachnospiraceae bacterium 7_1_58FAA |
| ZP_08334226.1 | Lachnospiraceae bacterium 9_1_43BFAA |
| ZP_09521413.1 | Lachnospiraceae bacterium ACC2 |
| VP_004581188.1 | Lacinutrix sp. 5H-3-7-4 |
| VP_004580596.1 | Lacinutrix sp. 5H-3-7-4 |
| VP_005858762.1 | Lactobacillus casei BD-11 |
| VP_005851623.1 | Lactobacillus delbrueckii subsp. bulgaricus 2038 |
| VP_005848993.1 | Lactobacillus fermentum CECT 5716 |
| VP_005850725.1 | Lactobacillus helveticus H10 |
| VP_005862099.1 | Lactobacillus johnsoni i DPC 6026 |
| VP_004890503.1 | Lactobacillus plantarum WCFSl |
| VP_00586S109.1 | Lactobacillus rhamnosus GG |
| ZP_08564335.1 | Lactobacillus ruminis SPM0211 |
| VP_396489.1 | Lactobacillus sakei subsp. sakei 23K |
| VP_005863385.1 | Lactobacillus salivarius CECT 5713 |
| ZP_08890725.1 | Lactobacillus sp. 7_1_47FAA |
| VP_005869785.1 | Lactococcus garvieae Lg2 |
| EHE92415.1 | Lactococcus lactis subsp. cremoris CNCM 1-1631 |
| NP_996684.1 | Lactococcus phage phiLC3 |
| ABD63859.1 | Lactococcus phage phismq86 |
| VP_00279S496.1 | Laribacter hongkongensis HLHK9 |
| VP_003998593.1 | Leadbetterella byssophila DSM 17132 |
| VP_003997999.1 | Leadbetterella byssophila DSM 17132 |
| CAC33455.1 | Legionella pneumophila |
| VP_003163767.1 | Leptotrichia buccalis C-1013-b |
| ZP_06011746.1 | Leptotrichia goodfellowii F0264 |
| VP_005174459.1 | Leuconostoc mesenteroides subsp. mesenteroides J18 |
| VP_004704993.1 | Leuconostoc sp. C2 |
| NP_471091.1 | Listeria innocua Clipl 262 |
| ZP_07873574.1 | Listeria ivanovii FSL F6-596 |
| ZP_05289346.1 | Listeria monocytogenes FSL F2-515 |
| ZP_05297269.1 | Listeria monocytogenes FSLJ2-003 |
| ZP_05299340.1 | Listeria monocytogenes FSL J2-003 |
| ZP_07075805.1 | Listeria monocytogenes FSL N1-017 |
| EEW20557.1 | Listeria monocytogenes FSL R2-503 |
| EEW2026S.1 | Listeria monocytogenes FSL R2-S03 |
| ZP_00235039.1 | Listeria monocytogenes str. 1/2a F6854 |
| VP_003464587.1 | Listeria seeligeri serovar 1/2b str. SLCC3954 |
| ZP_04979099.1 | Mannheimia haemolytica PHL213 |
| ZP_09732369.1 | Megamonas funiformis VIT 11815 |
| YP_004456677.1 | Melissococcus plutonius ATCC 35311 |
| YP_005318817.1 | Melissococcus plutonius DAT561 |
| BAB53634.1 | Mesorhizobium Ioli MAFF303099 |
| YP_002502020.1 | Methylobacterium nodulans ORS 2060 |
| YP_002501391.1 | Methylobacterium nodu/ans ORS 2060 |
| YP_002497906.1 | Methylobacterium nodulans ORS 2060 |
| YP_002498798.1 | Methylobacterium nodulans ORS 2060 |
| ZP_09899678.1 | Methylomicrobium album BG8 |
| YP_004915903.1 | Methylomicrobium alcaliphilum |
| yp_004512397.1 | Methylomonas methanica MC09 |
| CA090389.1 | Microcyst'1s aeruginosa PCC 7806 |
| ZP_07908870.1 | Mobiluncus curtisii ATCC 51333 |
| EGE24382.1 | Moraxella catarrhalis 101P3081 |
| EGE19718.1 | Moraxella catarrhalis 8Cl |
| yp_004789848.1 | Muricauda ruestringensis DSM 13258 |
| yp_004789429.1 | Muricauda ruestringensis DSM 13258 |
| EIC64366.1 | Mycobacterium abscessus M93 |
| EIC70403.1 | Mycobacterium abscessus M94 |
| yp_001852190.1 | Mycobacterium marinum M |
| 8AH69619.1 | Mycoplasma fermentans PG18 |
| yp_005888312.1 | Mycoplasma hyopneumoniae 168 |
| yp_003856575.1 | Mycoplasma hyorhinis HU8-l |
| yp_005905159.1 | Mycoplasma hyorhinis MCLD |
| ZP_09524021.l | Myroides adoratimimus CCUG 10230 |
| EH012592.1 | Myroides adoratimimus CIP 101113 |
| YP_004666545.1 | Myxacoccus fulvus HW-1 |
| VP_004669149.1 | Myxacoccus fulvus HW-1 |
| YP_630241.1 | Myxococcus xanthus DK 1622 |
| ZP_08247669.1 | Neisseria bacilliformis ATCC 8AA-1200 |
| ZP_03719260.1 | Neisseria flavescens NRL30031/H210 |
| yp_005889683.1 | Neisseria gonorrhoeae TCDC-NG08107 |
| yp_004048639.1 | Neisseria lactamica 020-06 |
| C8X21356.1 | Neisseria lactamica Y92-1009 |
| yp_005892581.1 | Neisseria meningitidis WUE 2594 |
| yp_005006679.1 | Niastella koreensis GR20-10 |
| yp_004168204.1 | Nitratifractor salsuginis DSM 16511 |
| ZP_09514368.1 | Oceanicola sp. Sl24 |
| VP_005092407.1 | Oceanimonas sp. GKl |
| yp_005093273.1 | Oceanimonas sp. GKl |
| NP_693974.1 | Oceanobacillus iheyensis HTE831 |
| ZP_09641960.1 | Odoribacter laneus YIT 12061 |
| ZP_09326776.1 | Oribacterium sp. ACBl |
| yp_004988339.1 | Owenweeksia hongkongensis DSM 17368 |
| yp_004988588.1 | Owenweeksia hongkongensis DSM 17368 |
| ZP_09077613.1 | Paenibacillus elgii 869 |
| ZP_09079484.1 | Paenibacillus elgii 869 |
| ZP_09071954.1 | Paenibacillus larvae subsp. larvae 8-3650 |
| ZP_09069307.1 | Paenibacillus larvae subsp. larvae B-3650 |
| ZP_09069904.1 | Paenibacillus larvae subsp. larvae 8-3650 |
| ZP_09072245.1 | Paenibacillus larvae subsp. larvae 8-3650 |
| ZP_02327781.1 | Paenibacillus larvae subsp. larvae 8RL-230010 |
| ZP_02326141.1 | Paenibacillus larvae subsp. larvae 8RL-230010 |
| YP_006190082.1 | Paenibacillus mucilaginasus K02 |
| yp_003010343.1 | Paenibacillus sp. JDR-2 |
| yp_003013704.1 | Paenibacillus sp. JDR-2 |
| ZP_04854710.1 | Paenibacillus sp. oral taxon 786 str. D14 |
| yp_005073546.1 | Paenibacillus terrae HPL-003 |
| VP_003519587.1 | Pantoea ananatis LMG 20103 |
| VP_005993583.1 | Pantoea ananatis PA13 |
| yp_005994280.1 | Pantoea ananatis PA13 |
| ZP_07380439.1 | Pantoea sp. aB |
| yp_003931279.1 | Pantoea vagans C9-1 |
| ZP_03477183.1 | Parabacteroides johnsonii DSM 18315 |
| ZP_03475832.1 | Parabacteroides johnsoni i DSM 18315 |
| ZP_02033987.1 | Parabacteroides merdae ATCC 43184 |
| ZP_02032292.1 | Parabacteroides merdae ATCC 43184 |
| ZP_06299504.1 | Parachlamydia acanthamoebae str. Hall's coccus |
| yp_917501.1 | Paracoccus denitrificans PD1222 |
| ZP_02094048.1 | Parvimonas micra ATCC 33270 |
| YP_051100.1 | Pectobacterium atrosepticum SCRI 1043 |
| ZP_07367149.1 | Pediococcus acidilactici DSM 20284 |
| yp_005005652.1 | Pediococcus claussenii ATCC BAA-344 |
| yp_004272912.1 | Pedobacter saltans DSM 12145 |
| yp_901551.1 | Pelobacter propionicus DSM 2379 |
| YP_903200.1 | Pelobacter propionicus DSM 2379 |
| ZP_07400314.1 | Peptoniphilus duerdenii ATCC BAA-1640 |
| yp_002731548.1 | Persephonella marina EX-Hl |
| NP_461568.2 | Phage Gifsy-1 |
| yp_005352506.1 | Photobacterium damselae subsp. damselae |
| ZP_06157475.1 | Photobacterium damselae subsp. damselae CIP 102761 |
| yp_132232.1 | Photobacterium profundum SS9 |
| NP_930169.1 | Photorhabdus luminescens subsp. laumondii TTOl |
| YP_004270153.1 | Planctomyces brasiliensis DSM 5305 |
| yp_001155602.1 | Polynucleobacter necessarius subsp. asymbioticus QLW-Pl DMWA-1 |
| ZP_09103791.1 | Prevotel/ a histico/a F0411 |
| ZP_09554100.1 | Prevotella maculosa OT 289 |
| ZP_09590809.1 | Prevote/la micans F0438 |
| ZP_09120679.1 | Prevotel/a sp. oral taxon 302 str. F0323 |
| ZP_03840270.1 | Proteus mirabi/is ATCC 29906 |
| ZP_03805638.1 | Proteus penneri ATCC 35198 |
| ZP_03804770.1 | Proteus penneri ATCC 35198 |
| ZP_03318967.1 | Providencia alca/ifaciens DSM 30120 |
| ZP_03320274.1 | Providencia alcalifaciens DSM 30120 |
| AAM08027.1 | Providencia rettgeri |
| ZP_06124041.2 | Providencia rettgeri DSM 1131 |
| EFE55836.1 | Providencia rettgeri DSM 1131 |
| yp_006217717.1 | Providencia stuartii MRSN 2154 |
| YP_661287.1 | Pseudoalteromonas atlantica T6c |
| YP_663733.1 | Pseudoalteromonas at/antica T6c |
| ZP_09236879.1 | Pseudoalteromonas sp. BSi20439 |
| ZP_09234821.1 | Pseudoa/teromonas sp. BSi20439 |
| ZP_09235053.1 | Pseudoalteromonas sp. BSi20439 |
| ZP_09243850.1 | Pseudoalteromonas sp. BSi20495 |
| YP_004846059.1 | Pseudogulbenkiania sp. NH8B |
| ACD38903.1 | Pseudomonas aeruginosa |
| ACD38827.1 | Pseudomonas aeruginosa |
| ZP_07797103.1 | Pseudomonas aeruginosa 39016 |
| YP_005975666.1 | Pseudomonas aeruginosa M18 |
| YP_004355228.1 | Pseudomonas brassicacearum subsp. brassicacearum NFM421 |
| YP_005208095.1 | Pseudomonas f/uorescens F113 |
| yp_348340.1 | Pseudomonas fluorescens PI0-1 |
| yp_002872416.1 | Pseudomonas fluorescens SBW2S |
| yp_004380540.1 | Pseudomonas mendocina NK-01 |
| YP_005098041.1 | Pseudomonas phage phi297 |
| yp_005930618.1 | Pseudomonas putida BIRD-1 |
| yp_001667964.1 | Pseudomonas putida GB-1 |
| yp_005938868.1 | Pseudomonas stutzeri DSM 4166 |
| YP_005937536.1 | Pseudomonas stutzeri DSM 4166 |
| EGH87883.1 | Pseudomonas syringae pv. lachrymans str. M30131S |
| EGH59138.1 | Pseudomonas syringae pv. maculicola str. ES4326 |
| yp_274842.1 | Pseudomonas syringae pv. phaseolicola 1448A |
| EGH43274.1 | Pseudomonas syringae pv. pisi str. 17048 |
| yp_235897.1 | Pseudomonas syringae pv. syringae B728a |
| YP_264268.1 | Psychrobacter arcticus 273-4 |
| yp_001280242.1 | Psychrobacter sp. PRwf-1 |
| yp_942451.1 | Psychromonas ingrahamii 37 |
| yp_944546.1 | Psychromonas ingrahamii 37 |
| yp_005199476.1 | Rahnella aquatilis CIP 78.6S = ATCC 33071 |
| yp_006029734.1 | Ralstonia solanacearum Po82 |
| yp_002281965.1 | Rhizobium leguminosarum bv. trifolii WSM2304 |
| yp_005087203.1 | Rhodococcus phage REQ3 |
| yp_004011824.1 | Rhodomicrobium vannielii ATCC 17100 |
| yp_006017652.1 | Riemerella anatipestifer RA-GD |
| yp_004838017.1 | Roseburia hominis A2-183 |
| ZP_03755701.1 | Roseburia inulinivorans DSM 16841 |
| ZP_03166627.1 | Ruminococcus lactaris ATCC 29176 |
| ZP_04855253.1 | Ruminococcus sp. S_1_39B_FAA |
| yp_004655025.1 | Runella slithyformis DSM 19S94 |
| YP_004653638.1 | Runella slithyformis DSM 19S94 |
| yp_527033.1 | Saccharophagus degradans 2-40 |
| yp_001571105.1 | Salmonella enterica subsp. arizonae serovar 62: z4, z23: — str. RSK2980 |
| EHC38944.1 | Salmonella enterica subsp. enterica serovar Gaminara str. A4-S67 |
| ZP_ 03220723.1 | Salmonella enterica subsp. enterica serovar Javiana str. GA_MM04042433 |
| EDZ19028.1 | Salmonella enterica subsp. enterica serovar Kentucky str. CDC 191 |
| EHC73330.1 | Salmonella enterica subsp. enterica serovar Mississippi str. A4-633 |
| EHL46421.1 | Salmonella enterica subsp. enterica serovar Montevideo str. SARB30 |
| EHC90429.1 | Salmonella enterica subsp. enterica serovar Rubislaw str. A4-6S3 |
| EHC91146.1 | Salmonella enterica subsp. enterica serovar Senftenberg str. A4-S43 |
| ZP_03338172.1 | Salmonella enterica subsp. enterica serovar Typhi str. 404ty |
| ZP_03340438.1 | Salmonella enterica subsp. enterica serovar Typhi str. 404ty |
| ZP_03341127.1 | Salmonella enterica subsp. enterica serovar Typhi str. 404ty |
| ZP_06538095.1 | Salmonella enterica subsp. enterica serovar Typhi str. AG3 |
| AAC26069.l | Salmonella enterica subsp. enterica serovar Typhimurium |
| EHY70960.1 | Salmonella enterica subsp. houtenae str. ATCC BAA-1581 |
| yp_005098013.1 | Salmonella phage SPNlS |
| yp_005321079.1 | Saprospira grandis str. Lewin |
| yp_005323878.1 | Saprospira grandis str. Lewin |
| yp_003308034.1 | Sebaldella termitidis ATCC 33386 |
| yp_003657357.1 | Segniliparus rotundus DSM 4498S |
| EHG25612.1 | Selenomonas noxia F0398 |
| yp_005433748.1 | Selenomonas ruminantium subsp. lactilytica TAM6421 |
| ZP_06640797.1 | Serratia odorifera DSM 4582 |
| VP_006024370.1 | Serratia sp. AS13 |
| ZP_08039150.1 | Serratia symbiotica str. Tucson |
| yp_927263.1 | Shewanella amazonensis SB2B |
| VP_006020740.1 | Shewanella baltica BA175 |
| VP_005272800.1 | Shewanella baltica 05678 |
| VP_001093802.1 | Shewanella Joihica PV-4 |
| NP_718112.1 | Shewanella oneidensis MR-1 |
| NP_717301.1 | Shewanella oneidensis MR-1 |
| VP_002312664.1 | Shewanella piezotolerans WP3 |
| yp_002311778.1 | Shewanella piezotolerans WP3 |
| VP_006011723.1 | Shewanella putrefaciens 200 |
| VP_001182934.1 | Shewanella putrefaciens CN-32 |
| VP_001473852.1 | Shewanella sediminis HAW-EB3 |
| VP_001473225.1 | Shewanella sediminis HAW-EB3 |
| VP_003556874.1 | Shewanella violacea DSS12 |
| VP_003557838.1 | Shewanella violacea DSS12 |
| YP_001760744.1 | Shewanella woodyi ATCC 51908 |
| AAF28115.l, | Shigella dvsenteriae |
| AF153317_10 | |
| ZP_07683610.1 | Shigella dysenteriae 1617 |
| EFW50178.1 | Shigella dysenteriae CDC 74-1112 |
| ZP_04454959.1 | Shuttleworthia satelles DSM 14600 |
| y p_004662889.1 | Simkania negevensis Z |
| VP_001327594.1 | Sinorhizobium medicae WSM419 |
| yp_001327785.1 | Sinorhizobium medicae WSM419 |
| VP_004549676.1 | Sinorhizobium meliloti AK83 |
| VP_004548149.1 | Sinorhizobium meliloti AK83 |
| EHK78038.l | Sinorhizobium meliloti CCNWSX0020 |
| VP_005719596.1 | Sinorhizobium meliloti SMll |
| YP_455377.1 | Sodalis glossinidius str. ‘morsitans' |
| VP_516203.l | Sodalis phage phiSGl |
| YP_001616277.1 | Sorangium cellulosum ‘So ce 56’ |
| YP_004247313.1 | Sphaerochaeta globus str. Buddy |
| YP_005061390.1 | Sphaerochaeta pleomorpha str. Grapes |
| VP_004317691.1 | Sphingobacterium sp. 21 |
| yp_004315662.1 | Sphingobacterium sp. 21 |
| YP_005474943.1 | Spirochaeta africana DSM 8902 |
| VP_004411234.1 | Spirochaeta coccoides DSM 17374 |
| YP_003804225.1 | Spirochaeta smaragdinae DSM 11293 |
| VP_003803689.1 | Spirochaeta smaragdinae DSM 11293 |
| ZP_08680759.1 | Sporosarcina newyorkensis 2681 |
| ZP_06324416.1 | Staphylococcus aureus subsp. aureus D139 |
| VP_005739407.1 | Staphylococcus aureus subsp. aureus JKD6159 |
| YP_253435.1 | Staphylococcus haemolyticus JCSC143S |
| YP_006015496.1 | Staphylococcus pseudintermedius ED99 |
| YP_003952450.1 | Stigmatella aurantiaca DW4/3-1 |
| yp_006035100.1 | Streptococcus gallolyticus subsp. gallolyticus ATCC 43143 |
| ZP_07463816.1 | Streptococcus gallolyticus subsp. gallolyticus TX20005 |
| EGV02688.1 | Streptococcus infantis SK970 |
| EHG11465.1 | Streptococcus intermedius F0395 |
| NP_722442.1 | Streptococcus mutans UA159 |
| VP_004479394.1 | Streptococcus parauberis KCTC 11537 |
| CBW39270.1 | Streptococcus phage 2167 |
| CBW39216.1 | Streptococcus phage 8140 |
| VP_596543.1 | Streptococcus phage 9429.2 |
| ABD48929.1 | Streptococcus phage M102 |
| VP_002995484.l | Streptococcus phage M102 |
| CBW38953.1 | Streptococcus phage V22 |
| EHE64346.1 | Streptococcus pneumoniae EU-NPOl |
| EHE76000.1 | Streptococcus pneumoniae GAI 1426 |
| VP_005412494.1 | Streptococcus pyogenes MGAS1882 |
| VP_001128217.1 | Streptococcus pyogenes str. Manfredo |
| CCB95242.1 | Streptococcus salivarius JIM8777 |
| ZP_03625981.1 | Streptococcus suis 89/1591 |
| yp_00608S650.1 | Streptococcus suis A7 |
| yp_006078894.1 | Streptococcus suis SS12 |
| EHE89971.1 | Streptococcus thermophilus CNCM 1-1630 |
| ZP_09342658.1 | Subdoligranulum sp. 4_3_54A2FAA |
| yp_005014978.1 | Tannerella forsythia ATCC 43037 |
| yp_005015967.1 | Tannerella forsythia ATCC 43037 |
| ZP_09337816.1 | Tannerella sp. 6_1_58FAA_CT1 |
| ZP_09337420.1 | Tannerella sp. 6_1_58FAA_CT1 |
| yp_004874525.1 | Tavlorella asinigenitalis MCE3 |
| YP_003477226.1 | Thermoanaerobacter italicus Ab9 |
| yp_004150652.1 | Thermovibrio ammonificans HB-1 |
| ZP_03497413.1 | Therm us aquaticus Y51MC23 |
| YP_313909.1 | Thiobacillus denitrificans ATCC 25259 |
| ZP_08768668.1 | Thiocapsa marina 5811 |
| ZP_08772750.1 | Thiocapsa marina 5811 |
| ZP_08926729.1 | Thiocystis violascens DSM 198 |
| ZP_08824405.1 | Thiorhodococcus drewsii AZl |
| ZP_08822326.1 | Thiorhodococcus drewsii AZl |
| ZP_08825220.1 | Thiorhodococcus drewsii All |
| ZP_09809048.1 | Thiorhodovibrio sp. 970 |
| ZP_09865744.1 | Thiorhodovibrio sp. 970 |
| ZP_09865982.1 | Thiorhodovibrio sp. 970 |
| ZP_09809289.1 | Thiorhodovi brio sp. 970 |
| yp_004438805.1 | Treponema brennaborense DSM 12168 |
| yp_005224087.1 | Treponema pallidum subsp. pallidum DAL-1 |
| yp_722968.1 | Trichodesmium erythraeum IMS101 |
| ZP_02971366.1 | Ureaplasma parvum serovar 6 str. ATCC 27818 |
| EDX53145.1 | Ureaplasma urealyticum serovar 12 str. ATCC 33696 |
| ZP_03771988.1 | Ureaplasma urea/yticum serovar 8 str. ATCC 27618 |
| ZP_01237386.1 | Vibrio angustum S14 |
| ZP_07741429.1 | Vibrio caribbenthicus ATCC BAA-2122 |
| ZP_01947910.1 | Vibrio cholerae 1587 |
| YP_005633953.1 | Vibrio cholerae LMA3984-4 |
| YP_005633301.1 | Vibrio cholerae LMA3984-4 |
| AAX89425.1 | Vibrio cholerae non-Ol/non-0139 |
| ABY28344.1 | Vibrio cholerae 0139 |
| ZP_05880433.1 | Vibrio furnissii CIP 102972 |
| yp_004993437.1 | Vibrio furnissii NCTC 11218 |
| ZP_06177392.1 | Vibrio harvevi 1DA3 |
| ZP_06174696.1 | Vibrio harvevi 1DA3 |
| VP_002417106.1 | Vibrio splendidus LGP32 |
| VP_004238525.1 | Weeksella virosa DSM 16922 |
| VP_004726213.1 | Weissella koreensis KACC 15510 |
| VP_001418489.1 | Xanthobacter autotrophicus Pv2 |
| yp_003467094.1 | Xenorhabdus bovienii SS-2004 |
| yp_003662329.1 | Xenorhabdus nematophila ATCC 19061 |
| yp_003325187.1 | xvlanimonas cellulosilvtica DSM 15894 |
| AAF84457.1, | Xvlella fastidiosa 9a5c |
| AE003991_9 | |
| ZP_00683037.1 | Xvlella fastidiosa Ann-1 |
| ZP_00682624.1 | Xvlella fastidiosa Ann-1 |
| ZP_00683132.1 | Xvlella fastidiosa Ann-1 |
| VP_006001389.1 | Xvlella fastidiosa subsp. fastidiosa GB514 |
| yp_006005235.1 | Versinia enterocolitica subsp. palearctica Vll |
| yp_070310.1 | Versinia pseudotuberculosis IP 32953 |
| yp_001101797.1 | Yersinia ruckeri |
| yp_004739144.1 | Zobellia galactanivorans |
| yp_004738450.1 | Zobellia galactanivorans |
| F9ZNV9 | Acidithiobacillus caldus |
| C6AM23 | Aggregatibacter aphrophilus |
| A6TRD8 | Alkaliphilus metalliredigens |
| D3RWF4 | Allochromatium vinosum |
| B9MNF3 | Anaerocellum thermophilum |
| C6ACZ8 | Bartonella grahamii |
| A91YA2 | Bartonella tribocorum |
| A91T52 | Bartonella tribocorum |
| E8MTD2 | Bifidobacterium longum subsp. infantis |
| A3NMOO | Burkholderia pseudomallei |
| E4QCOO | Caldicellulosi ruptor hvdrothermalis |
| E4S5A7 | Caldicellulosiruptor kristjanssonii |
| E4SEV3 | Caldicellulosiruptor kronotskyensis |
| A4XJG5 | Caldicellulosiruptor saccharolvticus |
| A4XMN9 | Caldicellulosiruptor saccharolvticus |
| A7HOW7 | Campylobacter curvus |
| A7H394 | Campylobacter jejuni subsp. doylei |
| Q3ABH5 | Carboxydothermus hydrogenoformans |
| D9SWGO | Clostridium cellulovorans |
| Q18AA7 | Clostridium difficile |
| H8MHN3 | Corallococcus coralloides |
| GOHB56 | Corynebacterium variabile |
| C7F4E8 | cvanophage pSS2 |
| F6AU64 | Delftia sp. |
| CG19468 | Drosophila melanogaster |
| GOEOR4 | Enterobacter aerogenes |
| D5CJK9 | Enterobacter cloacae subsp. cloacae |
| A4W7K6 | Enterobacter sp. |
| 81XAU6 | Escherichia coli |
| 87LNE9 | Escherichia fergusonii |
| H8L634 | Frateuria aurantia |
| Q5FN39 | Gluconobacter oxydans |
| B8F617 | Haemophilus parasuis serovar 5 |
| E4RNA4 | Halanaerobium sp. |
| P43351 | Homo sapiens |
| D3DGM9 | Hydrogenobacter thermophilus |
| Q9MBV8 | Lactotoccus phage ul16.2 |
| C1D7P7 | Laribacter hongkongensis |
| Q9AKZO | Legionella pneumophila |
| Q926Al | Listeria innocua serovar 6a |
| Q9T172 | Listeria phage A118 |
| F3YBJO | Melissococcus plutonius |
| B2HPl3 | Mycobacterium marinum |
| F8CQD8 | Myxococcus fulvus |
| Q1DAT2 | Myxococcus xanthus |
| E7BFDS | Neisseria meningitidis serogroup A |
| C6CX43 | Paenibacillus sp. |
| A1B8Gl | Paracoccus denitrificans |
| A1AQ73 | Pelobacter propionicus |
| COQSA2 | Persephonella marina |
| Q7N2Y8 | Photorhabdus luminescens |
| Q7N2Y8 | Photorhabdus luminescens subsp. laumondii |
| B4EWRS | Proteus mirabilis |
| C3KA14 | Pseudomonas fluorescens |
| ASWFSl | Psychrobacter sp. |
| Q984J6 | Rhizobium loti |
| YML032 | Saccharomyces cerevisiae |
| B4TDT1 | Salmonella heidelberg |
| A41VH9 | Salmonella newport |
| A9MSD6 | Salmonella paratyphi B |
| Q8Z7Y3 | Salmonella typhi |
| H6L1F4 | Saprospira grandis |
| E6XGY4 | Shewanella putrefaciens |
| AlRHSl | Shewanella sp. |
| Q32GM7 | Shigella dysenteriae serotype 1 |
| F6DWY3 | Sinorhizobium meliloti |
| F7X3Dl | Sinorhizobium meliloti |
| Q2NSA3 | Sodalis glossinidius |
| E1R1F4 | Spirochaeta smaragdinae |
| E1R3KO | Spirochaeta smaragdinae |
| Q08VK7 | Stigmatella aurantiaca |
| FSZL14 | Streptococcus parauberis |
| ClCGll | Streptococcus pneumoniae |
| EOTQLO | Streptococcus pneumoniae |
| Q9A029 | Streptococcus pyogenes serotype Ml |
| Q1CQT2 | Streptococcus pyogenes serotype M12 |
| A2RDQ6 | Streptococcus pyogenes serotype MS |
| F8LPH2 | Streptococcus salivarius |
| E8T306 | Thermovibrio ammonificans |
| Q8KQWO | Vibrio cholerae |
| C3NU24 | Vibrio cholerae serotype 01 |
| B7VNT1 | Vibrio splendidus |
| D3V014 | Xenorhabdus bovienii |
| D1BWP7 | Xylanimonas cellulosilytica |
| Q87CQ1 | Xylella fastidiosa |
| Q9PCV7 | Xylella fastidiosa |
| Q66Bl7 | Yersinia pseudotuberculosis |

TABLE 7
Single Stranded DNA-Binding Proteins.
SS DNA-BINDING PROTEINS

gi|802137314|emb|CQR83440.1|: 39-216 single-stranded DNA-binding
protein [Escherichia coli K-12]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 11)
gi|115515464|gb|ABJ03539.1|: 20-197 single strand DNA-binding protein
[Escherichia coli APEC O1]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 12)
gi|91075172|gb|ABE10053.1|: 22-199 single strand DNA-binding protein
[Escherichia coli UTI89]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 13)
gi|446090450|ref|WP_000168305.1|: 1-178 MULTISPECIES: ssDNA-binding
protein [Proteobacteria]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 14)
gi|446090454|ref|WP_000168309.1|: 1-178 MULTISPECIES: ssDNA-binding
protein [Escherichia]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNV
GGGQPQGGWGQPQQPQGGNQFSGGAQSRPQQSAPAAPSNEPPMDFDDDIPF (SEQ ID NO: 15)
gi|446090447|ref|WP_000168302.1|: 1-178 ssDNA-binding protein
[Shigella flexneri]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNKFSGGAQSRPQQSAPAAPSNEPPMDFDDDIPF (SEQ ID NO: 16)
gi|446079209|ref|WP_000157064.1|: 1-178 ssDNA-binding protein
[Escherichia coli]
MASKGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 17)
gi|953799395|ref|WP_058036055.1|: 1-178 single-stranded DNA-binding
protein [Escherichia coli]
MASRGVNKVILVGNLGQDPEVRYMPNGGAVANITLATSESWRDKATGEMKEQTEWHRVVLFGKL
AEVASEYLRKGSQVYLEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 18)
gi|766978151|ref|WP_044868983.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSAQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 19)
gi|446090443|ref|WP_000168298.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAAGNIG
GGQPQGGWGQPQQPQGGNQFSGGAQSRPQQSAPAAPSNEPPMDFDDDIPF (SEQ ID NO: 20)
gi|851928251|ref|WP_048220840.1|: 1-178 ssDNA-binding protein
[Escherichia coli]
MASRGVNKVILVGNLGHDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 21)
gi|754840036|ref|WP_042201710.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQSGWGQPQQPQGGNQFSGGAQSRPQQSAPAAPSNEPPMDFDDDIPF (SEQ ID NO: 22)
gi|896026319|ref|WP_049086626.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGSWGQPQQPQGGNQFSGGAQSRPQQSAPAAPSNEPPMDFDDDIPF (SEQ ID NO: 23)
gi|921979423|ref|WP_053271110.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGSAQSRPQQSAPAAPSNEPPMDFDDDIPF (SEQ ID NO: 24)
gi|585361910|ref|WP_024243502.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGSNQFSGGAQSRPQQSAPAAPSNEPPMDFDDDIPF (SEQ ID NO: 25)
gi|692999009|ref|WP_032185683.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGSNIG
GGQPQGGWGQPQQPQGGNQFSGGAQSRPQQSAPAAPSNEPPMDFDDDIPF (SEQ ID NO: 26)
gi|545166587|ref|WP_021521173.1|: 1-178 ssDNA-binding protein
[Escherichia coli]
MASRGVNKVILVGNLGQDPEVRYMPNSGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 27)
gi|446090455|ref|WP_000168310.1|: 1-178 MULTISPECIES: ssDNA-binding
protein [Escherichia]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQSGGAPAGGNIG
GGQPQGGWGQPQQPQGGNQFSGGAQSRPQQSAPAAPSNEPPMDFDDDIPF (SEQ ID NO: 28)
gi|585341793|ref|WP_024223419.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSTPAAPSNEPPMDFDDDIPF (SEQ ID NO: 29)
gi|757739421|ref|WP_042963455.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQSQQPQGGNQFSGGAQSRPQQSAPAAPSNEPPMDFDDDIPF (SEQ ID NO: 110)
gi|486058717|ref|WP_001508182.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSCGAQSRPQQSAPAAPSNEPPMDFDDDIPF (SEQ ID NO: 30)
gi|446090457|ref|WP_000168312.1|: 1-178 ssDNA-binding protein
[Escherichia coli]
MASRGVNKVILVGNLGQDPEVRYMPNGGAVANITLATSESWRDKATGEMKEQTEWHRVVLTGKL
AEVASEYLRKGSQVYIEGQLRTRKWTDQSGQDRYTTEVVVNVGGTMXMLGGRQGGGAPAGGNIG
GGQPQGGWGQPQQPQGGNQFSGGAQSRPQQSAPAAPSNEPPMDFDDDIPF (SEQ ID NO: 31)
gi|723112103|ref|WP_033557595.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVVSEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 32)
gi|693064509|ref|WP_032230375.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGVQSRPQQSAPAAPSNEPPMDFDDDIPF (SEQ ID NO: 33)
gi|844746903|ref|WP_047928938.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGDAPAGGNIG
GGQPQGGWGQPQQPQGGNQFSGGAQSRPQQSAPAAPSNEPPMDFDDDIPF (SEQ ID NO: 34)
gi|446090446|ref|WP_000168301.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQDGNQFSGGAQSRPQQSAPAAPSNEPPMDFDDDIPF (SEQ ID NO: 35)
gi|735676636|ref|WP_034167446.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYITEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 36)
gi|558576622|gb|AHA68760.1|: 22-199 Single-strand DNA bindingprotein
[Shigella dysenteriae 1617]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQLQGGWGQPQQPQGGNQFSGGAQSRPQQSAPAAPSNEPPMDFDDDIPF (SEQ ID NO: 37)
gi|974633789|ref|WP_059220463.1|: 1-178 single-stranded DNA-binding
protein [Escherichia albertii]
MASRGVNKVILVGNLGQDPEVRYMPNGGAVANITLATSESWRDKATGEMKEQTEWHRVVLFGKL
AEVASEYLRKGSQVYIEGQLRTRKWTDQSGLDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 38)
gi|961657168|emb|CRL87896.1|: 1-178 Single-stranded DNA-binding
protein [Escherichia coli]
MASRGVNKVILVGNLGQDPEVRYMPNGGAVANITLATSESWRDKATGEMKEQTEWHRVVLFGKL
AEVASEYLCKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 39)
gi|446090440|ref|WP_000168295.1|: 1-178 ssDNA-binding protein
[Escherichia coli]
MASRGVNKVILVGNLGLDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 40)
gi|446090449|ref|WP_000168304.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQPAPAAPSNEPPMDFDDDIPF (SEQ ID NO: 41)
gi|446090444|ref|WP_000168299.1|: 1-178 MULTISPECIES: ssDNA-binding
protein Enterobacteriaceae]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQLQGGWGQPQQPQGGNQFSGGAQSRPQQSAPAAPSNEPPMDFDDDIPF (SEQ ID NO: 42)
gi|585353006|ref|WP_024234614.1|: 1-178 ssDNA-binding protein
[Escherichia coli]
MASRGVNKVILVGNLGQDPEVRYMPNGGAVANITLATSESWRDKATGEMKDQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSTPAAPSNEPPMDFDDDIPF (SEQ ID NO: 43)
gi|823645439|ref|WP_047090144.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRLQQSAPAAPSNEPPMDFDDDIPF (SEQ ID NO: 44)
gi|545298006|ref|WP_021578210.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQLQGGNQFSGGAQSRPQQSAPAAPSNEPPMDFDDDIPF (SEQ ID NO: 45)
gi|446090456|ref|WP_000168311.1|: 1-178 MULTISPECIES: ssDNA-binding
protein [Escherichia]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQSGGAPTGGNIG
GGQPQGGWGQPQQPQGGNQFSGGAQSRPQQSAPAAPSNEPPMDFDDDIPF (SEQ ID NO: 46)
gi|487374574|ref|WP_001647924.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQGYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 47)
gi|485855265|ref|WP_001456829.1|: 1-178 ssDNA-binding protein
[Escherichia coli]
MASRGVNKVILVGNLGQDPEVRYMPNGGAVANITLATSESWRDKATGEMKEQTEWHRVVLFGKL
AEGASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMDFDDDIPF (SEQ ID NO: 48)
gi|974673892|ref|WP_059258066.1|: 1-178 single-stranded DNA-binding
protein [Escherichia albertii]
MASRGVNKVILVGNLGQDPEVRYMPNGGAVANITLATSESWRDKATGEMKEQTEWHRVVLFGKL
AEVASEYLRKGSLVYIEGQLRTRKWTDQSGQDRYTTEVVVNVGGTMQMLGGRQSGGAPAGGNIG
GGQPQGGWGQPQQPQGGNQFSGGAQSRPQQSAPAAPSNEPPMDFDDDIPF (SEQ ID NO: 49)
gi|446090442|ref|WP_000168297.1|: 1-178 ssDNA-binding protein
[Escherichia albertii]
MASRGVNKVILVGNLGQDPEVRYMPNGGAVANITLATSEFWRDKATGEMKEQTEWHRVVLFGKL
AEVASEYLRKGSQVYIEGQLRTRKWTDQSGQDRYTTEVVVNVGGTMQMLGGRQSGGAPAGGNIG
GGQPQGGWGQPQQPQGGNQFSGGAQSRPQQSAPAAPSNEPPMDFDDDIPF (SEQ ID NO: 50)
gi|446090451|ref|WP_000168306.1|: 1-188 ssDNA-binding protein
[Escherichia coli]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGWGQPQQPQGGNQFSGGAQSRPQQSAPAAPSNEPPMDFDDDIPF (SEQ
ID NO: 111)
gi|780016555|ref|WP_045438572.1|: 1-177 MULTISPECIES: ssDNA-binding
protein [Citrobacter]
MASRGVNKVILVGNLGQDPEVRYMPNGGAVANITLATSESWRDKATGEMKEQTEWHRVVLFGKL
AEVASEYLRKGSQVYIEGQLRTRKWTDQSGVEKYTTEVVVNVGGTMQMLGGRQGGGAPAGGNA
GGGQQGGWGQPQQPQGGNQFSGGAQSRPQQSAPAAPSNEPPMDFDDDIPF (SEQ ID NO: 51)
gi|835333245|ref|WP_047459655.1|: 1-178 ssDNA-binding protein
[Citrobacter koseri]
MASRGVNKVILVGNLGQDPEVRYMPNGGAVANITLATSESWRDKQTGEMKEQTEWHRVVLFGKL
AEVASEYLRKGSQVYIEGQLRTRKWTDQSGQDKYTTEVVVNVGGTMQMLGGRQGGGAPAGGNM
GGGQQQGGWGQPQQPQGGNQFSGGAQSRPQQQSAPAPSNEPPMDFDDDIPF (SEQ ID NO: 52)
gi|558738441|gb|EST86567.1|: 1-171 single-stranded DNA-binding
protein [Escherichia coli ECC-1470]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQGGWGQPQQPQGGNQFSGGAQSRPQQSAPAAPSNEPPMD (SEQ ID NO: 53)
gi|501084104|ref|WP_012134613.1|: 1-178 ssDNA-binding protein
[Citrobacter koseri]
MASRGVNKVILVGNLGQDPEVRYMPNGGAVANITLATSESWRDKQTGEMKEQTEWHRVVLFGKL
AEVASEYLRKGSQVYIEGQLRTRKWTDQSGQDKYTTEVVVNVGGTMQMLGGRQGGGVPAGGNM
GGGQQQGGWGQPQQPQGGNQFSGGAQSRPQQQSAPAPSNEPPMDFDDDIPF (SEQ ID NO: 54)
gi|673534660|emb|CDZ85363.1|: 1-178 single-stranded DNA-binding
protein [Citrobacter koseri]
MASRGVNKVILVGNLGQDPEVRYMPNGGAVANITLATSESWRDKQTGEMKEQTEWHRVVLFGKL
AEVASEYLRKGSQVYIEGQLRTRKWTDQSGQDKYITEVVVNVGGTMQMLGGRQGGGAPAGGNM
GGGQQQGGWGQPQQPQGGNQFSGGAQSRPQQQSAPAPSNEPPMDFDDDIPF (SEQ ID NO: 55)
gi|446090452|ref|WP_000168307.1|: 1-171 MULTISPECIES: ssDNA-binding
protein [Shigella]
MASRGVNKVILVGNLGQDPEVRYMPNGGAVANITLATSESWRDKATGEMKEQTEWHRVVLFGKL
AEVASEYLRKGSQVYIEGQLRTRKWTDQSGQDRYTTEVVVNVGGTMQMLGGRQGGGAPAGGNIG
GGQPQQPQGGNQFSGGAQSRPQQSAPAAPSNEPPMDFDDDIPF (SEQ ID NO: 56)
gi|489923275|ref|WP_003826621.1|: 1-174 MULTISPECIES: ssDNA-binding
protein Enterobacteriaceae]
MASRGVNKVILVGNLGQDPEVRYMPNGGAVANITLATSESWRDKATGEMKEQTEWHRVVLFGKL
AEVASEYLRKGSQVYIEGQLRTRKWTDQSGVEKYTTEVVVNVGGTMQMLGGRQGGGAPAGGGQ
QQGGWGQPQQPQGGNQFSGGAQSRPQQSAPAAPSNEPPMDFDDDIPF (SEQ ID NO: 57)
gi|817709260|ref|WP_046671365.1|: 1-174 MULTISPECIES: ssDNA-binding
protein [Citrobacter freundii complex]
MASRGVNKVILVGNLGQDPEVRYMPNGGAVANITLATSESWRDKATGEMKEQTEWHRVVLFGKL
AEVASEYLRKGSQVYIEGQLRTRKWTDQSGVEKYTTEVVVNVGGTMQMLGGRQGGGAPAGGGQ
QQGGWGQPQQPQGGNQFSGGGQSRPQQSAPAAPSNEPPMDFDDDIPF (SEQ ID NO: 58)
gi|489121902|ref|WP_003031723.1|: 1-174 MULTISPECIES: ssDNA-binding
protein [Citrobacter]
MASRGVNKVILVGNLGQDPEVRYMPNGGAVANITLATSESWRDKATGEMKEQTEWHRVVLFGKL
AEVASEYLRKGSQVYIEGQLRTRKWTDQSGVEKYTTEVVVNVGGTMQMLGGRQGGGAPAGGGQ
QQGGWGQPQQPQGGNQFSGGEQSRPQQSAPAAPSNEPPMDFDDDIPF (SEQ ID NO: 59)
gi|493739129|ref|WP_006688290.1|: 1-174 ssDNA-binding protein
[Citrobacter youngae]
MASRGVNKVILVGNLGQDPEVRYMPNGGAVANITLATSESWRDKATGEMKEQTEWHRVVLFGKL
AEVAGEYLRKGSQVYIEGQLRTRKWTDQSGVEKYTTEVVVNVGGTMQMLGGRQGGGAPAGGGQ
QQGGWGQPQQPQGGNQFSGGAQSRPQQSAPAAPSNEPPMDFDDDIPF (SEQ ID NO: 60)
gi|754956873|ref|WP_042312982.1|: 1-174 single-stranded DNA-binding
protein [Citrobacter werkmanii]
MASRGVNKVILVGNLGQDPEVRYMPNGGAVANITLATSESWRDKATGEMKEQTEWHRVVLFGKL
AEVASEYLRKGSQVYIEGQLRTRKWTDQSGVEKYTTEVVVNVGGTMQMLGGRQGGGAPAGGGQ
QQGGWGQPQQPQGGNQFSGGAQSRPQQSAPAAPSNEPSMDFDDDIPF (SEQ ID NO: 61)
gi|851983070|ref|WP_048241790.1|: 1-174 ssDNA-binding protein
[Citrobacter sp. MGH109]
MASRGVNKVILVGNLGQDPEVRYMPNGGAVANITLATSESWRDKATGEMKEQTEWHRVVLFGKL
AEVASEYLRKGSQVYIEGQLRTRKWTDQSGVEKYTTEVVVNVGGTMQMLGGRQGGGAPAGGGQ
QQGGWGQPQQPQGGNQFSGGAQSRLQQSAPAAPSNEPPMDFDDDIPF (SEQ ID NO: 62)
gi|507084615|ref|WP_016155359.1|: 1-175 MULTISPECIES: ssDNA-binding
protein Enterobacteriaceae]
MASRGVNKVILVGNLGQDPEVRYMPNGGAVANITLATSESWRDKATGEMKEQTEWHRVVLFGKL
AEVASEYLRKGSQVYIEGQLRTRKWTDQSGVEKYTTEVVVNVGGTMQMLGGRQGGGAPAGGGQ
QQGGWGQPQQPQGGNQFSGGAQSRPQQQSAPAAPSNEPPMDFDDDIPF (SEQ ID NO: 63)
gi|754934450|ref|WP_042291190.1|: 1-174 MULTISPECIES: ssDNA-binding
protein [Citrobacter]
MASRGVNKVILVGNLGQDPEVRYMPNGGAVANITLATSESWRDKQTGEMKEQTEWHRVVLFGKL
AEVASEYLRKGSQVYIEGQLRTRKWTDQSGQDKYTTEVVVNVGGTMQMLGGRQGGGAPAGGQQ
QQGGWGQPQQPQGGNQFSGGAQSRPQQQSAPAPSNEPPMDFDDDIPF (SEQ ID NO: 64)
gi|489956977|ref|WP_003860284.1|: 1-174 MULTISPECIES: ssDNA-binding
protein [Proteobacteria]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGQEKYTTEVVVNVGGTMQMLGGRQGGGAPAGGGQ
QQGGWGQPQQPQGGNQFSGGAQSRPQQQSAPAPSNEPPMDFDDDIPF (SEQ ID NO: 65)
gi|390676734|gb|EIN52819.1|: 1-155 single-stranded DNA-binding protein
[Escherichia coli PA5]
MPNGGAVANITLATSESWRDKATGEMKEQTEWHRVVLEGKLAEVASEYLRKGSQVYIEGQLRTRK
WTDQSGQDRYTTEVVVNVGGTMQMLGGRQGGGAPAGGNIGGGQPQGGWGQPQQPQGGNQFSGG
AQSRPQQSAPAAPSNEPPMDFDDDIPF (SEQ ID NO: 66)
gi|695799260|ref|WP_032713447.1|: 1-174 ssDNA-binding protein
[Enterobacter aerogenes]
MASRGVNKVILVGNLGQDPEVRYMPSGGAVANFTLATSESWRDKQTGEMKEQTEWHRVVLFGKL
AEVAGEYLRKGSQVYIEGQLRTRKWTDQSGQDKYTTEIVVNVGGTMQMLGGRQGGGAPASGGQQ
QGGWGQPQQPQGGNQFSGGAQSRPQQQAPAAPSNEPPMDFDDDIPF (SEQ ID NO: 67)
gi|695739375|ref|WP_032665749.1|: 1-176 ssDNA-binding protein
[Enterobacter cloacae]
MASKGVNKVILVGNLGQDPEVRYLPSGGAVCSVTLATSESWRDKATGELKEQTEWHRVVLFGKLA
EVAGEYLRKGSQVYIEGQLRTRKWTDQSGQEKYTTEVVVNVGGTMQMLGGRQGGGAPTGGSQNQ
QQGGWGRHQQPQGGNQFSGGAQSRPQQQSAPAPSNEPPMDLDDDIPF (SEQ ID NO: 68)
gi|895840578|ref|WP_048957472.1|: 1-174 ssDNA-binding protein
[Enterobacter cloacae]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGAEKYTTEVVVNVGGTMQMLGGRQGGGAPAGGSQ
QQGGWGQPQQPQGGNQFSGGAQSRPQQQSAPAPSNEPPMDFDDDIPF (SEQ ID NO: 69)
gi|959987279|gb|ALR75018.1|: 1-178 single-stranded DNA-binding
protein [Klebsiella sp. G5]
MASRGVNKVILVGNLGQDPEVRYMPSGGAVANITLATSESWRDKQTGEMKEQTEWHRVVLFGKL
AEVAGEYLRKGSQVYIEGQLRTRKWTDQSGQEKYTTEVVVNVGGTMQMLGGRQGGGAPAGGNM
GGGQQQGGWGQPQQPQGGNQFSGGAQSRPQQQSAPAPSNEPPMDFDDDIPF (SEQ ID NO: 70)
gi|896091886|ref|WP_049127706.1|: 1-175 ssDNA-binding protein
[Klebsiella oxytoca]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGQEKYTTEVVVNVGGTMQMLGGRQQGASAPAGGG
QQQGGWGQPQQPQGGNQFSGGAQSRPQQQAPAAPSNEPPMDFDDDIPF (SEQ ID NO: 71)
gi|745784477|ref|WP_039077898.1|: 1-178 MULTISPECIES: ssDNA-binding
protein [Enterobacteriaceae]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGAEKYTTEVVVNVGGTMQMLGGRQGGGAPAGGNM
GGGQGQQGGWGQPQQPQGGNQFSGGAQSRPQQSAPAPSNEPPMDFDDDIPF (SEQ ID NO: 72)
gi|503133400|ref|WP_013368061.1|: 1-178 ssDNA-binding protein
[Enterobacter lignolyticus]
MASRGVNKVILVGNLGQDPEVRYMPSGGAVANITLATSESWRDKQTGEMKEQTEWHRVVLFGKL
AEVAGEYLRKGSQVYIEGQLRTRKWTDQSGQEKYTTEVVVNVGGTMQMLGGRQGGGASAGGNM
GGGQQQGGWGQPQQPQGGNQFSGGAQSRPQQQSAPAPSNEPPMDFDDDIPF(SEQ ID NO: 73)
gi|727165443|ref|WP_033636191.1|: 1-176 ssDNA-binding protein
[Serratia marcescens]
MASRGVNKVILVGNLGQDPEVRYMPNGGAVANITLATSESWRDKATGEQKEKTEWHRVVLFGKL
AEVAGEYLRKGSQVYIEGSLQTRKWQDQSGQDRYTTEIVVNVGGTMQMLGGRQGGGAPAGQSAG
GQSGWGQPQQPQGGNQFSGGQQQSRPAQNSAPATSNEPPMDFDDDIPF (SEQ ID NO: 74)
gi|782727713|ref|WP_045620928.1|: 1-174 MULTISPECIES: ssDNA-binding
protein [Enterobacter cloacae complex]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGQEKYTTEVVVNVGGTMQMLGGRQGSGAPAGGGQ
QQGGWGQPQQPQGGNQFSGGAQSRPQQQSAPAPSNEPPMDFDDDIPF (SEQ ID NO: 75)
gi|695651336|ref|WP_032619399.1|: 1-176 MULTISPECIES: ssDNA-binding
protein [Enterobacter cloacae complex]
MASKGVNKVILVGNLGQDPEVRYLPSGGAVCSVTLATSESWRDKATGELKEQTEWHRVVLFGKLA
EVAGEYLRKGSQVYIEGQLRTRKWTDQSGQEKYTTEVVVNVGGTMQMLGGRQGGGAPAGGSQN
QQQGGWGQPQQPQGGNQFSGGAQSRPQQQSAPAPSNEPPMDFDDDIPF(SEQ ID NO: 76)
gi|490199293|ref|WP_004097799.1|: 1-175 MULTISPECIES: ssDNA-binding
protein [Enterobacteriaceae]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGQEKYTTEVVVNVGGTMQMLGGRQQGAGAPAGGG
QQQGGWGQPQQPQGGNQFSGGAQSRPQQQAPAAPSNEPPMDFDDDIPF (SEQ ID NO: 77)
gi|490994755|ref|WP_004856483.1|: 1-175 MULTISPECIES: ssDNA-binding
protein [Enterobacteriaceae]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGQEKYTILIVVNVGGTMQMLGGRQQGAGAPAGGGQ
QQGGWGQPQQPQGGNQFSGGAQSRPQQQAPAAPSNEPPMDFDDDIPF (SEQ ID NO: 78)
gi|980964768|ref|WP_059385087.1|: 1-176 single-stranded DNA-binding
protein [Enterobacter cloacae]
MASKGVNKVILVGNLGQDPEVRYLPSGSAVCSVTLATSESWRDKATGELKEQTEWHRVVLFGKLA
EVAGEYLRKGSQVYIEGQLRTRKWTDQSGQEKYTTEVVVNVGGTMQMLGGRQGGGAPAGGSQN
QQQGGWGQPQQPQGGNQFSGGAQSRPQQQSAPAPSNEPPMDFDDDIPF (SEQ ID NO: 79)
gi|896015843|ref|WP_049079199.1|: 1-175 ssDNA-binding protein
[Klebsiella oxytoca]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGQEKYTTEVVVNVGGTMQMLGGRQQGAGAPAGGG
QQQGGWGQPQQPQGGNQYSGGAQSRPQQQAPAAPSNEPPMDFDDDIPF (SEQ ID NO: 80)
gi|490229479|ref|WP_004127826.1|: 1-175 ssDNA-binding protein
[Klebsiella oxytoca]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGQEKYTTEVVVNVGGTMQMLGGRQQGAGAPAGGG
QQQGGWGQPQQPQGGNQFSGGAQSRPQQQTPAAPSNEPPMDFDDDIPF (SEQ ID NO: 81)
gi|727291496|ref|WP_033749160.1|: 1-184 MULTISPECIES: ssDNA-binding
protein [Pantoea]
MASRGVNKVILVGNLGQDPEVRYMPNGGAVANITLATSESWRDKQTGENKEITEWHRVVLFGKLA
EVAGEYLRKGSQVYIEGQLRTRKWQDQGGQDRYTTEVVVNVGGTMQMLGGRQQGGASAGGAPM
GGGQQSGGNNNGWGQPQQPQGGNQFSGGAQSRPQPQSAPASNNNEPPMDFDDDIPF (SEQ ID
NO: 82)
gi|950093073|ref|WP_057172980.1|: 1-175 single-stranded DNA-binding
protein [Klebsiella oxytoca]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGQEKYTTEVVVNVGGTMQMLGGRQQGAGAPAGGG
QQQGGWGQPQQPQGGNQFSGGAQSRPQQQAPAAPSNETPMDFDDDIPF (SEQ ID NO: 83)
gi|896291109|ref|WP_049273747.1|: 1-176 MULTISPECIES: ssDNA-binding
protein [Enterobacteriaceae]
MASRGVNKVILVGNLGQDPEVRYMPNGGAVANITLATSESWRDKATGEQKEKTEWHRVVLFGKL
AEVAGEYLRKGSQVYIEGSLQTRKWQDQSGQDRYTTEIVVNVGGTMQMLGGRQGGGAPAGQSAG
GQGGWGQPQQPQSGNQFSGGQQQSRPAQNSAPATSNEPPMDFDDDIPF (SEQ ID NO: 84)
gi|556473803|ref|WP_023325414.1|: 1-174 ssDNA-binding protein
[Klebsiella pneumoniae]
MASRGVNKVILVGNLGQDPEVRYMPSGGAVANFTLATSESWRDKHTGEMKEQTEWHRVVLFGKL
AEVAGEYLRKGSQVYIEGQLRTRKWTDQSGQDKYTTEVVVNVGGTMQMLGGRQGGGAPAGGGQ
QQGGWGQPQQPQGGNQFSGGAQSRPQQQAPAAPSNEPPMDFDDDIPF (SEQ ID NO: 85)
gi|983342195|ref|WP_060522659.1|: 1-171 single-stranded DNA-binding
protein [Klebsiella pneumoniae]
MASRGVNKVILVGNLGQDPEVRYMPSGGAVANFTLATSESWRDKQTGEMKEQTEWH
RVVLFGKLAEVAGEYLRKGSQVYIEGQLRTRKWTDQSGQDKYTTEVVVNVGGTMQMLGGRQGG
GAPAGGGQQQGGWGQPQGGNQFSGGAQSRPQQQAPAAPSNEPPMDFDDDIPF (SEQ ID NO: 86)
gi|490253751|ref|WP_004151744.1|: 1-174 MULTISPECIES: ssDNA-binding
protein [Enterobacteriaceae]
ASRGVNKVILVGNLGQDPEVRYMPSGGAVANFTLATSESWRDKQTGEMKEQT
EWHRVVLFGKLAEVAGEYLRKGSQVYIEGQLRTRKWTDQSGQDKYTTEVVVNVGGTMQMLGGR
QGGGAPAGGGQQQGGWGQPQQPQGGNQFSGGAQSRPQQQAPAAPSNEPPMDFDDDIPF (SEQ ID
NO: 87)
gi|896191520|ref|WP_049200586.1|: 3-176 ssDNA-binding protein
[Klebsiella pneumoniae]
MASRGVNKVILVGNLGQDPEVRYMPSGGAVANFTLATSESWRDKQTGEMKEQTEWHRVVLFGKL
AEVAGEYLRKGSQVYIEGQLRTRKWTDQSGQDKYTTEVVVNVGGTMQMLGGRQGGGAPAGGGQ
QQGGWGQPQQPQGGNQFSGGAQSRPQQQAPAAPSNEPPMDFDDDIPF (SEQ ID NO: 88)
gi|757780461|ref|WP_042999045.1|: 1-175 MULTISPECIES: ssDNA-binding
protein [Gammaproteobacteria]
MASRGVNKVILVGNLGQDPEVRYMPNGGAVANITLATSESWRDKQTGEMK
EQTEWHRVVLFGKLAEVASEYLRKGSQVYIEGQLRTRKWTDQSGVEKYTTEVVVNVGGTMQMLG
GRQGGGAPAGGQQQQGGWGQPQQPQGGNQFSGGAQSRPQQQSAPAAPSNEPPMDFDDDIPF (SEQ ID
NO: 89)
gi|779882983|ref|WP_045362433.1|: 1-173 ssDNA-binding protein
[Enterobacter aerogenes]
MASRGVNKVILVGNLGQDPEVRYMPSGGAVANFTLATSESWRDKQTGEMKEQTEWHRVVLFGKL
AEVAGEYLRKGSQVYIEGQLRTRKWTDQSGQDKYTTEIVVNVGGTMQMLGGRQGGGAPAGGQQQ
GGWGQPQQPQGGNQFSGGAQSRPQQQAPAAPSNEPPMDFDDDIPF (SEQ ID NO: 90)
gi|505181644|ref|WP_015368746.1|: 1-174 ssDNA-binding protein
[Enterobacter aerogenes]
MASRGVNKVILVGNLGQDPEVRYMPSGGAVANFTLATSESWRDKQTGEMKEQTEWHRVVLFGKL
AEVAGEYLRKGSQVYIEGQLRTRKWTDQSGQDKYTTEIVVNVGGTMQMLGGRQGGGAPAGGGQQ
QGGWGQPQQPQGGNQFSGGAQSRPQQQAPAAPSNEPPMDFDDDIPF (SEQ ID NO: 91)
gi|727192951|ref|WP_033654707.1|: 1-177 MULTISPECIES: ssDNA-binding
protein [Serratia]
MASRGVNKVILVGNLGQDPEVRYMPNGGAVANITLATSESWRDKATGEQKEKTEWHRVVLFGKL
AEVAGEYLRKGSQVYIEGSLQTRKWQDQSGQDRYTTEIVVNVGGTMQMLGGRQGGGAPAGQSAG
GQGGWGQPQQPQGGNQFSGGQQQSRPAQNSAPAASSNEPPMDFDDDIPF (SEQ ID NO: 92)
gi|493870158|ref|WP_006816705.1|: 1-174 ssDNA-binding protein
[Yokenella regensburgei]
MASRGVNKVILVGNLGQDPEVRYMPNGGAVANITLATSESWRDKATGEMKEQTEWHRVVLFGKL
AEVAGEYLRKGSQVYIEGQLRTRKWTDQSGQEKYTTEIVVNVGGTMQMLGGRQQGGAPAGGGQQ
QGGWGQPQQPQGGNQFSGGAQSRPQQQSAPAPSNEPPMDFDDDIPF (SEQ ID NO: 93)
gi|797191136|ref|WP_045853449.1|: 1-178 ssDNA-binding protein
[Raoultella terrigena]
MASRGVNKVILVGNLGQDPEVRYMPSGGAVANFTLATSESWRDKQTGEMKEQTEWHRVVLFGKL
AEVAGEYLRKGSQVYIEGQLRTRKWTDQSGAEKYTTEIVVNVGGTMQMLGGRQGGGAPAGGGQQ
QGGWGQPQQPQQQPQGGNQFSGGAQSRPQQQAPAAPSNEPPMDFDDDIPF (SEQ ID NO: 94)
gi|556230820|ref|WP_023284467.1|: 1-174 ssDNA-binding protein
[Klebsiella pneumoniae]
MASRGVNKVILVGNLGQDPEVRYMPSGGAVANFTLATSESWRDKQTGEMKEQTEWHRVVLFGKL
AEVAGEYLRKGSQVYIEGQLRTRKWTDQSGQDKYTTEVVVNVGGTMQMLGGRQGGGAPAGGGQ
QQGGWGQPQQPQGGNQFSGGAQSRPQQQAPSAPSNEPPMDFDDDIPF (SEQ ID NO: 95)
gi|518042119|ref|WP_019212327.1|: 1-181 MULTISPECIES: ssDNA-binding
protein [Yersinia]
MASRGVNKVILVGNLGQDPEVRYMPNGGAVANITLATSESWRDKATGEQKEKTEWHRVVLFGKL
AEVAGEYLRKGSQVYIEGALQTRKWQDQSGQERYTTEVVVNVGGTMQMLGGRQGGGAPAGGSQ
QDGGAQGGWGQPQQPQGGNQFSGGQTSRPAQSAPAAQPQGGNEPPMDFDDDIPF (SEQ ID NO: 96)
gi|556404007|ref|WP_023305049.1|: 1-174 ssDNA-binding protein
[Klebsiella pneumoniae]
MASRGVNKVILVGNLGQDPEVRYMPSGGAVANFTLATSESWRDKQTGEMKEQTEWHRVVLFGKL
AEVAGEYLRKGSQVYIEGQLRTRKWTDQSGQDKYTTEVVVNVSGTMQMLGGRQGGGAPAGGGQ
QQGGWGQPQQPQGGNQFSGGAQSRPQQQAPAAPSNEPPMDFDDDIPF (SEQ ID NO: 97)
gi|494949566|ref|WP_007675594.1|: 1-180 ssDNA-binding protein
[Cronobacter condimenti]
MASRGVNKVILVGNLGQDPEVRYMPNGGAVANLRLATSESWRDKQTGEMKEVFEWHSVVLYGKL
AEVAGEYLRKGSQIYIEGQLRTRKWQDQSGQDRYSTEVVVNVGGTMQMLGGRQGGGAPAGGNM
GGGQQQGGWGQPQQPQQQSGGAQFSGGAQSRPQQQAPAPSNEPPMDFDDDIPF (SEQ ID NO: 98)
gi|647265972|ref|WP_025713817.1|: 1-174 ssDNA-binding protein
[Klebsiella sp. 10982]
MASRGVNKVILVGNLGQDPEVRYMPSGGAVANFTLATSESWRDKQTGEMKEQTEWHRVVLFGKL
AEVAGEYLRKGSQVYIEGQLRTRKWTDQSGQDKYTTEVVVNVGGTMQMLGGRQGGGAPAGGGQ
QQGGWGQPQQPQGGSQFSGGAQSRPQQQAPAAPSNEPPMDFDDDIPF (SEQ ID NO: 99)
gi|694077789|ref|WP_032423210.1|: 1-174 single-stranded DNA-binding
protein [Klebsiella pneumoniae]
MASRGVNKVILVGNLGQDPEVRYMPSGGAVANFTLATSESWRDKQTGEMKEQTEWHRVVLFGKL
AEVAGEYLRKGSQVYIEGQLRTRKWTDQSGQDKYTTEVVVNVGGTMQMLGGRQGGGAPAGGGQ
QQGGWGQPQQPQGGNQFSGGAQSRPQQQAPAAPSNETPMDFDDDIPF (SEQ ID NO: 100)
gi|740321204|ref|WP_038158358.1|: 1-176 ssDNA-binding protein
[Trabulsiella guamensis]
MASRGVNKVILVGNLGQDPEVRYMPNGGAVANITLATSESWRDKQTGEMKEQTEWHRVVLFGKL
AEVASEYLRKGSQVYIEGQLRTRKWTDQSGVEKYTTEVVVNVGGTMQMLGGRQQGAGAPAGGG
QQQQGGWGQPQQPQGGAQFSGGAQSRPQQQSAPAPSNEPPMDFDDDIPF (SEQ ID NO: 101)
gi|798873041|ref|WP_045897887.1|: 1-176 ssDNA-binding protein
[Enterobacter cloacae]
MASKGVNKVILVGNLGQDPEVRYLPSGGAVCSVTLATSESWRDKATGELKEQTEWHRIVLFGKLA
EVAGEYLRKGSQVYIEGQLRTRKWTDQSGQEKYTTEVVVNVGGTMQMLGGRQGGGAPAGGGQS
QQHGGWGQYQHPQVGNQFSGGAQSRPQQQSAPAPSNEPPMDFDDDIPF (SEQ ID NO: 102)
gi|930179010|ref|WP_054179758.1|: 1-177 ssDNA-binding protein
[Trabulsiella odontotermitis]
MASRGVNKVILVGNLGQDPEVRYMPNGGAVANITLATSESWRDKQTGEMKEQTEWHRVVLFGKL
AEVASEYLRKGSQVYIEGQLRTRKWTDQSGVEKYTTEVVVNVGGTMQMLGGRQQGAGAPAGGG
QPQQQGGWGQPQQPQGGAQFSGGAQSRPQQQSAPAPSNEPPMDFDDDIPF (SEQ ID NO: 103)
gi|908733191|ref|WP_049855775.1|: 1-177 ssDNA-binding protein
[Trabulsiella odontotermitis]
MASRGVNKVILVGNLGQDPEVRYMPNGGAVANITLATSESWRDKQTGEMKEQTEWHRVVLFGKL
AEVASEYLRKGSQVYIEGQLRTRKWTDQSGVEKYTTEVVVNVGGTMQMLGGRQQGAGAPAGGG
QQQGGWGQPQQPQQQGGAQFSGGAQSRPQQQSAPAPSNEPPMDFDDDIPF (SEQ ID NO: 104)
gi|763074203|ref|WP_043955685.1|: 1-174 ssDNA-binding protein
[Kosakonia radicincitans]
MASRGVNKVILVGNLGQDPEVRYMPSGGAVANITLATSESWRDKQTGEMKEQTEWHRVVLFGKL
AEVAGEYLRKGSQVYIEGQLRTRKWTDQSGQEKYTTEVVVNVGGTMQMLGGRQGGGAPAGGGQ
QQGGWGQPQQPQGGNQFSGGAQSRPQQSSAPAPSNEPPMDFDDDIPF (SEQ ID NO: 105)
gi|983140349|ref|WP_060448016.1|: 1-177 single-stranded DNA-binding
protein [Serratia marcescens]
MASRGVNKVILVGNLGQDPEVRYMPNGGAVANITLATSESWRDKATGEQKEKTEWHRVVLFGKL
AEVAGEYLRKGSQVYIEGSLQTRKWTDQAGVEKYTTEVVVNVGGTMQMLGGRQGGGAPAGQSA
GGQGGWGQPQQPQGGNQFSGGQQQSRPAQNSAPAASSNEPPMDFDDDIPF (SEQ ID NO: 106)
gi|737931947|ref|WP_035896752.1|: 1-175 MULTISPECIES: ssDNA-binding
protein [Kluyvera]
MASRGVNKVILVGNLGQDPEVRYMPNGGAVANITLATSESWRDKATGEMKEQTEWHRVVLFGKL
AEVAGEYLRKGSQVYIEGQLRTRKWTDQSGAEKYTTEVVVNVGGTMQMLGGRQGGGAPAGGGQ
QQQGGWGQPQQPQGGNQFSGGAQSRPQQQSAPAPSNEPPMDFDDDIPF (SEQ ID NO: 107)
gi|949705074|ref|WP_057057886.1|: 1-175 ssDNA-binding protein
[Enterobacter asburiae]
MASRGVNKVILVGNLGQDPEVRYMPSGGAVANITLATSESWRDKATGEMKEQTEWHRVVLFGKL
AEVAGEYLRKGSQVYIEGQLRTRKWTDQSGAEKYTTEVVVNVGGTMQMLGGRQGGGTPAGGGQ
QQQGGWGQPQQPQGGNQFSGGAQSRPQQQSAPAPSNEPPMDFDDDIPF (SEQ ID NO: 112)

TABLE 8
SSB Genes Corresponding to the Protein Sequences Referenced by Uniprot IDs.

| | | | |
|---|---|---|---|
| SSB_ECOLI | I6F672_SHIFL | Q327X2_SHIDS | A0A0A7A3L7_SHIDY |
| A8AN55_CITK8 | I6G8M5_SHIFL | A0A0G3QH78_KLUIN | J1YNM2_9ENTR |
| A6TGW7_KLEP7 | A0A0F6JWW6_ENTAE | L0M840_ENTBF | A0A089UTS5_9ENTR |
| K8BJX4_9ENTR | A4W5H1_ENT38 | A0A0A2VXI7_BEABA | V5CXL1_ENTCL |
| I2BDV0_SHIBC | A7MPL6_CROS8 | D2TRS6_CITRI | Q6D0V9_PECAS |
| W8TVY4_YEREN | L0MKB5_SERMA | SSB_YERPE | W0L7W3_9ENTR |
| SSB1_SALTY | A8GK93_SERP5 | G7LX00_9ENTR | A0A0K0HGJ5_SALBC |
| A0A089PKR4_PLUGE | E0SFA5_DICD3 | A9MGM8_SALAR | H2IV20_RAHAC |
| A0A0A1B0L7_9ENTR | A0A0M2KCH9_9ENTR | W0I221_9ENTR | E1SAY5_PANVC |
| D4GHC5_PANAM | A0A097R6T3_HAFAL | C0ARM9_9ENTR | E6WHP7_PANSA |
| D8MLY1_ERWBE | A0A0A0Z1Q4_9ENTR | B2VKC4_ERWT9 | U3TS91_9ENTR |
| B4EYJ2_PROMH | Q2NR22_SODGM | D4BZF6_PRORE | D3VBV3_XENNA |
| J7TBA8_MORMO | K8WA41_9ENTR | Q7MZE3_PHOLL | A0A068QYL1_9ENTR |
| D3V5U1_XENBS | B7VI95_VIBTL | A0A068R687_9ENTR | I0DSY1_PROSM |
| A0A0C5W765_9GAMM | C5BF00_EDWI9 | A6ARK4_VIBCY | A8T927_9VIBR |
| Q5E7Z8_VIBF1 | SSB_VIBPA | U4K8W8_9VIBR | F0LWT6_VIBFN |
| F9TAJ8_9VIBR | A0A0F5VDV1_9GAMM | D0Z1S1_PHODD | F7YM47_VIBA7 |
| A0A0A5JJ92_9VIBR | E3BJW7_9VIBR | SSB_VIBCH | F9RB03_VIBSN |
| A0A097QHM9_9VIBR | Q7MHB8_VIBVY | I2B9U6_SHIBC | A0A0D8MYY3_PHOLE |
| A0A0D8PTJ3_9GAMM | W0LBZ8_9ENTR | A0A0M2KBM0_9ENTR | A8G8I2_SERP5 |
| A8GJB0_SERP5 | Q1ZNU1_PHOAS | A0A068QYS8_9ENTR | C9P2V1_VIBME |
| A6D019_9VIBR | G2GZS2_9ENTR | A0A068R4G7_9ENTR | E0WRV0_9ENTR |
| F9T6P1_9VIBR | A0A0M2K592_9ENTR | Q6LM83_PHOPR | G7LLY5_9ENTR |
| U3U422_9ENTR | F7XX66_MOREP | A0A089PM27_PLUGE | A0A0F5ARE6_9GAMM |
| J3Z626_9ENTR | A0A090IIF7_9GAMM | D8MJG0_ERWBE | A0A0A7S1F2_9GAMM |
| R4I1N0_9ENTR | SSB_BPP1 | A0A0A1B773_9ENTR | A0A0A1B773_9ENTR |
| J3TXW1_9ENTR | J3TXW1_9ENTR | SSB2_SALTY | U3U1Y0_9ENTR |
| D3V012_XENBS | A6AUY7_VIBCY | H2FYH8_OCESG | J1QW29_9ENTR |
| A0A0F6AES3_9GAMM | A0A0F6AES3_9GAMM | A0KQ40_AERHH | A0KQ40_AERHH |
| X2H798_9GAMM | A0A0F5ZR91_9GAMM | W8URY6_YEREN | Q1LTY5_BAUCH |
| A0A0F4PQ78_9GAMM | A1S2X0_SHEAM | C4K6G2_HAMD5 | A6TID9_KLEP7 |
| I1E095_9GAMM | I1E095_9GAMM | A0A068QPW4_9ENTR | A6TIR6_KLEP7 |
| A0A0F4QKR0_9GAMM | A0A0E3U3R3_9ENTR | C7R765_KANKD | H0JIY9_9PSED |
| A0A068QP58_9ENTR | SSB_SHEON | A0A0A7EC64_9GAMM | A0A0A7EC64_9GAMM |
| I2JLX2_9GAMM | D4G909_RIEPU | SSB_BUCAI | A0A0F4PA03_PSEO7 |
| A8H012_SHEPA | G4QFE0_GLANF | A0A0F6TPU7_9GAMM | W8KKP5_HALHR |
| SSB_HAEIN | F7WZQ6_9ENTR | A0A0F7M398_9GAMM | Q12IY7_SHEDO |
| D3V4B9_XENBS | A0A0F6JV15_ENTAE | D3VA12_XENNA | K4KIK2_SIMAS |
| D3VAC0_XENNA | M4XAW4_PSEDE | D3V9E7_XENNA | C4L8J0_TOLAT |
| C5BQ98_TERTT | A0A024HBS5_PSEKB | F7NR52_9GAMM | F7NR52_9GAMM |
| SSB_WIGBR | A0A0M2VIU0_9GAMM | A4A656_9GAMM | E4PMP4_MARAH |
| A0A0K1QHA3_PSEFL | SSB_PSEPK | C0AX13_9ENTR | C0AX13_9ENTR |
| F9U8B2_9GAMM | J2VMH4_9BURK | F5ZFB7_ALTSS | F5ZFB7_ALTSS |
| Q7N7R9_PHOLL | A0A068R6L2_9ENTR | Q3J6T1_NITOC | J3DHJ7_9BURK |
| C4KC21_THASP | Q21M21_SACD2 | Q056Y9_BUCCC | A3N0E5_ACTP2 |
| SSB_PSEAE | I3DML2_HAEPH | A1TYM6_MARHV | W0E0Y1_MARPU |
| F3KGP2_9GAMM | L0H1Y6_9GAMM | G2DXR4_9GAMM | Q2S941_HAHCH |
| Q2S941_HAHCH | D3V0M3_XENBS | K0C3K4_CYCSP | D3RQK6_ALLVD |
| I3YBP2_THIV6 | S6BGJ6_9GAMM | J4UTR0_9PAST | D8ISL6_HERSS |
| A1WEN0_VEREI | K6Z560_9ALTE | Q5QWB9_IDILO | H0PUH7_9RHOO |
| C3K2U5_PSEFS | W0DPI3_9GAMM | Q0VSH0_ALCBS | M4R5F5_BIBTR |
| G8PZ64_PSEFL | C1DKP2_ AZOVD | B4EVH3_PROMH | B4EVH3_PROMH |
| Q15ZK8_PSEA6 | Q15ZK8_PSEA6 | K0CKB3_ALCDB | SSB_BUCBP |
| A0A077F648_9PSED | Q4K563_PSEF5 | A0A024EEI5_9PSED | K6XV41_9ALTE |
| A0A0B4XJ04_9GAMM | K6YUU7_9ALTE | B7S2V8_9GAMM | G8M3I5_9BURK |
| A0A0E3Y7R2_9ENTR | Q487T6_COLP3 | Q1YRM7_9GAMM | B8F401_HAEPS |
| K2IWX5_9GAMM | K2IWX5_9GAMM | W8RQ74_PSEST | H8Z2A9_9GAMM |
| I4MXA9_9PSED | I0DXT5_PROSM | Q7VQT7_BLOFL | Q2BLP5_NEPCE |
| F5RHX5_METUF | N6Y596_9RHOO | W0DY92_MARPU | Q603V6_METCA |
| U7U669_9BURK | A0A090ARY2_9ENTR | Q3IJB1_PSEHT | Q1LRM5_CUPMC |
| G3IXZ4_9GAMM | G4T2U7_META2 | F3KYD2_9GAMM | E5ALT6_BURRH |
| Q65V18_MANSM | G4MIH5_9BURK | D3RW92_ALLVD | A0A0F7K1E3_9GAMM |
| SSB_HAEDU | M9X210_MANHA | A0A089YSB8_9PSED | A0A089WKU8_9PSED |
| D0KVW4_HALNC | D5X2Y7_THIK1 | A1WZ36_HALHL | SSB_CUPNH |
| D7DNA2_METV0 | F2G3Q0_ALTMD | A0LDV7_MAGMM | F6AJD7_PSEF1 |
| A4C7X8_9GAMM | E1V5N6_HALED | W0TJZ1_9GAMM | D5AUB1_RHOCB |
| F9Q6N5_9PAST | Q5P300_AROAE | A0A0F5F0Y4_AVIPA | D4X548_9BURK |
| H8FXS8_PHAMO | A4XZ57_PSEMY | A0A0D5EH59_9BURK | A1VTY7_POLNA |
| SSB_COXBU | M1FC50_9ALTE | A0A052IQ42_9BORD | Q0A501_ALKEH |
| SSB_BORPE | A0A0F2P7X2_9GAMM | W0SH75_9RHOO | W0SH75_9RHOO |
| V5UG93_9BURK | A9IHN6_BORPD | G0AGI2_COLFT | F4HFM6_GALAU |
| I8I5Z0_9GAMM | E3HRP1_ACHXA | H3NWG8_9GAMM | A0A089WTH0_9PSED |
| A0A0H4VYA7_9BORD | U2FYN2_9GAMM | J2TVV7_9BURK | B8GV27_THISH |
| D5C3D3_NITHN | B8KWJ2_9GAMM | S6ALR8_PSERE | E1VGN3_9GAMM |
| A4G9L8_HERAR | A0A060B5W2_9GAMM | A0A077LD02_9PSED | B2JCR5_BURP8 |
| W8WTJ8_CASDE | Q145U6_BURXL | C3XAY9_OXAFO | A0A0C4Y4K1_9BURK |
| D5WBB4_BURSC | Q31IV0_THICR | I0HY81_RUBGI | E1T6K9_BURSG |
| A0A0F2RU44_9RHOB | F2LSN1_BURGS | B3PK66_CELJU | SSB_PSESM |
| C6WZ85_METML | A0A0D5V8B5_9BURK | Q122U3_POLSJ | H8GRA6_METAL |
| I3I6A3_9GAMM | Q5QF93_9CAUD | Q2L1W7_BORA1 | G2FAV3_9GAMM |
| I3UH71_ADVKW | Q166G4_ROSDO | A0A0F5QES3_9RHIZ | G8MQ43_9BURK |
| A0A0F4QUZ6_9GAMM | A0A0A1H9H4_9BURK | A0A0M2VEY2_9GAMM | A0Z169_9GAMM |
| I4W834_9GAMM | B1XS47_POLNS | G9ZF80_9GAMM | A0YGK8_9GAMM |
| SSB_PASMU | Q1GYJ8_METFK | F4GAZ0_ALIDK | G8QL94_DECSP |
| A4T051_POLSQ | C9R230_AGGAD | I3BZ36_9GAMM | A4JUL0_BURVG |
| A0A090BUS7_9GAMM | I9KSJ0_9RALS | A0A085BUP3_9RHOB | Q0KEL9_CUPNH |
| A4VHR0_PSEU5 | M4U7M8_9GAMM | M4U7M8_9GAMM | H5WKW5_9BURK |
| A1TWB0_ACIAC | B7J938_ACIF2 | N6YC46_9RHOO | B1XYU6_LEPCP |
| Q6V7S6_9CAUD | H1G1C8_9GAMM | A0A0F5FLE3_9RHIZ | D0IYB1_COMT2 |
| A0A0F5LKK9_9RHIZ | E1SN23_FERBD | I3CFN7_9GAMM | F4QR90_9CAUL |
| A0A0F3LFK2_9CAUL | A9BS53_DELAS | A3K1S1_9RHOB | K2C0P8_9BACT |
| H1S7M6_9BURK | K2JQQ4_9PROT | A0A0F5PUU2_9RHIZ | B8IV04_METNO |
| Q6F764_ACIAD | C5CYD8_VARPS | A0A0H4L8C9_9RHOB | A4JTB6_BURVG |
| A1T012_PSYIN | A1KAZ5_AZOSB | D4ZCF2_SHEVD | Q2RTK0_RHORT |
| K0I6X4_9BURK | A0A070BZW0_BURCE | M4SGM9_9SPHN | E8RTB6_ASTEC |
| S5XVG2_PARAH | Q5NV32_CUPMC | W0PHB6_9BURK | K2DV93_9BACT |
| SSB_RHOS4 | A0A0B5CJL8_NEIEG | Q0BST4_GRABC | N8TJ62_ACIGI |
| N8TJ62_ACIGI | C5TD29_ACIDE | D3SCQ7_THISK | Q07WS7_SHEFN |
| SSB_CAUCR | J5PF93_9RHOB | Q6N604_RHOPA | A0A0F3IJQ8_9GAMM |
| A2SBY4_METPP | C7RIZ8_ACCPU | E3DH61_9PAST | D0D283_9RHOB |
| A0A098G838_9GAMM | A0A0F2QS65_9PSED | B0UMW1_METS4 | A0A0A1B6N6_9ENTR |
| Q21RY1_RHOFT | A4JHX0_BURVG | C6RQL8_ACIRA | K2B9K7_9BACT |
| A1WD45_ACISJ | D3HPN2_LEGLN | T2L5K1_9GAMM | E3I2T1_RHOVT |
| R4VPZ6_9GAMM | B2UCN7_RALPJ | F4GWZ8_PUSST | J0KHY6_9BURK |
| G2E4K5_9GAMM | B9NT08_9RHOB | A6VQI4_ACTSZ | A4EQZ7_9RHOB |
| G2IQI4_9SPHN | A0A0F5LBK0_9RHIZ | K2IPE2_9RHOB | I3DDC4_9PAST |
| B9JF98_AGRRK | A1AWF7_RUTMC | K1ZXB5_9BACT | A0A0M2WTC2_9BURK |
| F2LCY9_BURGS | A3V5P2_9RHOB | F0GF15_9BURK | Q0ANG1_MARMM |
| A0A0D5LSJ4_9RHIZ | SSB_NITEU | A0A0F5FT04_9RHIZ | I1XGC7_METNJ |
| S6AF57_9PROT | A0A0F4RGQ0_9GAMM | W0UWN0_9BURK | SSB_XANCP |
| W6K6N2_9PROT | N8ZEX2_9GAMM | N8ZEX2_9GAMM | L0E0L4_THIND |
| A2SNI7_METPP | B6AXZ3_9RHOB | Q2W4P2_MAGSA | K2Q2L3_9RHIZ |
| U5T0Q8_9GAMM | A0A076K549_9RHOB | A6FSR7_9RHOB | B1KDD5_SHEWM |
| J9DXI6_9PROT | A5WGZ1_PSYWF | B9JWR9_AGRVS | R5PPS2_9BURK |
| I4VXH8_9GAMM | A8LKY4_DINSH | A0A0F2QJX0_9RHOB | D5WNY1_BURSC |
| A0A0A8K3F9_9RHIZ | S6AQQ7_PSERE | C6XJ14_HIRBI | A4BT12_9GAMM |
| C4GH79_9NEIS | G9ESV4_9GAMM | B4EAT8_BURCJ | F5SZ57_9GAMM |
| A0A0A8UPM3_LEGHA | I4YXR6_9RHIZ | G2DYM8_9GAMM | D0CS74_RUELI |
| A3XAJ4_9RHOB | I1B1H0_9RHOB | A0A098GEA4_TATMI | A0A0F7KCW2_9PROT |
| Q63XJ3_BURPS | D9QLP4_BRESC | A0A0F3INJ0_9PROT | Q47IU1_DECAR |
| Q5ZYL6_LEGPH | A3QAA9_SHELP | K2BZG4_9BACT | B1LX07_METRJ |
| W0TJW4_9GAMM | F4R1K1_BREDI | A4YV28_BRASO | A1VVC7_POLNA |
| A0A0D6TGJ7_9RHOB | A0A0M2RDE4_9PROT | A1WP86_VEREI | A3UDK5_9RHOB |
| C6XAV2_METGS | A0A0J6RP75_9RHIZ | A0A0A3VYR5_9GAMM | A5PEQ1_9SPHN |
| A0A060QI44_9PROT | G2DKU1_9NEIS | W0BBC7_9GAMM | A3TWT6_OCEBH |
| A0A099GHI8_9RHOB | V2USK2_9GAMM | A0A0F5PFM8_9SPHN | Q0FQ09_PELBH |
| A4EMD4_9RHOB | Q1GNS9_SPHAL | D0UIT2_AGGAD | A4BEE4_9GAMM |
| M4NB34_9GAMM | D3SGM4_THISK | A0A023NV45_9GAMM | A0A023NV45_9GAMM |
| Q1GII7_RUEST | A0A024EMC9_9PSED | A0A0G3LPP7_XANCT | H0HVG1_9RHIZ |
| K2DU46_9BACT | E1VAE8_HALED | E8TB16_MESCW | A0A0C5KYX1_9SPHN |
| Q3SSB4_NITWN | A0A0E9MSC8_9SPHN | C5ADI0_BURGB | A0A0M3AV39_9SPHN |
| Q5NM94_ZYMMO | Q1YHZ0_AURMS | A0A0G1AXI5_9BACT | A0A0F3IMV0_9GAMM |
| G9ZIW0_9GAMM | Q1QLM1_NITHX | G4CQ97_9NEIS | F5R876_METUF |
| A3JSR5_9RHOB | B4R9P1_PHEZH | D4YXQ1_SPHJU | G2KQL9_MICAA |
| E3EYD9_KETVY | R6I5E6_9PROT | B0UTC9_HISS2 | Q0BYK0_HYPNA |
| B7JAE5_ACIF2 | A8TKW1_9PROT | D5RPM4_9PROT | J2PJG0_9SPHN |
| D9SK11_GALCS | Q213Y3_RHOPB | G4E3E4_9GAMM | SSB_RHIME |
| C5APZ6_METEA | F7QMR1_9BRAD | SSB_RALSO | M9X080_MANHA |
| B0UVW4_HISS2 | F1ZBN2_9SPHN | B3QY18_CHLT3 | Q1R0E6_CHRSD |
| D5CP98_SIDLE | V5SF15_9RHIZ | A0A0D6JBI9_9RHIZ | N9GT97_ACIHA |
| C0N5M9_9GAMM | A0A0F2RFG2_9RHOB | H6SQ65_RHOPH | G6Y9B4_9RHIZ |
| R6J5K6_9PROT | F7VE14_9PROT | A0A0F3K8X6_9GAMM | Q1N941_SPHSS |
| R5R624_9PROT | H0TF15_9BRAD | K5Z2Q0_9PROT | C8NAM6_9GAMM |
| B0SZT3_CAUSK | C3MD07_RHISN | Q2Y6V2_NITMU | X5MNE3_9RHIZ |
| I1YK77_METFJ | H8L1R7_FRAAD | B0VN74_ACIBS | SSB_BRADU |
| G9ZXQ9_9PROT | I3Y923_THIV6 | M5DVK8_9GAMM | A3SNZ2_ROSNI |
| I9L8W8_9SPHN | Q3B682_CHLL7 | B7RFT1_9RHOB | G6EYN2_9PROT |
| G6XK36_9PROT | R5XPX2_9PROT | C7DFD9_9RHOB | A0A077C4P4_9RICK |
| Q11II2_CHESB | A0A0B5E2T4_9RHOB | E8RPM9_ASTEC | B6JFC9_OLICO |
| A3VJM3_9RHOB | SSB_RHILO | G3Z3H6_9NEIS | D2ZVB2_NEIMU |
| A0A0B4X3B3_9RHIZ | D2UDJ2_XANAP | J2WCL8_9SPHN | Q5H2X8_XANOR |
| J8VZF2_9SPHN | F8J558_HYPSM | B7J8U2_ACIF2 | F2JX52_MARM1 |
| F8GEK6_NITSI | D7MZ59_9NEIS | F2BBV5_9NEIS | F9ZVP9_METMM |
| D5BQS9_PUNMI | Q2G4D9_NOVAD | W5YCT8_KOMXY | E3F1U4_KETVY |
| V6F1P2_9PROT | A6X111_OCHA4 | Q2W772_MAGSA | N0B880_9RHIZ |
| B9QZI9_LABAD | I4W169_9GAMM | Q5LSX7_RUEPO | F3SAM2_9PROT |
| A9HM62_GLUDA | W8RVW7_9RHOB | F8GKP0_NITSI | B6ISP2_RHOCS |
| B2UKJ9_RALPJ | B4S530_PROA2 | A7INE3_XANP2 | C0DRP8_EIKCO |
| SSB_BRUA2 | Q0FGL9_9RHOB | Q4FR27_PSYA2 | Q3SLL6_THIDA |
| A9D9K8_HOEPD | J1JWK2_9RHIZ | B6JI03_OLICO | F9ZMD5_ACICS |
| Q2K8E4_RHIEC | B8F4I1_HAEPS | A0A0F2QVS7_9DELT | Q1GG07_RUEST |
| A7TDK0_NEMVE | I4MIC5_9BURK | W0A7X0_9SPHN | A0A0F5ESE3_AVIPA |
| A0A0G2ZM37_9DELT | A0A0A8E100_9XANT | A1B8M8_PARDP | V9VTI7_9RHOB |
| F8GG32_NITSI | Q1DDE2_MYXXD | E6WQZ6_PSEUU | K2FHM2_9BACT |
| F5T0V4_9GAMM | K2H7N0_9RHOB | W6RH93_9RHIZ | U1XXH6_9BURK |
| SSB2_XYLFA | A0A0F4RMX3_9RHOB | SSB_AGRFC | A0A0M3ARW7_9SPHN |
| C1D7M8_LARHH | F9ZLD2_ACICS | U7GLV1_9RHOB | F5Y3Y9_RAMTT |
| Q5FSE3_GLUOX | A9I2Z8_BORPD | F2A5K5_RHIET | G7URR0_PSEUP |
| Q0FLP9_PELBH | A0A0F2NW53_9FLAO | A0A0F5EX66_AVIPA | H8L133_FRAAD |
| B9TDA8_RICCO | Q2NDW0_ERYLH | Q1K3E6_DESAC | M4NHD3_9GAMM |
| B2UEU3_RALPJ | I5AYV2_9DELT | D5V9T9_MORCR | S5RPZ6_9PROT |
| SSB_NEIMB | H8YVI5_9GAMM | E5UI11_NEIMU | H0Q1C2_9RHOO |
| A0A0B4XXZ8_9PROT | A0A0M2LVA1_9SPHN | Q5F7Y0_NEIG1 | G8PH47_PSEUV |
| D5QCC8_KOMHA | G5ZW55_9PROT | A0A0J7KQJ3_LASNI | A0A0F5P8V5_9SPHN |
| A0A0A6CZ02_9SPHN | S9UWZ3_9TRYP | F6IL26_9SPHN | A0A0F5K5M6_9BURK |
| Q08U58_STIAD | A5EWP4_DICNV | Q1GGQ8_RUEST | R5Q9P3_9PROT |
| F9ZU52_ACICS | R6VC65_9BACT | Q28R60_JANSC | H8YW24_9GAMM |
| C6W2S7_DYAFD | A0A0E9M483_9PROT | M1NYV3_BARAA | G4R9S1_PELHB |
| L7U4K1_MYXSD | A7HXM1_PARL1 | H8MMS9_CORCM | F0F0Z9_9NEIS |
| A5G056_ACICJ | A0A0G3BHN5_9BURK | E8UF32_TAYEM | G4CGE7_9NEIS |
| A6W363_MARMS | A0A0M3BUH8_9RHIZ | U5NBX9_9BURK | E0TIG3_PARBH |
| A0A0D5LKG4_9RHIZ | B3E213_GEOLS | A5EUY7_DICNV | A0A0E3Z3E7_9GAMM |
| K0PP83_9RHIZ | V9TR38_9PROT | A0A060QDZ1_9PROT | X2HHK0_9NEIS |
| D5CTA8_SIDLE | A0A097EIF6_9SPHN | M1LT42_9PROT | A0A0G2B146_9BACT |
| A0A0G2B146_9BACT | I4YX49_9RHIZ | A0A017HB59_9RHOB | A1UST1_BARBK |
| Q747Z8_GEOSL | R7KPC2_9BURK | A9E9R6_9RHOB | I7EYX3_PHAIB |
| W0SEU3_9RHOO | A0A0F4QM95_9GAMM | J1K3E2_9RHIZ | M1LV26_9PROT |
| J1TI96_9RHIZ | Q4FLZ6_PELUB | B2FSL1_STRMK | A0A090N7K3_AFIFE |
| A6WZ49_OCHA4 | A0A0F2R6Z2_9PROT | Q0G045_9RHIZ | Q1IPH6_KORVE |
| E0MKP9_9RHOB | E8RFQ0_DESPD | D1KBN2_9GAMM | B2IFV1_BEII9 |
| M1NU57_BARAA | A0A0B5FQA0_9DELT | Q39YR9_GEOMG | A0A0C5V225_9XANT |
| Q7VML3_HAEDU | A0A068T9T5_RHIGA | M9X4F7_MANHA | A0A023Y6B6_9GAMM |
| B9KI69_ANAMF | A8ID59_AZOC5 | H0A2S4_9PROT | M1NS08_BARAA |
| A5V8E1_SPHWW | A0A0B5FX24_9DELT | Q11LW6_CHESB | C7JGA1_ACEP3 |
| M1PSQ3_DESSD | J7QSP6_METSZ | A7HHA9_ANADF | A0A0F2NLG2_9DELT |
| A0A075MK72_9RICK | A1BIR8_CHLPD | SSB1_XYLFA | A0A0F5MP96_9RICK |
| A6FSA2_9RHOB | D0RNY6_9PROT | A0A077AYK7_9RICK | R6Y4F9_9BACT |
| V4KTJ5_9DELT | R7JQ74_9BACT | M9WYF9_MANHA | Q1NJ37_9DELT |
| SSB1_CHLTE | L0EU44_LIBCB | E5Y6K0_BILWA | B8ET29_METSB |
| U6B7F1_9RHIZ | E1QIQ1_DESB2 | A0A0A7PM00_9SPHN | D5X2Q2_THIK1 |
| A7BTL5_9GAMM | B8FAA1_DESAA | E4TPK4_MARTH | A5G9S2_GEOUR |
| Q1N2F3_9GAMM | A8ZRT7_DESOH | A0A0C5W171_9GAMM | A0A0C7DVJ9_9GAMM |
| C8X289_DESRD | K1PF24_CRAGI | Q2GKN5_ANAPZ | A0A0F5PBJ2_9SPHN |
| K1YYU4_9BACT | D0LVJ8_HALO1 | Q6AK18_DESPS | B9M7M5_GEODF |
| G6F3F3_9PROT | A0LIN0_SYNFM | H3KDP2_9BURK | I3YP43_ALIFI |
| B3ENL6_CHLPB | Q2CE60_OCEGH | J9Z216_9PROT | K0NCL2_DESTT |
| Q2S4M7_SALRD | R5WQX3_9BACT | M9RDW4_9RHOB | Q3A6V5_PELCD |
| F5S835_9NEIS | A0A0A1H1W5_9BURK | C6XHB3_LIBAP | Q74B90_GEOSL |
| A0A0C9MSV5_SPHPI | A0A0G3XCA5_9SPHN | Q2S565_SALRD | Q6MMP4_BDEBA |
| A5V945_SPHWW | A0A0F7KXN7_9SPHN | Q2GG22_EHRCR | F2IZR2_POLGS |
| A0A0A1H3N2_9BURK | A0A0G3I1G7_LIBAF | R7C2Q7_9BURK | D7AAH9_STAND |
| B3EB00_GEOLS | D0MG88_RHOM4 | A0A0H4VTP9_9BACT | B5EG54_GEOBB |
| A1AKG6_PELPD | R5EC07_9BURK | C1D7P5_LARHH | A6FSV9_9RHOB |
| F8AB87_THEID | F8ACN0_THEID | R6EXV2_9BACE | F2IBW4_FLUTR |
| Q5FHD4_EHRRG | Q11NU5_CYTH3 | R5I4N3_9BACT | D6Z5C9_DESAT |
| J0LLJ2_9BACT | G5H7V8_9BACT | B8DL66_DESVM | A0A077FMS8_9RICK |
| K7SBZ0_GLUOY | E4U6H4_OCEP5 | E4U6H4_OCEP5 | A0A099TDX5_9RHOB |
| F2I194_PELSM | F0JDI6_DESDE | Q2GD12_NEOSM | C4XMH4_DESMR |
| C1F3G8_ACIC5 | A0A0F3NLM9_9RICK | A0A077DE37_9BURK | R6X338_9BACT |
| A0A0F7JLQ0_9DEIO | A0A0F7JLQ0_9DEIO | A0A0E3UW87_9BACT | H8K3H6_RICAG |
| X5H4M2_9RICK | A3WGB5_9SPHN | R5ELR6_9GAMM | Q1J216_DEIGD |
| Q1J216_DEIGD | Q0YQQ4_9CHLB | I7IU14_9RICK | D3PSV4_MEIRD |
| D3PSV4_MEIRD | R5RTT1_9BACE | A0A0M0BIG3_9ARCH | D7BBU5_MEISD |
| D7BBU5_MEISD | SSB_RICPR | C6C019_DESAD | E6VS39_DESAO |
| B6WVX8_9DELT | A9B460_HERA2 | G7QAM4_9DELT | B4SFF9_PELPB |
| A0A0A1H6Y8_9BURK | F8EMG1_RUNSL | R5UZH0_9PORP | B8FNJ8_DESAA |
| Q3AU24_CHLCH | B0VUJ3_ACIBS | R5NYK0_9PORP | Q2IG69_ANADE |
| K1JZE8_9BURK | M4V949_9DELT | F7XVZ1_MIDMI | I2EUM7_EMTOG |
| A0A0G1C0M7_9BACT | A9WH08_CHLAA | Q9PCW1_XYLFA | SSB_BACTN |
| C1CXK1_DEIDV | C1CXK1_DEIDV | A0A0A7KJ72_9DEIO | A0A0A7KJ72_9DEIO |
| H8GPM1_METAL | I3TKW7_TISMK | E2CQP0_9RHOB | Q311Q4_DESAG |
| R5BSB1_9BACE | A0A0G1LQS6_9BACT | W5WYE9_BDEBC | Q9PG20_XYLFA |
| U6KT59_EIMTE | E1WYM5_HALMS | L0FWX8_ECHVK | I5C772_9BACT |
| A0A0G1LK57_9BACT | B5JYB4_9GAMM | A0A0G0ET98_9BACT | A0A0C3RCU9_9PORP |
| U6MFA4_9EIME | F0SN41_RUBBR | E8U544_DEIML | E8U544_DEIML |
| R5E1D3_9BURK | D6D4Z2_9BACE | A0A0F3MRG0_9RICK | R7IIR5_9BURK |
| F4HBY7_GALAU | A0A0F2NAQ0_9DELT | H8GSU7_DEIGI | H8GSU7_DEIGI |
| F9ZRW3_ACICS | I0K538_9BACT | A0A0A7LI72_9BACT | W8F2H2_9BACT |
| E8V7N3_TERSS | A0A068JL68_9DELT | A0A0F3PJ45_ORITS | G8R207_OWEHD |
| F2NKT7_MARHT | F2NKT7_MARHT | K1YDW1_9BACT | R4YT41_OLEAN |
| A0A077DAY8_9BURK | J5MVP4_PASMD | K1Y243_9BACT | A0A0F3QLF9_ORITS |
| A5CE60_ORITB | A0A0F3MAH9_ORITS | I4C0I0_DESTA | A0A0G1U4Z8_9BACT |
| F2NJT4_DESAR | W0EUN4_9PORP | D9SEK8_GALCS | F9Z3N4_ODOSD |
| Q2LVL9_SYNAS | X5DYT2_9BACT | K2E296_9BACT | K2BHI4_9BACT |
| A0A0F9Z3H3_9BACT | E8RHY9_DESPD | A0A0F2N9J8_9DELT | U6GLJ7_EIMAC |
| Q5DY59_VIBF1 | H8KSH1_SOLCM | R7DJS5_9PORP | A6LDG4_PARD8 |
| A0A078KHX1_9GAMM | A0A0E9LW73_9BACT | R5IPM2_9PORP | A0A0G1GC11_9BACT |
| W5YLS9_KOMXY | B8J4A9_DESDA | Q729X5_DESVH | L0A2S5_DEIPD |
| L0A2S5_DEIPD | L3ZDQ8_TERRK | E8RFA5_DESPD | D6SQC2_9DELT |
| E8UF33_TAYEM | G9ZJG1_9GAMM | C7PL90_CHIPD | I4AKI7_FLELS |
| R6KSH1_9BACE | T2GG32_DESGI | A7ILF7_XANP2 | W0RJ13_9BACT |
| A0A0G1EUE3_9BACT | C1A4G5_GEMAT | A0A0G1CIP0_9BACT | A0A0E9MWL8_9SPHI |
| M1WUK7_DESPC | C9RPL0_FIBSS | B3SED6_TRIAD | A0A0G1KPV8_9BACT |
| R6T4B7_9BACE | F3Z0I0_DESAF | A1AL61_PELPD | B3EUI3_AMOA5 |
| H8H1V2_DEIGI | H8H1V2_DEIGI | F0RJS5_DEIPM | F0RJS5_DEIPM |
| R4YJJ6_OLEAN | A0A0G0RHW4_9BACT | A6CAL9_9PLAN | F3PPY3_9BACE |
| C7LVB0_DESBD | U5Q9T7_9BACT | R6DQS0_9BACE | A0A0G0G4A2_9BACT |
| A5UYQ6_ROSS1 | R5U903_9BACE | M9X2N2_MANHA | F0SAP4_PSESL |
| F8WWX3_9PORP | R6S0F0_9BACE | A0A0G0EYQ7_9BACT | A0A0G1M3J1_9BACT |
| D2QT14_SPILD | E8WX41_GRATM | A0A0G1B6J4_9BACT | F0RQ97_DEIPM |
| F0RQ97_DEIPM | A0A0G1WDY0_9BACT | A0A0G1XZ90_9BACT | A0A0G1GD47_9BACT |
| A0A0G0QXB5_9BACT | F6GHH8_LACS5 | Q1MR30_LAWIP | G8P175_GRAMM |
| A0A0G3Q6R7_KLUIN | A0A0G1NBX6_9BACT | A0A0G1LCA4_9BACT | E8MY37_ANATU |
| F8C2X7_THEGP | K1Z192_9BACT | A0A0G1PFQ8_9BACT | A0A0G1QNL8_9BACT |
| R5J536_9BACE | A0A0G1ZBQ4_9BACT | A0A0G1QIF9_9BACT | Q5LF22_BACFN |
| A0A075WVB3_9BACT | A0A0G0FRM6_9BACT | A0A088EU06_9SPHI | A0A0G1S3A5_9BACT |
| A0A0G1H5P6_9BACT | A0A0G1WM19_9BACT | A0A0G1S5L4_9BACT | F4CDG5_SPHS2 |
| A0A0M3CBN8_9SPHI | R6LH35_9BACE | K2F210_9BACT | A0A0G0NGV8_9BACT |
| A0A0G1D0J1_9BACT | A0A0G0R7V0_9BACT | A0A0G0AR01_9BACT | G1UTS6_9DELT |
| K2GD81_9BACT | A3HUW7_9BACT | A0A0G0ZFU3_9BACT | A0A0G1DJ74_9BACT |
| D7VHY4_9SPHI | F5J436_9PORP | A0A0G0VD89_9BACT | A0A0G1BMC4_9BACT |
| I2GP99_9BACT | Q5NE96_FRATT | K2CK60_9BACT | A0A0G0ITC8_9BACT |
| A0A0G1AFR7_9BACT | A0A0G0P9E8_9BACT | G8TNT5_NIAKG | K2FL67_9BACT |
| A0A0G1Y159_9BACT | A5URI9_ROSS1 | E6SPC5_BACT6 | A0A0G0UQH2_9BACT |
| SSB_THET8 | SSB_THET8 | A0A0G1EQ71_9BACT | V5RU84_9BACT |
| K2AY54_9BACT | A0A0M2XR79_9SPHI | I4EDL5_9CHLR | A0A0F9Z444_9BACT |
| D7CWQ9_TRURR | D7CWQ9_TRURR | A0A0G0AM08_9BACT | W0F027_9SPHI |
| A0A0F9YYB5_9BACT | R5YWC8_9BACE | A0A0G1TXK3_9BACT | K2ECV4_9BACT |
| A0A0G0HE85_9BACT | C0BIE9_FLABM | A0A0G1ZAW4_9BACT | E4RV70_LEAB4 |
| E0TIQ6_ZINIC | SSB_DEIRA | SSB_DEIRA | G0IVW4_CYCMS |
| A0A0G1F613_9BACT | A0A0G0X959_9BACT | A0A0G0AR32_9BACT | G1UNE4_9DELT |
| F3S3G5_9PROT | R6YPN2_9BACE | E4T8K4_PALPW | A0A0G1GDN2_9BACT |
| A0A0G0IUI9_9BACT | E5Y6F6_BILWA | A0A0G1VM78_9BACT | R5NF50_9BACT |
| A0A0G0K483_9BACT | A0A0G1WKB5_9BACT | Q01NH0_SOLUE | A0A0G1IWE9_9BACT |
| K1KIB0_9BURK | K2DBA5_9BACT | A0A0G0FD70_9BACT | A0A0G1L815_9BACT |
| A0A0G0TAY3_9BACT | I2K4V5_9PROT | A0A0G1GTA0_9BACT | A0A0G0BES3_9BACT |
| A9AWI3_HERA2 | A0A0G0VFF3_9BACT | A0A0G0Z742_9BACT | T2KR13_9FLAO |
| A0A0G1RWB7_9BACT | K2DHT4_9BACT | K2FUX9_9BACT | A6EB39_9SPHI |
| A0A0G0M9E2_9BACT | A0A0G0MVY3_9BACT | A0A0G0QNI7_9BACT | C6XY89_PEDHD |
| F4B1C6_DOKS4 | S2DJ49_9BACT | A0R7W8_PELPD | A0A0G0F0A3_9BACT |
| I4AL65_FLELS | R6WXR8_9PORP | I3Z3D3_BELBD | R7EGY2_9BACE |
| K2CX72_9BACT | A0A0G1MUL2_9BACT | A0A0G0WVC5_9BACT | A0A0G0XAK9_9BACT |
| K2ECW7_9BACT | A0A0G0DA92_9BACT | A0A0G0Z340_9BACT | A6EM42_9BACT |
| A0A0G1CSF5_9BACT | D1Y258_9BACT | A0A0G1XSH5_9BACT | A0A0A7LLS2_9BACT |
| E6X251_NITSE | A0A0G0RUZ2_9BACT | A0A0G0KLW8_9BACT | A0A0G0MIW7_9BACT |
| A0M7C0_GRAFK | A0A0G0L6I2_9BACT | A0A0G0JKX6_9BACT | A0A024FGJ0_9FLAO |
| A0A0G0MT32_9BACT | A3XQ83_LEEBM | K2AGX2_9BACT | A0A0G1KDM0_9BACT |
| K1L8G3_9BACT | A0A0G0IL11_9BACT | H6L379_SAPGL | A0A0G0UDL8_9BACT |
| H1XZM6_9SPHI | F4L5F7_HALH1 | C3XLE9_9HELI | F8X2D4_9PORP |
| S0GEM3_9PORP | A0A0G1XKI1_9BACT | A0A0G1YIP2_9BACT | H1XTH2_9BACT |
| A0A0G0ZVR8_9BACT | F3ZU02_9BACE | A0A0G1KCC6_9BACT | A0A0G1KMS5_9BACT |
| A0A0G1PCV8_9BACT | K2CTZ4_9BACT | A0A0G1BVU4_9BACT | A0A0G1QUX1_9BACT |
| A9FZR5_SORC5 | A0A0G1RE30_9BACT | C1A431_GEMAT | A0A0G1RNU5_9BACT |
| K2AKT1_9BACT | A0A0G1T277_9BACT | K1ZC65_9BACT | X5DJQ1_9BACT |
| A0A0G0Q4T9_9BACT | A0A0G1EN32_9BACT | A0A0G1HYN1_9BACT | B4CYA0_9BACT |
| A0A0G0H3K7_9BACT | A0A0G0JR80_9BACT | I3C4X1_9FLAO | A0A0G0ICH2_9BACT |
| A0A0G0Q0Z9_9BACT | Q26HH7_FLABB | F8X1E8_9PORP | K1Z5D0_9BACT |
| A0A0G1PK08_9BACT | A0A0G0QT25_9BACT | A0A0G0VCD2_9BACT | A0A0G0WJ75_9BACT |
| A0A0G0QRF0_9BACT | A0A0G0XSM8_9BACT | A0A0G1IVQ6_9BACT | A0A0G1U187_9BACT |
| A0A0G1FGT8_9BACT | A0A0F9YZ74_9BACT | A0A0G1KD14_9BACT | K2GCJ2_9BACT |
| F9TDK3_9VIBR | K2AA51_9BACT | A0A0G1M0Y6_9BACT | K1ZD39_9BACT |
| A0A0G4B559_9BACT | A0A0G1QXE6_9BACT | A0A0G1VT24_9BACT | A0A0G0K489_9BACT |
| A0A098BVY9_9PORP | K2AQC8_9BACT | A0A0G1MW79_9BACT | A0A0G1V0T6_9BACT |
| R7PAL0_9BACT | K2G2N2_9BACT | A0A0G0BK79_9BACT | A0A0G0GUJ0_9BACT |
| F8EP30_RUNSL | A0A0G0HDB0_9BACT | A0A0G0M3V5_9BACT | A0A0G1HM27_9BACT |
| A0A0G0T5R8_9BACT | A0A0G1CE07_9BACT | A6G1X8_9DELT | A0A0G0YX12_9BACT |
| A0A0G0UM02_9BACT | A0A0G1YNF5_9BACT | E6X5Q0_CELAD | A0A0G1WNB0_9BACT |
| A0A0G0YK69_9BACT | A0A0G1VSA9_9BACT | A0A0G0M6X1_9BACT | A0A0G1P2X8_9BACT |
| X2H7A7_9NEIS | A3U5V5_CROAH | A0A0G1I503_9BACT | K2GR66_9BACT |
| SSB_RHOBA | A0A0G1TA05_9BACT | R5JRY1_9BACE | A7GXQ3_CAMC5 |
| A0A0G1TVB4_9BACT | A0A0G1R3J3_9BACT | F0R206_BACSH | A0A0G1I1J8_9BACT |
| A0A0G1N1Y0_9BACT | A0A0G0DFF8_9BACT | A0A0G0HJ81_9BACT | A0A0G0L6C8_9BACT |
| G8UNV5_TANFA | A0A0G0WNN8_9BACT | A0A0G0T067_9BACT | A0A0G1TTV3_9BACT |
| R6FJU2_9BACE | K2Q6V2_9FLAO | G9PZM7_9BACT | A0A0G1EVI1_9BACT |
| K2CX80_9BACT | D2ZSZ5_NEIMU | A0A0G0MJX6_9BACT | A0A0G1UL86_9BACT |
| A0A0G1T611_9BACT | A0A0G1NY98_9BACT | A0A0G1K096_9BACT | A0A0G1LVX9_9BACT |
| A0A0G2AUH0_9BACT | A0A0F9ZKM8_9BACT | A0A0G0GFH0_9BACT | A0A0G0D4N4_9BACT |
| A0A0G1KT99_9BACT | R7LF57_9BACT | G2EHB5_9FLAO | A0A0F9YE88_9BACT |
| A0A0G1LER9_9BACT | A0A0G1E5Q5_9BACT | K2DE88_9BACT | A0A0G0QWW2_9BACT |
| A0A0G1YHP1_9BACT | Q30PZ3_SULDN | A0A0G1YJ06_9BACT | A0A0G0RMN5_9BACT |
| H2BRZ7_9FLAO | D5SQI8_PLAL2 | A0A0G0RM55_9BACT | D1B1C3_SULD5 |
| A0A0G0IK26_9BACT | A0A0G0RPA2_9BACT | K2ES05_9BACT | A0A0D5YSC5_9FLAO |
| A0A0G0FI04_9BACT | A0A0G0JPG5_9BACT | A4V7V9_PSEFS | L7W8Z0_NONDD |
| A0A0G0ZNV6_9BACT | K2B5H2_9BACT | A0A0G1GWI5_9BACT | A0A0G0VHY0_9BACT |
| A0A0G0F0G2_9BACT | W2CHQ2_9PORP | A0A0G1C1H8_9BACT | A0A0G1VPU9_9BACT |
| A0A0G1Y128_9BACT | A0A0C5W6B5_9FLAO | W5YUV6_9ALTE | A0A0G0QW90_9BACT |
| F0P2N0_WEEVC | A0A0G1WRU3_9BACT | V6F187_9PROT | V6F187_9PROT |
| G0L3S2_ZOBGA | A0A0G1NZT4_9BACT | B3JPX7_9BACE | A0A0A8E408_9GAMM |
| A0A0G0F4Z5_9BACT | K2CZW8_9BACT | R6EFS7_9BACE | R7JPX4_9PORP |
| A0A0G0B811_9BACT | R6NQA9_9BACE | A0A0G0HF46_9BACT | A0A0G0TF81_9BACT |
| A0A0G1DZP7_9BACT | K2BD14_9BACT | A0A0G2BH32_9BACT | R6CU93_9BACE |
| H0ULU9_9BACT | A0A0G1R5Q2_9BACT | D2Z2X8_9BACT | C9LG13_9BACT |
| A0A0G1G841_9BACT | A0A0G1DYE2_9BACT | A0A0G1MXS6_9BACT | A9WIF6_CHLAA |
| V6F590_9PROT | V6F590_9PROT | A0A0G1KC57_9BACT | A0A0G0ZZ46_9BACT |
| A0A0G1KHX6_9BACT | A0A0G1LKX5_9BACT | A0A0G0Y5Y6_9BACT | R6ZUD7_9BACE |
| E4U134_SULKY | D8K0D1_DEHLB | A0A0G1E8Q7_9BACT | A0A0G0WUP2_9BACT |
| A0A0G1RR84_9BACT | C0ZA46_BREBN | L0EUZ9_LIBCB | A0A0G0UEZ2_9BACT |
| A9G766_SORC5 | A0A074SUT6_HAMHA | A0A0G0HVX2_9BACT | A0A0G0JF99_9BACT |
| I3UHZ1_ADVKW | A0A0F9Z7C7_9BACT | J2HR04_9BACL | K4IS14_PSYTT |
| D5ERI0_PRER2 | A0A0G1U855_9BACT | A0A0G1W834_9BACT | A3U5F1_CROAH |
| F9YQ81_CAPCC | B9L5W8_NAUPA | D5EJK6_CORAD | A0A0G0NQR7_9BACT |
| A6L7N8_BACV8 | G2LEN2_CHLTF | A0A0G1LDB3_9BACT | A0A0G0WK72_9BACT |
| A0A0G1XPK7_9BACT | R7NMI1_9BACE | A0A0G1UAR8_9BACT | R7HTQ0_9BACT |
| I3YV25_AEQSU | C0BLH8_9BACT | K1ZZU0_9BACT | F0EYB2_9NEIS |
| A0A0G0LTD2_9BACT | F6GGX7_LACS5 | K1Z9M5_9BACT | A0A0G1R9V2_9BACT |
| H1Y6M1_9SPHI | K1ZMT3_9BACT | A0A0G0IWK6_9BACT | A0A0G0JF01_9BACT |
| A0A0G0GJN5_9BACT | A0A0G0L264_9BACT | A4BXL3_9FLAO | F4KZC1_HALH1 |
| A0A0G0P2K6_9BACT | A6Q7C8_SULNB | A0A0G0WB84_9BACT | A0A0G1YS56_9BACT |
| A0A0F0CQF3_9BACT | Q4XMV9_PLACH | A0A0G1NYY3_9BACT | B6YQX2_AZOPC |
| A0A0G1BF33_9BACT | A4AWE0_MARSH | A0A0G1NN17_9BACT | D6THA2_9CHLR |
| SSBA_BACSU | L8AVZ9_BACIU | A0A0G1T421_9BACT | A0A0G0U7M6_9BACT |
| SSB_WOLSU | R5BM18_9FIRM | A0A0G1C6D3_9BACT | A0A0G2AWX5_9BACT |
| I4A267_ORNRL | R5GFV4_9BACT | K6UKM8_9APIC | D1BR58_VEIPT |
| J2S2V5_9FLAO | A0A0G1HLF4_9BACT | A7ZEF7_CAMC1 | H7FN74_9FLAO |
| A0A098GHM2_TATMI | A0A0E9MZ46_9SPHI | W7AIE7_PLAVN | A8ZR98_DEIGD |
| A8ZR98_DEIGD | I0IET8_PHYMF | B9L1I8_THERP | A0A0G0M358_9BACT |
| A0A0G0RPU1_9BACT | V6S8T9_9FLAO | E0UPU6_SULAO | A0A0G1BTT0_9BACT |
| A0A0G1FMR0_9BACT | A0A0G0QLF7_9BACT | A0A0G0BZ87_9BACT | A0A0G1FFL8_9BACT |
| A0A0G0CXZ0_9BACT | A0A0F9ZUR4_9BACT | A0A0G1FVR4_9BACT | A0A0C1GCK9_9FLAO |
| A0A0G0MGM9_9BACT | A0A0G1WN93_9BACT | A6LAC3_PARD8 | G2PM60_MURRD |
| A0A0G0GC72_9BACT | B6BIM9_SULGG | A0A0G1WQA3_9BACT | D7JFM1_9BACT |
| C5ZV90_9HELI | A0A0G1QS53_9BACT | L7W5D1_NONDD | G2Z2B5_FLABF |
| Q8I415_PLAF7 | A0A024VHA1_PLAFA | A0A024WBS3_PLAFA | A0A024WWR3_PLAFA |
| D1B9D4_THEAS | A0A0G0BWT4_9BACT | A2TXU5_9FLAO | A0A0G1AL26_9BACT |
| A0A0G0UT33_9BACT | A0A0G1XX97_9BACT | A9DL76_9FLAO | A0A0G0L6C1_9BACT |
| A0A0G0IFR9_9BACT | Q7PDM6_PLAYO | A0A0G1NEE6_9BACT | A0A0G1RE35_9BACT |
| A0A0G0IKV8_9BACT | G0LBI4_ZOBGA | A0A0G0Q0U8_9BACT | AIZRR4_9BACT |
| A0A0G0K4P7_9BACT | C8PLH8_9PROT | W8VWG1_9FLAO | A0A0G0C6A8_9BACT |
| A8ERE3_ARCB4 | A0A0G1FD97_9BACT | A0A095ZH18_9BACT | A3J2K3_9FLAO |
| Q4Z5Q5_PLABA | A0A0G1IAL7_9BACT | I0WK08_9FLAO | A5FLM3_FLAJ1 |
| A0A0F9ZAH0_9BACT | D1PYR4_9BACT | A4CIT5_ROBBH | A0A0G0G6E6_9BACT |
| W7A536_9APIC | R5AVX0_9BACE | A0A0F2NZ96_9FLAO | A0A0G1PPZ1_9BACT |
| G2Z404_FLABF | A0A0G1P7K3_9BACT | A0A060R893_9BACT | A0A0G1N320_9BACT |
| W8VVV0_9FLAO | K2A8G9_9BACT | K2E6G7_9BACT | A0A0G0RN27_9BACT |
| A0A0G1H3J6_9BACT | A5KB16_PLAVS | K0P2K0_9BACT | D5V760_ARCNC |
| A0A0G0YX77_9BACT | A0A0G0MWK9_9BACT | Q3Z7N8_DEHM1 | A0A024FGV6_9FLAO |
| Q5ZWV8_LEGPH | A0A0G1W343_9BACT | A0A0G0E4G6_9BACT | W0J168_9BACT |
| D1CFJ0_THET1 | A0A0G0VDL7_9BACT | S6A3X9_9SPIO | A0A0G1N6K0_9BACT |
| E6TZX5_BACCJ | B3L6I1_PLAKH | F0IH13_9FLAO | E3CX16_9BACT |
| L0D9A4_SINAD | A0A0G1QE90_9BACT | A0A0G1ZNC1_9BACT | D5EG56_AMICL |
| E6K7H1_9BACT | A0A0G1ZLY4_9BACT | T1JEX3_STRMM | T1JEX3_STRMM |
| A0A094WDC8_BACAO | SSB_HELHP | G8X6H6_FLACA | H1Y6X3_9SPHI |
| A0A0B5RL43_9FLAO | A0A0G0R2B1_9BACT | G8R7E5_OWEHD | T2KIN2_9FLAO |
| C2M318_CAPGI | R7D092_9BACE | A0A0C5W9G8_9FLAO | Q7MXD2_PORGI |
| A0A0G1L2P3_9BACT | K6D849_BACAZ | W0F1G0_9SPHI | A6H058_FLAPJ |
| G2PIF9_MURRD | A0A0G0VWD9_9BACT | A0A0G0MTU3_9BACT | I3C2X3_9FLAO |
| C3J7T3_POREA | D5B9K6_ZUNPS | F0RQ90_DEIPM | F0RQ90_DEIPM |
| X2GU58_9BACI | A0A089NRR5_9BACL | F4B204_DOKS4 | A0A0G0J2C4_9BACT |
| Q65CP4_BACLD | A4CH56_ROBBH | A0A0G1CG12_9BACT | A0A0M2V033_9BACT |
| A0A0G1X8Y6_9BACT | A0A0G1W4C5_9BACT | K2AP50_9BACT | R5AJM2_9BACT |
| Q26FF9_FLABB | F0RE93_CELLC | A0A0G1DJJ4_9BACT | J3C846_9FLAO |
| A6Q465_NITSB | I3C4Z1_9FLAO | A0A0G0XVB6_9BACT | A0A0G0GYK1_9BACT |
| H0E9H1_9ACTN | I4BUS1_ANAMD | A0A0G0UV05_9BACT | D1VYB4_9BACT |
| C8PFI3_9PROT | F4XGC9_9FIRM | K2F5S8_9BACT | A0A0G0XSG0_9BACT |
| SSB_BACHD | A0A0F6SHL5_9DELT | A5FJQ5_FLAJ1 | A0A0G0CPI5_9BACT |
| V4IHX5_9GAMM | C6RCT1_9PROT | C6D836_PAESJ | B9KCI7_CAMLR |
| H8XRQ3_FLAIG | H8XQA3_FLAIG | A0A0G1N5F5_9BACT | G2PPT0_MURRD |
| A0A0G0RBE5_9BACT | A0A0G1LMR6_9BACT | A7I007_CAMHC | A3J3H8_9FLAO |
| A0A098GC49_TATMI | F0RQ78_DEIPM | F0RQ78_DEIPM | A1U8M6_MARHV |
| R5ZXU7_9BACT | A0A0G1TFJ3_9BACT | A0A0G1D250_9BACT | A0A0C1G1B6_9FLAO |
| K2D8C8_9BACT | G2ED13_9FLAO | A8FJE6_BACP2 | D3FQF9_BACPE |
| V4Z7I0_TOXGO | H1ZEI1_MYROD | A0A0G0AIB5_9BACT | V6SCA1_9FLAO |
| A4ANB7_MARSH | A0A0B4SE50_9BACI | A0A0G0XMP0_9BACT | I7HEB8_9HELI |
| A0A0G1MV34_9BACT | H2BXS6_9FLAO | H3SKZ8_9BACL | A6EQD6_9BACT |
| I0JTK6_HALH3 | A0A0G0KMQ6_9BACT | J9QTR6_RIEAN | A0A0G0X1K5_9BACT |
| E6X1S0_NITSE | A0A089MFZ3_9BACL | A0A059NVS1_9BACI | D8DWS8_PREBR |
| A0A0K0G707_9FIRM | K2C4X2_9BACT | I3C1I5_9FLAO | A0A0G1QFF4_9BACT |
| G2PQ85_MURRD | A0A0G0R1X3_9BACT | H8H312_DEIGI | H8H312_DEIGI |
| B7GMU8_ANOFW | K4RIT0_HELHE | SSB_CAMJE | G9ERB0_9GAMM |
| R5BTT7_9FIRM | I3C0K2_9FLAO | A3J3H9_9FLAO | A0A0G1A645_9BACT |
| A0A0G0EG92_9BACT | K1XT39_9BACT | A0A0G0NJ04_9BACT | A0A0G0E1X4_9BACT |
| A0A0G0DA43_9BACT | A0A0G1R810_9BACT | A0A0G1KQT7_9BACT | A0A0G1KYP1_9BACT |
| C2WF66_BACCE | G9ZIP3_9GAMM | A3XH90_LEEBM | I3C290_9FLAO |
| K2F3J1_9BACT | A6L491_BACV8 | I3C2L6_9FLAO | W8VX23_9FLAO |
| I0TC03_9BACT | B4RIS2_PHEZH | G9EJ05_9GAMM | K1YY81_9BACT |
| A0A0G1G5A2_9BACT | A0A0G0YPJ7_9BACT | D8PBE8_9BACT | H2BTI1_9FLAO |
| H7F723_9LIST | SSB_BACCR | R6Q4G8_9BACT | D5BB32_ZUNPS |
| C0QIM1_DESAH | I3CB97_9FLAO | K1JX68_9BURK | A0A0G0AW48_9BACT |
| I3II60_9BACT | G9YQV9_9FIRM | R6E6X3_9BACT | K9P7I4_CYAGP |
| R6EX32_9BACT | A0A0F7CTW4_9CHLR | H8XRQ4_FLAIG | R5PDP4_9BACT |
| B9D1K8_CAMRE | R6YS16_9BACT | B5IJJ3_9CHRO | SSB_BACAN |
| D9RRS8_PREMB | D3UJ32_HELM1 | K2GWT5_9BACT | F2KTM6_PREDF |
| G8T6V2_NIAKG | A3XJ25_LEEBM | A0A0M2PGH3_9BACI | E0I9A3_9BACL |
| G9EQ89_9GAMM | C7MM82_CRYCD | E0NPE2_9BACT | D7NFG4_9BACT |
| C7M3U9_CAPOD | G3Z1H6_9NEIS | Q5KU70_GEOKA | E6MT69_9BACT |
| G7V667_THELD | R7F3A0_9BACT | R6CIQ6_9BACT | E4PJ71_MARAH |
| D6D7Q4_9BACE | K2F7X5_9BACT | F2LU75_HIPMA | A0A0G1TBB5_9BACT |
| A0A0G3EDN0_9BACT | D5BAB2_ZUNPS | B9XFD3_PEDPL | A2TY45_9FLAO |
| C9LYI1_SELS3 | A0A089M198_9BACL | R6AVL5_9BACT | A0M128_GRAFK |
| A0A075RC04_BRELA | E7RSX3_9BACT | R5VBW7_9BACE | I3C8E4_9FLAO |
| A7GVN6_BACCN | A5GI32_SYNPW | I3C6Z7_9FLAO | R7RV76_FLAPJ |
| R5TDE7_9FIRM | G8X8S0_FLACA | A0A099UZI7_9HELI | A0M0Q6_GRAFK |
| I0WHP7_9FLAO | F8N6Q2_9BACT | A0A075LKI3_9BACI | A0A099WCC6_9LIST |
| A3Z466_9SYNE | R6WUN6_9BACT | A0A0C2UAE7_BACBA | C5CD95_KOSOT |
| H2BWP6_9FLAO | B3E0Q5_METI4 | A0A0F5PBD5_9SPHN | I8J2Y5_9BACI |
| R6WM24_9BACT | A0A0F5I2L0_9BACI | F4GLF7_SPHCD | A0A0G1YCE6_9BACT |
| SSB_OCEIH | A0A0G1JU97_9BACT | K2CKU7_9BACT | K6Y9X8_9ALTE |
| I3YUH0_AEQSU | A0A0G0R4I9_9BACT | A0A088AIU7_APIME | J7J598_DESMD |
| K2QGZ2_9FLAO | L0G1L9_ECHVK | A9DQ41_9FLAO | T2KN55_9FLAO |
| I3C1Q8_9FLAO | E3EC34_PAEPS | I1YFJ8_METFJ | A0RQ07_CAMFF |
| G7WI54_DESOD | A0A0D5NIC7_9BACL | A0A0F2SDU7_9FIRM | D5BLX7_ZUNPS |
| U5L4I2_9BACI | A0A0G1NI47_9BACT | D2R6N7_PIRSD | A0A0G1GT67_9BACT |
| B4EWR6_PROMH | X4ZJY3_9BACL | R5KHV0_9BACT | D3LTM2_9FIRM |
| B9D160_CAMRE | E1KTQ3_9BACT | D5BB33_ZUNPS | I3C289_9FLAO |
| A0A077KJT9_9FLAO | Q05UI4_9SYNE | A0A0B5AYD2_9BACL | A3ZL43_9PLAN |
| A3YWU7_9SYNE | F0RBC8_CELLC | D3IHP9_9BACT | J2ZK91_9LACO |
| B9DIB9_STACT | A0A0G1DV39_9BACT | K1ZYT4_9BACT | A0A0E3UIY2_9BACT |
| E8N1Q1_ANATU | L0ELE3_THECK | L8HBV6_ACACA | K2FTU8_9BACT |
| A0A075R7Q5_BRELA | K0J855_AMPXN | J5KD76_PASMD | A0A0D1LP27_9LACT |
| I0I2P5_CALAS | D4KHG9_9FIRM | F2F271_SOLSS | C6IWW3_9BACL |
| A0A0G1EHZ2_9BACT | B2UN66_AKKM8 | I0IMR3_LEPFC | J3CHS7_9FLAO |
| A6M3L1_CLOB8 | R7EVC4_9FIRM | G2KTA2_LACSM | Q5M2Q4_STRT2 |
| R5HF76_9FIRM | A0M601_GRAFK | Q0IDX5_SYNS3 | E6X7W5_CELAD |
| Q11BM6_CHESB | H6NIY5_9BACL | A0A0G0V6P2_9BACT | R4PN35_9BACT |
| L0JB55_PREDD | A0A0G1B4T5_9BACT | A0A0A1GUA2_9LACO | G4RDQ8_PELHB |
| E3GR78_EUBLK | E3GR78_EUBLK | A5GQ52_SYNR3 | L3TXZ4_ENTFC |
| F3ZYJ8_MAHA5 | G4HDI8_9BACL | H1HQJ5_9BACT | R6G607_9CLOT |
| J9ZDX0_LEPFM | R7KS27_9FIRM | D4MAR0_9BACT | B5YK70_THEYD |
| G2TIH3_BACCO | A5CY51_PELTS | A3I0E4_9BACT | Q03I53_PEDPA |
| H5VBY2_HELBI | F2LV66_HIPMA | G2FTW8_9FIRM | D6Y1K5_BACIE |
| C4L009_EXISA | R7GGC3_9CLOT | A0A0F3RSN3_9LACO | S1P033_9ENTE |
| Q5WAH4_BACSK | Q4L341_STAHJ | W0JD18_DESAE | K1LGI4_9BACT |
| Q73M33_TREDE | R2T735_9ENTE | K1XET1_9BACT | T2KQ25_9FLAO |
| F8KQW0_HELBC | A0A0F2J7A3_9FIRM | A0A0F3RVR0_9LACO | S0L007_9ENTE |
| G2FNN2_9FIRM | V4IXS5_9GAMM | B1YG98_EXIS2 | B2V6V6_SULSY |
| I7A7D4_MELRP | F0P2C4_WEEVC | A0A0G1IEC5_9BACT | T2KP72_9FLAO |
| F5LHP3_9BACL | I0AJV0_IGNAJ | R5B767_9CLOT | R7CMF9_9FIRM |
| R5A556_9CLOT | G1WB69_9BACT | R6FRH6_9CLOT | F4KP50_PORAD |
| E7NW85_TREPH | A7H0W9_CAMC5 | R7KDB0_9CLOT | I4Z6P8_9BACT |
| K0FER8_9NOCA | V5X6S5_MYCNE | G6EMD8_STRTR | A4BWV3_9FLAO |
| F2BXW4_9FIRM | I4EL78_9CHLR | E8R1N7_ISOPI | A0A0G3V8F0_9ACTN |
| A0A077ELF4_9FLAO | B1ZPW4_OPITP | R6W1H8_9FIRM | D5XDR1_THEPJ |
| L0EKJ7_THECK | A8L8T3_FRASN | H0DJQ8_9STAP | A0A0H4L2E2_9BACI |
| Q5HRZ4_STAEQ | K2BME1_9BACT | Q7U9Y1_SYNPX | A0A0H5Q455_SYNPZ |
| A0A0F9Z426_9BACT | G0VMW5_MEGEL | I4DCJ9_DESAJ | C4IMS9_CLOBU |
| A0A0M2SW57_9BACI | Q24MB8_DESHY | A0A024Q6M4_9BACI | R7MDV2_9CLOT |
| A0A0G0JLJ7_9BACT | A0A0A2E6F1_9PORP | SSB_THEMA | R7IZP9_9BACT |
| K6DNP2_9BACI | A0A0F2S1Q7_9RHOB | SSB1_STRA5 | A0A077J7B4_9BACI |
| C2CT55_CORST | F8B6R8_FRADG | Q2G112_STAA8 | C6X107_FLAB3 |
| I0RDU7_MYCPH | A0A060M243_9BACI | W1SCF8_9BACI | A0A0A2U562_9BACL |
| E4KZJ9_9FIRM | K8E172_CARML | Q7NHP8_GLOVI | E8SHU3_STAPH |
| A4VXG0_STRSY | E3C6V5_9LACO | C1DTM9_SULAA | B0P5X9_9FIRM |
| A0A0G0B0I9_9BACT | SSB2_LACLA | A0A075JU30_9BACI | K2BLN7_9BACT |
| K4L900_9FIRM | L0FCF1_DESDL | D5WXD0_KYRT2 | H6LCF9_ACEWD |
| S1N2V3_9ENTE | R5C9B9_9BACT | C4IHH5_CLOBU | A0A023CJV1_GEOSE |
| D3EJA6_GEOS4 | A0A075TYJ5_9LACT | A0A0G0YSF5_9BACT | I4F5M2_MODMB |
| A0A0G1VJD5_9BACT | R5Z2P9_9CLOT | A9WVL4_RENSM | B9E8Y6_MACCJ |
| F0T2X6_SYNGF | E6W1E1_DESIS | H2J4V1_MARPK | R5KVV6_9CLOT |
| M1MNX6_9CLOT | R2PID4_9ENTE | Q7V923_PROMM | I0RXI5_MYCXE |
| Q0F0E0_9PROT | G9YN40_9FIRM | Q5YN16_NOCFA | R6AK08_9FIRM |
| G8LTQ4_CLOCD | S0JGT9_9ENTE | I7JHF2_9LACT | F5YAZ8_TREAZ |
| Q24VF6_DESHY | T0TSW0_9STRE | C2EU98_9LACO | R7MCD0_9CLOT |
| R5RR34_9FIRM | R5LR09_9SPIR | R7IS09_9CLOT | R6SXG9_9CLOT |
| K4ZHE9_PAEAL | G8RLC3_MYCRN | Q5LCZ3_BACFN | H7FPY3_9FLAO |
| R7PT35_9FIRM | D6GT00_FILAD | D4Z2M0_SPHJU | A9BD67_PROM4 |
| A0A0B5RTH7_9FLAO | W0EFP3_9FIRM | A6NVZ6_9FIRM | D3G1D7_BACPE |
| A0A0F9Z6S5_9BACT | A0A023CI44_GEOSE | H2CE09_9LEPT | C5D8F8_GEOSW |
| V4QQS1_STRIN | I1YV15_PREI7 | S0SK23_9ENTE | D9SPC1_CLOC7 |
| K5BDX9_9MYCO | F4C309_SPHS2 | I4DCP3_DESAJ | C0ZV44_RHOE4 |
| G8PE86_PEDCP | D1C3R8_SPHTD | N0B1V2_9BACI | A1UP85_MYCSK |
| C5RAY6_WEIPA | C5D9X5_GEOSW | U5QLX6_9CYAN | C7NGT6_KYTSD |
| Q49UQ1_STAS1 | A0A0M2VUR8_9BACL | Q38ZR7_LACSS | B9CLT9_9ACTN |
| B0VFZ2_CLOAI | F4F596_VERMA | A3I0F7_9BACT | I0GSV3_SELRL |
| I1D897_9PSEU | A0A0G2ZH51_9BACT | D6ZB10_SEGRD | D5UP28_TSUPD |
| A0A0G0FVV6_9BACT | A0A0A8JFR8_BACSX | A0A0G2B6Q3_9BACT | E1R296_SPISS |
| A0A0B6TWN0_9CORY | V5WHR9_9SPIO | R6TSJ9_9FIRM | F8B065_FRADG |
| C0QQW8_PERMH | F9VMA2_ARTSS | R6PML2_9CLOT | A7HZQ9_CAMHC |
| SSB_MYCS2 | SSB_STRP1 | R5W1M5_9BACE | K1X5S5_9BACT |
| SSB1_LISMO | SSB_ENTFA | Q67J49_SYMTH | C9KMR8_9FIRM |
| M1MYD5_9CLOT | D7CKA0_SYNLT | R6YHT0_9CLOT | R5YGH9_9CLOT |
| A8F5G1_PSELT | F4LN90_TREBD | A9WB21_CHLAA | A1SQS3_NOCSJ |
| G5JZW3_9STRE | Q1QEX4_NITHX | D5BDH4_ZUNPS | G7MBJ2_9CLOT |
| T0U9E3_9ENTE | E5WH96_9BACI | Q0ATX7_SYNWW | H6R9N9_NOCCG |
| R6UNI3_9FIRM | E1R0L2_9ACTN | F0H2E9_9FIRM | K6TY13_9CLOT |
| B9DVK3_STRU0 | G5K7U2_9STRE | R3X593_9ENTE | R7FCY1_9CLOT |
| F9HJG1_9STRE | G1WKH2_9ACTN | V9WD61_9BACL | E2ZB85_9FIRM |
| S5QZ71_9STRE | J9SSA7_9ACTN | H1D1U0_9FIRM | SSB_HELPY |
| R7L899_9CLOT | A0A0D3VG85_9BACL | U5QBR9_9CYAN | R6H9V2_9FIRM |
| R6MT98_9FIRM | L0J4W0_MYCSM | A0A090MGC5_AFIFE | R6SVH2_9CLOT |
| U5N134_CLOSA | C3PKC9_CORA7 | SSB_STRMU | A0A0F5N0E8_9MYCO |
| Q03UD7_LACBA | U5WR80_MYCKA | Q5FN08_LACAC | R6M3F2_9FIRM |
| R5NQF3_9CLOT | A0A0F2J3B9_9BACT | D0L8B0_GORB4 | E8MYY2_ANATU |
| F9DV30_9BACL | A0A0H4LAQ5_9LACO | A0A0G1W755_9BACT | A0A0H4P943_9BACI |
| A0A0G3ITE4_9MYCO | E8UYL8_TERSS | A0A0G1HV64_9BACT | I6SBF0_ENTHA |
| G0J0R1_CYCMS | K9PZ35_9CYAN | K6VEM2_9MICO | D4IXN6_BUTFI |
| Q5KV10_GEOKA | M1V543_CYAME | I3EBY4_BACMT | R5VRL4_9FIRM |
| C6HX91_9BACT | A8AZD6_STRGC | B2HIB8_MYCMM | Q3AG26_CARHZ |
| G5F284_9ACTN | J4WCB5_9FIRM | A0A0B5E162_9RHOB | K2IBX4_9RHOB |
| A3VKS9_9RHOB | I3Z9F8_BELBD | K4LED7_THEPS | D7GS93_9FIRM |
| B2GJD7_KOCRD | A0A0A1H2Y5_9BURK | H3NDL1_9LACT | F5YR52_TREPZ |
| A9BH63_PETMO | C2BHH0_9FIRM | A0A0M3CEV4_9SPHI | S6CKB8_9ACTN |
| C8W8M1_ATOPD | H6LDD9_ACEWD | Q7NWU0_CHRVO | SSB_CLOPE |
| Q0SB52_RHOJR | A0A0D6DVT4_9LACT | D3Q1K1_STANL | R5M0T5_9MOLU |
| C5C7V0_MICLC | A0A0A6D3P2_9SPHN | C6HTW2_9BACT | G0GBN6_SPITZ |
| Q04HQ6_OENOB | F6CKP8_DESK7 | G7CIJ8_MYCTH | F6FWN5_ISOV2 |
| D5MK04_9BACT | A0A0A1FWC9_9MYCO | A6DQM0_9BACT | K9V7L7_9CYAN |
| R6T680_9FIRM | H9UJF3_SPIAZ | F9VGA9_LACGL | R7F241_9BACI |
| A0A0F2PHJ2_9FIRM | R6SGE0_9CLOT | D2S814_GEOOG | C0W4X0_9ACTO |
| B0TA59_HELMI | S1RRM8_9ENTE | E6MIL0_9FIRM | F7YUV7_9THEM |
| F6B6R2_DESCC | F3ZWZ1_MAHA5 | F2NYC3_TRES6 | F2IIU9_FLUTR |
| A0A0M2HUHL_9MICO | F7NE40_9FIRM | A0A060QK02_9PROT | A0A0M2SZ93_9BACI |
| A0A099WPE6_9LIST | R4KWF9_9FIRM | R6XT61_9CLOT | C7MS26_SACVD |
| I7L720_9LACO | J6IJK1_9ACTN | A0A0A0X2I0_9SPIO | G5KHN3_9STRE |
| E3EKM5_PAEPS | C7MPK4_CRYCD | A0A0G0I018_9BACT | D6CK72_XANAP |
| F9MV75_9FIRM | A8YW48_LACH4 | E1L0M3_9ACTN | A0A0F0KZA8_9MICO |
| B6GAM6_9ACTN | L0KBU6_HALHC | F8EJI2_RUNSL | B2TRG6_CLOBB |
| A0A0G3WHL9_9BACT | A0A0A2TPR6_9BACL | A0A0M2CX95_9MICC | R6M0Y4_9CLOT |
| A0A0B4RFI6_9BACL | C5RA89_WEIPA | H8E981_9MICO | D4YSR8_9LACO |
| G7QC93_9DELT | A0A0I9Y3H7_9MYCO | D5H0C0_LACCS | C9A8S5_ENTCA |
| R5CPV5_9BACT | C9ZFB4_STRSW | R5SAB4_9GAMM | D3F3T6_CONWI |
| U5S6H1_9LACT | SSB_TREPA | B2TP00_CLOBB | W9ELJ4_9LACO |
| A0A0M3D3N6_9MICO | K7VWI7_9NOST | R7FKB8_9CLOT | H6RVY1_BLASD |
| A0A0A7FWL2_9CLOT | A0A0M2U8X9_9FIRM | Q2RM71_MOOTA | A0A0M2VQ29_9BACL |
| X2HCF9_9GAMM | U2Q8L6_9FIRM | H5XC54_9PSEU | R5PVH7_9CLOT |
| Q74M27_LACJO | X2GVH9_9BACI | A0A0D5AEV9_9NOCA | R5Q9E2_9FIRM |
| Q2JQ03_SYNJB | S2DXU5_9BACT | K1ZW69_9BACT | B1MMI1_MYCA9 |
| R6XQU8_9FIRM | R5AUK9_9FIRM | K6C9N5_BACAZ | K2EUR8_9BACT |
| C0MEM0_STRS7 | E3IZT7_FRASU | D7GH49_PROFC | R6U666_9CLOT |
| C7XXJ4_9LACO | S1RPU4_9ENTE | C8WIP6_EGGLE | R5HK63_9FIRM |
| W5WNL7_9PSEU | R7L882_9BACT | E0RXF1_BUTPB | F5YVQ0_MYCSD |
| R7NEZ3_9MOLU | D4S6J8_9FIRM | R6NRQ8_9FIRM | R7NN10_9FIRM |
| R5CX84_9FIRM | A4FR31_SACEN | B0SSW1_LEPBP | A1RDC5_ARTAT |
| A0A0M2ZFM8_9MYCO | R6ECK9_9FIRM | R6NDS1_9CLOT | B1C975_9FIRM |
| S0SMI5_ENTAV | K9X4B7_9NOST | Q6A5N0_PROAC | A6X3T2_OCHA4 |
| D3P9P9_DEFDS | D6E866_9ACTN | F2J196_POLGS | R5ME01_9MOLU |
| R2Q3W5_9ENTE | A1THY6_MYCVP | B1I6T0_DESAP | K2AEK0_9BACT |
| R5Y805_9MOLU | Q01FS1_OSTTA | B0T9B6_CAUSK | V5XKD4_ENTMU |
| R6UV64_9FIRM | A0A0F2C1D9_9MICO | R7D361_9ACTN | A0A0A1CZK3_9MICC |
| S0EYS7_CHTCT | R6B7C1_9CLOT | D9T411_MICAI | R5THR6_9CLOT |
| E9SVY8_RHOHA | A3DHF9_CLOTH | C0GI94_9FIRM | A0A0F5NGW8_9MYCO |
| D3IAG1_9BACT | Q0FUG1_PELBH | B9DJE5_STACT | A0A0F0LDG7_9MICO |
| A0A0F0KNI0_9MICO | R6H510_9FIRM | A0A0G3XKH1_9SPHN | A0A0A7PS75_9SPHN |
| R7ANG4_9ACTN | R7M6R0_9CLOT | C8WV95_ALIAD | R6DRY6_9CLOT |
| SSB_MYCTU | R5FLX6_9BACT | A0A0E9MUR3_9SPHI | W8TJI2_EUBAC |
| A0A088ET40_9SPHI | R6LVK0_9FIRM | A4RRU9_OSTLU | F8HY91_WEIKK |
| A0A068NVF8_9BACT | S2W6P3_9ACTN | E8X1F5_GRATM | K6VPE8_9ACTN |
| F1SRQ7_PIG | H1PJB0_9FIRM | K0Z6V8_9ACTN | G9YJ09_9FIRM |
| F2I5E0_AERUA | U4Q9S1_TEPAE | I2EVM7_EMTOG | I5AVT5_EUBCE |
| A0A086YV23_9FIRM | H1Z8W1_MYROD | A0A0M2RD12_9ACTN | E8SHY8_STAPH |
| R6GTK3_9FIRM | R7N7X3_9FIRM | E2RST4_CANLF | A0A0F5P9A7_9SPHN |
| A3DF53_CLOTH | B2TSA5_CLOBB | K9Y789_HALP7 | M3ZQC4_XIPMA |
| B1X1C2_CYAA5 | D3EPD2_ATETH | G9L570_MUSPF | F1N1S0_BOVIN |
| F1T5Q2_9ACTN | F6RJN1_HORSE | A0A0C2TU04_BACBA | A0A0J6YHL0_9MYCO |
| G1M9E9_AILME | A5VHG0_LACRD | Q744V5_MYCPA | C4FAZ9_9ACTN |
| F4CXT6_PSEUX | F6CF29_LACKZ | F7MSD7_CLOBO | L9L0W8_TUPCH |
| G4KXN0_OSCVS | D9QUL1_ACEAZ | H0XFVO_OTOGA | K7E8E7_ORNAN |
| B8I3W7_CLOCE | I0LDK8_9ACTN | I4B8K2_TURPD | E0NIS3_9FIRM |
| F7C414_ORNAN | G3T7N4_LOXAF | I0ILW6_LEPFC | R6Z1V7_9ACTN |
| R6WMS7_9FIRM | R7G0F0_9FIRM | E8RVU3_ASTEC | K6WB53_9MICO |
| D1PJS6_9FIRM | K9XY78_STAC7 | A4XDG7_SALTO | R5WIJ2_9DELT |
| R5QD10_9FIRM | R5T0D9_9CLOT | R6DCZ8_9FIRM | C8W049_DESAS |
| D9SWF8_CLOC7 | A8MED5_ALKOO | E7AD95_HELFC | R4K5X2_CLOPA |
| I3EBG5_BACMT | R5DJN3_9FIRM | A0A0F6R208_9CORY | R6HES5_9CLOT |
| W5PX87_SHEEP | D9VB97_9ACTN | F8EXJ2_TRECH | K9T956_9CYAN |
| K8GNK7_9CYAN | C7RH78_ANAPD | A0A0M3AIU4_9SPHN | K4IG43_PSYTT |
| Q46II8_PROMT | A0PX88_CLONN | R7AXP0_9FIRM | SSB2_LISMO |
| L8ALM1_BACIU | SSB1_SYNY3 | D2BED7_STRRD | G9WF10_9LACT |
| H2J429_MARPK | R4M2D8_9ACTN | U3J0M0_ANAPL | U5WCD4_9ACTN |
| R5VMD3_9FIRM | Q03D46_LACC3 | C4ZDL5_EUBR3 | H2MQE2_ORYLA |
| R6BYC9_9CLOT | R5EAX7_9FIRM | R5T2Q1_9CLOT | R7R157_9FIRM |
| A4J9Q4_DESRM | A8UWG2_9AQUI | K9W323_9CYAN | D1BGE6_SANKS |
| A0A0D8I5T9_9CLOT | S1NV36_9ENTE | I0YSZ5_9CHLO | M1S8U3_MORMO |
| M1S8U3_MORMO | S1NK24_9ENTE | A4AEU3_9ACTN | SSB_MYCLE |
| G3Q9G8_GASAC | K0NMK9_9LACO | R7DBY1_9FIRM | F9DY26_9BACL |
| R6DN65_9CLOT | C9LU67_SELS3 | R5TRH8_9FIRM | M7AI56_CHEMY |
| Q31RI6_SYNE7 | B7KF71_CYAP7 | A3K4Q5_9RHOB | D7VRI1_9SPHI |
| C8WYC0_ALIAD | G1TFS5_RABIT | A0A0G0M5P2_9BACT | D4J7E9_9FIRM |
| A0A087Y2Y2_POEFO | B1MWV1_LEUCK | G8T9U4_NIAKG | R6ERI5_9FIRM |
| A0A0D6A0Q8_9LACO | F6DRE2_DESRL | R7GDI2_9CLOT | A0A076NIV4_9CORY |
| F7KMP1_9FIRM | K4Z6N1_PAEAL | F0RVP0_SPHGB | D5BC93_ZUNPS |
| F6YE54_CALJA | R5P5H2_9BACT | R6ZJ99_9FIRM | K1ERU3_9MICO |
| A0A0M2UA92_9FIRM | D4JEM5_9FIRM | W5K2H1_ASTMX | K1YBF9_9BACT |
| A1HSI9_9FIRM | R6FJ83_9FIRM | R7J855_9FUSO | G5JXG8_9STRE |
| C8XDE2_NAKMY | C2L0W5_9FIRM | E1W135_ARTAR | G8QXZ1_SPHPG |
| K0YPC2_9ACTO | SSBP_RABIT | H0ZMY7_TAEGU | G1SKF0_RABIT |
| E7GNQ2_CLOSY | G1TWH4_RABIT | E3PS07_CLOSD | A0A0F0H116_NOCAE |
| H2U9B8_TAKRU | U2KG41_9STRE | A0A0G0CQI7_9BACT | A0A0B5D669_9CORY |
| R5DSD5_9FIRM | A0A0B5DZS3_9RHOB | A4XJ53_CALS8 | G1SAX7_NOMLE |
| W5YBI8_9CORY | E8LE33_9FIRM | A0A0F2NNB1_9FIRM | R5TE43_9FIRM |
| A0A0F9Z9P0_9BACT | U3JMD2_FICAL | C3PIE7_CORA7 | A0A0A2U240_9BACL |
| M3W650_FELCA | F4LSB8_TEPAE | C7HV65_9FIRM | R5BGZ2_9FIRM |
| C7Q4G0_CATAD | A5VH17_SPHWW | E1R210_SPISS | K9UNF6_9CHRO |
| F0H1S8_9FIRM | F3Y7N0_MELPT | C7N448_SLAHD | I7KKM2_9LACO |
| A1HR42_9FIRM | R5H238_9SPIR | E6S7C6_INTC7 | W5TTM8_9NOCA |
| R5KQL9_9FIRM | R6K193_9FIRM | R7HRX6_9CLOT | R5ZCW9_9ACTN |
| C0R077_BRAHW | R5AJ65_9FIRM | K1XZ04_9BACT | A0A0D9R6X7_CHLSB |
| I2F404_9BACT | I3M8X8_ICTTR | A0A096MTD7_PAPAN | F6X3C0_MACMU |
| G7P1E9_MACFA | W8U9E7_EUBAC | I0X6L5_9SPIO | E6SLI9_THEM7 |
| SSBP_PONAB | H2QVI4_PANTR | SSBP_HUMAN | G3R2E1_GORGO |
| R6PGX1_9FIRM | R6Q6X2_9FIRM | B4WRL5_9SYNE | SSBP_BOVIN |
| B0RDN7_CLAMS | I3KN30_ORENI | F7MJE1_CLOBO | A0A0G0J791_9BACT |
| R5ADK0_9FIRM | H3NIJ2_9LACT | A0A0F0LRP6_9MICO | R6HA90_9ACTN |
| Q7NCN6_GLOVI | E7EUY5_HUMAN | C8NI09_9LACT | K9S5Q5_9CYAN |
| R5U6P8_9FIRM | E3HXL9_ACHXA | SSB2_CLOAB | A0A0C7NPU9_9BACT |
| A0A0F3FVR6_9LACO | A0A022LVI6_9MICO | C2CYC8_LACBR | I7KJX8_9CORY |
| R6SP51_9FIRM | D0L1A3_HALNC | D4M166_9FIRM | A0A0G1YPM3_9BACT |
| R5SNS5_9FIRM | J9H7C5_9ACTN | R5ZLV4_9FIRM | SSBB_BACSU |
| L8AVU2_BACIU | F8E7P3_FLESM | G1QPH4_NOMLE | A0A0F4L1N9_9LACO |
| B8A5I7_DANRE | R6C2Z2_9CLOT | B9E7S6_MACCJ | F9Q480_STROR |
| E8V197_TERSS | I7LFZ3_9CLOT | R7C795_9CLOT | A0A0G2JLD8_HUMAN |
| R5CH32_9FIRM | M5A794_9ACTN | B0CEQ2_ACAM1 | D3DG88_HYDTT |
| W1SK53_9BACI | C1F4R9_ACIC5 | R6AVI9_9FIRM | E3PVY2_CLOSD |
| R5HBE8_9FIRM | E8N9Q1_MICTS | I0XMM5_9LEPT | K2BN93_9BACT |
| A0A0F2PZW6_9FIRM | F7K7Z8_9FIRM | D4LNF3_9FIRM | A0A089ZK63_9LACO |
| E1R6X0_SPISS | G1P8K1_MYOLU | B6GDU1_9ACTN | C1F5R8_ACIC5 |
| F7V2Z0_CLOSS | C6LLT2_9FIRM | R7AVA4_9FIRM | R5DTA3_9FIRM |
| D4W5D5_9FIRM | R5MZ80_9FIRM | U5Q7H7_9BACT | R7CDZ3_9FIRM |
| A8UZL7_9AQUI | B2A455_NATTJ | R6E0A2_9FIRM | A0A0N8JYM3_9TELE |
| W0JDR4_9BACT | R6WEN5_9FIRM | G2SPI9_LACRR | B7C9Y1_9FIRM |
| I0GI75_CALEA | R4K4U6_CLOPA | G6F027_9PROT | R5HV83_9FIRM |
| R5FJB4_9ACTN | K8EIA4_9FIRM | R7K0K6_9CLOT | R7FFT2_9FIRM |
| B5CQ72_9FIRM | E0UMC2_CYAP2 | F3ANA9_9FIRM | C7RGC0_ANAPD |
| R4SXQ7_AMYOR | F9ED05_9ACTO | D9VNU3_9ACTN | K9SIA7_9CYAN |
| R6PPR1_9FIRM | A0A0F5P757_9SPHN | D9R1Y1_CLOSW | A0A0M3AMT0_9SPHN |
| E1BTE0_CHICK | A0A0F0CNJ0_9CLOT | G9RVU9_9FIRM | A5Z4B4_9FIRM |
| F9ZPK3_ACICS | T1ZBR8_STRIT | D9XNX1_9ACTN | B0K8G3_THEP3 |
| R5VQV8_9FIRM | K2GHD5_9BACT | G9PGJ7_9ACTO | E0UA33_CYAP2 |
| A1WDQ0_ACISJ | A0A0M2H829_9MICO | Q1NC23_SPHSS | G2IU50_9SPHN |
| D4YY34_SPHJU | D7E202_NOSA0 | A0A0M2NCL8_9FIRM | H9UKK5_SPIAZ |
| K9R8A5_9CYAN | I7J5V6_9CLOT | K9WD85_9CYAN | K9PEK3_9CYAN |
| A0A099WEX7_9LIST | SSB3_CLOAB | C3XP75_9HELI | A0A059MK36_9NOCA |
| K1M765_9FLAO | D6S3J9_9LACO | Q65E30_BACLD | R9YIK7_9PROT |
| F2IIU8_FLUTR | Q9CFP4_LACLA | Q4C4A2_CROWT | G9WKY9_9FIRM |
| F9PDK0_9STRE | A0A068SFW7_9FUNG | Q82YK1_ENTFA | A0A023WZ22_9ACTN |
| J4UBY6_9FIRM | V5XV83_ENTMU | R6D7W9_9FIRM | D4J8W6_9FIRM |
| F7UXA5_EEGSY | Q1GW29_SPHAL | R5X759_9FIRM | C7H6E3_9FIRM |
| A0A0F4JZ00_9ACTN | Q8DNH4_STRR6 | D2NQ89_ROTMD | G0FZG1_AMYMS |
| A0A0G1DY79_9BACT | R6DY76_9FIRM | M1E510_9FIRM | F9PCR2_9STRE |
| D6YAJ5_THEBD | Q03UT2_LEUMM | D3D6I3_9ACTN | F9PZY5_STROR |
| A5UU58_ROSS1 | A0A0F0ESI1_9MICO | A6LKQ0_THEM4 | R4K910_CLOPA |
| A0A0F4LYD6_9LACO | R5A082_9CLOT | SSB_LACPL | C8WNE4_EGGLE |
| D6E8L3_9ACTN | K7RSG7_PROA4 | R5HIX1_9MOLU | R5R0I9_9FIRM |
| D6E9A6_9ACTN | R5FWS9_9FIRM | F3ALU5_9FIRM | G4L9D5_IETHN |
| C9ZAN4_STRSW | R7BN60_9ACTN | A6EHG8_9SPHI | C4ZBQ4_EUBR3 |
| A6TRB9_ALKMQ | D4M0T8_9FIRM | E8V518_TERSS | F5WV62_ERYRF |
| R7MTA7_9FIRM | B7K610_CYAP8 | U3GW76_9CORY | U5L431_9BACI |
| A0A0A1H454_9BURK | B1XLH4_SYNP2 | A7HKU6_FERNB | D3SNS2_THEAH |
| L5KQV0_PTEAL | K1WZW4_9BACT | A0A0F7HHS8_9STAP | R5BRV9_9FIRM |
| R5EDC6_9CLOT | R6JYD3_9CLOT | E6WYD3_NITSE | R5JVR8_9FIRM |
| D4LX16_9FIRM | Q7V9P5_PROMA | SSB_CALS4 | G2THW2_BACCO |
| A0A0G1ZRH2_9BACT | SSB_THEEB | E4TF90_CALNY | B4U7M9_HYDS0 |
| I0HJX3_ACTM4 | A0A0G2K747_RAT | Q08D46_XENTR | G3V7K6_RAT |
| E6U6D8_ETHHY | D4MTT0_ANAHA | A0A0M2H3M8_9MICO | R7BHP7_9FIRM |
| R6HJ30_9ACTN | SSBP_RAT | R6G7P4_9FIRM | F6ZGI9_XENTR |
| R7FPV5_9FIRM | K9SYX5_9SYNE | D4YHA4_9LACT | K9VAE1_9CYAN |
| SSBP_MOUSE | C4Z4Q5_EUBE2 | R7H9I6_9FIRM | E1M8N3_9STRE |
| D9S186_THEOJ | R7DAW4_9ACTN | A4J362_DESRM | U2EED0_9BACT |
| G3HGL0_CRIGR | R6HDB5_9CLOT | Q8R2K3_MOUSE | A9AYW2_HERA2 |
| G2P799_STRVO | R7A8H1_9CLOT | R6Z8U9_9CLOT | A0A088T1M9_9MOLU |
| R5QXZ4_9FIRM | B2TMX5_CLOBB | B2J2S0_NOSP7 | B1H012_UNCTG |
| A0A0A0X352_9SPIO | E8JIW4_9ACTO | D2S7J2_GEOOG | G4L1H2_OSCVS |
| H0QKG9_ARTGO | K9ZC28_ANACC | F5SHA9_9BACL | D2RN22_ACIFV |
| B1C4J5_9FIRM | K0J1F5_AMPXN | C5ESW7_9FIRM | G4D4Z7_9FIRM |
| R4KD06_CLOPA | R5ZLD1_9FIRM | R5C772_9FIRM | E9T0A1_RHOHA |
| K6DEM7_9BACI | A0A0A1DSS4_NOCSI | C6Q0F7_9CLOT | R5FA57_9CLOT |
| R4TJ11_AMYOR | D4MX05_ANAHA | R6DH82_9CLOT | Q7UZN5_PROMP |
| A8TCG9_9VIBR | R5P3I4_9FIRM | D4L214_9FIRM | D4LVA5_9FIRM |
| B7C9D1_9FIRM | Q8XNP5_CLOPE | U5RXM5_9CLOT | B5GXB2_STRC2 |
| W0EY94_9SPHI | R6NH05_9FIRM | H5TVR1_9ACTN | H6N236_GORPV |
| Q4L5H4_STAHJ | A1AY96_PARDP | K9QNQ4_NOSS7 | R5I4T8_9FIRM |
| R5U7M0_9FIRM | A0A0E3M802_CLOSL | A0A0H3J879_CLOPA | D4YKW8_9MICO |
| SSB1_LACLA | A6TJA8_ALKMQ | R5SUP9_9CLOT | D0UIR6_AGGAD |
| A0A062XK97_LEUPS | SSB_STRR6 | A0A0F9YFD2_9BACT | V6K9R3_STRRC |
| R5QDS7_9FIRM | A0A0M2PPY3_PROHO | F2N9C8_CORGP | A0A0C1T6X8_9ACTN |
| A9KKC9_CLOPH | R6IDK3_9FIRM | V4ITH1_9ACTN | E8SDU6_STAPH |
| F5XIS8_MICPN | R6PZ59_9CLOT | K0B4K0_CLOA9 | E1QXP3_OLSUV |
| G5B580_HETGA | J5HB87_9FIRM | G1KLA3_ANOCA | B8E0J1_DICTD |
| G3VF75_SARHA | A0A075TTH1_9CORY | B5Y875_COPPD | A5I800_CLOBH |
| G3IU60_9GAMM | E6U5C2_ETHHY | R5K868_9CLOT | R6M217_9CLOT |
| G7GKY9_9ACTN | H7C6V9_ENTFA | A0A0F5VSZ8_9ACTN | Q17Q33_AEDAE |
| C3X7T9_OXAFO | R7B0S7_9BACE | V9XJ27_9NOCA | G5GFZ2_9FIRM |
| L0KAK0_HALHC | A0A0C5FYX7_9ACTN | B9EBP8_MACCJ | C7MB48_BRAFD |
| C4GC71_9FIRM | B0T9Q9_CAUSK | A0A0F6TCB0_9CORY | R4K1E0_CLOPA |
| R6JMM3_9CLOT | G5K4M6_9STRE | G4L3L8_TETHN | I0AH49_IGNAJ |
| B5GHS9_STRSH | Q1WVN8_LACS1 | A0A0G3XK67_9SPHN | I3WC09_THESW |
| U7L398_9CORY | R6FHU1_9FIRM | Q6ABW8_LEIXX | R5UU53_9FIRM |
| A0A0H4C5K0_9ACTN | F9VBM4_LACGL | H1BI13_9FIRM | R6SCI7_9FIRM |
| SSB_CLOTE | D9QD46_CORP2 | G9ZQA9_9LACO | D4K3Q1_9FIRM |
| R7R317_9FIRM | A0A078KJD7_9FIRM | Q2G694_NOVAD | A0A0M3AM79_9SPHN |
| F0YW24_9CLOT | V6DFC0_9DELT | K2GY73_9BACT | F8E3G1_CORRG |
| N2BLT6_9ACTN | Q8F5K5_LEPIN | Q2NBL0_ERYLH | F6ENV5_AMYSD |
| C7H6V9_9FIRM | R7HZX7_9CLOT | A5F9Q3_CLOK5 | R7G9H7_9FIRM |
| D4L1G5_9FIRM | F6VVL1_MONDO | A0A0M3AYD6_9RHIZ | D4LM12_9FIRM |
| K0KGN7_SACES | K1LY02_9LACT | SSB1_NOSS1 | A0A0A8B1Q7_9ACTN |
| R6BI07_9FIRM | A0A0G3AI68_9ACTN | C0CTZ5_9FIRM | E6UEW0_RUMA7 |
| U5MXG3_CLOSA | D4CKG8_9FIRM | D7B132_NOCDD | C0C5L8_9FIRM |
| A0A0D4DPL9_9ACTN | H2RHH4_PANTR | C9K0U8_HUMAN | A3TN96_9MICO |
| F0H1Q3_9FIRM | R5D149_9FIRM | D3D9X6_9ACTN | D7N542_9FIRM |
| Q6NEQ1_CORDI | A0A091DHX3_FUKDA | D0BLJ2_9LACT | A0A0F0HG07_9ACTN |
| W6S615_9CLOT | K0YVT6_9ACTO | T0TUT1_9STRE | I5C950_9BACT |
| R5YEH2_9FIRM | K9Q960_9NOSO | K9U3S1_9CYAN | A0A073CAT4_PLAAG |
| C7M1H0_ACIFD | Q2J4C5_FRASC | A0A0M2XVT1_9SPHI | Q9CIG8_LACLA |
| T1ZFH7_STRIT | A0A075KIH8_9FIRM | L8EF91_STRRM | F8JTF2_STREN |
| Q01P66_SOLUE | A0K2H4_ARTS2 | E7NQ91_TREPH | R5X9Q1_9CLOT |
| J6HF95_9FIRM | C3X998_OXAFO | J0UXX0_9HELI | M1NQN4_9CORY |
| A0A0M2JJ61_9ACTN | A0A0H3JAY7_CLOPA | A0A0G4AZY9_9BACT | Q81E38_BACCR |
| W0PCJ2_9BURK | R6QBY0_9FIRM | A0A075JDJ9_9MICO | F0SA65_PSESL |
| V8PCQ9_OPHHA | D5ZR07_9ACTN | G2T4Z3_ROSHA | U2IJE4_9STRE |
| F3Z785_9ACTN | J5UH88_9FIRM | R5ICL5_9FIRM | A0A0G0I4S4_9BACT |
| Q1B002_RUBXD | R5KD39_9CLOT | A0A0F4KNF1_9ACTN | B8D1C8_HALOH |
| C4XPY4_DESMR | I7KGV3_9LACO | A0A0G1V5K9_9BACT | F8I9E4_SULAT |
| A0A099WMF3_9LIST | Q4JSG0_CORJK | Q81R86_BACAN | Q1GC36_LACDA |
| E1M5B2_9STRE | E4KMD8_9LACT | A0A0G1PY19_9BACT | A0A0A1SB08_CLOSO |
| C2W7H2_BACCE | D8LMF5_ECTSI | R6QZ78_9CLOT | H6R954_NOCCG |
| L0RV23_MYCC1 | I8RHV1_9FIRM | R7I585_9FIRM | A0YMQ0_LYNSP |
| R5AMA1_9FIRM | D9VGM5_9ACTN | A8MHY9_ALKOO | C6WSG2_ACTMD |
| C8WA62_ATOPD | A0A0F5YJ18_9CYAN | A0A0F0LWI2_9MICO | R5YE83_9FIRM |
| B4V9L5_9ACTN | A1B6V4_PARDP | R6C7H6_9FIRM | R6TQE9_9STAP |
| R6AYP4_9CLOT | F3NK91_9ACTN | A4VSM6_STRSY | J1F2K6_9LACO |
| Q2N5L5_ERYLH | B8HPH8_CYAP4 | SSB2_STRA5 | Q8DS68_STRMU |
| B9DT16_STRUO | H0V0N9_CAVPO | B0S4C2_FINM2 | R6RS34_9FIRM |
| A5P8J7_9SPHN | SSB3_STRA5 | H7C718_ENTFA | R6Z0P3_9FIRM |
| K8EJP0_CARML | E2S8L8_9ACTN | A0A0A8B2R2_9ACTN | G5IMP8_9CLOT |
| V6K1U9_STRNV | R7XTM4_9ACTN | H1LHE4_9LACO | C7PVD3_CATAD |
| B0PAM7_9FIRM | K2BW59_9BACT | V5RWF6_9BACT | E7GFL6_9FIRM |
| A0A0A8EN42_9ACTN | A9KQ32_CLOPH | C2GFP6_9CORY | A0A0F3K3Z3_9NEIS |
| V5XVL3_ENTMU | R5IVW3_9CLOT | Q2NDL0_ERYLH | M4SAH7_9SPHN |
| B0WQG1_CULQU | R5IVB6_9FIRM | A0A0G1R8N2_9BACT | E2ZG54_9FIRM |
| K9Z8A2_CYAAP | K9XBI7_9CHRO | C4LKJ5_CORK4 | R7K9F5_9FIRM |
| R7GK56_9FIRM | D1A6N9_THECD | D8G2X3_9CYAN | F8B488_FRADG |
| A5N439_CLOK5 | SSB_UREPA | X4QZF0_9ACTO | A0A0B4S0X3_9FIRM |
| R5J759_9CLOT | D6A5L5_9ACTN | G8SFP7_ACTS5 | K1XM58_9BACT |
| S0RKA5_9ENTE | C5C660_BEUC1 | A0ZBN5_NODSP | E2ZBE7_9FIRM |
| G1D5D5_9CAUD | D5UE91_CELFN | R7BU22_9FIRM | R5J3X1_9FIRM |
| R6AJ00_9CLOT | R7P3M7_9CLOT | R5I982_9BACT | A1B7E0_PARDP |
| A0A077JG98_9CYAN | G5KEQ7_9STRE | R5ITS9_9CLOT | SSB2_NOSS1 |
| H2K3N5_STRHJ | A0A0H4BZY2_9ACIN | Q47K95_THEFY | C7Q5K4_CATAD |
| I3ZJ61_TERRK | A0A0G3XEQ1_9SPHN | D1PNB5_9FIRM | Q181R6_PEPD6 |
| C0XQX6_9CORY | R4K0U4_CLOPA | R6JHK2_9CLOT | D4K2R3_9FIRM |
| A0A0F2G8L1_9ACIN | A0A0M2LZS7_9MICO | B3QVR6_CHLT3 | D6ZJY0_MOBCV |
| C8PB10_9LACO | D4KCM1_9FIRM | R6ES87_9FIRM | D7C8S4_STRBB |
| A0A089Z0G9_STRGA | C9RC58_AMMDK | A0A0M2XMB8_9CORY | A0LWU4_ACIC1 |
| A0A0B5D8I7_9CORY | R7K5T4_9FIRM | E1R3J5_SPISS | N0B1A9_9BACI |
| E0YPL4_9CAUD | Q116R3_TRIEI | C0E890_9FIRM | D1VUS3_9FIRM |
| K9X8U2_9NOST | E1R1F8_SPISS | R5XKF2_9FIRM | F6ILS3_9SPHN |
| B4W099_9CYAN | B8HJJ1_ARTCA | A8ZYK1_DESOH | A0A077HMZ9_9CORY |
| E5WGI9_9BACI | A0A0A8JKP8_BACSX | A3W9Z9_9SPHN | A0A0D6E047_9LACT |
| G0FLH8_AMYMS | A0A0M3DHT1_9CLOT | B0PHZ0_9FIRM | H1LEZ9_9LACO |
| A0A0F2TJA1_9ACTN | E0S4M7_BUTPB | K4ZGF4_PAEAL | A0A0H4C1Z5_9ACTN |
| G7MAP9_9CLOT | S4G5V5_GARVA | G5JV28_9STRE | A0A0F9Z0F5_9BACT |
| K9YM97_CYASC | Q82YU2_ENTFA | F9HJI2_9STRE | A8AZF8_STRGC |
| X5DQY1_9CORY | A3WDG7_9SPHN | C9RAG8_AMMDK | SSB2_CHLTE |
| B6GDJ1_9ACTN | U2DZE5_9BACT | A0A0A7FYU6_9CLOT | R6LLA3_9FIRM |
| F6CGA7_LACKZ | R5LYQ9_9FIRM | D4H166_DENA2 | C4IMP1_CLOBU |
| K6U627_9CLOT | B9Y3H0_9FIRM | Q3Y114_ENTFC | I7JFJ5_9LACT |
| B0S2G8_FINM2 | D9X019_STRVR | B4VV03_9CYAN | H1BPB1_9FIRM |
| K4QY06_9ACTN | A8KY48_FRASN | Q3B6M6_CHLL7 | D3QYV9_MAGIU |
| H7EPC4_9SPIO | G0HB64_CORVD | SSB1_TROWT | K7SSA5_GLUOY |
| A6M304_CLOB8 | Q857R0_9CAUD | Q5M2H8_STRT2 | Q3ANR4_CHLCH |
| B4S3C4_PROA2 | M4S4J6_9SPHN | J3JC89_9LACO | X4ZWN4_9BACL |
| R5AH99_9CLOT | R6R1Y2_9FIRM | Q2NBN4_ERYLH | C0W5T8_9ACTO |
| R6QI05_9FIRM | A0A0M2N1B0_9ACTN | B4NKZ7_DROWI | H4GIG3_9LACO |
| D4JTJ3_9FIRM | B0JQ04_MICAN | B4KA00_DROMO | R6ULN2_9FIRM |
| D4LDT4_RUMC1 | D7BLE9_ARCHD | I3VSR0_THESW | R5KII9_9CLOT |
| A0A0M2GYF5_9MICO | J1ZWV2_9ACTN | E8X7E8_GRATM | K9RR91_SYNP3 |
| G5K2B2_9STRE | A7GP66_BACCN | F5Z5L2_ALTSS | G6EMZ8_STRTR |
| B8H840_ARTCA | A0A084WRC1_ANOSI | R6Y2B4_9CLOT | B3MTU7_DROAN |
| L7LMG5_9ACTN | C8NI83_9LACT | B0PEV9_9FIRM | S5QY48_9STRE |
| D9W8L5_9ACTN | R5F117_9CLOT | U3JCL9_FICAL | B4SB43_PELPB |
| E3H7R3_ILYPC | A0A0A1GU37_9LACO | B4IBZ2_DROSE | SSB_CORGL |
| B4QX63_DROSI | SSBP_DROME | I7J8Z5_BABMI | E8WBV8_STRFA |
| E0E595_9FIRM | B4LXZ6_DROVI | G2G710_9ACTN | A0A0F2PDU0_9FIRM |
| T0UUE9_9STRE | E3H340_ROTDC | C5NVZ7_9BACL | C7XUW1_9LACO |
| A1BJC2_CHLPD | A0A075KAQ6_9FIRM | A1WDI7_ACISJ | R5FUF7_9FIRM |
| R7HLY7_9FIRM | D4LEZ6_RUMC1 | SSB2_STRCO | R6JNI5_9CLOT |
| R5MMP1_9FIRM | W7TIN7_9STRA | L8F0E9_STRRM | B4GMJ5_DROPE |
| Q294W1_DROPS | E6KSY5_9ACTO | G0FHG4_AMYMS | Q7QC69_ANOGA |
| K2D879_9BACT | K1LWN4_9LACT | A0ZFI4_NODSP | C7NDG9_LEPBD |
| K2F787_9BACT | Q0RAR5_FRAAA | Q48AK8_COLP3 | E0E1I1_9FIRM |
| D1A8E9_THECD | R7HIN3_9MOLU | R6MZ43_9CLOT | C4WSH6_ACYPI |
| B4JG16_DROGR | U2N105_TRESO | Q6NEI4_CORDI | A0A0L0C938_LUCCU |
| D6K6Y6_9ACTN | E0UNQ0_CYAP2 | A0A075KE67_9FIRM | R6UNN6_9CLOT |
| M1N7D9_9CLOT | R5VJF6_9FIRM | Q0YNZ3_9CHLB | A0A077HLA7_9CORY |
| R5BQM1_9FIRM | B8I053_CLOCE | J1H3J1_9CLOT | SSB4_STRA5 |
| M1CAV8_SOLTU | U2KMT7_TRESO | F0S465_DESTD | I7A1K0_MELRP |
| G0G277_AMYMS | A0A0G1KEH6_9BACT | SSB_AQUAE | R6RG83_9FIRM |
| M1CAV7_SOLTU | A0A0C5S293_9MOLU | R7FWV6_9PROT | E3H1M9_ROTDC |
| I9LJM8_9FIRM | A0A0F5I6R0_9BACI | SSB_LEPIN | U4KR14_9MOLU |
| F4GH00_ALIDK | E7MR23_9FIRM | A0A0C2YAV4_BACBA | K0YNW0_9ACTO |
| G8SF46_ACTS5 | K4B142_SOLLC | W5JL12_ANODA | R5VM87_9CLOT |
| A0A067R5S5_ZOONE | E8T4D8_THEA1 | N2BLQ3_9ACTN | B5H606_STRPR |
| V5WCX2_9SPIO | C4F8E6_9ACTN | A0A059B337_EUCGR | A0A0B5AQD2_9BACL |
| A0A0A8W776_CLOSO | C4LGG0_CORK4 | A7SEZ5_NEMVE | E0UNI7_CYAP2 |
| F8JWV7_STREN | R6XRZ0_9CLOT | K6DKB5_BACAZ | A0A0M2Z6A1_9ACTN |
| K9ZS22_ANACC | R5JB91_9FIRM | B9STF2_RICCO | S8C5W4_9LAMI |
| G5K9Z6_9STRE | A0A0F5W5C6_9ACTN | D2A2D3_TRICA | Q8Y4C7_LISMO |
| U4KLQ5_9MOLU | C0MFZ9_STRS7 | R6NWZ1_9CLOT | C7M1D3_ACIFD |
| B7GDH9_PHATC | R6LWH9_9CLOT | U2Q8T2_9FIRM | F8I2Y8_SULAT |
| A0A077J6V4_9BACI | R6YDA7_9CLOT | D6Z9K0_SEGRD | M0TZ66_MUSAM |
| W4WGZ6_ATTCE | U5DC96_AMBTC | A0A0F7N7R7_9ACTN | A1RC69_ARTAT |
| G6FVY8_9CYAN | R5Y2C2_9CLOT | R7MDV0_9CLOT | G1NKN7_MELGA |
| B0SP31_LEPBP | B0S487_FINM2 | A0A0A1CW74_9MICC | R5TB82_9CLOT |
| R5GS36_9FIRM | B3R0K2_PHYMT | B3EKG0_CHLPB | R6FRM9_9CLOT |
| Q8R8F1_CALS4 | A9T9Z2_PHYPA | D0GIJ6_9FUSO | G7I9F0_MEDTR |
| A0A0G3HFP3_9CORY | I0XTS8_9LEPT | R6E141_9FIRM | D6YRN8_WADCW |
| Q6YRK4_ONYPE | E1R7U9_SPISS | F2JRY8_CELLD | C4GAT4_9FIRM |
| I1A6Q6_9MOLU | A9HT87_GLUDA | F9PLE9_9ACTO | E2A4E2_CAMFO |
| J7L8W1_NOCAA | A0A0F7CP49_9ACTN | R2S0D0_9ENTE | C6VZY4_DYAFD |
| H9IUP7_BOMMO | A0A072VNU3_MEDTR | I1CFQ3_RHIO9 | K9U4I7_9CYAN |
| M2XDC8_GALSU | G2J0V5_PSEUL | A0A061ABV0_9MOLU | SSB_BORBU |
| D9WWZ7_9ACTN | B0S470_FINM2 | J3JWK6_DENPD | A9NEP0_ACHLI |
| R5KNI9_9CLOT | E2C7L8_HARSA | A0A0M2HMR7_9MICO | A0A0G1VV93_9BACT |
| R6H4K2_9FIRM | F4XCR8_9FIRM | D7BCU9_MEISD | D7BCU9_MEISD |
| C4ICW4_CLOBU | D0WL44_9ACTO | A0A0M3DGJ3_9CLOT | D2VRD1_NAEGR |
| D0BM39_9LACT | D1AJZ4_SEBTE | R6MUJ6_9FIRM | B5I5T8_9ACTN |
| A1QYR3_BORT9 | A0A022REF4_ERYGU | W5A3T7_WHEAT | S5LST4_9MOLU |
| K1YZ76_9BACT | T1G5H7_HELRO | B2TQY8_CLOBB | A0A0D6DYW6_9LACT |
| F4LPJ2_TREBD | W0PFC5_9BURK | M0TS40_MUSAM | M1M7M3_9CLOT |
| J9W2T3_LACBU | A6CPQ2_9BACI | I7L846_9LACT | W5AER3_WHEAT |
| A0A068NH60_9ACTO | J9W267_LACBU | U6E388_9MOLU | A0A058ZA14_9EUKA |
| F7ZZC6_CELGA | E8MXY3_ANATU | D7U9Z5_VITVI | I2N1Y8_9ACTN |
| K4ZCE8_PAEAL | A0A0B5EZL0_9ACTN | R0HD12_9BRAS | A0A061FAS7_THECC |
| C3WBN3_FUSMR | D1BRT7_XYLCX | A0A0D4CJ99_9LACO | E2ZNS8_9FIRM |
| G6DF00_DANPL | A0A061F3U5_THECC | A0A0G3XN39_9SPHN | D4BM44_BIFBR |
| R6I6X7_9FIRM | R7BIL2_9FIRM | A0A087GPT5_ARAAL | G5KEE4_9STRE |
| S2K7U9_MUCC1 | M5WHL0_PRUPE | D4MVL9_ANAHA | I3UGJ2_ADVKW |
| F2EFX4_HORVD | G2NXT3_STRVO | I1BS51_RHIO9 | F4WAN6_ACREC |
| F8L069_PARAV | A0A0A0LVY2_CUCSA | R5MS63_9MOLU | A4JV78_BURVG |
| E0DH66_9CORY | A0A023CPK7_GEOSE | F4GK80_SPHCD | A0A0A7I4Q3_9BIFI |
| I6XYA9_PROPF | SSBP_ARATH | R6H221_9FIRM | E2ZAS2_9FIRM |
| K0SQT6_THAOC | E4RL94_HALHG | A0A067L436_JATCU | F4H5G5_CELFA |
| D5AVD7_RHOCB | G2NI88_STREK | A0A0K9NWZ7_ZOSMR | F9MRH9_9FIRM |
| K1M9H1_9LACT | A0A061E958_THECC | M0XSF7_HORVD | A4XE47_NOVAD |
| K2EF75_9BACT | E3IUL2_FRASU | E4PRR2_MARAH | R6R906_9FIRM |
| D9WPL2_9ACTN | A0A0G0T216_9BACT | A0A0M2HGC3_9MICO | SSB1_CLOAB |
| E8NDK7_MICTS | J0NGW5_9ACTO | T1L9G1_TRIUA | A0A0A7I9B8_9BIFI |
| Q6MC71_PARUW | A0A0D3G9M1_9ORYZ | A0A0E0PPT0_ORYRU | A0A0E0A1G2_9ORYZ |
| R5HKG1_9MOLU | F8L7J6_SIMNZ | U7MT71_9CORY | F2ICX2_FLUTR |
| V4QNQ3_STRIN | F2RD73_STRVP | A0A0A8EXI3_9ACTN | D7EYW0_ARALL |
| D4BRU5_BIFBR | W4ZRU4_WHEAT | C4IYS0_MAIZE | I4YF78_WALMC |
| A0A0G3HFE7_9CORY | A0A0E7LNC1_9NEOP | R5A412_9CLOT | I1EB73_SOYBN |
| I1QKS2_ORYGL | Q6E4W0_ORYSJ | B9I7A1_POPTR | A0A0A7PEE5_9BACT |
| K3Z9P1_SETIT | C6VVF4_DYAFD | D1NWJ3_9BIFI | A0A097IIX8_9CORY |
| V7BK76_PHAVU | A0A0E0L4I0_ORYPU | G9WE70_9FIRM | A0A0D3E947_BRAOL |
| A0A096QAV3_MAIZE | K2BAD0_9BACT | A0AW08_ARTS2 | A0A0M2HYL8_9MICO |
| R6BKT7_9CLOT | I0GQE5_SELRE | R6GPN1_9FIRM | F2NT26_TRES6 |
| H8E4K3_9MICO | I4EAU6_METSZ | B1VPC3_STRGG | R6CL18_9CLOT |
| M4F2F9_BRARP | D7W9B0_9CORY | A0A0H4P3T7_9BACI | D7BJJ9_MEISD |
| C2WZR6_BACCE | A0A078DHL3_BRANA | E6W6W1_DESIS | I1HHT5_BRADI |
| A0A0M2EPP6_9SPHN | D2Q3W6_KRIFD | R6Z1D4_9FIRM | K7J2W0_NASVI |
| C4K426_HAMD5 | B8AZT8_ORYSI | E3DS65_HALPG | A0A0D2R9N2_GOSRA |
| A0A0D2R335_GOSRA | C4GHF6_9NEIS | A0A0D2S506_GOSRA | I1HS48_BRADI |
| C7R3A8_JONDD | H3NE41_9FIRM | K9U8Y9_9CYAN | F8AYN8_FRADG |
| A0A0F0HK54_9PSEU | R6E0F3_9FIRM | A0A0F5P9A5_9SPHN | D4K451_9FIRM |
| A0A059NWA3_9BACI | A0A0M2UAZ0_9FIRM | H2CBY9_9LEPT | A0A078JKM8_BRANA |
| R6D5E3_9CLOT | A0A0D2PHD8_GOSRA | C6JRG5_FUSVA | B9EBK8_MACCJ |
| J3M8R3_ORYBR | E9SD49_RUMAL | A0A016PN37_GIBZA | A6WG62_KINRD |
| R5W069_9FIRM | A1A3I0_BIFAA | E5BEX3_9FUSO | R4KEJ9_CEOPA |
| I3WG37_BIFBI | D9SSV4_CLOC7 | A0A0H4L230_9BACI | I1LB74_SOYBN |
| A0A0F7JQ78_9SPHN | A0A0C9M3T0_SPHPI | B2KEG1_ELUMP | I1HS47_BRADI |
| D4BUG1_PRORE | D4BUG1_PRORE | R7BP32_9ACTN | R5TEL9_9CLOT |
| R5VZQ8_9FIRM | A0A077RTF1_WHEAT | A4F8T1_SACEN | BIV954PHYAS |
| F8J185_9CAUD | D1AW03_STRM9 | W5D8T8_WHEAT | R5J8V9_9CLOT |
| Q21QI3_RHOFT | A0A0E0KPE8_ORYPU | I1YEN3_METFJ | W9R5P5_9ROSA |
| W9R5P5_9ROSA | W9S301_9ROSA | A0A068R5E5_9ENTR | K6TTT2_9CLOT |
| K3VSP0_FUSPC | S5SQD8_9PROT | R5TNB7_9CLOT | A0A0G3XNP5_9SPHN |
| Q6YQ11_ONYPE | E7NXU2_TREPH | D2Q6Q0_BIFDB | A0A0M2H5S7_9MICO |
| D1ANB3_SEBTE | A0A0A0LTL6_CUCSA | B8C9K7_THAPS | A0A0M2EMI0_9MICO |
| B7Q357_IXOSC | D8RH51_SELML | D8RAR6_SELML | A0A0F2T3P0_9ACTN |
| Q6YPM6_ONYPE | D3CUK6_9ACTN | SSB_FUSNN | E7VKB8_CEOSH |
| F9EEE1_FUSNU | W4KF50_9HOMO | M0SIR5_MUSAM | MTSS1_CAEEL |
| J3E2A2_ORYBR | A0A0D9WIG4_9ORYZ | S5YJB8_PARAH | B6JK37_OLICO |
| A0A0D3FUN2_9ORYZ | Q2NV17_SODGM | W5WTH1_9CORY | F9VI82_ARTSS |
| A0A090JPR1_9FIRM | K4IZG0_BIFAP | A8X3C7_CAEBR | G0MGH4_CAEBE |
| V4L410_EUTSA | K7N5B5_SOYBN | R6AMR3_9CLOT | Q6YQE4_ONYPE |
| H1D838_9FUSO | Q6YPJ9_ONYPE | Q1QFG2_NITHX | E3N038_CAERE |
| I2GU76_9BACT | D6YW20_WADCW | V5FI32_BYSSN | A0PZ40_CLONN |
| E9IM03_SOLIN | K0JPX9_SACES | Q6A791_PROAC | A0A077KLD4_9FLAO |
| F8KZF0_PARAV | A0A090LE13_STRRB | R5MQA9_9FIRM | C5NV94_9BACL |
| E3D770_GARV3 | F0RTU0_SPHGB | X2GTB7_9BACI | I1NJ41_SOYBN |
| R5BXI6_9BACE | A0A088T2E7_9MOLU | R5IVN3_9FIRM | A0A0E9LYC1_9BACT |
| R7EPH8_9BACE | A0A0D9MH74_9EURO | R4ECR5_9ACTN | R7HH66_9MOLU |
| Q6YQH7_ONYPE | A0A088A2R7_APIME | F9DX54_9BACL | Q6YQ63_ONYPE |
| C8NHW9_9EACT | D3PMG9_MEIRD | F2F3Y0_SOLSS | J3EX07_ORYBR |
| K3XLP5_SETIT | M4RAQ1_9BIFI | A9TAN1_PHYPA | R9ACF2_WALI9 |
| A0A088QJJ4_9CORY | A0A059VZJ4_STRA9 | W5CFS3_WHEAT | A0A0F0I7X5_ASPPA |
| R6B869_9CLOT | A0A0G3GUQ1_9CORY | E7MQM1_9FIRM | B5JNU0_9BACT |
| A0A0D9WIG3_9ORYZ | G1XDC6_ARTOA | I9C2C6_9SPHN | U6PK61_HAECO |
| G0HI79_CORVD | A0AWV3_ARTS2 | A2WT49_ORYSI | B8PEC7_POSPM |
| Q6YQK9_ONYPE | K3XM68_SETIT | B7KMH3_CYAP7 | A0A0E0JLP0_ORYPU |
| A0A060SPG2_PYCCI | K2FDS2_9BACT | A0A0B5I737_9ACTN | R7R2A3_9FIRM |
| I2JIG7_9GAMM | A0A0M2CUY5_9MICC | B9YD18_9FIRM | A0A0D6T7V8_9RHOB |
| A0A0A7G012_9CLOT | Q24LE5_BPPCD | A0A067GSU9_CITSI | K0F5S9_9NOCA |
| D4H4K0_DENA2 | K0F0D9_9NOCA | S8ANE7_DACHA | F6FJB9_MYCHI |
| A0A0G0D266_9BACT | Q184D1_PEPD6 | A0A0K0CZ86_ANGCA | D2MMI2_9FIRM |
| SSB_MYCPE | A0A0F4JXZ9_9ACTN | S5LYV5_9MOLU | E5BDE2_9FUSO |
| R7HJM0_9FIRM | T0U3E9_9ENTE | V5RJ91_SPIAP | C4IGU4_CLOBU |
| A0A077RA69_WHEAT | A0A0F0KC91_9MICO | A9JQS8_ONYPE | R5K3H0_9CLOT |
| B1VIZ7_CORU7 | J8VIP9_9SPHN | A5VGC6_SPHWW | W8GEA7_9MOLU |
| R5X4S7_9CLOT | V4SGF4_9ROSI | A0A067GQ74_CITSI | T1H2Z8_MEGSC |
| R5N754_9FIRM | E9HG91_DAPPU | A0A0J8BBZ0_BETVU | R7DQ35_9FIRM |
| J6H3K8_9PORP | K4DEI6_SOLLC | B8NEG5_ASPFN | W6NRG3_HAECO |
| A0A0L1JBF4_ASPNO | W6QPG1_PENRO | A0A0D9V3F9_9ORYZ | A0A061G1I5_THECC |
| E6JBU4_9ACTN | W5IGQ0_SCAIO | Q5VP75_ORYSJ | U1M323_ASCSU |
| Q2FWF7_STAA8 | I1NQ20_ORYGL | A0A0D3ERR1_9ORYZ | A0A087DDF0_9BIFI |
| F8A0N0_CELGA | W9EFH9_9LACO | Q6YPS0_ONYPE | A0A088QF08_9CORY |
| Q6YRH4_ONYPE | A0A0F0L4E0_9MICO | A0A067NLS3_PLEOS | F7W361_SORMK |
| L0G3E7_ECHVK | A0A0D9YC93_9ORYZ | M5FQH0_DACSP | E6TS66_BACCJ |
| U3U3T1_9ENTR | G4UQY2_NEUT9 | M7YFY9_TRIUA | Q6YQG3_ONYPE |
| A0A0G1MMF6_9BACT | B9RE51_RICCO | Q2U7U2_ASPOR | R6X8C2_9BACT |
| E5WQN5_9BACI | A4J7S4_DESRM | D2NTP1_ROTMD | M4E0N7_BRARP |
| T1KFW6_TETUR | U4KKW9_9MOLU | R5MG88_9MOLU | E6K1V8_PARDN |
| SSB1_STRAW | A0A0G3HKR4_9CORY | D7VT17_9SPHI | R6SIP2_9FIRM |
| A0A078DUV6_BRANA | C5XF43_SORBI | H9UM39_SPIAZ | E1VWT1_ARTAR |
| K6V5A9_9MICO | F2IIU7_FLUTR | B2GG11_KOCRD | D3NUP5_AZOS1 |
| A0A0F2FYC5_9ACTN | G4ELW3_MYCIO | A9RL19_PHYPA | M5WD92_PRUPE |
| M0XU19_HORVD | W7IFB9_9PEZI | A0A0F8UQU1_9EURO | R6Y4Q4_9FIRM |
| H0DGD7_9STAP | A0A0F0KXD3_9MICO | S8C8U9_9LAMI | C0BP77_9BACT |
| I1K195_SOYBN | W7LZR9_GIBM7 | I1HPU9_BRADI | Q5HMC5_STAEQ |
| G8QX45_SPHPG | W5XWC6_9CORY | B4FRF4_MAIZE | C4FDE6_9BIFI |
| W6KQ01_9PROT | D7U3W8_VITVI | A5HYT7_CLOBH | D4H4T7_DENA2 |
| Q893X5_CLOTE | U1FKZ5_TRESO | A9SBZ1_PHYPA | K8E772_CARML |
| A0A0D9VWL3_9ORYZ | E0S4T0_BUTPB | G8T8J7_NIAKG | M5BIN7_THACB |
| A0A0E0KGK4_ORYPU | E6UKA6_RUMA7 | B6HQ01_PENRW | C5CAV8_MICLC |
| J4H3J7_9APHY | A0A0B4REG2_9BACL | S8FNG6_FOMPI | A0A067FQ03_CITSI |
| A0A085BWI4_9RHOB | E4KMV6_9LACT | V4VMU2_9ROSI | A0A0B2UZX5_TOXCA |
| U5VVL1_9ACTN | A0A0F2C284_9MICO | A0A077EL86_9FLAO | F0ICS7_9FLAO |
| F2NUF3_TRES6 | A0A078EES3_BRANA | B9E8E1_MACCJ | S0DXT4_GIBF5 |
| V2XJ24_MONRO | U4KRJ4_9MOLU | D4ZVZ7_ARTPN | A0A0B5DNE3_9ACTN |
| A0A0K9PNM3_ZOSMR | A5FFM4_FLAJ1 | D4YQV9_9MICO | G9ZFM2_9GAMM |
| U9W337_NEUCR | F8B2W0_FRADG | B9DMG3_STACT | A0A0G3H624_9CORY |
| C1N4Z6_MICPC | R7GJ76_9CLOT | J6CJM6_PASMD | M0XU18_HORVD |
| J3CM39_9FLAO | D8T9F3_SELML | F2L6K8_PSEUX | Q5WE28_BACSK |
| S5VQJ5_STRC3 | H5UT03_9MICO | D7L8J7_ARALL | C1EJB6_MICSR |
| D1KBN3_9GAMM | D3LVH7_9FIRM | A0A0A0WYH3_9SPIO | R7M578_9CLOT |
| I1PDQ5_ORYGL | A2XJV7_ORYSI | A0A0E0NYJ9_ORYRU | Q75GK2_ORYSJ |
| N4U305_FUSC1 | A0A0D2XE17_FUSO4 | X0K659_FUSOX | N1RE73_FUSC4 |
| K9GIC0_PEND2 | A0A0M1V0K5_CLOSO | V3ZV46_LOTGI | R7FPD1_9CLOT |
| F9FB83_FUSOF | A0A0E0BI39_9ORYZ | R7EZE5_9BACI | A0A0A2LCU3_PENIT |
| C7N828_SLAHD | A0A0F0CDT0_9CLOT | A0A0D9VWL2_9ORYZ | T5AB61_OPHSC |
| A0A088EZ19_9SPHI | Q0JDX1_ORYSJ | J2P0J4_9SPHN | W9SGS7_9ROSA |
| A0A0E0BI38_9ORYZ | A0A067H2H7_CITSI | X0DC39_FUSOX | A0A0M2HIA8_9MICO |
| D7WDW1_9CORY | A0A0D3CIU1_BRAOL | A0A0A8JGP8_BACSX | H7F3I4_9LIST |
| U5CZX7_AMBTC | E4MZB2_KITSK | V4M6U7_EUTSA | W5NKI1_LEPOC |
| A0A022R6Z5_ERYGU | R6QMS7_9FIRM | R6A7P3_9CLOT | W6AFA9_9MOLU |
| A0A087HAQ7_ARAAL | A0A0B0MES2_GOSAR | R0G708_9BRAS | D2BE00_STRRD |
| A0A024QAI4_9BACI | A9WUZ2_RENSM | C7R1E8_JONDD | A0A067TU98_9AGAR |
| A0A0D3ACJ2_BRAOL | A0A078E4T6_BRANA | G2X781_VERDV | Q9LII1_ARATH |
| D6DG89_CLOSC | D4MS05_9FIRM | SSB_ONYPE | R4UJY5_9MOLU |
| Q2NU12_SODGM | A0A0J8CQH7_BETVU | A0A078F1K6_BRANA | R5LJ38_9MOLU |
| I3WJ92_BIFBI | Q0F348_9PROT | A0A078G1R5_BRANA | M4F913_BRARP |
| A0A022LIZ9_9MICO | A0A0M3DCH8_9MICO | A0A0D9VWL4_9ORYZ | A0A0F0LRE6_9MICO |
| A0A093YQ38_9PEZI | A0A093XU41_9PEZI | A0A094DK53_9PEZI | D4LHA6_9FIRM |
| K5X983_PHACS | B4FZD7_MAIZE | F5YIL0_TREPZ | E8T6W4_THEA1 |
| S7QK86_GLOTA | Q49Z44_STAS1 | B9S4S1_RICCO | A0A0D9VWL1_9ORYZ |
| H5U4M8_9ACTN | A0A067J8I3_JATCU | W6S2J0_9CLOT | Q0RPP0_FRAAA |
| L3ZIZ7_TERRK | W0GPN8_9MOLU | E6S6A4_INTC7 | E4RTC4_LEAB4 |
| J0WYQ3_9BIFI | V7CAJ9_PHAVU | M7SRB7_EUTLA | U5H6L2_USTV1 |
| C8XGL8_NAKMY | A0LVD2_ACIC1 | R5W4M2_9DELT | E1ZTB4_CHLVA |
| V4TA61_9ROSI | A0A067FYN2_CITSI | C7N7Z7_SLAHD | D1BWP5_XYLCX |
| SSB_BIFLO | A9B964_HERA2 | D8SFR0_SELML | B9H8G9_POPTR |
| Q03D48_LACC3 | H7FW30_9FLAO | L8GA42_PSED2 | A0A094F509_9PEZI |
| A0A094HM06_9PEZI | X4QWZ2_9ACTO | Q03G53_PEDPA | D4Z8L4_SPHJU |
| A0A094B2F4_9PEZI | A0A0A1DMG2_NOCSI | C9SUV3_VERA1 | A0A059D0P9_EUCGR |
| Q2JDW8_FRASC | K4AD55_SETIT | A0A090MT83_AFIFE | T1H8H8_RHOPR |
| C8X3A1_DESRD | A0A067FZB1_CITSI | H5WK22_9BURK | A0A097IHU6_9CORY |
| A0A094GM29_9PEZI | A0A094IWM0_9PEZI | A0A0A0K4W2_CUCSA | A0A0H3XHB7_9MOLU |
| M5VXM6_PRUPE | E4NIA2_KITSK | A0A0G0Q3B7_9BACT | A0A067QFN4_9HOMO |
| D4IIW7_9BACT | C5WS51_SORBI | I4A2G1_ORNRL | R5SDG5_9BACE |
| A0A067FDX5_CITSI | A0A067FQW6_CITSI | M0XCC1_HORVD | J4KP78_BEAB2 |
| Q0CD97_ASPTN | K7RUL3_PROA4 | G9ZIX5_9GAMM | A0A077EIP5_9FLAO |
| F5YMJ3_TREPZ | U5QC65_9CYAN | R5ANK4_9FIRM | E0QT23_9ACTO |
| U1FYT2_ENDPU | F4C9N9_SPHS2 | D2PTH5_KRIFD | R5YPD3_9MOLU |
| R7MFT7_9CLOT | C0NDW9_AJECG | A6R018_AJECN | C6HSV8_AJECH |
| F0UF36_AJEC8 | A0A0B2WTV8_9HYPO | B3SC42_TRIAD | A0A077EDG6_9FLAO |
| C7PL40_CHIPD | I0X6F3_9SPIO | Q5P8P3_AROAE | A0A066WG02_9HOMO |
| A0A094AQP8_9PEZI | A0A0A2W292_BEABA | A0A0G2JBY2_9EURO | A0A097SSW6_9MOLU |
| A0A010QSU9_9PEZI | F7MPL6_CLOBO | A8ZKY5_ACAM1 | Q03FE5_PEDPA |
| R6SPT9_9BACE | A0A086THP7_ACRCH | R6E5D7_9BACE | F5Y8L5_TREAZ |
| I0GZV4_ACTM4 | C1GEW2_PARBD | I0GQD0_SELRL | C2CRM2_CORST |
| A0A094GW59_9PEZI | E0TAY4_PARBH | A0A093ZSQ0_9PEZI | A0A067JC37_JATCU |
| F2LSU6_BURGS | F9UKF3_9MOLU | K0Z2M9_9ACTO | V4SPD0_STRIN |
| S8A0M0_PENO1 | R5A5S8_9CLOT | Q1N8V8_SPHSS | A0A0D5A5J9_9NOCA |
| W2RY87_9EURO | Q9PD22_XYLFA | G5K072_9STRE | B6JK20_OLICO |
| D4W570_9FIRM | F4NVX4_BATDJ | B9GS72_POPTR | C4J9S9_MAIZE |
| F4H5R8_CELFA | R7P7T6_9CLOT | A0A0G3H9R2_9CORY | F2E2H8_HORVD |
| C7YR37_NECH7 | D7VNJ1_9SPHI | D5BFB1_ZUNPS | D4XUZ9_9MOLU |
| D7SQJ9_VITVI | A0A0G0W5V6_9BACT | A0A088F4M3_9SPHI | Q1QF97_NITHX |
| F8JDH4_HYPSM | G7JRY2_MEDTR | M1MZX1_9CORY | W5F8K4_WHEAT |
| A0A0A7IC81_9BIFI | R5L4X4_9SPIR | Q6MUK5_MYCMS | A0A0D2LUX2_GOSRA |
| F2TIZ1_AJEDA | R6XJW6_9FIRM | D0WRE4_9ACTO | F8EZF4_TRECH |
| B4RER7_PHEZH | B1V9N0_PHYAS | R8BU94_TOGMI | I1GQ61_BRADI |
| H3NC17_9LACT | E0QN28_9ACTO | D0MKQ0_RHOM4 | A0A090MW97_AFIFE |
| M0YQ13_HORVD | E9DSL1_METAQ | E9F0Z1_METRA | A0A0B4IAG4_9HYPO |
| M8AIR4_TRIUA | A0A0D2QVE0_GOSRA | R7GSL2_9FIRM | K7LH59_SOYBN |
| K4IMF4_BIFAP | A0A0E3ZA78_9FUSO | B1VB25_PHYAS | D0WRY6_9ACTO |
| A0A0B0PBD4_GOSAR | S5SWW6_9CORY | D3PV08_STANL | A0A0D2PW77_GOSRA |
| V4SIA0_STRIN | G3J5D2_CORMM | A0A063BLE3_9HYPO | Q4L7X8_STAHJ |
| K7LH60_SOYBN | B8DSK5_BIFA0 | B2TRS9_CLOBB | B8DTM4_BIFA0 |
| A0A067MGX8_9HOMO | K1LS53_9LACT | A0A0A7I4M9_9BIFI | K7LH58_SOYBN |
| A0A0A1CZ89_9MICC | A0A0F0EW23_9MICO | A0A066W773_9BASI | A0A084QXU9_9HYPO |
| A0A088ESD6_9SPHI | D6RMG8_COPC7 | F0QPZ9_MYCSL | A7H892_ANADF |
| S5ZZ26_9SPIO | Q1QFW4_NITHX | F4F6Z0_VERMA | U5SE12_9LACT |
| W5CE79_WHEAT | A0A084GBY0_9PEZI | SSB_COREF | A2SGD7_METPP |
| A0A0M3ALN3_9SPHN | A0A0K0FGF9_9BILA | C5GXP5_AJEDR | M9LXA2_PSEA3 |
| I2FTH5_USTH4 | F7NPM0_9FIRM | C8XHE1_NAKMY | G4T7B4_PIRID |
| H3FS13_PRIPA | R6U9Q4_9FIRM | T0UME1_9STRE | I0LAN6_9ACTN |
| E7GEJ9_9FIRM | C2BD76_9FIRM | F9EHW5_9ACTO | F0XPU4_GROCL |
| C5J6F1_MYCCR | T0TBX8_9STRE | A0A0L0NL54_9HYPO | R7C6V1_9CLOT |
| H5WPE4_9BURK | H6R5I0_NOCCG | F2JIG6_CELLD | A0A037UQR7_SPIME |
| F0R7K6_BACSH | W5FXE8_WHEAT | E9T4B9_RHOHA | B6Q2D6_TALMQ |
| F6EQ88_AMYSD | M9WE64_9MOLU | A0A0M2S2W8_9ACTN | D7W9Q4_9CORY |
| C8X8N7_NAKMY | B9KGJ2_CAMLR | E6ZQH1_SPORE | S5LTQ4_9MOLU |
| H1YGZ3_9SPHI | K9TDS1_9CYAN | Q0FXP6_9RHIZ | H1BK61_9FIRM |
| D4LZV0_9FIRM | N1JMK0_BLUG1 | W8KH25_HALHR | G4N784_MAGO7 |
| A5D5Z3_PELTS | C7R2W4_JONDD | C3PI90_CORA7 | G8YBW3_PICSO |
| K0ES78_9NOCA | G3B1D7_CANTC | H7EJV8_9SPIO | I0RGY2_MYCPH |
| H5UVB8_9MICO | D4LFP3_9FIRM | R7XZW8_9ACTN | C4ZE71_EUBR3 |
| A0A061DX46_THECC | F6FQG2_ISOV2 | A8L219_FRASN | F4QG08_9CAUL |
| G8YEB3_PICSO | C5BXK5_BEUC1 | G9N9S1_HYPVG | V9RA86_9MOLU |
| E4Q016_MYCBG | U1YGS7_9BURK | Q6BT23_DEBHA | A0A022PQZ4_ERYGU |
| B2AX21_PODAN | M4NN48_9GAMM | A4XJE9_CALS8 | A0A066XHG8_COLSU |
| F3YBI5_MELPT | Q7NNH8_GLOVI | A0A0M2R463_9PROT | R7G6B7_9FIRM |
| K9S5P0_9CYAN | I2GEP0_9BACT | A0A052IL92_9BORD | A4XAN8_SALTO |
| K4CUL0_SOLLC | M1BPA7_SOLTU | D3D1A8_9ACTN | E3QCN2_COLGM |
| D1BSB0_XYLCX | W9QJM0_9ROSA | C7MDW3_BRAFD | A4G6I6_HERAR |
| F8PQM8_SERL3 | E5BI50_9FUSO | E3J6W1_FRASU | A9NG30_ACHLI |
| A0A061DQ94_THECC | E0DGS1_9CORY | F9PD58_9STRE | R6SVK7_9CLOT |
| A0A0F9XNN0_TRIHA | K0Q6P7_9RHIZ | R5RTI4_9BACE | B1VAT5_PHYAS |
| A0A077HJG7_9CORY | A1R7B8_ARTAT | U7PR10_SPOS1 | A0A0F2M9S7_SPOSC |
| U7MYH3_9CORY | W1P0H8_AMBTC | B3QZH8_PHYMT | J9FJ05_WUCBA |
| S3C183_OPHP1 | I7HG55_9HELI | A0A0K0JYI2_BRUMA | J0NPM8_9ACTO |
| U1YE40_9BURK | V5EZ65_PSEBG | I7BIF1_MYCHA | |

### REFERENCES

The following references may be referred to within the specification by author and date and are incorporated herein by reference in their entireties at the location within the specification where they are referenced.
1. Binder, S., Siedler, S., Marienhagen, J., Bott, M., and Eggeling, L. (2013). Recombineering in Corynebacterium glutamicum combined with optical nanosensors: a general strategy for fast producer strain generation. Nucleic Acids Res.
2. Carr, P. A., and Church, G. M. (2009). Genome engineering. Nat. Biotechnol. 27, 1151-1162.
3. Carr, P. A., Wang, H. H., Sterling, B., Isaacs, F. J., Lajoie, M. J., Xu, G., Church, G. M., and Jacobson, J. M. (2012). Enhanced multiplex genome engineering through co-operative oligonucleotide co-selection. Nucleic Acids Res. 40, e132-e132.
4. Choe, W., Chandrasegaran, S., and Ostermeier, M. (2005). Protein fragment complementation in M.HhaI DNA methyltransferase. Biochem. Biophys. Res. Commun. 334, 1233-1240.
5. Court, D. L., Sawitzke, J. A., and Thomason, L. C. (2002). Genetic Engineering Using Homologous Recombination. Annu. Rev. Genet. 36, 361-388.
6. Datsenko, K. A., and Wanner, B. L. (2000). One-step inactivation of chromosomal genes in Escherichia coli K-12 using PCR products (National Acad Sciences).
7. Datta, S., Costantino, N., Zhou, X., and Court, D. L. (2008). Identification and analysis of recombineering functions from Gram-negative and Gram-positive bacteria and their phages. Proc. Natl. Acad. Sci. 105, 1626-1631.
8. Eisen, A., and Camerini-Otero, R. D. (1988). A recombinase from Drosophila melanogaster embryos. Proc. Natl. Acad. Sci. U.S.A. 85, 7481-7485.
9. Ellis, H. M., Yu, D., and DiTizio, T. (2001a). High efficiency mutagenesis, repair, and engineering of chromosomal DNA using single-stranded oligonucleotides. Proc. Natl. Acad. Sci. 98, 6742.
10. Ellis, H. M., Yu, D., DiTizio, T., and Court, D. L. (2001b). High efficiency mutagenesis, repair, and engineering of chromosomal DNA using single-stranded oligonucleotides. Proc. Natl. Acad. Sci. 98, 6742-6746.
11. Finn, R. D., Clements, J., and Eddy, S. R. (2011). HMMER web server: interactive sequence similarity searching. Nucleic Acids Res. 39, W29-W37.
12. Gibson, D. G., Young, L., Chuang, R.-Y., Venter, J. C., Hutchison, C. A., and Smith, H. O. (2009). Enzymatic assembly of DNA molecules up to several hundred kilobases. Nat. Methods 6, 343-345.
13. Gregg, C. J., Lajoie, M. J., Napolitano, M. G., Mosberg, J. A., Goodman, D. B., Aach, J., Isaacs, F. J., and Church, G. M. (2014). Rational optimization of tolC as a powerful dual selectable marker for genome engineering. Nucleic Acids Res. 42, 4779-4790.
14. Iyer, L., Koonin, E., and Aravind, L. (2002). Classification and evolutionary history of the single-strand annealing proteins, RecT, Redbeta, ERF and RAD52. BMC Genomics 3, 8.
15. Karakousis, G., Ye, N., Li, Z., Chiu, S., Reddy, G., and Radding, C. (1998). The beta protein of phage λ binds preferentially to an intermediate in DNA renaturation. J. Mol. Biol. 276, 721-731.
16. Van Kessel, J. C., and Hatfull, G. F. (2007). Recombineering in Mycobacterium tuberculosis. Nat. Methods 4, 147-152.
17. Kmiec, E., and Holloman, W. K. (1981). Beta protein of bacteriophage lambda promotes renaturation of DNA. J. Biol. Chem. 256, 12636-12639.
18. Kosuri, S., Eroshenko, N., Leproust, E. M., Super, M., Way, J., Li, J. B., and Church, G. M. (2010). Scalable gene synthesis by selective amplification of DNA pools from high-fidelity microchips. Nat. Biotechnol. 28, 1295-1299.
19. Lajoie, M. J., Gregg, C. J., Mosberg, J. A., Washington, G. C., and Church, G. M. (2012). Manipulating replisome dynamics to enhance lambda Red-mediated multiplex genome engineering. Nucleic Acids Res. 40, e170-e170.
20. Lajoie, M. J., Rovner, A. J., Goodman, D. B., Aerni, H.-R., Haimovich, A. D., Kuznetsov, G., Mercer, J. A., Wang, H. H., Carr, P. A., Mosberg, J. A., et al. (2013). Genomically Recoded Organisms Expand Biological Functions. Science 342, 357-360.
21. Langston, L. D., Indiani, C., and O'Donnell, M. (2009). Whither the replisome: emerging perspectives on the dynamic nature of the DNA replication machinery. Cell Cycle Georget. Tex 8, 2686-2691.
22. Lennox, E. S. (1955). Transduction of linked genetic characters of the host by bacteriophage P1. Virology 1, 190-206.
23. Li, Z., Karakousis, G., Chiu, S. K., Reddy, G., and Radding, C. M. (1998). The beta protein of phage [lambda] promotes strand exchange. J. Mol. Biol. 276, 733-744.
24. Lopes, A., Amarir-Bouhram, J., Faure, G., Petit, M.-A., and Guerois, R. (2010). Detection of novel recombinases in bacteriophage genomes unveils Rad52, Rad51 and Gp2.5 remote homologs. Nucl Acids Res gkq096.
25. Miki, T., Yamamoto, Y., and Matsuda, H. (2008). A novel, simple, high-throughput method for isolation of genome-wide transposon insertion mutants of Escherichia coli K-12. Methods Mol. Biol. Clifton N. J. 416, 195-204.
26. Mosberg, J. A., Lajoie, M. J., and Church, G. M. (2010). Lambda Red Recombineering in Escherichia coli Occurs Through a Fully Single-Stranded Intermediate. Genetics 186, 791-799.
27. Mosberg, J. A., Gregg, C. J., Lajoie, M. J., Wang, H. H., and Church, G. M. (2012). Improving Lambda Red Genome Engineering in Escherichia coli via Rational Removal of Endogenous Nucleases. PLoS ONE 7, e44638.
28. Mythili, E., Kumar, K. A., and Muniyappa, K. (1996). Characterization of the DNA-binding domain of [beta] protein, a component of phage [lambda] Red-pathway, by UV catalyzed cross-linking. Gene 182, 81-87.
29. New, J. H., Sugiyama, T., Zaitseva, E., and Kowalczykowski, S. C. (1998). Rad52 protein stimulates DNA strand exchange by Rad5 1 and replication protein A. Nature 391, 407-410.
30. Van Pijkeren, J.-P., and Britton, R. A. (2012). High Efficiency Recombineering in Lactic Acid Bacteria. Nucleic Acids Res. 40, e76-e76.
31. Van Pijkeren, J.-P., Neoh, K. M., Sirias, D., Findley, A. S., and Britton, R. A. (2012). Exploring optimization parameters to increase ssDNA recombineering in Lactococcus lactis and Lactobacillus reuteri. Bioengineered 3, 209-217.
32. Poteete, A. R. (2001). What makes the bacteriophage λ\, Red system useful for genetic engineering: molecular mechanism and biological function. FEMS Microbiol. Lett. 201, 9-14.
33. Raman, S., Rogers, J. K., Taylor, N. D., and Church, G. M. (2014). Evolution-guided optimization of biosynthetic pathways. Proc. Natl. Acad. Sci. 111, 17803-17808.
34. Rohland, N., and Reich, D. (2012). Cost-effective, high-throughput DNA sequencing libraries for multiplexed target capture. Genome Res. 22, 939-946.
35. Rybalchenko, N., Golub, E. I., Bi, B., and Radding, C. M. (2004). Strand invasion promoted by recombination protein β of coliphage λ. Proc. Natl. Acad. Sci. U.S.A. 101, 17056-17060.
36. Ryzhikov, M., Koroleva, O., Postnov, D., Tran, A., and Korolev, S. (2011). Mechanism of RecO recruitment to DNA by single-stranded DNA binding protein. Nucleic Acids Res. 39, 6305-6314.
37. Shinohara, A., and Ogawa, T. (1998). Stimulation by Rad52 of yeast Rad51-mediated recombination. Nature 391, 404-407.
38. Sullivan, M. B., Krastins, B., Hughes, J. L., Kelly, L., Chase, M., Sarracino, D., and Chisholm, S. W. (2009). The genome and structural proteome of an ocean siphovirus: a new window into the cyanobacterial “mobilome.” Environ. Microbiol. 11, 2935-2951.
39. Sung, P. (1997). Function of Yeast Rad52 Protein as a Mediator between Replication Protein A and the Rad51 Recombinase. J. Biol. Chem. 272, 28194-28197.
40. Swingle, B., Bao, Z., Markel, E., Chambers, A., and Cartinhour, S. (2010). Recombineering Using RecTE from Pseudomonas syringae. Appl Env. Microbiol 76, 4960-4968.
41. Wang, H. H., Isaacs, F. J., Carr, P. A., Sun, Z. Z., Xu, G., Forest, C. R., and Church, G. M. (2009). Programming cells by multiplex genome engineering and accelerated evolution. Nature 460, 894-898.
42. Wang, H. H., Xu, G., Vonner, A. J., and Church, G. (2011). Modified Bases Enable High-Efficiency Oligonucleotide-Mediated Allelic Replacement Via Mismatch Repair Evasion. Nucleic Acids Res.
43. Wu, Z., Xing, X., Bohl, C. E., Wisler, J. W., Dalton, J. T., and Bell, C. E. (2006). Domain Structure and DNA Binding Regions of β Protein from Bacteriophage λ. J. Biol. Chem. 281, 25205-25214.
44. Xu, Q., Schlabach, M. R., Hannon, G. J., and Elledge, S. J. (2009). Design of 240,000 orthogonal 25mer DNA barcode probes. Proc. Natl. Acad. Sci. U.S.A. 106, 2289-2294.

1. A method of altering a target nucleic acid sequence within a cell comprising
providing the cell with a donor nucleic acid,
providing the cell with a single strand annealing protein, and
providing the cell with a single strand DNA binding protein,
wherein one or more or both of the single strand annealing protein and the single strand DNA binding protein is foreign to the cell, and
wherein the donor nucleic acid is recombined into the target nucleic acid.

2. The method of claim 1 wherein the single strand annealing protein and the single strand DNA binding protein are co-evolved from the same organism and are foreign to the cell.

3. The method of claim 1 wherein the target nucleic acid sequence is a replicating nucleic acid.

4. The method of claim 1 wherein the target nucleic acid is genomic DNA, mitochondrial DNA, viral DNA, exogenous DNA, a plasmid, a cosmid or an episome.

5. The method of claim 1 wherein the cell is a prokaryotic cell or a eukaryotic cell.

6. The method of claim 1 wherein the cell is a prokaryotic cell selected from the group consisting of bacteria and archaea.

7. The method of claim 1 wherein the cell is an animal cell, plant cell, fungal cell, bacteria cell, archaeal cell, eubacterial cell, yeast cell, mammalian cell, mouse cell, rat cell, elephant cell, human cell, stem cell, pluripotent stem cell, or human induced pluripotent stem cell.

8. The method of claim 1 wherein the donor nucleic acid is a single stranded nucleic acid or a double stranded nucleic acid.

9. The method of claim 1 wherein the single strand annealing protein is a member selected from the group consisting of members listed in Tables 1-6.

10. The method of claim 1 wherein the single strand annealing protein is a member selected from the group consisting of members listed in Table 7.

11. The method of claim 1 wherein the single strand annealing protein is a homolog of λβ recombinase.

12. The method of claim 1 wherein the single strand DNA binding protein is E. coli single strand binding protein, a homolog of E. coli single strand binding protein or a protein functionally similar to E. coli single strand binding protein.

13. The method of claim 1 wherein the wherein the single strand annealing protein is λβ recombinase, a homolog of λβ recombinase or a protein functionally similar to λβ recombinase.

14. The method of claim 1 wherein the single strand annealing protein is a homolog of λβ recombinase, wherein the single strand DNA binding protein is a homolog of E. coli single strand binding protein, and wherein the homolog of λβ recombinase and the homolog of E. coli single strand binding protein interact with the cell's replication mechanism to recombine the donor nucleic acid into the target nucleic acid.

15. The method of claim 1 wherein the single strand annealing protein and the single strand DNA binding protein interact with the cell's replication mechanism to facilitate recombination of the donor nucleic acid into the target nucleic acid.

16. The method of claim 1 wherein the single strand annealing protein and the single strand DNA binding protein specifically interact with each other to recombine the donor nucleic acid into the target nucleic acid.

17. The method of claim 1 wherein the single strand annealing protein and the single strand DNA binding protein are from the same foreign organism.

18. The method of claim 1 wherein the single strand annealing protein and the single strand DNA binding protein are designed or evolved to interact with each other.

19. The method of claim 1 wherein the single strand annealing protein and the single strand DNA binding protein are non-natural or synthetic proteins that have been computationally or rationally designed or evolved to function to facilitate recombination of the donor nucleic acid into the target nucleic acid.

20. The method of claim 1 wherein the single stranded annealing protein and/or the single stranded DNA binding protein is a chimera of two or more naturally occurring sequences to achieve novel interaction between the recombinase and the single stranded DNA binding protein or achieve novel facilitation of a donor nucleic acid into the target nucleic acid.

21. The method of claim 1 wherein the single strand DNA binding protein is a chimeric single strand DNA binding protein.
